(19) United States
(12) Patent Application Publication
(10) Pub. No.: US 2019/0110476 A1
(43) Pub. Date: Apr. 18, 2019

(54) NOVEL ISOTHIAZOLAMIDES, PROCESSES FOR THEIR PREPARATION AND THEIR USE AS FUNGICIDES

(71) Applicants: DAVID BERNIER, LYON (FR); PIERRE CRISTAU, LYON (FR); TOMOKI TSUCHIYA, LYON (FR); PHILIPPE RINOLFI, CHATILLON D AZERGUES (FR); THOMAS DROEGE, LANGENFELD (DE); SIMON MAECHLING, LYON (FR); JAN PETER SCHMIDT, FOLSOM, CA (US); JOACHIM TELSER, WUPPERTAL (DE); UWE DOELLER, RODGAU (DE); MARC MOSRIN, KOELN (DE); JULLIEN REY, BASEL (CH); JOERG TIEBES, FRANKFURT (DE); ULRIKE WACHENDORFF-NEUMANN, NEUWIED (DE)

(72) Inventors: DAVID BERNIER, LYON (FR); PIERRE CRISTAU, LYON (FR); TOMOKI TSUCHIYA, LYON (FR); PHILIPPE RINOLFI, CHATILLON D AZERGUES (FR); THOMAS DROEGE, LANGENFELD (DE); SIMON MAECHLING, LYON (FR); JAN PETER SCHMIDT, FOLSOM, CA (US); JOACHIM TELSER, WUPPERTAL (DE); UWE DOELLER, RODGAU (DE); MARC MOSRIN, KOELN (DE); JULLIEN REY, BASEL (CH); JOERG TIEBES, FRANKFURT (DE); ULRIKE WACHENDORFF-NEUMANN, NEUWIED (DE)

(73) Assignee: BAYER CROPSCIENCE AKTIENGESELLSCHAFT, MONHEIM AM RHEIN (DE)

(21) Appl. No.: 15/537,261

(22) PCT Filed: Dec. 21, 2015
(86) PCT No.: PCT/EP2015/080739

§ 371 Date: Jun. 16, 2017

(30) Foreign Application Priority Data
Dec. 22, 2014 (EP) 14199548.0

(51) Int. Cl. A01N 43/80; A01N 47/18; C07D 275/03

(52) U.S. Cl. CPC A01N 43/80; C07D 417/12; C07D 275/03; A01N 47/18

(57) ABSTRACT

The present invention relates to novel isothiazolamide derivatives, to processes for preparing these compounds, to compositions comprising these compounds, and to the use thereof as biologically active compounds for controlling harmful microorganisms in crop protection and in the protection of materials, especially for controlling fungi, preferably for controlling fungi in plants or plant seeds.

The present invention relates to novel isothiazolamide derivatives, to processes for preparing these compounds, to compositions comprising these compounds, and to the use thereof as biologically active compounds for controlling harmful microorganisms in crop protection and in the protection of materials, especially for controlling fungi, preferably for controlling fungi in plants or plant seeds.

The control of harmful microorganisms in crop protection is very important for achieving high crop efficiency. Plant disease damage to ornamental, vegetable, field, cereal or fruit crops can cause significant reduction in productivity. There are many active ingredients available today for controlling harmful microorganisms in crop protection, but there continues to be a need for new active ingredients for controlling harmful microorganisms.

Since the ecological and economic demands made on modern active ingredients, for example fungicides, are increasing constantly, for example with respect to activity spectrum, toxicity, selectivity, application rate, formation of residues and favorable manufacture, and there can also be problems, for example, with resistances, there is a constant need to develop novel fungicides and fungicidal compositions which preferably have one or more advantages over the known compounds or compositions at least in some areas.

The prior art discloses several isothiazoles and isothiazolamides.

Arch. Pharm. (Weinheim) 1987, 320, 43-50 reports on the condensation of 2-morpholino- or 2-piperidino-dithiooxalic O-esters with malondinitrile or cyanoacetate, and inter alia discloses 3-amino-5-(4-morpholinylthioxomethyl)-4-isothiazolecarbonitrile (IUPAC-name: 3-amino-5-(morpholin-4-ylcarbonothioyl)-1,2-thiazole-4-carbonitrile).

JP 2007-302617 discloses various heterocyclic compounds and their use as insecticides, inter alia several amino isothiazoles.

US 2011/0201687 A1 discloses various amide derivatives as pest control agents.

U.S. Pat. No. 3,563,985 relates to a process for preparing certain acylaminoisothiazoles and mentions the use of said isothiazole derivatives as herbicides.

U.S. Pat. No. 4,075,001 mentions the herbicidal activity of certain 1-alkyl- and 1,1-dialkyl-3-(4-substituted-3-amino-5-isothiazolypureas and N-(4-substituted-3-amino-5-isothiazolyl)-alkanamides.

WO 2007/128410 relates to heteroaromatic compounds and their use as insecticides.

WO 2007/014290 discloses various fungicidal carboxamides.

EP 0761654 discloses certain isoxazole- and isothiazole-5-carboxamide derivatives and their use as herbicides.

For the reasons given above, it is therefore an objective of the present invention to provide further biologically active compounds, especially for controlling harmful microorganisms in crop protection and in the protection of materials, and which preferably have one or more advantages over the known compounds or compositions at least in some areas.

It has now been found that the compounds of the following formula (G) and/or the salts thereof meet said objective(s).

The present invention primarily relates to the use of one or more compounds of the formula (G) and/or salts thereof

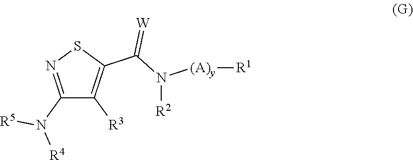

in which
A is CR⁶R⁷,
W is O or S,
R¹¹ is hydrogen, (C₁-C₁₂)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, NR³R¹⁴, R¹³R¹⁴N—(C₁-C₆)-alkyl, (C₁-C₆)-alkoxy, (C₁-C₆)-haloalkoxy, (C₂-C₆)-haloalkoxy-(C₁-C₃)-alkyl, (C₁-C₆)-alkoxy-(C₁-C₃)-alkyl, (C₁-C₆)-alkoxy-(C₂-C₆)-alkoxy-(C₁-C₃)-alkyl, (C₁-C₄)-alkylthio, (C₁-C₄)-alkylsulphoxy, alkylsulphonyl, (C₁-C₄)-haloalkylthio, (C₁-C₄)-haloalkylsulphoxy, (C₁-C₄)-haloalkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-haloalkylthio-(C₁-C₃)-alkyl, (C₁-C₄)-haloalkylsulphoxy-(C₁-C₃)-alkyl, (C₁-C₄)-haloalkylsulphonyl-(C₁-C₃)-alkyl, (C₃-C₁₂)-cycloalkyl, (C₃-C₈)-cycloalkenyl, (C₃-C₁₂)-cycloalkyl-(C₁C₆)-alkyl, (C₃-C₈)-cycloalkenyl-(C₁-C₆)-alkyl, (C₃-C₈)-cycloalkoxy, (C₃-C₈)-cycloalkyl-(C₁C₆)-alkoxy, aryl, aryl-(C₁-C₃)-alkyl, heteroaryl, heteroaryl-(C₁-C₃)-alkyl, heterocyclyl, heterocyclyl-(C₁-C₃)-alkyl, aryloxy, heteroaryloxy, heterocyclyloxy, a bicyclic or a heterobicyclic residue, wherein each of the last-mentioned 17 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alky lsulphoxy , (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxy carbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxy carbonyl-(C₁-C₄)-alkyl, hydroxycarbonyl, hydroxycarbonyl-(C₁-C₄)-alkyl, R¹³R¹⁴N-carbonyl, and wherein heterocyclyl has q oxo groups, and wherein each of the aforementioned heterocyclic residues, in addition to the carbon atoms, has in each case p ring members from the group consisting of N(R¹²)ₘ, O and S(O)ₙ,
R² is hydrogen, (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₄)-alkylsulphonyl, (C₁-C₄)-haloalkylsulphonyl, (C₁-C₆)-alkylcarbonyl, (C₂-C₆)-alkenylcarbonyl, (C₂-C₆)-alkynylcarbonyl, (C₁-C₆)-haloalkylcarbonyl, (C₂-C₆)-haloalkenylcarbonyl, (C₂-C₆)-haloalkynylcarbonyl, (C₁-C₆)-alkoxycarbonyl, di((C₁-C₆)-alkyl)aminocarbonyl, (C₃-C₈)-cycloalkyl, (C₃-C₈)-cycloalkyl-(C₁-C₆)-alkyl, (C₃-C₈)-cycloalkylcarbonyl, (C₃-C₈)-cycloalkyl-(C₁-C₆)-alkylcarbonyl, heteroarylcarbonyl, or arylcarbonyl, wherein each of the last-mentioned 6 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl,
or
R¹ and R², together with the nitrogen atom and (A)_{y} attached thereto (i.e. the group R²—N-(A)_{y}-R¹), form a 5- or 6-membered heterocyclic or heteroaromatic ring, which comprises in each case, in addition to the carbon atoms and the nitrogen atom, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cy loalkyl, (C₃-C₆)-cycloalkyl-(C₁-C₆)-alkyl, (C₁-C₄)-alkoxy carbonyl-(C₁C₄)-alkyl, hydroxycarbonyl, hydroxycarbonyl-(C₁-C₄)-alkyl, R¹³R¹⁴N-carbonyl and has q oxo groups,
R³ is hydrogen, halogen, azido, isocyanate, isothiocyanate, nitro, cyano, hydroxyl, NR¹³R¹⁴, tri(C₁-C₆)-alkylsilyl, (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₁-C₆)-haloalkoxy, (C₁-C₆)-haloalkoxy-(C₁C₃)-alkyl, (C₁-C₆)-alkoxy-(C₁-C₃)-alkyl, (C₁-C₆)-alkoxy-(C₂-C₆)-alkoxy-(C₁-C₃)-alkyl, (C₁-C₆)-alkylcarbonyloxy, (C₁-C₆)-haloalkylcarbonyloxy, (C₂-C₆)-alkenylcarbonyloxy, (C₂-C₆)-alkynylcarbonyloxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-haloalkylthio-(C₁-C₃)-alkyl, (C₁-C₄)-haloalkylsulphoxy-(C₁C₃)-alkyl, (C₁-C₄)-haloalkylsulphonyl-(C₁-C₃)-alkyl, (C₁-C₆)-alkoxycarbonyl, (C₁-C₆)-haloalkoxycarbonyl, (C₂-C₆)-alkenyloxycarbonyl, (C₂-C₆)-haloalkenyloxycarbonyl, (C₂-C₆)-alkynyloxycarbonyl, (C₂-C₆)-haloalkynyloxycarbonyl, (C₁-C₆)-alkylcarbonyl, (C₁-C₆)-haloalkylcarbonyl, (C₂-C₆)-alkenylcarbonyl, (C₂-C₆)-haloalkenylcarbonyl, (C₂-C₆)-alkynylcarbonyl, (C₂-C₆)-haloalkynylcarbonyl, R¹³R¹⁴N-carbonyl, arylthio, arylsulphoxy, arylsulphonyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₃-C₈)-cycloalkoxy, aryl, aryloxy, arylcarbonyloxy, aryl-(C₁-C₃)-alkyl, heteroaryl, heteroaryloxy, heteroaryl-(C₁-C₃)-alkyl, heterocyclyl, heterocyclyloxy, or heterocyclyl-(C₁-C₃)-alkyl, wherein each of the last-mentioned 18 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl, and wherein heterocyclyl has q oxo groups,
R⁴, R⁵ are each independently hydrogen, (C₁-C₁₂)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, (C₁-C₆)-alkoxy-(C₁-C₃)-alkyl, (C₁-C₆)-alkoxy-(C₂-C₆)-alkoxy-(C₁-C₃)-alkyl, (C₁-C₆)-alkoxy-(C₁-C₃)-alkylcarbonyl, (C₁-C₆)-alkoxy-(C₂-C₆)-alkoxy-(C₁-C₃)-alky lcarbonyl, (C₁-C₆)-alkoxycarbonyl, (C₁-C₆)-haloalkoxycarbonyl, (C₂-C₆)-alkenyloxycarbonyl, (C₂-C₆)-haloalkenyloxycarbonyl, (C₂-C₆)-alkynyloxycarbonyl, (C₂-C₆)-haloalkynyloxycarbonyl, (C₁-C₆)-alkylcarbonyl, (C₁-C₆)-haloalkylcarbonyl, (C₂-C₆)-alkenylcarbonyl, (C₂-C₆)-haloalkenylcarbonyl, (C₂-C₆)-alkynylcarbonyl, (C₂-C₆)-haloalkynylcarbonyl, R¹³R¹⁴N-carbonyl, (C₁-C₄)-alkylthio, (C₁-C₄)-haloalkylthio, (C₁-C₈)-alkylthiocarbonyl, (C₁-C₈)-haloalkylthiocarbonyl, (C₁-C₄)-alkylsulphoxy, (C₁-C₄)-haloalkylsulphoxy, (C₁-C₄)-alkylsulphonyl, (C₁-C₄)-haloalkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-alkylthio-(C₁-C₃)-alkylcarbonyl, (C₁-C₄)-alkylsulphoxy-(C₁-C₃)-alkylcarbonyl, (C₁-C₄)-alkylsulphonyl-(C₁-C₃)-alkylcarbonyl, (C₁-C₁₂)-alkylcarbonyl, (C₁-C₁₂)-haloalkylcarbonyl, (C₂-C₁₂)-alkenylcarbonyl, (C₂-C₁₂)-haloalkenylcarbonyl, (C₂-C₁₂)-alkynylcarbonyl, (C₂-C₁₂)-haloalkynylcarbonyl, (C₁-C₁₂)-alkoxycarbonylcarbonyl, (C₁-C₁₂)-alkoxycarbonyl-(C₁-C₃)-alkylcarbonyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₃-C₈)-cycloalkylcarbonyl, (C₃-C₈)-cycloalkenylcarbonyl, (C₃-C₈)-cycloalkyl-(C₁-C₆)-alkylcarbonyl, (C₃-C₈)-cycloalkenyl-(C₁-C₆)-alkylcarbonyl, aryl, aryl-(C₁-C₃)-alkyl, heteroaryl, heteroaryl-(C₁-C₃)-alkyl, heterocyclyl, heterocyclyl-(C₁-C₃)-alkyl, arylcarbonyl, aryl-(C₁-C₆)-alkylcarbonyl, heteroarylcarbonyl, heteroaryl-(C₁-C₆)-alkylcarbonyl, heterocyclylcarbonyl, or heterocyclyl-(C₁-C₆)-alkylcarbonyl, wherein each of the last-mentioned 20 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl, and wherein heterocyclyl has q oxo groups,
   or
   NR⁴R⁵ is —N═CR⁸R⁹ or —N═S(O)ₙR¹⁰R¹¹,
R⁶, R⁷ are each independently hydrogen, cyano, halogen, (C₁-C₆)-alkyl, (C₂-C₆)-alkenyl, (C₂-C₆)-alkynyl, or (C₃-C₈)-cycloalkyl,
   or
   R⁶ and R⁷, together with the carbon atom to which they are attached, form a 3-6-membered carbocyclic or heterocyclic ring, which comprises in each case, in addition to the carbon atoms, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy , (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy , (C₁-C₄)-haloalky lsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
R⁸, R⁹ are each independently hydrogen, (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₁-C₆)-haloalkoxy, (C₁-C₆)-haloalkoxy-(C₁-C₃)-alkyl, (C₂-C₆)-alkenyloxy, (C₂-C₆)-haloalkenyloxy, (C₂-C₆)-alkynyloxy, (C₂-C₆)-haloalkynyloxy, NR¹³R¹⁴, (C₁-C₆)-alkoxy-(C₁-C₃)-alkyl, halogen-(C₁-C₆)-alkoxy-(C₁-C₆)-alkyl, (C₁-C₆)-alkoxy-(C₂-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, aryl, aryl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 10 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
   or
   R⁸ and R⁹, together with the carbon atom to which they are attached, form a 3- to 8-membered unsaturated, partially saturated or saturated ring, which comprises in each case, in addition to the carbon atoms, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
R¹⁰, R¹¹ are each independently (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₆)-alkoxy-(C₁-C₃)-alkyl, halogen-(C₁-C₆)-alkoxy-(C₁-C₆)-alkyl, (C₁-C₆)-alkoxy-(C₂-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, aryl, aryl-(C₁-C₃)-alkyl, heteroaryl, heteroaryl-(C₁-C₃)-alkyl, heterocyclyl or heterocyclyl-(C₁-C₃)-alkyl, wherein each of the last-mentioned 10 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and wherein heterocyclyl has q oxo groups,
   or
   R¹⁰ and R¹¹, together with the sulphur atom to which they are attached, form a 3- to 8-membered unsaturated, partially saturated or saturated ring, which comprises in each case, in addition to the carbon atoms and in addition to the sulphur atom, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
R¹² is hydrogen, (C₁-C₁₂)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, (C₃-C₈)-cycloalkyl, (C₃-C₈)-halocycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₁-C₁₂)-alkylcarbonyl or (C₁-C₁₂)-haloalkylcarbonyl,
R¹³, R¹⁴ are each independently hydrogen, (C₁-C₁₂)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, (C₁-C₁₂)-alkylcarbonyl, (C₂-C₁₂)-alkenylcarbonyl, (C₂-C₁₂)-alkynylcarbonyl, (C₁-C₁₂)-haloalkylcarbonyl, (C₁-C₄)-alkylsulphonyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₃-C₈)-cycloalkylcarbonyl, (C₃-C₈)-cycloalkenylcarbonyl, (C₃-C₈)-cycloalkyl-(C₁-C₆)-alkylcarbonyl, (C₃-C₈)-cycloalkenyl-(C₁-C₆)-alkylcarbonyl, aryl, arylcarbonyl, arylsulphonyl, hetaryl, hetarylcarbonyl, hetarylsulphonyl, heterocyclyl, heterocyclylcarbonyl, heterocyclylsulphonyl, wherein each of the last-mentioned 17 residues is unsubstituted or is substituted by one or more residues from the group consisting of halogen, nitro, hydroxyl, cyano, NH₂, (C₁-C₆)-alkylamine, (C₁-C₆)-dialkylamine, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl and wherein heterocyclyl has q oxo groups,
   or
   R¹³ and R¹⁴, together with the nitrogen atom to which they are attached, form a 3- to 8-membered unsaturated, partially saturated or saturated ring, which comprises in each case, in addition to the carbon atoms and in addition to the nitrogen atom, p ring members from the group consisting of N(R¹²)ₘ, O and S(O)ₙ and wherein said ring is unsubstituted or is substituted by one or more residues from the group consisting of halogen, nitro, hydroxyl, cyano, NH₂, (C₁-C₆)-alkylamine, (C₁-C₆)-dialkylamine, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl and has q oxo groups,
n is independently selected from 0, 1 or 2,
m is independently selected from 0 or 1,
p is independently selected from 0, 1, 2 or 3,
q is independently selected from 0, 1 or 2,
Y is 0 or 1,
for controlling harmful microorganisms in crop protection and in the protection of materials, preferably for controlling fungi, especially for controlling fungi in plants or plant seeds.

The compounds of the above formula (G) and/or salts thereof used in accordance with the present invention show a higher fungicidal activity against and/or show a broader activity, i.e. a fungicidal activity against a larger number of different fungi species, in comparison to fungicidally active compounds disclosed in the prior art having a structural similarity.

In own experiments it was found that the compounds disclosed in WO 2007/014290 essentially only show fungicidal activity against Oomycetes. In contrast thereto, the compounds of the above formula (G) and/or salts thereof used in accordance with the present invention exhibit a much broader fungicidal activity spectrum. Further, in comparison to structurally similar fungicidal thiazoles, the isothiazolamides of the above formula (G) and/or salts thereof used in accordance with the present invention show a broader fungicidal activity spectrum and higher fungicidal activity.

The compounds of the formula (G) used according to the invention include all stereoisomers which can occur on the basis of the centres of asymmetry or double bonds in the molecule whose configuration is not designated specifically in the formula or which are not specified explicitly, and mixtures thereof, including the racemic compounds and the mixtures enriched partly with particular stereoisomers. The invention also includes all tautomers, such as keto and enol tautomers, and their mixtures and salts, if appropriate functional groups are present.

In the case of suitable acidic substituents, the compounds of the formula (G) are able to form salts by reaction with bases where the acidic hydrogen is replaced by an agriculturally suitable cation.

By addition of a suitable inorganic or organic acid onto a basic group, such as, for example, amino or alkylamino, the compounds of the formula (G) are able to form salts. Suitable acidic groups present, such as, for example, carboxylic acid groups, are able to form inner salts with groups which for their part can be protonated, such as amino groups.

The compounds of the formula (G) may preferably be present in the form of agriculturally usable salts, where the type of salt is otherwise immaterial. In general, suitable salts are the salts of those cations or the acid additions salts of those acids whose cations and anions, respectively, have no adverse effect on the biological activity, in particular on the fungicidal activity, of the compounds of formula (G).

Suitable cations are in particular the ions of the alkali metals, preferably lithium, sodium or potassium, of the alkaline earth metals, preferably calcium or magnesium, and of the transition metals, preferably manganese, copper, zinc or iron. The cation used may also be ammonium or substituted ammonium, where one to four hydrogen atoms may be replaced by (C₁-C₄)-alkyl, hydroxy-(C₁-C₄)-alkyl, (C₁-C₄)-alkoxy-(C₁-C₄)-alkyl, hydroxy-(C₁-C₄)-alkoxy-(C₁-C₄)-alkyl, phenyl or benzyl, preferably ammonium, dimethylammonium, diisopropylammonium, tetramethylammonium, tetrabutylammonium, 2-(2-hydroxyeth-1-oxy)eth-1-ylammonium, di(2-hydroxyeth-1-yl)ammonium, trimethylbenzylammonium.

Also suitable are phosphonium ions, sulphonium ions, preferably tri(C₁-C₄)methylsulphonium, or sulphoxonium ions, preferably tri(C₁-C₄)methylsulphoxonium.

Anions of useful acid addition salts are primarily chloride, bromide, fluoride, hydrogensulphate, sulphate, dihydrogenphosphate, hydrogenphosphate, nitrate, bicarbonate, carbonate, hexafluorosilicate, hexafluorophosphate, benzoate and also the anions of (C₁-C₄)-alkanoic acids, preferably formate, acetate, propionate, butyrate or trifluoroacetate.

The indexes n, m, p and q are used in the definitions of different structural elements which may be present in residues R¹, R², R³, R⁴, R⁵ and A, and are independently selected from the indexes n, m, p and q, respectively, which are optionally present in the respective other residues R¹, R², R³, R⁴, R⁵ and A. For example, q may be 1 in residue R¹, q may be 0 in residue R², and q may be 2 in residue R³.

In formula (G) and in all subsequent formulae, chemical radicals or substituents are referred to by names which are collective terms for the enumeration of individual group members or specifically refer to individual chemical radicals or substituents. In general, terms are used which are familiar to the person skilled in the art and/or in particular have the meanings illustrated below.

A hydrocarbon radical is an aliphatic, cycloaliphatic or aromatic monocyclic or, in the case of an optionally substituted hydrocarbon radical, also a bicyclic or polycyclic organic radical based on the elements carbon and hydrogen, including, for example, the radicals alkyl, alkenyl, alkynyl, cycloalkyl, cycloalkenyl, aryl, phenyl, naphthyl, indanyl, indenyl, etc.; this applies correspondingly to hydrocarbon radicals in composite meanings, such as hydrocarbonoxy radicals or other hydrocarbon radicals attached via heteroatom groups.

Unless defined in more detail, the hydrocarbon radicals preferably have 1 to 20 carbon atoms, more preferably 1 to 16 carbon atoms, in particular 1 to 12 carbon atoms. The hydrocarbon radicals, also in the special radicals alkyl, alkoxy, haloalkyl, haloalkoxy, alkylamino and alkylthio, and also the corresponding unsaturated and/or substituted radicals may in each case be straight-chain or branched in the carbon skeleton.

The expression “(C₁-C₄)-alkyl” is a brief notation for alkyl having from 1 to 4 carbon atoms, i.e. encompasses the methyl, ethyl, 1-propyl, 2-propyl, 1-butyl, 2-butyl, 2-methylpropyl or tert-butyl radicals. General alkyl radicals with a larger specified range of carbon atoms, e.g. “(C₁-C₆)-alkyl”, correspondingly also encompass straight-chain or branched alkyl radicals with a greater number of carbon atoms, i.e. according to the example also the alkyl radicals having 5 and 6 carbon atoms.

Unless stated specifically, preference is given to the lower carbon skeletons, for example having from 1 to 6 carbon atoms, or having from 2 to 6 carbon atoms in the case of unsaturated groups, in the case of the hydrocarbyl radicals such as alkyl, alkenyl and alkynyl radicals, including in composite radicals. Alkyl radicals, including in the combined definitions such as alkoxy, haloalkyl, etc., are, for example, methyl, ethyl, n- or i-propyl, n-, t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals; alkenyl is, for example, vinyl, allyl, 1-methyl-2-propenyl, 2-methyl-2-propenyl, 2-butenyl, pentenyl, 2-methylpentenyl or hexenyl group, preferably allyl, 1-methylprop-2-en-1-yl, 2-methylprop-2-en-1-yl, but-2-en-1-yl, but-3-en-1-yl, 1-methylbut-3-en-1-yl or 1-methylbut-2-en-1-yl.

Alkenyl also includes in particular straight-chain or branched hydrocarbon radicals having more than one double bond, such as 1,3-butadienyl and 1,4-pentadienyl, but also allenyl or cumulenyl radicals having one or more cumulated double bonds, for example allenyl (1,2-propadienyl), 1,2-butadienyl and 1,2,3-pentatrienyl.

Alkynyl is, for example, propargyl, but-2-yn-1-yl, but-3-yn-1-yl, 1-methylbut-3-yn-1-yl.

Alkynyl also includes, in particular, straight-chain or branched hydrocarbon radicals having more than one triple bond or else having one or more triple bonds and one or more double bonds, for example 1,3-butatrienyl or 3-penten-1-yn-1-yl.

A 3- to 9-membered carbocyclic ring is (C₃-C₉)-cycloalkyl or (C₅-C₉)-cycloalkenyl.

(C₃-C₉)-Cycloalkyl is a carbocyclic saturated ring system having preferably 3-9 carbon atoms, for example cyclopropyl, cyclobutyl, cyclopentyl, cyclohexyl, cycloheptyl, cyclooctyl or cyclononyl. In the case of substituted cycloalkyl, cyclic systems with substituents are included, where the substituents may also be bonded by a double bond on the cycloalkyl radical, for example an alkylidene group such as methylidene.

(C₅-C₉)-Cycloalkenyl is a carbocyclic, nonaromatic, partially unsaturated ring system having 5-9 carbon atoms, for example 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl. In the case of substituted cycloalkenyl, the explanations for substituted cycloalkyl apply correspondingly.

Alkylidene, for example also in the form of (C₁-Cio)-alkylidene, is the radical of a straight-chain or branched alkane which is bonded via a double bond, the position of the binding site not being fixed. In the case of a branched alkane, the only positions possible are, of course, those in which two hydrogen atoms can be replaced by the double bond; radicals are, for example, ═CH₂, ═CH—CH₃, ═C(CH₃)—CH₃, ═C(CH₃)—C₂H₅ or ═C(C₂H₅)—C₂H₅.

Halogen is, for example, fluorine, chlorine, bromine or iodine. Haloalkyl, -alkenyl and -alkynyl are alkyl, alkenyl and alkynyl, respectively, which are partially or fully substituted by identical or different halogen atoms, preferably from the group consisting of fluorine, chlorine, bromine and iodine, in particular from the group consisting of fluorine, chlorine and bromine, very particularly from the group consisting of fluorine and chlorine, for example monohaloalkyl, perhaloalkyl, CF₃, CHF₂, CH₂F, CF₃CF₂, CH₂FCHCl, CCl₃, CHCl₂, CH₂CH₂Cl; haloalkoxy is, for example, OCF₃, OCHF₂, OCH₂F, CF₃CF₂O, OCH₂CF₃ and OCH₂CH₂Cl; this applies correspondingly to haloalkenyl and other halogen-substituted radicals such as, for example, halocycloalkyl.

Aryl is a mono-, bi- or polycyclic aromatic system, for example phenyl, naphthyl, tetrahydronaphthyl, indenyl, indanyl, pentalenyl, fluorenyl and the like, preferably phenyl.

Optionally substituted aryl also includes polycyclic systems, such as tetrahydronaphthyl, indenyl, indanyl, fluorenyl, biphenylyl, where the point of attachment is at the aromatic system.

A heterocyclic radical (heterocyclyl) comprises at least one heterocyclic ring (=carbocyclic ring in which at least one carbon atom is replaced by a heteroatom, preferably by a heteroatom from the group consisting of N, O, S, P, B, Si, Se), which is saturated, unsaturated or heteroaromatic and may be unsubstituted or substituted, where the point of attachment is located at a ring atom.

Unless defined otherwise it preferably contains one or more, in particular 1, 2 or 3, heteroatoms in the heterocyclic ring, preferably from the group consisting of N, O, and S; it is preferably an aliphatic heterocyclyl radical having 3 to 7 ring atoms or a heteroaromatic radical having 5 or 6 ring atoms. The heterocyclic radical may, for example, be a heteroaromatic radical or ring (heteroaryl), such as, for example, a monocyclic, bicyclic or polycyclic aromatic system in which at least 1 ring contains one or more heteroatoms.

If the heterocyclyl radical or the heterocyclic ring is optionally substituted, it can be fused to other carbocyclic or heterocyclic rings. Preference is given to benzo-fused heterocyclic or heteroaromatic rings.

Optionally substituted heterocyclyl also includes polycyclic systems, such as, for example, 8-aza-bicyclo[3.2.1]octanyl or 1-aza-bicyclo[2.2.1]heptyl.

Optionally substituted heterocyclyl also includes spirocyclic systems, such as, for example, 1-oxa-5-aza-spiro[2.3]hexyl.

It is preferably a radical of a heteroaromatic ring having a heteroatom from the group consisting of N, O and S, for example the radical of a five- or six-membered ring, such as pyridyl, pyrrolyl, thienyl or furyl; it is furthermore preferably a radical of a corresponding heteroaromatic ring having 2, 3 or 4 heteroatoms, for example pyrimidinyl, pyridazinyl, pyrazinyl, triazinyl, tetrazinyl, thiazolyl, thiadiazolyl, oxazolyl, isoxazolyl, pyrazolyl, imidazolyl or triazolyl or tetrazolyl.

Here, preference is given to a radical of a heteroaromatic five- or six-membered ring having 1 to 4 heteroatoms, such as, for example, 1,2,3-triazolyl, 1,2,4-triazolyl, tetrazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, tetrazolyl, 1,2,3-triazinyl, 1,2,4-triazinyl, 1,3,5-triazinyl, 1,2,3,4-tetrazinyl, 1,2,3,5-tetrazinyl, 1,2,4,5-tetrazinyl, thiazolyl, isothiazolyl, oxazolyl, isoxazolyl, pyrazolyl, imidazolyl.

More preference is given here to heteroaromatic radicals of five-membered heterocycles having 3 nitrogen atoms, such as 1,2,3-triazol-1-yl, 1,2,3-triazol-4-yl, 1,2,3-triazol-5-yl, 1,2,5-triazol-1-yl, 1,2,5-triazol-3-yl, 1,3,4-triazol-1-yl, 1,3,4-triazol-2-yl, 1,2,4-triazol-3-yl, 1,2,4-triazol-5-yl;

more preference is also given here to heteroaromatic radicals of six-membered heterocycles having 3 nitrogen atoms, such as 1,3,5-triazin-2-yl, 1,2,4-triazin-3-yl, 1,2,4-triazin-5-yl, 1,2,4-triazin-6-yl, 1,2,3-triazin-4-yl, 1,2,3-triazin-5-yl;

more preference is also given here to heteroaromatic radicals of five-membered heterocycles having two nitrogen atoms and one oxygen atom, such as 1,2,4-oxadiazol-3-yl; 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl,

more preference is also given here to heteroaromatic radicals of five-membered heterocycles having two nitrogen atoms and one sulphur atom, such as 1,2,4-thiadiazol-3-yl, 1,2,4-thiadiazol-5-yl, 1,3,4-thiadiazol-2-yl, 1,2,3-thiadiazol-4-yl, 1,2,3-thiadiazol-5-yl, 1,2,5-thiadiazol-3-yl;

more preference is also given here to heteroaromatic radicals of five-membered heterocycles having four nitrogen atoms, such as 1,2,3,4-tetrazol-1-yl, 1,2,3,4-tetrazol-5-yl, 1,2,3,5-tetrazol-1-yl, 1,2,3,5-tetrazol-4-yl, 2H-1,2,3,4-tetrazol-5-yl, 1H-1,2,3,4-tetrazol-5-yl,

more preference is also given here to heteroaromatic radicals of six-membered heterocycles such as 1,2,4,5-tetrazin-3-yl;

more preference is also given here to heteroaromatic radicals of five-membered heterocycles having three nitrogen atoms and one oxygen or sulphur atom, such as 1,2,3,4-oxatriazol-5-yl; 1,2,3,5-oxatriazol-4-yl; 1,2,3,4-thiatriazol-5-yl; 1,2,3,5-thiatriazol-4-yl;

more preference is also given here to heteroaromatic radicals of six-membered heterocycles such as, for example, 1,2,4,6-thiatriazin-1-yl; 1,2,4,6-thiatriazin-3-yl; 1,2,4,6-thiatriazin-5-yl.

Furthermore preferably, the heterocyclic radical or ring is a partially or fully hydrogenated heterocyclic radical having one heteroatom from the group consisting of N, O and S, for example oxiranyl, oxetanyl, oxolanyl (=tetrahydrofuryl), oxanyl,pyrrolinyl, pyrrolidyl or piperidyl.

It is also preferably a partially or fully hydrogenated heterocyclic radical having 2 heteroatoms from the group consisting of N, O and S, for example piperazinyl, dioxolanyl, oxazolinyl, isoxazolinyl, oxazolidinyl, isoxazolidinyl and morpholinyl. Suitable substituents for a substituted heterocyclic radical are the substituents specified later on below, and additionally also oxo. The oxo group may also occur on the hetero-ring atoms which are able to exist in different oxidation states, as in the case of N and S, for example.

Preferred examples of heterocyclyl are a heterocyclic radical having from 3 to 6 ring atoms from the group consisting of pyridyl, thienyl, furyl, pyrrolyl, oxiranyl, 2-oxetanyl, 3-oxetanyl, oxolanyl (=tetrahydrofuryl), pyrrolidyl, piperidyl, especially oxiranyl, 2-oxetanyl, 3-oxetanyl or oxolanyl, or is a heterocyclic radical having two or three heteroatoms, for example pyrimidinyl, pyridazinyl, pyrazinyl, triazinyl, thiazolyl, thiadiazolyl, oxazolyl, isoxazolyl, pyrazolyl, triazolyl, piperazinyl, dioxolanyl, oxazolinyl, isoxazolinyl, oxazolidinyl, isoxazolidinyl or morpholinyl.

Preferred heterocyclic radicals are also benzo-fused heteroaromatic rings, for example benzofuryl, benzisofuryl, benzothiophenyl, benzisothiophenyl, isobenzothiophenyl, indolyl, isoindolyl, indazolyl, benzimidazolyl, benzotriazolyl, benzoxazolyl, 1,2-benzisoxazolyl, 2,1-benzisoxazolyl, benzothiazolyl, 1,2-benzisothiazolyl, 2,1-benzisothiazolyl, 1,2,3-benzoxadiazolyl, 2,1,3-benzoxadiazolyl, 1,2,3-benzothiadiazolyl, 2,1,3-benzothiadiazolyl, quinolyl (quinolinyl), isoquinolyl (isoquinolinyl), quinnolinyl, phthalazinyl, quinazolinyl, quinoxalinyl, naphthyridinyl, benzotriazinyl, purinyl, pteridinyl, indolizinyl, benzo-1,3-dioxylyl, 4H-benzo-1,3-dioxinyl and 4H-benzo-1,4-dioxinyl, and, where possible, N-oxides and salts thereof.

When a base structure is substituted by one or more radicals from a list of radicals (=group) or a generically defined group of radicals, this in each case includes simultaneous substitution by a plurality of identical and/or structurally different radicals.

Substituted radicals, such as a substituted alkyl, alkenyl, alkynyl, cycloalkyl, aryl, phenyl, benzyl, heterocyclyl and heteroaryl radical, are, for example, a substituted radical derived from the unsubstituted base structure, where the substituents are, for example, one or more, preferably 1, 2 or 3, radicals from the group consisting of halogen, alkoxy, alkylthio, hydroxyl, amino, nitro, carboxyl, cyano, azido, alkoxycarbonyl, alkylcarbonyl, formyl, carbamoyl, mono- and dialkylaminocarbonyl, substituted amino such as acylamino, mono- and dialkylamino, and alkylsulphinyl, alkylsulphonyl and, in the case of cyclic radicals, also alkyl, haloalkyl, alkylthioalkyl, alkoxyalkyl, optionally substituted mono- and dialkylaminoalkyl and hydroxyalkyl; in the term “substituted radicals”, such as substituted alkyl, etc., substituents include, in addition to the saturated hydrocarbon radicals mentioned, corresponding unsaturated aliphatic and aromatic radicals, such as optionally substituted alkenyl, alkynyl, alkenyloxy, alkynyloxy, phenyl and phenoxy. In the case of substituted cyclic radicals having aliphatic moieties in the ring, cyclic systems with those substituents which are bonded on the ring by a double bond are also included, for example substituted by an alkylidene group such as methylidene or ethylidene.

Unless defined in more detail, optionally substituted phenyl is preferably phenyl or phenyl which is unsubstituted or substituted by one or more radicals from the group consisting of halogen, cyano, (C₁-C₄)-alkyl, (C₁-C₄)-haloalkyl, (C₁-C₄)-alkoxy-(C₁-C₄)alkyl, (C₁-C₄)-alkoxy, (C₁-C₄)-haloalkoxy, (C₁-C₄)-alkoxy-(C₁-C₄)-alkoxy, (C₁-C₄)-alkylthio and nitro, in particular phenyl which is optionally substituted by one or more radicals from the group consisting of halogen, (C₁-C₄)-alkyl, (C₁-C₄)-haloalkyl and (C₁-C₄)-alkoxy.

In the case of radicals having carbon atoms, preference is given to those having 1 to 6 carbon atoms, preferably 1 to 4 carbon atoms. Preference is generally given to substituents from the group consisting of halogen, e.g. fluorine and chlorine, (C₁-C₄)-alkyl, preferably methyl or ethyl, (C₁-C₄)-haloalkyl, preferably trifluoromethyl, (C₁-C₄)-alkoxy, preferably methoxy or ethoxy, (C₁-C₄)-haloalkoxy, nitro and cyano. Particular preference is given here to the substituents methyl, methoxy, fluorine and chlorine.

Substituted amino, such as mono- or disubstituted amino, is a radical from the group consisting of the substituted amino radicals which are N-substituted, for example, by one or two identical or different radicals from the group consisting of alkyl, alkoxy, acyl and aryl; preferably mono- and dialkylamino, mono- and diarylamino, acylamino, N-alkyl-N-arylamino, N-alkyl-N-acylamino and N-heterocycles; preference is given to alkyl radicals having from 1 to 4 carbon atoms; aryl is preferably phenyl or substituted phenyl; acyl is as defined below, preferably (C₁-C₄)-alkanoyl. The same applies to substituted hydroxylamino or hydrazino.

Acyl is a radical of an organic acid which arises in a formal sense by removal of a hydroxyl group on the acid function, and the organic radical in the acid may also be bonded to the acid function via a heteroatom. Examples of acyl are the —CO—R radical of a carboxylic acid HO—CO—R and radicals of acids derived therefrom, such as those of thiocarboxylic acid, optionally N-substituted iminocarboxylic acids or the radical of carbonic monoesters, N-substituted carbamic acid, sulphonic acids, sulphinic acids, N-substituted sulphonamide acids, phosphonic acids or phosphinic acids.

Acyl is, for example, formyl, alkylcarbonyl such as [(C₁-C₄)-alkyl]carbonyl, phenylcarbonyl, alkyloxycarbonyl, phenyloxycarbonyl, benzyloxycarbonyl, alkylsulphonyl, alkylsulphinyl, N-alkyl-1-iminoalkyl and other radicals of organic acids. The radicals may each be substituted further in the alkyl or phenyl moiety, for example in the alkyl moiety by one or more radicals from the group consisting of halogen, alkoxy, phenyl and phenoxy; examples of substituents in the phenyl moiety are the substituents already mentioned above in general for substituted phenyl.

Acyl is preferably an acyl radical in the narrower sense, i.e. a radical of an organic acid in which the acid group is bonded directly to the carbon atom of an organic radical, for example formyl, alkylcarbonyl such as acetyl or [(C₁-C₄)-alkyl]carbonyl, phenylcarbonyl, alkylsulphonyl, alkylsulphinyl and other radicals of organic acids.

More preferably, acyl is an alkanoyl radical having 1 to 6 carbon atoms, in particular 1 to 4 carbon atoms. Here, (C₁-C₄)-alkanoyl is the radical of an alkanoic acid having 1 to 4 carbon atoms formed after removal of the OH group of the acid group, i.e. formyl, acetyl, n-propionyl, isopropionyl or n-, sec- or tert-butanoyl.

The “yl position” of a radical denotes the carbon atom having the free bond.

Compounds of the formula (G) according to the invention and compounds of the formula (G) used according to the invention and/or salts thereof are in short also referred to as “compounds (G)”.

The invention also provides all stereoisomers which are encompassed by formula (G) and mixtures thereof. Such compounds of the formula (G) may contain one or more asymmetric carbon atoms or may contain double bonds which are not stated separately in the general formulae (G). The possible stereoisomers defined by their specific three-dimensional shape, such as enantiomers, diastereomers, Z- and E-isomers, are all encompassed by the formula (G) and can be obtained from mixtures of the stereoisomers by customary methods or else prepared by stereoselective reactions in combination with the use of stereochemically pure starting materials.

The present invention also relates to a compound of the formula (G) and/or a salt thereof,

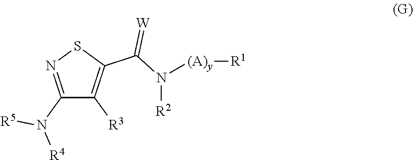

in which
A is CR⁶R⁷,
W is O or S,
R¹ is hydrogen, (C₁-C₁₂)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, NR¹³R¹⁴, R¹³R¹⁴N—(C₁-C₆)-alkyl, (C₁-C₆)-alkoxy, (C₁-C₆)-haloalkoxy, (C₁-C₆)-haloalkoxy-(C₁-C₃)-alkyl, (C₁-C₆)-alkoxy-(C₁-C₃)-alkyl, (C₁-C₆)-alkoxy-(C₂-C₆)-alkoxy-(C₁-C₃)-alkyl, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-haloalkylthio-(C₁-C₃)-alkyl, (C₁-C₄)-haloalkylsulphoxy-(C₁-C₃)-alkyl, (C₁-C₄)-haloalkylsulphonyl-(C₁-C₃)-alkyl, (C₃-C₁₂)-cycloalkyl, (C₃-C₈)-cycloalkenyl, (C₃-C₁₂)-cycloalkyl-(C₁-C₆)-alkyl, (C₃-C₈)-cycloalkenyl-(C₁-C₆)-alkyl, (C₃-C₈)-cycloalkoxy, (C₃-C₈)-cycloalkyl-(C₁-C₆)-alkoxy, aryl, aryl-(C₁-C₃)-alkyl, heteroaryl, heteroaryl-(C₁-C₃)-alkyl, heterocyclyl, heterocyclyl-(C₁-C₃)-alkyl, aryloxy, heteroaryloxy, heterocyclyloxy, a bicyclic or a heterobicyclic residue, wherein each of the last-mentioned 17 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl, and wherein heterocyclyl has q oxo groups, and wherein each of the aforementioned heterocyclic residues, in addition to the carbon atoms, has in each case p ring members from the group consisting of N(R¹²)ₘ, O and S(O)ₙ,
R² is hydrogen, (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₄)-alkylsulphonyl, (C₁-C₄)-haloalkylsulphonyl, (C₁-C₆)-alkylcarbonyl, (C₂-C₆)-alkenylcarbonyl, (C₂-C₆)-alkynylcarbonyl, (C₁-C₆)-haloalkylcarbonyl, (C₂-C₆)-haloalkenylcarbonyl, (C₂-C₆)-haloalkynylcarbonyl, (C₁-C₆)-alkoxycarbonyl, di((C₁-C₆)-alkyl)aminocarbonyl, (C₃-C₈)-cycloalkyl, (C₃-C₈)-cycloalkyl-(C₁-C₆)-alkyl, (C₃-C₈)-cycloalkylcarbonyl, (C₃-C₈)-cycloalkyl-(C₁-C₆)-alkylcarbonyl, heteroarylcarbonyl, or arylcarbonyl, wherein each of the last-mentioned 6 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl,
   or
   R¹ and R², together with the nitrogen atom and (A)_{y} attached thereto (i.e. the group R²—N-(A)_{y}-R¹), form a 5- or 6-membered heterocyclic or heteroaromatic ring, which comprises in each case, in addition to the carbon atoms and the nitrogen atom, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-C₄)-alkyl, (C ₁-C₄)-haloalkyl, (C₁-C₄)-alkoxy, (C₁-C₄)-haloalkoxy , (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
R³ is hydrogen, halogen, azido, isocyanate, isothiocyanate, nitro, cyano, hydroxyl, NR¹³R¹⁴, tri(C₁-C₆)-alkylsilyl, (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₁-C₆)-haloalkoxy, (C₁-C₆)-haloalkoxy-(C₁-C₃)-alkyl, (C₁-C₆)-alkoxy-(C₁-C₃)-alkyl, (C₁-C₆)-alkoxy-(C₂-C₆)-alkoxy-(C₁-C₃)-alkyl, (C₁-C₆)-alkylcarbonyloxy, (C₁-C₆)-haloalkylcarbonyloxy, (C₂-C₆)-alkenylcarbonyloxy, (C₂-C₆)-alkynylcarbonyloxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-haloalkylthio-(C₁-C₃)-alkyl, (C₁-C₄)-haloalkylsulphoxy-(C₁-C₃)-alkyl, (C₁-C₄)-haloalkylsulphonyl-(C₁-C₃)-alkyl, (C₁-C₆)-alkoxycarbonyl, (C₁-C₆)-haloalkoxycarbonyl, (C₂-C₆)-alkenyloxycarbonyl, (C₂-C₆)-haloalkenyloxycarbonyl, (C₂-C₆)-alkynyloxycarbonyl, (C₂-C₆)-haloalkynyloxycarbonyl, (C₁-C₆)-alkylcarbonyl, (C₁-C₆)-haloalkylcarbonyl, (C₂-C₆)-alkenylcarbonyl, (C₂-C₆)-haloalkenylcarbonyl, (C₂-C₆)-alkynylcarbonyl, (C₂-C₆)-haloalkynylcarbonyl, R¹³R¹⁴N-carbonyl, arylthio, arylsulphoxy, arylsulphonyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₃-C₈)-cycloalkoxy, aryl, aryloxy, arylcarbonyloxy, aryl-(C₁-C₃)-alkyl, heteroaryl, heteroaryloxy, heteroaryl-(C₁-C₃)-alkyl, heterocyclyl, heterocyclyloxy, or heterocyclyl-(C₁-C₃)-alkyl, wherein each of the last-mentioned 18 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl, and wherein heterocyclyl has q oxo groups,
R⁴, R⁵ are each independently hydrogen, (C₁-C₄)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, (C₁-C₆)-alkoxy-(C₁-C₃)-alkyl, (C₁-C₆)-alkoxy-(C₂-C₆)-alkoxy-(C₁-C₃)-alkyl, (C₁-C₆)-alkoxy-(C₁-C₃)-alkylcarbonyl, (C₁-C₆)-alkoxy-(C₂-C₆)-alkoxy-(C₁-C₃)-alkylcarbonyl, (C₁-C₆)-alkoxycarbonyl, (C₁-C₆)-haloalkoxycarbonyl, (C₂-C₆)-alkenyloxycarbonyl, (C₂-C₆)-haloalkenyloxycarbonyl, (C₂-C₆)-alkynyloxycarbonyl, (C₂-C₆)-haloalkynyloxycarbonyl, (C₁-C₆)-alkylcarbonyl, (C₁-C₆)-haloalkylcarbonyl, (C₂-C₆)-alkenylcarbonyl, (C₂-C₆)-haloalkenylcarbonyl, (C₂-C₆)-alkynylcarbonyl, (C₂-C₆)-haloalkynylcarbonyl, R¹³R¹⁴N-carbonyl, (C₁-C₄)-alkylthio, (C₁-C₄)-haloalkylthio, (C₁-C₈)-alkylthiocarbonyl, (C₁-C₈)-haloalkylthiocarbonyl, (C₁-C₄)-alkylsulphoxy, (C₁-C₄)-haloalkylsulphoxy, (C₁-C₄)-alkylsulphonyl, (C₁-C₄)-haloalkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-alkylthio-(C₁-C₃)-alkylcarbonyl, (C₁-C₄)-alkylsulphoxy-(C₁-C₃)-alkylcarbonyl, (C₁-C₄)-alkylsulphonyl-(C₁-C₃)-alkylcarbonyl, (C₁-C₁₂)-alkylcarbonyl, (C₁-C₁₂)-haloalkylcarbonyl, (C₂-C₁₂)-alkenylcarbonyl, (C₂-C₁₂)-haloalkenylcarbonyl, (C₂-C₁₂)-alkynylcarbonyl, (C₂-C₁₂)-haloalkynylcarbonyl, (C₁-C₁₂)-alkoxycarbonylcarbonyl, (C₁-C₁₂)-alkoxycarbonyl-(C₁-C₃)-alkylcarbonyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₃-C₈)-cycloalkylcarbonyl, (C₃-C₈)-cycloalkenylcarbonyl, (C₃-C₈)-cycloalkyl-(C₁-C₆)-alkylcarbonyl, (C₃-C₈)-cycloalkenyl-(C₁-C₆)-alkylcarbonyl, aryl, aryl-(C₁-C₃)-alkyl, heteroaryl, heteroaryl-(C₁-C₃)-alkyl, heterocyclyl, heterocyclyl-(C₁-C₃)-alkyl, arylcarbonyl, aryl-(C₁-C₆)-alkylcarbonyl, heteroarylcarbonyl, heteroaryl-(C₁-C₆)-alkylcarbonyl, heterocyclylcarbonyl, or heterocyclyl-(C₁-C₆)-alkylcarbonyl, wherein each of the last-mentioned 20 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl, and wherein heterocyclyl has q oxo groups, wherein preferably R⁴ and R⁵ are not both an alkyl residue, and more preferably R⁴ and R⁵ are not both an (C₁-C₁₂)-alkyl residue,
   or
   NR⁴R⁵ is —N═CR⁸R⁹ or —N═S(O)ₙR¹⁰R¹¹,
R⁶, R⁷ are each independently hydrogen, cyano, halogen, (C₁-C₆)-alkyl, (C₂-C₆)-alkenyl, (C₂-C₆)-alkynyl, or (C₃-C₈)-cycloalkyl,
   or
   R⁶ and R⁷, together with the carbon atom to which they are attached, form a 3-6-membered carbocyclic or heterocyclic ring, which comprises in each case, in addition to the carbon atoms, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
R⁸, R⁹ are each independently hydrogen, (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₁-C₆)-haloalkoxy, (C₁-C₆)-haloalkoxy-(C₁-C₃)-alkyl, (C₂-C₆)-alkenyloxy, (C₂-C₆)-haloalkenyloxy, (C₂-C₆)-alkynyloxy, (C₂-C₆)-haloalkynyloxy, NR¹³R¹⁴, (C₁-C₆)-alkoxy-(C₁-C₃)-alkyl, halogen-(C₁-C₆)-alkoxy-(C₁-C₆)-alkyl, (C₁-C₆)-alkoxy-(C₂-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, aryl, aryl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 10 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alky lcarboxy , (C₃-C₆)-cycloalkyl, (C₃-C₆)-cycloalkyl-(C₁-C₆)-alkyl, (C₁-C₄)-alkoxy carbonyl-(C₁-C₄)-alkyl, hydroxycarbonyl, hydroxycarbonyl-(C₁-C₄)-alkyl, R¹³R¹⁴N-carbonyl and has q oxo groups,
   or
   R⁸ and R⁹, together with the carbon atom to which they are attached, form a 3- to 8-membered unsaturated, partially saturated or saturated ring, which comprises in each case, in addition to the carbon atoms, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
R¹⁰, R¹¹ are each independently (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₆)-alkoxy-(C₁-C₃)-alkyl, halogen-(C₁-C₆)-alkoxy-(C₁-C₆)-alkyl, (C₁-C₆)-alkoxy-(C₂-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, aryl, aryl-(C₁-C₃)-alkyl, heteroaryl, heteroaryl-(C₁-C₃)-alkyl, heterocyclyl or heterocyclyl-(C₁-C₃)-alkyl, wherein each of the last-mentioned 10 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and wherein heterocyclyl has q oxo groups,
   or
   R¹⁰ and R¹¹, together with the sulphur atom to which they are attached, form a 3- to 8-membered unsaturated, partially saturated or saturated ring, which comprises in each case, in addition to the carbon atoms and in addition to the sulphur atom, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
R¹² is hydrogen, (C₁-C₁₂)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, (C₃-C₈)-cycloalkyl, (C₃-C₈)-halocycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₁-C₁₂)-alkylcarbonyl or (C₁-C₁₂)-haloalkylcarbonyl,
R¹³, R¹⁴ are each independently hydrogen, (C₁-C₁₂)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, (C₁-C₁₂)-alkylcarbonyl, (C₂-C₁₂)-alkenylcarbonyl, (C₂-C₁₂)-alkynylcarbonyl, (C₁-C₁₂)-haloalkylcarbonyl, (C₁-C₄)-alkylsulphonyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₃-C₈)-cycloalkylcarbonyl, (C₃-C₈)-cycloalkenylcarbonyl, (C₃-C₈)-cycloalkyl-(C₁-C₆)-alkylcarbonyl, (C₃-C₈)-cycloalkenyl-(C₁-C₆)-alkylcarbonyl, aryl, arylcarbonyl, arylsulphonyl, hetaryl, hetarylcarbonyl, hetarylsulphonyl, heterocyclyl, heterocyclylcarbonyl, heterocyclylsulphonyl, wherein each of the last-mentioned 17 residues is unsubstituted or is substituted by one or more residues from the group consisting of halogen, nitro, hydroxyl, cyano, NH₂, (C₁-C₆)-alkylamine, (C₁-C₆)-dialkylamine, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl and wherein heterocyclyl has q oxo groups,
   or
   R¹³ and R¹⁴, together with the nitrogen atom to which they are attached, form a 3- to 8-membered unsaturated, partially saturated or saturated ring, which comprises in each case, in addition to the carbon atoms and in addition to the nitrogen atom, p ring members from the group consisting of N(R¹²)ₘ, O and S(O)ₙ and wherein said ring is unsubstituted or is substituted by one or more residues from the group consisting of halogen, nitro, hydroxyl, cyano, NH₂, (C₁-C₆)-alkylamine, (C₁-C₆)-dialkylamine, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl and has q oxo groups,
n is independently selected from 0, 1 or 2,
m is independently selected from 0 or 1,
p is independently selected from 0, 1, 2 or 3,
q is independently selected from 0, 1 or 2,
y is 0 or 1,
with the proviso that:
the compound of formula (G) is not 3-amino-5-(morpholin-4-ylcarbonothioyl)-1,2-thiazole-4-carbonitrile (i.e. not the compound of formula (G), wherein R²—N-(A)_{y}-le together form a morpholin-4-yl ring, W is S, R³ is CN, R⁴ is H and R⁵ is H),
and
y is 1, if R¹ is a substituted 4-heptafluoroisopropylphenyl residue, a substituted 4-(nonafluoro-2-butyl)phenyl residue, a substituted 4-(1,1,2,3,3,3-hexafluoropropoxy)phenyl residue, a 2-bromo-4-methyl-6-(heptafluoroisopropyl)pyridin-3-yl residue or a 2-bromo-4-methyl-6-(2,2,2-trifluoro-1-trifluoromethylethoxy)pyridin-3-yl residue.

Preferred compounds according to the present invention correspond to the formula (G) and/or salts thereof, wherein
R³ is hydrogen, halogen, cyano, hydroxyl, NR¹³R¹⁴, tri(C₁-C₆)-alkylsilyl, (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₁-C₆)-haloalkoxy, (C₁-C₄)-alkylthio, (C₃-C₈)-cycloalkyl, aryl, heterocyclyl, wherein each of the last-mentioned 3 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl, and wherein heterocyclyl has q oxo groups,
   wherein R¹³, R¹⁴ and q each have, independently from one another, the meaning as defined above in the context of the formula (G), preferably each have, independently from one another, the meaning as defined above in one of the preferred, more preferred, or particularly preferred embodiments mentioned hereinbefore or hereinafter.

More preferred compounds according to the present invention correspond to the formula (G), wherein

R³ is not hydrogen,

and wherein the other structural elements in the formula (G) each have, independently from one another, the meaning as defined above in the context of the formula (G), preferably each have, independently from one another, the meaning as defined above in one of the preferred, more preferred, or particularly preferred embodiments mentioned hereinbefore or hereinafter.

Preferred compounds according to the present invention correspond to the formula (G) and/or salts thereof, wherein
R⁴, R⁵ are preferably each independently hydrogen, (C₁-C₄)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, (C₁-C₆)-alkoxy-(C₁-C₃)-alkyl, (C₁-C₆)-alkoxy-(C₁-C₃)-alkylcarbonyl, (C₁-C₆)-alkoxycarbonyl, (C₁-C₆)-haloalkoxycarbonyl, (C₂-C₆)-alkenyloxycarbonyl, (C₂-C₆)-alkynyloxycarbonyl, (C₁-C₆)-alkylcarbonyl, (C₁-C₆)-haloalkylcarbonyl, R¹³R¹⁴N-carbonyl, (C₁-C₄)-alkylthio , (C₁-C₄)-haloalkylthio, (C₁-C₁₂)-alkoxycarbonylcarbonyl, (C₁-C₁₂)-alkoxycarbonyl-(C₁-C₃)-alkylcarbonyl, (C₃-C₈)-cycloalkylcarbonyl, (C₃-C₈)-cycloalkenylcarbonyl, (C₃-C₈)-cycloalkyl-(C₁-C₆)-alkylcarbonyl, (C₃-C₈)-cycloalkenyl-(C₁-C₆)-alkylcarbonyl, arylcarbonyl, aryl-(C₁-C₆)-alkylcarbonyl, heteroarylcarbonyl, heteroaryl-(C₁-C₆)-alkylcarbonyl, heterocyclylcarbonyl, or heterocyclyl-(C₁-C₆)-alkylcarbonyl, wherein each of the last-mentioned 10 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, and hydroxycarbonyl,
   or
   NR⁴R⁵ is —N═CR⁸R⁹ or —N═S(O)ₙR¹⁰R¹¹,
   wherein R⁸, R⁹, R¹⁰, R¹¹, R¹³ and R¹⁴ each have, independently from one another, the meaning as defined above in the context of the formula (G), preferably each have, independently from one another, the meaning as defined above in one of the preferred, more preferred, or particularly preferred embodiments mentioned hereinbefore or hereinafter.

Preferred compounds according to the present invention correspond to the formula (G) and/or salts thereof, wherein

A is CR⁶R⁷, wherein

R⁶, R⁷ are each independently hydrogen, cyano, halogen, (C₁-C₃)-alkyl, (C₃-C₈)-cycloalkyl,
or
R⁶ and R⁷, together with the carbon atom to which they are attached, form a 3-6-membered carbocyclic ring, wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, and
y is 1,
wherein R¹³ and R¹⁴ each have, independently from one another, the meaning as defined above in the context of the formula (G), preferably each have, independently from one another, the meaning as defined above in one of the preferred, more preferred, or particularly preferred embodiments mentioned hereinbefore or hereinafter.

According to the present invention, compounds of the formula (G) and/or a salt thereof are preferred, in which
A is CR⁶R⁷,
W is O or S,
R¹ is hydrogen, (C₁-C₆)-alkyl, (C₁-C₆)-haloalkyl, (C₂-C₆)-alkenyl, (C₂-C₆)-alkynyl, NR¹³R¹⁴, R¹³R¹⁴N-(C₁-C₆)-alkyl, (C₁-C₆)-alkoxy, (C₁-C₆)-haloalkoxy, (C₁-C₆)-haloalkoxy-(C₁-C₃)-alkyl, (C₁-C₆)-alkoxy-(C₁-C₃)-alkyl, (C₁-C₆)-alkoxy-(C₂-C₆)-alkoxy-(C₁-C₃)-alkyl, (C₁-C₄)-alkylthio, (C₁-C₄)-alkylsulphoxy, (C₁-C₄)-alkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₆)-cycloalkyl, (C₃-C₆)-cycloalkenyl, (C₃-C₆)-cycloalkyl-(C₁-C₃)-alkyl, (C₃-C₆)-cycloalkenyl-(C₁-C₃)-alkyl, (C₃-C₆)-cycloalkoxy, phenyl, heteroaryl, heterocyclyl, phenoxy, heteroaryloxy, heterocyclyloxy or a carbobicyclic residue, wherein each of the last-mentioned 12 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
R² is hydrogen, (C₁-C₆)-alkyl, (C₂-C₆)-alkenyl, (C₂-C₆)-alkynyl, (C₁-C₄)-alkylsulphonyl, (C₁-C₄)-haloalkylsulphonyl, (C₁-C₆)-alkylcarbonyl, (C₂-C₆)-alkenylcarbonyl, (C₂-C₆)-alkynylcarbonyl, (C₁-C₆)-alkoxycarbonyl, di((C₁-C₆)-alkyl)aminocarbonyl, (C₃-C₈)-cycloalkylcarbonyl, heteroarylcarbonyl or phenylcarbonyl, wherein each of the last-mentioned 3 residues is unsubstituted or is substituted by one or more residues from the group consisting of halogen, nitro, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl,
R³ is halogen, (C₁-C₄)-alkyl, (C₁-C₄)-haloalkyl, (C₂-C₄)-alkenyl, (C₂-C₄)-haloalkenyl, (C₂-C₄)-alkynyl, (C₁-C₆)-alkoxy, (C₁-C₆)-haloalkoxy, (C₁-C₆)-haloalkoxy-(C₁-C₃)-alkyl, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₃-C₈)-cycloalkyl, phenyl, phenyloxy, phenylthio, phenylsulphoxy, phenylsulphonyl, wherein each of the last-mentioned 6 residues is unsubstituted or is substituted by one or more residues from the group consisting of halogen, (C₁-C₄)-alkyl, (C₁-C₄)-haloalkyl, (C₁-C₄)-alkoxy, (C₁-C₄)-haloalkoxy,
R⁴, R⁵ are each independently hydrogen, (C₁-C₆)-alkyl, (C₁-C₆)-haloalkyl, (C₂-C₆)-alkenyl, (C₂-C₆)-alkynyl, (C₁-C₆)-alkoxy-(C₁-C₃)-alkyl, (C₁-C₆)-alkoxy-(C₁-C₃)-alkylcarbonyl, (C₁-C₄)-alkylthio, (C₁-C₄)-haloalkylthio, (C₁-C₄)-alkylthiocarbonyl, (C₁-C₄)-haloalkylthiocarbonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-alkylthio-(C₁-C₃)-alkylcarbonyl, (C₁-C₄)-alkylsulphoxy-(C₁-C₃)-alkylcarbonyl, (C₁-C₄)-alkylsulphonyl-(C₁-C₃)-alkylcarbonyl, (C₁-C₆)-alkylcarbonyl, (C₁-C₆)-haloalkylcarbonyl, (C₂-C₆)-alkenylcarbonyl, (C₂-C₆)-alkynylcarbonyl, (C₁-C₆)-alkoxycarbonylcarbonyl, (C₁-C₆)-alkoxycarbonyl-(C₁-C₃)-alkylcarbonyl, (C₁-C₆)-alkoxycarbonyl, (C₁-C₆)-haloalkoxycarbonyl, (C₂-C₆)-alkenyloxycarbonyl, (C₃-C₆)-cycloalkyl, (C₃-C₆)-cycloalkyl-(C₁-C₆)-alkyl, (C₃-C₆)-cycloalkylcarbonyl, (C₃-C₆)-cycloalkyl-(C₁-C₆)-alkylcarbonyl, phenyl, phenyl-(C₁-C₃)-alkyl, heteroaryl, heteroaryl-(C₁-C₃)-alkyl, heterocyclyl, heterocyclyl-(C₁-C₃)-alkyl, phenylcarbonyl, phenyl-(C₁-C₆)-alkylcarbonyl, hetarylcarbonyl, hetaryl-(C₁-C₆)-alkylcarbonyl, heterocyclylcarbonyl, heterocyclyl-(C₁-C₆)-alkylcarbonyl, wherein each of the last-mentioned 16 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
   or
   NR⁴R⁵ is —N═CR⁸R⁹ or —N═S(O)ₙR¹⁰R¹¹,
R⁶, R⁷ are each independently hydrogen or (C₁-C₆)-alkyl,
R⁸, R⁹ are each independently hydrogen, (C₁-C₆)-alkyl, (C₁-C₆)-haloalkyl, (C₂-C₆)-alkenyl, (C₂-C₆)-alkynyl, (C₁-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₆)-alkoxy, (C₁-C₆)-haloalkoxy, (C₂-C₆)-alkenyloxy, NR¹³R¹⁴, (C₃-C₈)-cycloalkyl, (C₃-C₈)-cycloalkyl-(C₁-C₆)-alkyl, phenyl, phenyl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 8 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
   or
   R⁸ and R⁹, together with the carbon atom to which they are attached, form a 3- to 6-membered unsaturated, partially saturated or saturated ring, which comprises in each case, in addition to the carbon atoms, p ring members from the group consisting of N(R¹²)ₘ, O and S(O)ₙ, and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
R¹⁰, R¹¹ are each independently, (C₁-C₆)-alkyl, (C₂-C₆)-alkenyl, (C₂-C₆)-alkynyl, (C₁-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₈)-cycloalkyl, (C₃-C₈)-cycloalkyl-(C₁-C₆)-alkyl, phenyl, phenyl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 8 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
   or
   R¹⁰ and R¹¹, together with the sulphur atom to which they are attached, form a 3- to 6-membered unsaturated, partially saturated or saturated ring, which comprises in each case, in addition to the carbon atoms and in addition to the sulphur atom, p ring members from the group consisting of N(R¹²)ₘ, O and S(O)ₙ, and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy or (C₁-C₄)-alkylsulphonyl, and wherein heterocyclyl has q oxo groups,
R¹² is hydrogen, (C₁-C₆)-alkyl or (C₁-C₆)-alkylcarbonyl,
R¹³, R¹⁴ are each independently hydrogen, (C₁-C₆)-alkyl, (C₁-C₆)-alkylcarbonyl, (C₁-C₄)-alkylsulphonyl, phenyl, phenylcarbonyl, wherein each of the last-mentioned two residues is unsubstituted or is substituted by one or more residues from the group consisting of halogen, (C₁-C₄)-alkyl, (C ₁-C₄)-haloalkyl, (C₁-C₄)-alkoxy, (C₁-C₄)-haloalkoxy , (C₁-C₄)-alkylthio, (C₁-C₄)-alkylsulphoxy, (C₁-C₄)-alkylsulphonyl,
   or
   R¹³ and R¹⁴, together with the nitrogen atom to which they are attached, form a 3- to 8-membered unsaturated, partially saturated or saturated ring, which comprises in each case, in addition to the carbon atoms and in addition to the nitrogen atom, p ring members from the group consisting of N(R¹²)ₘ, O and S(O)ₙ, and wherein said ring is unsubstituted or is substituted by one or more residues from the group consisting of halogen, hydroxyl, (C₁-C₄)-alkyl, (C₁-C₄)-haloalkyl, (C₁-C₄)-alkoxy, (C₁-C₄)-haloalkoxy, and has q oxo groups,
n is independently selected from 0, 1 or 2,
m is independently selected from 0 or 1,
p is independently selected from 0, 1 or 2,
q is independently selected from 0, 1 or 2,
y is 0 or 1,
with the proviso that:
y is 1, if R¹ is a substituted 4-heptafluoroisopropylphenyl residue, a substituted 4-(nonafluoro-2-butyl)phenyl residue, a substituted 4-(1,1,2,3,3,3-hexafluoropropoxy)phenyl residue, a 2-bromo-4-methyl-6-(heptafluoroisopropyl)pyridin-3-yl residue or a 2-bromo-4-methyl-6-(2,2,2-trifluoro-1-trifluoromethylethoxy)pyridin-3-ylresidue.

Preferred compounds according to the present invention correspond to the formula (G) as defined hereinabove, wherein

R¹ is hydrogen, (C₁-C₆)-alkyl, (C₁-C₆)-haloalkyl, (C₂-C₆)-alkenyl, (C₂-C₆)-alkynyl, NR¹³R¹⁴, R¹³R¹⁴N—(C₁-C₆)-alkyl, (C₁-C₆)-alkoxy, (C₁-C₆)-haloalkoxy, (C₁-C₆)-haloalkoxy-(C₁-C₃)-alkyl, (C₁-C₆)-alkoxy-(C₁-C₃)-alkyl, (C₁-C₆)-alkoxy-(C₂-C₆)-alkoxy-(C₁-C₃)-alkyl, (C₁-C₄)-alkylthio, (C₁-C₄)-alkylsulphoxy, (C₁-C₄)-alkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₆)-cycloalkyl, (C₃-C₆)-cycloalkenyl, (C₃-C₆)-cycloalkyl-(C₁-C₃)-alkyl, (C₃-C₆)-cycloalkenyl-(C₁-C₃)-alkyl, (C₃-C₆)-cycloalkoxy, phenyl, heteroaryl, heterocyclyl, phenoxy, heteroaryloxy, heterocyclyloxy or a carbobicyclic residue, wherein each of the last-mentioned 12 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
and wherein A, W, R², R³, R⁴, R⁵, n, m, p, q and y each have the meaning defined hereinabove,
or
R¹ is an optionally substituted carbocyclic ring, preferably a monocyclic or bicyclic carbocyclic ring, more preferably a monocyclic or bicyclic carbocyclic ring with a total of 6 to 12 ring carbon atoms, more preferably with a total of 6 to 10 ring carbon atoms (i.e. only taking into account the carbon atoms of the carbocyclic ring, and excluding the carbon atoms of the optionally present substituents on the carbocyclic ring),
   and
y is 0,
and wherein A, W, R², R³, R⁴, R⁵, n, m, p and q each have the meaning defined hereinabove.

Preferred compounds according to the present invention correspond to the formula (G), wherein
y is 1, if R¹ is a substituted phenyl residue or a substituted pyridin-3-yl residue, and wherein the other structural elements in the formula (G) each have, independently from one another, the meaning as defined above in the context of the formula (G), preferably each have, independently from one another, the meaning as defined above in one of the preferred, more preferred, or particularly preferred embodiments.

According to the present invention, compounds of the formula (G) and/or a salt thereof are preferred, in which
A is CR⁶R⁷,
W is O or S,
R¹ is (C₁-C₆)-alkyl, (C₁-C₆)-haloalkyl, (C₂-C₆)-alkenyl, (C₂-C₆)-alkynyl, NR¹³R¹⁴, R¹³R¹⁴N-(C₁-C₆)-alkyl, (C₁-C₆)-alkoxy, (C₁-C₆)-haloalkoxy, (C₁-C₆)-alkoxy-(C₁-C₃)-alkyl, (C₁-C₆)-alkoxy-(C₂-C₆)-alkoxy-(C₁-C₃)-alkyl, (C₁-C₄)-alkylthio, (C₁-C₄)-alkylsulphoxy, (C₁-C₄)-alkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₆)-cycloalkyl, (C₃-C₆)-cycloalkenyl, (C₃-C₆)-cycloalkyl-(C₁-C₃)-alkyl, (C₃-C₆)-cycloalkenyl-(C₁-C₃)-alkyl, (C₃-C₆)-cycloalkoxy, phenyl, heteroaryl, heterocyclyl, phenoxy, heteroaryloxy, heterocyclyloxy or a carbobicyclic residue, wherein each of the last-mentioned 12 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
R² is hydrogen, (C₁-C₆)-alkyl, (C₂-C₆)-alkenyl, (C₂-C₆)-alkynyl, (C₁-C₄)-alkylsulphonyl, (C₁-C₄)-haloalkylsulphonyl, (C₁-C₆)-alkylcarbonyl, (C₂-C₆)-alkenylcarbonyl, (C₂-C₆)-alkynylcarbonyl, (C₁-C₆)-alkoxycarbonyl, di((C₁-C₆)-alkyl)aminocarbonyl, (C₃-C₈)-cycloalkylcarbonyl, heteroarylcarbonyl or phenylcarbonyl, wherein each of the last-mentioned 3 residues is unsubstituted or is substituted by one or more residues from the group consisting of halogen, nitro, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl,
R³ is halogen, (C₁-C₄)-haloalkyl, (C₂-C₄)-alkynyl, (C₁-C₄)-alkoxy, (C₁-C₄)-haloalkoxy, methylthio, (C₃-C₈)-cycloalkyl, phenyl, phenyloxy, wherein each of the last-mentioned 3 residues is unsubstituted or is substituted by one or more residues from the group consisting of halogen, (C₁-C₄)-alkyl, (C₁-C₄)-haloalkyl, (C₁-C₄)-alkoxy, (C₁-C₄)-haloalkoxy,
R⁴, R⁵ are each independently hydrogen, (C₁-C₆)-alkyl, (C₁-C₆)-haloalkyl, (C₂-C₆)-alkenyl, (C₂-C₆)-alkynyl, (C₁-C₆)-alkoxy-(C₁-C₃)-alkyl, (C₁-C₆)-alkoxy-(C₁-C₃)-alkylcarbonyl, (C₁-C₄)-alkylthio, (C₁-C₄)-haloalkylthio, (C₁-C₄)-alkylthiocarbonyl, (C₁-C₄)-haloalkylthiocarbonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-alkylthio-(C₁-C₃)-alkylcarbonyl, (C₁-C₄)-alkylsulphoxy-(C₁-C₃)-alkylcarbonyl, (C₁-C₄)-alkylsulphonyl-(C₁-C₃)-alkylcarbonyl, (C₁-C₆)-alkylcarbonyl, (C₁-C₆)-haloalkylcarbonyl, (C₂-C₆)-alkenylcarbonyl, (C₂-C₆)-alkynylcarbonyl, (C₁-C₆)-alkoxycarbonylcarbonyl, (C₁-C₆)-alkoxycarbonyl-(C₁-C₃)-alkylcarbonyl, (C₁-C₆)-alkoxycarbonyl, (C₁-C₆)-haloalkoxycarbonyl, (C₂-C₆)-alkenyloxycarbonyl, (C₃-C₆)-cycloalkyl, (C₃-C₆)-cycloalkyl-(C₁-C₆)-alkyl, (C₃-C₆)-cycloalkylcarbonyl, (C₃-C₆)-cycloalkyl-(C₁-C₆)-alkylcarbonyl, phenyl, phenyl-(C₁-C₃)-alkyl, heteroaryl, heteroaryl-(C₁-C₃)-alkyl, heterocyclyl, heterocyclyl-(C₁-C₃)-alkyl, phenylcarbonyl, phenyl-(C₁-C₆)-alkylcarbonyl, hetarylcarbonyl, hetaryl-(C₁-C₆)-alkylcarbonyl, heterocyclylcarbonyl, heterocyclyl-(C₁-C₆)-alkylcarbonyl, wherein each of the last-mentioned 16 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
   or
   NR⁴R⁵ is —N═CR⁸R⁹ or —N═S(O)ₙR¹⁰R¹¹,
R⁶, R⁷ are each independently hydrogen or (C₁-C₄)-alkyl, preferably R⁶ and R⁷ independently are hydrogen or methyl,
R⁸, R⁹ are each independently hydrogen, (C₁-C₆)-alkyl, (C₁-C₆)-haloalkyl, (C₂-C₆)-alkenyl, (C₂-C₆)-alkynyl, (C₁-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₆)-alkoxy, (C₁-C₆)-haloalkoxy, (C₂-C₆)-alkenyloxy, NR¹³R¹⁴, (C₃-C₈)-cycloalkyl, (C₃-C₈)-cycloalkyl-(C₁-C₆)-alkyl, phenyl, phenyl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 8 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
   or
   R⁸ and R⁹, together with the carbon atom to which they are attached, form a 3- to 6-membered unsaturated, partially saturated or saturated ring, which comprises in each case, in addition to the carbon atoms, p ring members from the group consisting of N(R¹²)ₘ, O and S(O)ₙ, and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
R¹⁰, R¹¹ are each independently, (C₁-C₆)-alkyl, (C₂-C₆)-alkenyl, (C₂-C₆)-alkynyl, (C₁-C₆)-alkoxy-(C₁-C₃)-alkyl, (C₁-C₄)-alkylthio-(C₁-C₃)-alkyl, (C₁-C₄)-alky lsulphoxy-(C₁-C₃)-alkyl, (C₁-C₄)-alkylsulphonyl-(C₁-C₃)-alkyl, (C₃-C₈)-cycloalkyl, (C₃-C₈)-cycloalkyl-(C₁-C₆)-alkyl, phenyl, phenyl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 8 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
   or
   R¹⁰ and R¹¹, together with the sulphur atom to which they are attached, form a 3- to 6-membered unsaturated, partially saturated or saturated ring, which comprises in each case, in addition to the carbon atoms and in addition to the sulphur atom, p ring members from the group consisting of N(R¹²)ₘ, O and S(O)ₙ, and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy or (C₁-C₄)-alkylsulphonyl, and wherein heterocyclyl has q oxo groups,
R¹² is hydrogen, (C₁-C₆)-alkyl or (C₁-C₆)-alkylcarbonyl,
R¹³, R¹⁴ are each independently hydrogen, (C₁-C₆)-alkyl, (C₁-C₆)-alkylcarbonyl, (C₁-C₄)-alkylsulphonyl, phenyl, phenylcarbonyl, wherein each of the last-mentioned two residues is unsubstituted or is substituted by one or more residues from the group consisting of halogen, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl,
   or
   R¹³ and R¹⁴, together with the nitrogen atom to which they are attached, form a 3- to 8-membered unsaturated, partially saturated or saturated ring, which comprises in each case, in addition to the carbon atoms and in addition to the nitrogen atom, p ring members from the group consisting of N(R¹²)ₘ, O and S(O)ₙ, and wherein said ring is unsubstituted or is substituted by one or more residues from the group consisting of halogen, hydroxyl, (C₁-C₄)-alkyl, (C₁-C₄)-haloalkyl, (C₁-C₄)-alkoxy, (C₁-C₄)-haloalkoxy, and has q oxo groups,
n is independently selected from 0, 1 or 2,
m is independently selected from 0 or 1,
p is independently selected from 0, 1 or 2,
q is independently selected from 0 or 1,
y is 0 or 1,
with the proviso that:
y is 1, if R¹ is a substituted phenyl residue or a substituted pyridin-3-yl residue.

In all of the above mentioned aspects, preferred compounds of the formula (G) and/or a salt thereof according to the present invention are those, wherein R⁴ and R⁵ are not both an alkyl residue, more preferably R⁴ and R⁵ are not both an (C₁-C₁₂)-alkyl residue, and more specifically, R⁴ and R⁵ are not both an (C₁-C₆)-alkyl residue.

According to the present invention, compounds of the formula (G) and/or a salt thereof are more preferred, in which
A is CR⁶R⁷,
W is O or S,
R¹ is (C₁-C₆)-alkyl, (C₁-C₆)-haloalkyl, (C₂-C₆)-alkenyl, (C₂-C₆)-alkynyl, NR¹³R¹⁴, R¹³R¹⁴N—(C₁-C₆)-alkyl, (C₁-C₆)-alkoxy, (C₁-C₆)-haloalkoxy, (C₁-C₆)-alkoxy-(C₁-C₃)-alkyl, (C₁-C₆)-alkoxy-(C₂-C₆)-alkoxy-(C₁-C₃)-alkyl, (C₁-C₄)-alkylthio, (C₁-C₄)-alkylsulphoxy, (C₁-C₄)-alkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₆)-cycloalkyl, (C₃-C₆)-cycloalkenyl, (C₃-C₆)-cycloalkyl-(C₁-C₃)-alkyl, (C₃-C₆)-cycloalkenyl-(C₁-C₃)-alkyl, (C₃-C₆)-cycloalkoxy, phenyl, heteroaryl, heterocyclyl, phenoxy, heteroaryloxy, heterocyclyloxy or a carbobicyclic residue, wherein each of the last-mentioned 12 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
R² is hydrogen, (C₁-C₆)-alkylcarbonyl, (C₂-C₆)-alkenylcarbonyl, (C₂-C₆)-alkynylcarbonyl, (C₁-C₆)-alkoxycarbonyl, (C₃-C₆)-cycloalkylcarbonyl, heteroarylcarbonyl, or phenylcarbonyl, wherein each of the last-mentioned 3 residues is unsubstituted or is substituted by one or more residues from the group consisting of halogen, nitro, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl,
R³ is halogen, methyl, difluoromethyl (CHF₂), trifluoromethyl (CF₃) or (C₂-C₃)-alkynyl,
R⁴, R⁵ are each independently hydrogen, (C₂-C₆)-alkynyl, (C₁-C₄)-alkylthio, (C₁-C₄)-haloalkylthio, (C₁-C₄)-alkylthiocarbonyl, (C₁-C₄)-haloalkylthiocarbonyl, (C₁-C₆)-alkoxy-(C₁-C₃)-alkylcarbonyl, (C₁-C₄)-alkylthio-(C₁-C₃)-alkylcarbonyl, (C₁-C₄)-alkylsulphoxy-(C₁-C₃)-alkylcarbonyl, (C₁-C₄)-alkylsulphonyl-(C₁-C₃)-alkylcarbonyl, (C₁-C₆)-alkylcarbonyl, (C₁-C₆)-haloalkylcarbonyl, (C₂-C₆)-alkenylcarbonyl, (C₂-C₆)-alkynylcarbonyl, (C₁-C₆)-alkoxycarbonylcarbonyl, (C₁-C₆)-alkoxycarbonyl-(C₁-C₃)-alkylcarbonyl, (C₁-C₆)-alkoxycarbonyl, (C₁-C₆)-haloalkoxycarbonyl, (C₂-C₆)-alkenyloxycarbonyl, (C₃-C₆)-cycloalkylcarbonyl, (C₃-C₆)-cycloalkyl-(C₁-C₆)-alkylcarbonyl, phenylcarbonyl, phenyl-(C₁-C₆)-alkylcarbonyl, hetarylcarbonyl, hetaryl-(C₁-C₆)-alkylcarbonyl, heterocyclylcarbonyl, heterocyclyl-(C₁-C₆)-alkylcarbonyl, wherein each of the last-mentioned 8 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
   or
   NR⁴R⁵ is —N═CR⁸R⁹ or —N═S(O)ₙR¹⁰R¹¹,
R⁶ is hydrogen,
R⁷ is hydrogen or methyl,
R⁸, R⁹ are each independently hydrogen, (C₁-C₆)-alkyl, (C₁-C₆)-haloalkyl, (C₂-C₆)-alkenyl, (C₂-C₆)-alkynyl, (C₁-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₆)-alkoxy, (C₁-C₆)-haloalkoxy, (C₂-C₆)-alkenyloxy, NR¹³R¹⁴, (C₃-C₈)-cycloalkyl, (C₃-C₈)-cycloalkyl-(C₁-C₆)-alkyl, phenyl, phenyl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 8 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
   or
   R⁸ and R⁹, together with the carbon atom to which they are attached, form a 3- to 6-membered unsaturated, partially saturated or saturated ring, which comprises in each case, in addition to the carbon atoms, p ring members from the group consisting of N(R¹²)ₘ, O and S(O)ₙ, and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
R¹⁰, R¹¹ are each independently, (C₁-C₆)-alkyl, (C₂-C₆)-alkenyl, (C₂-C₆)-alkynyl, (C₁-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₈)-cycloalkyl, (C₃-C₈)-cycloalkyl-(C₁-C₆)-alkyl, phenyl, phenyl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 8 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
   or
   R¹⁰ and R¹¹, together with the sulphur atom to which they are attached, form a 3- to 6-membered unsaturated, partially saturated or saturated ring, which comprises in each case, in addition to the carbon atoms and in addition to the sulphur atom, p ring members from the group consisting of N(R¹²)ₘ, O and S(O)ₙ, and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy or (C₁-C₄)-alkylsulphonyl, and wherein heterocyclyl has q oxo groups,
R¹² is hydrogen, (C₁-C₆)-alkyl or (C₁-C₆)-alkylcarbonyl,
R¹³, R¹⁴ are each independently hydrogen, (C₁-C₆)-alkyl, (C₁-C₆)-alkylcarbonyl, (C₁-C₄)-alkylsulphonyl, phenyl, phenylcarbonyl, wherein each of the last-mentioned two residues is unsubstituted or is substituted by one or more residues from the group consisting of halogen, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl,
   or
   R¹³ and R¹⁴, together with the nitrogen atom to which they are attached, form a 3- to 8-membered unsaturated, partially saturated or saturated ring, which comprises in each case, in addition to the carbon atoms and in addition to the nitrogen atom, p ring members from the group consisting of N(R¹²)ₘ, O and S(O)ₙ, and wherein said ring is unsubstituted or is substituted by one or more residues from the group consisting of halogen, hydroxyl, (C₁-C₄)-alkyl, (C₁-C₄)-haloalkyl, (C₁-C₄)-alkoxy, (C₁-C₄)-haloalkoxy, and has q oxo groups,
n is independently selected from 0, 1 or 2,
m is independently selected from 0 or 1,
p is independently selected from 0, 1 or 2,
q is independently selected from 0 or 1,
y is 0 or 1,
with the proviso that:
y is 1, if R¹ is a substituted phenyl residue or a substituted pyridin-3-yl residue.

According to the present invention, compounds of the formula (G) and/or a salt thereof are even more preferred, in which
R³ is halogen, trifluoromethyl or ethynyl.

According to the present invention, compounds of the formula (G) and/or a salt thereof are even more preferred, in which
R³ is F, Cl, Br, I, trifluoromethyl or ethynyl.

If R³ is Cl, in preferred compounds according to the present invention corresponding to the formula (G), then R¹ is not a substituted 4-heptafluoroisopropylphenyl residue.

Particularly preferred compounds according to the present invention correspond to the formula (G), wherein y=1.

Particularly preferred compounds according to the present invention correspond to the formula (G), wherein y=1, and wherein W, R¹, R², R³, R⁴, R⁵ and A each have, independently from one another, the meaning as defined above in the context of the formula (G), preferably each have, independently from one another, the meaning as defined above in one of the preferred, more preferred, or particularly preferred embodiments.

Particularly preferred compounds according to the present invention correspond to the formula (G), wherein y=1, A is CHR⁷ (i.e. R⁶═H), wherein R⁷ is hydrogen or methyl,

and

wherein W, R¹, R², R³, R⁴, and R⁵ each have, independently from one another, the meaning as defined above in the context of the formula (G), preferably each have, independently from one another, the meaning as defined above in one of the preferred, more preferred, or particularly preferred embodiments.

In a preferred embodiment, the compounds according to the present invention correspond to the formula (G), wherein
R⁴, R⁵ are each independently hydrogen, (C₂-C₆)-alkynyl, (C₁-C₄)-alkylthio, (C₁-C₄)-haloalkylthio (wherein (C₁-C₄)-haloalkylthio more preferably is SCF), (C₁-C₆)-alkoxy-(C₁-C₃)-alkylcarbonyl, (C₁-C₄)-alkylthio-(C₁-C₃)-alkylcarbonyl, (C₁-C₄)-alkylsulphoxy-(C₁-C₃)-alkylcarbonyl, (C₁-C₄)-alkylsulphonyl-(C₁-C₃)-alkylcarbonyl, (C₁-C₆)-alkylcarbonyl, (C₁-C₆)-haloalkylcarbonyl, (C₂-C₆)-alkenylcarbonyl, (C₂-C₆)-alkynylcarbonyl, (C₁-C₆)-alkoxycarbonylcarbonyl, (C₁-C₆)-alkoxycarbonyl-(C₁-C₃)-alkylcarbonyl, (C₁-C₆)-alkoxycarbonyl, (C₁-C₆)-haloalkoxycarbonyl, (C₂-C₆)-alkenyloxycarbonyl, (C₃-C₆)-cycloalkylcarbonyl, (C₃-C₆)-cycloalkyl-(C₁-C₆)-alkylcarbonyl, phenylcarbonyl, phenyl-(C₁-C₆)-alkylcarbonyl, hetarylcarbonyl, hetaryl-(C₁-C₆)-alkylcarbonyl, heterocyclylcarbonyl, heterocyclyl-(C₁-C₆)-alkylcarbonyl, wherein each of the last-mentioned 8 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
   or
   NR⁴R⁵ is —N═CR⁸R⁹, wherein R⁸ and R⁹ each have, independently from one another, the meaning as defined above in the context of the formula (G), preferably each have, independently from one another, the meaning as defined above in one of the preferred, more preferred, or particularly preferred embodiments,
      and wherein the other structural elements in the formula (G) each have, independently from one another, the meaning as defined above in the context of the formula (G), preferably each have, independently from one another, the meaning as defined above in one of the preferred, more preferred, or particularly preferred embodiments.

Preferred compounds according to the present invention correspond to the formula (G), wherein

R² is not methyl,

and wherein the other structural elements in the formula (G) each have, independently from one another, the meaning as defined above in the context of the formula (G), preferably each have, independently from one another, the meaning as defined above in one of the preferred, more preferred, or particularly preferred embodiments.

Preferred compounds according to the present invention correspond to the formula (G) and/or salts thereof, wherein
R¹ is (C₄-C₈)-cycloalkyl, (C₄-C₈)-cycloalkenyl, aryl, heteroaryl, heterocyclyl, a bicyclic or a heterobicyclic residue, wherein each of the mentioned residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl, and wherein heterocyclyl has q oxo groups, and wherein each of the aforementioned heterocyclic residues, in addition to the carbon atoms, has in each case p ring members from the group consisting of N(R¹²)ₘ, O and S(O)ₙ,
   wherein R¹², R¹³, R¹⁴, p, q, m and n each have, independently from one another, the meaning as defined above in the context of the formula (G), preferably each have, independently from one another, the meaning as defined above in one of the preferred, more preferred, or particularly preferred embodiments mentioned hereinbefore or hereinafter.

More preferred compounds according to the present invention correspond to the formula (G) and/or salts thereof, wherein
R¹ is (C₄-C₈)-cycloalkyl, (C₄-C₈)-cycloalkenyl, aryl, heteroaryl, heterocyclyl, a bicyclic or a heterobicyclic residue, wherein each of the mentioned residues is unsubstituted or is substituted by one or more residues from the group consisting of halogen, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkoxycarbonyl, (C₁-C₄)-haloalkoxycarbonyl, (C₃-C₆)-cycloalkyl,
   wherein R¹³ and R¹⁴ each have, independently from one another, the meaning as defined above in the context of the formula (G), preferably each have, independently from one another, the meaning as defined above in one of the preferred, more preferred, or particularly preferred embodiments mentioned hereinbefore or hereinafter.

Preferred compounds according to the present invention correspond to the formula (G) and/or salts thereof, wherein
R² is hydrogen, (C₁-C₆)-alkylcarbonyl, (C₂-C₆)-alkenylcarbonyl, (C₂-C₆)-alkynylcarbonyl, (C₁-C₆)-haloalkylcarbonyl, (C₂-C₆)-haloalkenylcarbonyl, (C₂-C₆)-haloalkynylcarbonyl, (C₁-C₆)-alkoxycarbonyl, (C₃-C₈)-cycloalkylcarbonyl, (C₃-C₈)-cycloalkyl-(C₁-C₆)-alkylcarbonyl, heteroarylcarbonyl, or arylcarbonyl, wherein each of the last-mentioned 4 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl,
   wherein R¹¹ and R¹⁴ each have, independently from one another, the meaning as defined above in the context of the formula (G), preferably each have, independently from one another, the meaning as defined above in one of the preferred, more preferred, or particularly preferred embodiments mentioned hereinbefore or hereinafter.

More preferred compounds according to the present invention correspond to the formula (G) and/or salts thereof, wherein
R¹ is (C₄-C₈)-cycloalkyl, (C₄-C₈)-cycloalkenyl, aryl, heteroaryl, heterocyclyl, a bicyclic or a heterobicyclic residue, wherein each of the mentioned residues is unsubstituted or is substituted by one or more residues from the group consisting of halogen, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkoxycarbonyl, (C₁-C₄)-haloalkoxycarbonyl, (C₃-C₆)-cycloalkyl,
and
R² is hydrogen, (C₁-C₆)-alkylcarbonyl, (C₂-C₆)-alkenylcarbonyl, (C₂-C₆)-alkynylcarbonyl, (C₁-C₆)-haloalkylcarbonyl, (C₂-C₆)-haloalkenylcarbonyl, (C₂-C₆)-haloalkynylcarbonyl, (C₁-C₆)-alkoxycarbonyl, (C₃-C₈)-cycloalkylcarbonyl, (C₃-C₈)-cycloalkyl-(C₁-C₆)-alkylcarbonyl, heteroarylcarbonyl, or arylcarbonyl, wherein each of the last-mentioned 4 residues is unsubstituted or is substituted by one or more residues from the group consisting of halogen, nitro, hydroxyl, cyano, NR¹³R¹⁴ (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl,
   wherein R1³ and R¹⁴ each have, independently from one another, the meaning as defined above in the context of the formula (G), preferably each have, independently from one another, the meaning as defined above in one of the preferred, more preferred, or particularly preferred embodiments mentioned hereinbefore or hereinafter.

According to the present invention, compounds of the formula (G) and/or a salt thereof are even more preferred, wherein

R² is H (hydrogen).

In another preferred embodiment, the compounds of the formula (G) and/or a salt thereof according to the present invention are even more preferred, wherein

y is 0, and preferably R¹ is an optionally substituted carbocyclic ring, more preferably R¹ is a monocyclic or bicyclic carbocyclic ring, even more preferably R¹ is a monocyclic or bicyclic carbocyclic ring with a total of 6 to 12 carbon atoms, and particularly preferably R¹ is a monocyclic or bicyclic carbocyclic ring with a total of 6 to 10 carbon atoms, excluding the optionally present substituents.

In another preferred embodiment, the compounds of the formula (G) and/or a salt thereof according to the present invention are even more preferred, wherein

y is 0, and

R¹ is an optionally substituted monocyclic or bicyclic carbocyclic ring with a total of 6 to 12 carbon atoms, and particularly preferably R¹ is a monocyclic or bicyclic carbocyclic ring with a total of 6 to 10 carbon atoms, excluding the optionally present substituents.

More preferred compounds according to the present invention correspond to formula the (G), wherein R²═H, and wherein W, R¹, R³, R⁴, R⁵, A and y each have, independently from one another, the meaning as defined above in the context of formula the (G), preferably each have, independently from one another, the meaning as defined above in one of the preferred, more preferred, or particularly preferred embodiments.

In all of the above embodiments, preferred compounds according to the present invention correspond to the formula (G), wherein
n is independently selected from 0, 1 or 2, preferably independently selected from 0 or 1, more preferably n is 0,
m is independently selected from 0 or 1, preferably m is 0,
p is independently selected from 0, 1 or 2, preferably p is independently selected from 0 or 1, and
q is independently selected from 0 or 1, preferably q is 0.

In all of the above embodiments, more preferred compounds according to the present invention correspond to the formula (G), wherein
n is independently selected from 0 or 1, preferably n is 0,
m is independently selected from 0 or 1, preferably m is 0,
p is independently selected from 0 or 1, preferably p is independently selected from 0 or 1, and
q is independently selected from 0 or 1, preferably q is 0.

The following compounds of the formulae (I), (II), (III), (IV), (V), (VI), (VI-a) and (VII) are preferred compounds of the formula (G) according to the present invention.

Preferred compounds according to the present invention correspond to the formula (G), wherein W═O, R²═H, R⁴═H, R⁵═H, y=1, and A=CH₂.

These preferred compounds of the formula (G) are compounds of the formula (I):

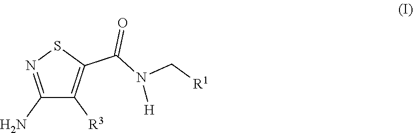

wherein R¹ and R³ each have, independently from one another, the meaning as defined above in the context of the formula (G), preferably each have, independently from one another, the meaning as defined above in one of the preferred, more preferred, or particularly preferred embodiments.

Preferred compounds according to the present invention correspond to the formula (G), wherein W═O, R²═H, y=1, and A=CH₂.

These preferred compounds of formula (G) are compounds of the formula (II):

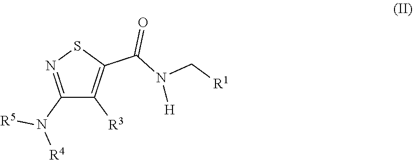

wherein R¹, R³, R⁴ and R⁵ each have, independently from one another, the meaning as defined above in the context of the formula (G), preferably each have, independently from one another, the meaning as defined above in one of the preferred, more preferred, or particularly preferred embodiments.

Preferred compounds according to the present invention correspond to the formula (G), wherein R²═H, i.e. compounds of the formula (III):

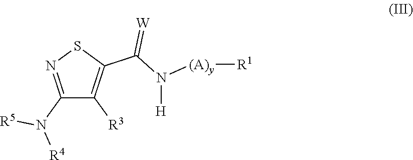

wherein R¹, R³, R⁴, R⁵, A and y each have, independently from one another, the meaning as defined above in the context of the formula (G), preferably each have, independently from one another, the meaning as defined above in one of the preferred, more preferred, or particularly preferred embodiments.

Preferred compounds according to the present invention correspond to the formula (G), wherein W═S, R²═H, R³═Cl, y=1, and A=CH₂.

These preferred compounds of formula (G) are compounds of the formula (IV):

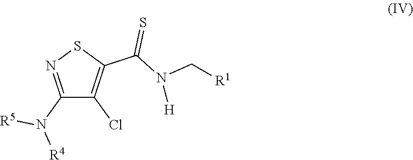

wherein R¹, R⁴ and R⁵ each have, independently from one another, the meaning as defined above in the context of the formula (G), preferably each have, independently from one another, the meaning as defined above in one of the preferred, more preferred, or particularly preferred embodiments.

Preferred compounds according to the present invention correspond to the formula (G), wherein W═O, R³═Cl, R⁴═H, R⁵═H, y=1, and A=CH₂, i.e. compounds of the formula (V):

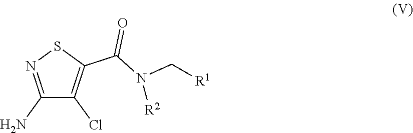

wherein R¹ and R⁵ each have, independently from one another, the meaning as defined above in the context of the formula (G), preferably each have, independently from one another, the meaning as defined above in one of the preferred, more preferred, or particularly preferred embodiments.

Preferred compounds according to the present invention correspond to the formula (G), wherein W═O, and R³═Cl, i.e. compounds of the formula (VI):

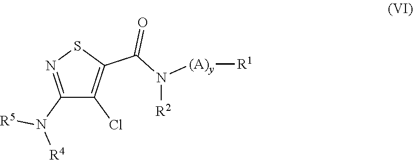

wherein R¹, R², R⁴, R⁵, A and y each have, independently from one another, the meaning as defined above in the context of the formula (G), preferably each have, independently from one another, the meaning as defined above in one of the preferred, more preferred, or particularly preferred embodiments.

Preferred compounds according to the present invention correspond to the formula (G), wherein W═O, and R³═F, i.e. compounds of the formula (VI-a):

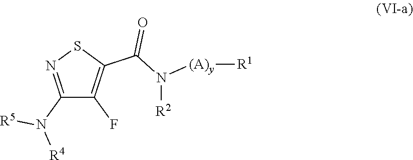

wherein R¹, R², R⁴, R⁵, A and y each have, independently from one another, the meaning as defined above in the context of the formula (G), preferably each have, independently from one another, the meaning as defined above in one of the preferred, more preferred, or particularly preferred embodiments.

Preferred compounds according to the present invention correspond to the formula (G), wherein W═O, and y=0 (i.e. A is not present), i.e. compounds of the formula (VII):

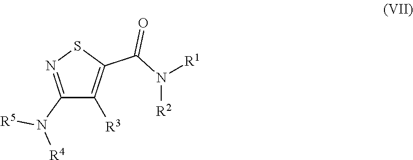

wherein R¹, R², R³, R⁴, and R⁵ each have, independently from one another, the meaning as defined above in the context of the formula (G), preferably each have, independently from one another, the meaning as defined above in one of the preferred, more preferred, or particularly preferred embodiments.

In another preferred embodiment, the compounds of the formulae (G) amd (VII) according to the present invention are even more preferred, wherein
R¹ is an optionally substituted monocyclic or bicyclic carbocyclic ring with a total of 6 to 12 carbon atoms, and particularly preferably R¹ is a monocyclic or bicyclic carbocyclic ring with a total of 6 to 10 carbon atoms, excluding the optionally present substituents, and
R² is hydrogen,
wherein R³, R⁴, and R⁵ each have, independently from one another, the meaning as defined above in the context of the formula (G), preferably each have, independently from one another, the meaning as defined above in one of the preferred, more preferred, or particularly preferred embodiments.

Specific and preferred definitions, independently from one another, of the moieties W, R¹, R², R³, R⁴, R⁵, A and y in the context of the formulae of the present invention are mentioned in the following Table 1.

R¹ in the context of the formulae (G), (I), (II), (III), (IV), (V), (VI), (VI-a) and (VII) according to the present invention particularly preferably is selected from the group consisting of the moieties mentioned in Table 1 for R¹.

R² in the context of the formulae (G), (V), (VI), (VI-a) and (VII) according to the present invention particularly preferably is selected from the group consisting of the moieties mentioned in Table 1 for R².

R³ in the context of the formulae (G), (I), (II), (III) and (VII) according to the present invention particularly preferably is selected from the group consisting of the moieties mentioned in Table 1 for R³.

R⁴ in the context of the formulae (G), (II), (III), (IV), (VI), (VI-a) and (VII) according to the present invention particularly preferably is selected from the group consisting of the moieties mentioned in Table 1 for R⁴.

R⁵ in the context of the formulae (G), (II), (III), (IV), (VI), (VI-a) and (VII) according to the present invention particularly preferably is selected from the group consisting of the moieties mentioned in Table 1 for R⁵.

A in the context of the formulae (G), (III), (VI), (VI-a) according to the present invention particularly preferably is selected from the group consisting of the moieties mentioned in Table 1 for A.

In the following Table 2 and Table 2a specific and preferred definitions of NR⁴R⁵ and N[R²](A)yR¹ in the context of the present invention are mentioned.

NR⁴R⁵in the context of the formulae (G), (II), (III), (IV), (VI), (VI-a) and (VII) according to the present invention particularly preferably is selected from the group consisting of the moieties mentioned in Table 2 and Table 2a for NR⁴R⁵.

N[R²](A)yR¹ in the context of the formulae (G), (VI) and (VI-a) according to the present invention particularly preferably is selected from the group consisting of the moieties mentioned in Table 2 for N [R²](A)yR¹.

The abbreviations and numerations of the substituent positions used in the context of the present invention and in the following Tables are explained in detail in the section Examples hereinafter. Also, the LogP values indicated in the following Tables are explained in detail in the section Examples hereinafter.

The examples in the following Tables 1 to 4 are numbered and the example number abbreviated in the Tables as “Ex N°”.

In case LogP values for different diastereoisomers of a specific compound are indictaed, said LogP values are separtated by a “+”. For example, for the compound of example number I-055 four LogP values are indicated in Table 1 for the four different diastereomers in the format “5,17+5,36+5,28+5,11”.

Specific and preferred definitions, independently from one another, of the moieties W, R¹, R², R³, R⁴, R⁵, A and y in the context of the formulae of the present invention and specific preferred compounds of the formula (G) are shown in the following Table 1.

TABLE 1
Preferred definitions of the moieties W, R¹, R², R³, R⁴, R⁵, A and y and preferred compounds of the formula (G)

| Ex No | R⁵ | R⁴ | R³ | W | R² | y | A | R¹ | LogP |
|---|---|---|---|---|---|---|---|---|---|
| I-001 | H | H | Cl | O | H | 0 | — | cyclohexyl | 2.10[a] |
| I-002 | H | H | Cl | O | H | 1 | CH₂ | cyclohexyl | 2.40[a] |
| I-003 | H | H | Cl | O | H | 1 | (S)—CHMe | cyclohexyl | 2.50[a] |
| I-004 | H | H | Cl | O | H | 1 | CH₂ | phenyl | 1.69[a] |
| I-005 | H | H | H | O | H | 1 | CH₂ | cyclohexyl | 1.92[a] |
| I-006 | H | H | H | O | H | 1 | (S)—CHMe | cyclohexyl | 2.09[a] |
| I-007 | H | H | H | O | H | 0 | — | cyclohexyl | 1.49[a] |
| I-008 | H | H | H | O | H | 1 | CH₂ | phenyl | 1.32[a] |
| I-009 | H | H | Cl | O | H | 1 | CH₂ | 2-chlorophenyl | 2.12[a] |
| I-010 | H | H | Cl | O | H | 1 | CH₂ | tetrahydro-2H-pyran-3-yl | 1.07[a] |
| I-011 | H | H | Cl | O | H | 1 | CH₂ | tetrahydrofuran-3-yl | 0.73[a] |
| I-012 | H | H | Cl | O | H | 1 | CH₂ | tetrahydro-2H-pyran-4-yl | 0.90[a] |
| I-013 | H | H | Cl | O | H | 1 | CH₂ | tetrahydrofuran-2-yl | 1.02[a] |
| I-014 | H | H | Cl | O | H | 1 | CH₂ | tetrahydro-2H-pyran-2-yl | 1.47[a] |
| I-015 | H | H | Cl | O | H | 1 | CH₂ | 2,5-difluorophenyl | 1.88[a] |
| I-016 | H | H | Cl | O | H | 1 | CH₂ | 2-fluorophenyl | 1.92[a]; |
| | | | | | | | | | 1.90[c] |
| I-017 | H | H | Cl | O | H | 1 | CH₂ | 2,4-difluorophenyl | 1.95[a]; |
| | | | | | | | | | 2.00[c] |
| I-018 | H | H | Cl | O | H | 1 | CH₂ | 2,4-dichlorophenyl | 2.62[a] |
| I-019 | H | H | Cl | O | H | 1 | CH₂ | 2,3-dichlorophenyl | 2.49[a] |
| I-020 | H | H | Cl | O | H | 1 | CH₂ | 4-chlorophenyl | 2.16[a] |
| I-021 | H | H | Cl | O | H | 1 | CH₂ | 3-chlorophenyl | 2.15[a] |
| I-022 | H | H | Cl | O | H | 1 | CH₂ | 3-methoxyphenyl | 1.75[a] |
| I-023 | H | H | Cl | O | H | 1 | —C(CH₂)₂— | 4-fluorophenyl | 2.02[a] |
| I-024 | H | H | Cl | O | H | 1 | CH₂ | 3,5-difluorophenyl | 2.11[a] |
| I-025 | H | H | Cl | O | H | 1 | CH₂ | 4-fluorophenyl | 1.83[a] |
| I-026 | H | H | Cl | O | H | 1 | CH₂ | 3-fluorophenyl | 1.84[a] |
| I-027 | H | H | Cl | O | H | 1 | CH₂ | 3,5-dichlorophenyl | 2.63[a] |
| I-028 | H | H | H | O | H | 1 | CH₂ | 4-chlorophenyl | 1.75[a] |
| I-029 | H | H | H | O | H | 1 | CH₂ | 4-fluorophenyl | 1.27[a] |
| I-030 | H | H | H | O | H | 1 | CH₂ | 2,5-difluorophenyl | 1.49[a] |
| I-031 | H | H | H | O | H | 1 | CH₂ | 2,4-difluorophenyl | 1.56[a] |
| I-032 | H | H | Cl | O | H | 1 | CH₂ | pyridin-4-yl | |
| I-033 | H | H | Cl | O | H | 1 | CH₂ | 1-methyl-1H-pyrazol-4-yl | 0.59[a] |
| I-034 | H | H | Cl | O | H | 1 | CH₂ | 1,3-dimethyl-1H-pyrazol-4-yl | 0.76[a] |
| I-035 | H | H | Cl | O | H | 1 | CH₂ | pyridin-3-yl | |
| I-036 | H | H | Cl | O | H | 1 | CH₂ | 2-chloropyridin-3-yl | 1.22[a] |
| I-037 | H | H | Cl | O | H | 1 | CH₂ | 5-fluoropyridin-3-yl | 0.92[a] |
| I-038 | H | H | Cl | O | H | 1 | CH₂ | 2-fluoropyridin-4-yl | 1.04[a] |
| I-039 | H | H | Cl | O | H | 1 | CH₂ | 4-methylpyrimidin-2-yl | 0.83[a] |
| I-040 | H | benzyl | Cl | O | H | 1 | (S)—CHMe | cyclohexyl | 4.74[a] |
| I-041 | H | H | Me | O | H | 1 | CH₂ | 2,4-difluorophenyl | 1.64[a] |
| I-042 | H | H | Me | O | H | 1 | CH₂ | cyclohexyl | 2.01[a] |
| I-043 | 4-methoxybenzyl | H | Cl | O | H | 1 | CH₂ | 2-fluorophenyl | 3.46[a] |
| I-044 | 4-methoxybenzyl | H | Cl | O | H | 1 | CH₂ | cyclopentyl | 4.04[a] |
| I-045 | 4-methoxybenzyl | H | Cl | O | H | 1 | CH₂ | cycloheptyl | 4.69[a] |
| I-046 | H | benzyl | Cl | O | H | 0 | — | decahydronaphthalen-1-yl | 5.23[a] |
| I-047 | H | benzyl | 1- | O | H | 0 | — | decahydronaphthalen-1-yl | 5.65[a] |
| | | | phenylmethanamino | | | | | | |
| I-048 | H | benzyl | Cl | O | H | 1 | —C(Me)₂— | cyclohexyl | 5.35[a] |
| I-049 | H | H | Me | O | H | 1 | CH₂ | 4-methoxyphenyl | 1.42[a] |
| I-050 | H | H | Me | O | H | 1 | CH₂ | 4-fluorophenyl | 1.52[a] |
| I-051 | H | H | Me | O | H | 1 | CH₂ | 4-chlorophenyl | 1.86[a] |
| I-052 | H | H | H | O | H | 1 | CH₂ | 4-methoxyphenyl | 1.28[a] |
| I-053 | H | H | Cl | O | cyclopropyl | 1 | CH₂ | 2,4-difluorophenyl | 2.62[a] |
| I-054 | H | H | Cl | O | H | 1 | CH₂ | cycloheptyl | 2.86[a] |
| I-055 | H | 4-methoxybenzyl | Cl | O | H | 0 | — | decahydronaphthalen-1-yl | 5.17 + 5.36 + |
| | | | | | | | | | 5.28 + 5.11[a] |
| I-056 | 4-methoxybenzyl | H | Cl | O | H | 1 | CH₂ | 2,2-dimethylcyclohexyl | 4.95[a] |
| I-057 | cyclopropyl | H | Cl | O | H | 1 | CH₂ | 2-fluorophenyl | 2.66[a] |
| I-058 | prop-2-yn-1-yl | H | Cl | O | H | 1 | CH₂ | 2-fluorophenyl | 2.54[a] |
| I-059 | 4-fluorophenyl | H | Cl | O | H | 1 | CH₂ | 2-fluorophenyl | 3.68[a] |
| I-060 | 4-methoxyphenyl | H | Cl | O | H | 1 | CH₂ | 2-fluorophenyl | 3.46[a] |
| I-061 | H | cyclopropyl | Cl | O | H | 1 | (S)—CHMe | cyclohexyl | 3.75[a] |
| I-062 | H | prop-2-yn-1-yl | Cl | O | H | 1 | (S)—CHMe | cyclohexyl | 3.56[a] |
| I-063 | H | 4-fluorophenyl | Cl | O | H | 1 | (S)—CHMe | cyclohexyl | 4.81[a] |
| I-064 | H | phenyl | Cl | O | H | 1 | (S)—CHMe | cyclohexyl | 4.78[a] |
| I-065 | H | 4-methoxybenzyl | Cl | O | H | 1 | (S)—CHMe | cyclohexyl | 4.66[a] |
| I-066 | H | allyl | Cl | O | H | 1 | (S)—CHMe | cyclohexyl | 4.02[a] |
| I-067 | phenyl | H | Cl | O | H | 1 | CH₂ | 2-fluorophenyl | 3.63[a] |
| I-068 | H | 4-methoxyphenyl | Cl | O | H | 1 | (S)—CHMe | cyclohexyl | 4.59[a] |
| I-069 | benzyl | H | Cl | O | H | 1 | CH₂ | 2-fluorophenyl | 3.56[a] |
| I-070 | H | 4-methoxybenzyl | Cl | O | H | 1 | (R)—CHMe | cyclohexyl | 4.56[a] |
| I-071 | allyl | H | Cl | O | H | 1 | CH₂ | 2-fluorophenyl | 2.89[a] |
| I-072 | 4-methoxybenzyl | [((R)-1- | Cl | O | H | 1 | (R)—CHMe | cyclohexyl | 4.54[a] |
| | | cyclohexylethyl)ami- | | | | | | | |
| | | no](oxo)acetyl | | | | | | | |
| I-073 | H | H | Cl | O | H | 1 | CH₂ | cyclopentyl | 2.15[a] |
| I-074 | H | H | Cl | O | H | 0 | — | decahydronaphthalen-1-yl | 3.37[a] |
| I-075 | H | 4-methoxybenzyl | Cl | O | H | 0 | — | 2,2-dimethylcyclohexyl | 4.64[a] |
| I-076 | 4-methoxybenzyl | H | Cl | O | H | 1 | CH₂ | 2,2,6-trimethylcyclohexyl | 5.48[a] |
| I-077 | H | H | Cl | O | H | 1 | (R)—CHMe | cyclohexyl | 2.78[a] |
| I-078 | 4-methoxybenzyl | H | Cl | O | H | 1 | CH₂ | cyclohex-3-en-1-yl | 3.90[a] |
| I-079 | 4-methoxybenzyl | H | Cl | O | H | 1 | CH₂ | 1-methylcyclohexyl | 4.69[a] |
| I-080 | H | H | Cl | O | H | 1 | CH₂ | 2,2-dimethylcyclohexyl | 3.05[a] |
| I-081 | H | H | Cl | O | cyclopropyl | 1 | CH₂ | 2,2,6-trimethylcyclohexyl | 4.08[a] |
| I-082 | H | H | Cl | O | H | 0 | — | 2,2-dimethylcyclohexyl | 2.66[a] |
| I-083 | H | H | Cl | O | H | 1 | CH₂ | 2,2,6-trimethylcyclohexyl | 3.54[a] |
| I-084 | 4-methoxybenzyl | H | Cl | O | H | 1 | CH₂ | cyclohex-1-en-1-yl | 4.02[a] |
| I-085 | H | H | Cl | O | H | 1 | CH₂ | cyclohex-3-en-1-yl | 2.14[a] |
| I-086 | H | H | Cl | O | H | 1 | CH₂ | 1-methylcyclohexyl | 2.84[a] |
| I-087 | 4-methoxybenzyl | Me | Cl | O | H | 1 | (S)—CHMe | cyclohexyl | 5.51[a] |
| I-088 | Me | 4-methoxybenzyl | Cl | O | H | 1 | CH₂ | 2-fluorophenyl | 4.21[a] |
| I-089 | H | H | Cl | O | H | 1 | CH₂ | cyclohex-1-en-1-yl | 2.30[a] |
| I-090 | H | cyclopropylcarbonyl | Cl | O | H | 1 | (S)—CHMe | cyclohexyl | 2.99[a] |
| I-091 | H | benzoyl | Cl | O | H | 1 | (S)—CHMe | cyclohexyl | 3.64[a] |
| I-092 | Me | Me | cyano | O | H | 1 | CH₂ | cyclohexyl | |
| I-093 | cyclopropylcarbonyl | H | Cl | O | H | 1 | CH₂ | 2-fluorophenyl | 2.12[a] |
| I-094 | acetyl | acetyl | Cl | O | H | 1 | (S)—CHMe | cyclohexyl | 3.73[a] |
| I-095 | Me | H | Cl | O | H | 1 | CH₂ | cyclohexyl | |
| I-096 | Me | Me | Cl | O | H | 1 | CH₂ | cyclohexyl | |
| I-097 | H | acetyl | Cl | O | H | 1 | (S)—CHMe | cyclohexyl | 2.51[a] |
| I-098 | 4-methoxybenzyl | H | Cl | O | H | 1 | CH₂ | cyclohexyl | 4.20[a] |
| I-099 | H | H | H | O | H | 1 | CH₂ | 6-methoxypyridin-3-yl | 0.86[a] |
| I-100 | acetyl | H | Cl | O | H | 1 | CH₂ | 2-fluorophenyl | 1.70[a] |
| I-101 | acetyl | acetyl | Cl | O | H | 1 | CH₂ | 2-fluorophenyl | 2.64[a] |
| I-102 | 4-methoxybenzyl | H | Cl | O | H | 1 | CH₂ | 4-methoxyphenyl | 3.46[a] |
| I-103 | methylsulfonyl | methylsulfonyl | Cl | O | H | 1 | (S)—CHMe | cyclohexyl | 3.76[a] |
| I-104 | H | H | Me | O | H | 1 | CH₂ | 2-fluoro-4-(trifluoromethyl)phenyl | 2.18[a] |
| I-105 | 4-methoxybenzyl | H | Cl | O | H | 1 | CH₂ | 1-hydroxycyclohexyl | 3.09[a] |
| I-106 | 4-methoxybenzyl | H | Cl | O | H | 1 | CH₂ | 3-(trifluoromethyl)cyclohexyl | 4.33[a] |
| I-107 | 4-methoxybenzyl | H | Cl | O | H | 1 | CH₂ | 4-(trifluoromethyl)cyclohexyl | 4.30[a] |
| I-108 | H | methoxycarbonyl | Cl | O | H | 1 | (S)—CHMe | cyclohexyl | 2.84[a] |
| I-109 | propionyl | H | Cl | O | H | 1 | CH₂ | cyclohexyl | 2.50[a] |
| I-110 | H | H | Me | O | H | 1 | CH₂ | 6-chloropyridin-3-yl | 1.09[a] |
| I-111 | H | 4-methoxybenzyl | Cl | O | H | 0 | — | 2,3-dihydro-1H-inden-1-yl | 4.04[a] |
| I-112 | H | 4-methoxybenzyl | Cl | O | H | 1 | CHMe | 2-chlorophenyl | 4.16[a] |
| I-113 | H | 4-methoxybenzyl | Cl | O | H | 1 | —C(CH₂)₂— | cyclohexyl | 4.69[a] |
| I-114 | H | H | Cl | O | H | 1 | CH₂ | 6-methoxypyridin-3-yl | 1.23[a] |
| I-115 | H | H | Cl | O | H | 0 | — | cyclopropyl | 0.84[a] |
| I-116 | H | H | Cl | O | cyclopropyl | 1 | CH₂ | mesityl | 3.46[a] |
| I-117 | H | H | Cl | O | cyclopropyl | 1 | CH₂ | 3,5-dimethylphenyl | 3.15[a] |
| I-118 | H | H | Cl | O | cyclopropyl | 1 | CH₂ | 2-isopropylphenyl | 3.33[a] |
| I-119 | H | H | Cl | O | cyclopropyl | 1 | CH₂ | 2,5-dimethylphenyl | 3.04[a] |
| I-120 | H | H | Cl | O | H | 1 | CH₂ | 2,3-difluorophenyl | 1.91[a] |
| I-121 | H | H | Cl | O | H | 1 | CH₂ | 2,3,4-trifluorophenyl | 2.09[a] |
| I-122 | H | H | Cl | O | H | 1 | CH₂ | 6-chloropyridin-3-yl | 1.31[a] |
| I-123 | H | H | Cl | O | H | 1 | CH₂ | 2,3-difluoro-4-methylphenyl | 2.26[a] |
| I-124 | H | H | Cl | O | H | 1 | CH₂ | 2-(difluoromethyl)phenyl | 2.01[a] |
| I-125 | H | H | Cl | O | H | 1 | CH₂ | 3,4-difluorophenyl | 1.93[a] |
| I-126 | H | H | Cl | O | H | 1 | CH₂ | 2-fluoro-4-methoxyphenyl | 1.88[a] |
| I-127 | H | H | Cl | O | H | 1 | CH₂ | 5-fluoro-2-methylphenyl | 2.08[a] |
| I-128 | H | H | Cl | O | H | 1 | CH₂ | 2,6-difluorophenyl | 1.81[a] |
| I-129 | H | H | Cl | O | H | 1 | CH₂ | 2,3,6-trifluorophenyl | 1.91[a] |
| I-130 | H | H | Cl | O | H | 1 | CH₂ | 2,3,5-trifluorophenyl | 2.03[a] |
| I-131 | H | H | Cl | O | H | 1 | CH₂ | 3,4,5-trifluorophenyl | 2.13[a] |
| I-132 | H | H | Cl | O | H | 1 | CH₂ | 2-chloro-6-fluorophenyl | 2.06[a] |
| I-133 | H | H | Cl | O | H | 1 | CH₂ | 4-(difluoromethyl)phenyl | 1.91[a] |
| I-134 | H | H | Cl | O | H | 1 | CH₂ | 2,4,6-trifluorophenyl | 1.96[a] |
| I-135 | (prop-2-yn-1- | (prop-2-yn-1- | Cl | O | H | 1 | (S)—CHMe | cyclohexyl | 3.96[a] |
| | yloxy)carbonyl | yloxy)carbonyl | | | | | | | |
| I-136 | H | H | Me | O | H | 1 | CH₂ | 6-methoxypyridin-3-yl | 0.96[a] |
| I-137 | H | (prop-2-yn-1- | Cl | O | H | 1 | (S)—CHMe | cyclohexyl | 3.17[a] |
| | | yloxy)carbonyl | | | | | | | |
| I-138 | H | H | Cl | O | H | 1 | CH₂ | 1-hydroxycyclohexyl | 1.51[a] |
| I-139 | H | H | Cl | O | H | 1 | CH₂ | 3-(trifluoromethyl)cyclohexyl | 2.66[a] |
| I-140 | H | H | Cl | O | H | 0 | — | 2,3-dihydro-1H-inden-1-yl | 2.30[a] |
| I-141 | H | H | Cl | O | H | 1 | CH₂ | 4-(trifluoromethyl)cyclohexyl | 2.71[a] |
| I-142 | H | H | Cl | O | H | 1 | CHMe | 2-chlorophenyl | 2.46[a] |
| I-143 | H | H | Cl | O | H | 1 | —C(CH₂)₂— | cyclohexyl | 2.82[a] |
| I-144 | H | H | Me | O | H | 1 | CH₂ | 5-fluoro-2-methylphenyl | |
| I-145 | H | H | Me | O | H | 1 | CH₂ | 2-fluoro-4-methylphenyl | 1.81[a] |
| I-146 | H | H | Me | O | H | 1 | CH₂ | 2,6-difluorophenyl | 1.49[a] |
| I-147 | H | H | Me | O | H | 1 | CH₂ | 2,3-difluorophenyl | 1.64[a] |
| I-148 | H | H | Me | O | H | 1 | CH₂ | 2,3-difluoro-4-methylphenyl | |
| I-149 | H | H | Me | O | H | 1 | CH₂ | 3,4-difluorophenyl | 1.69[a] |
| I-150 | H | H | Me | O | H | 1 | CH₂ | 4-cyano-2-fluorophenyl | 1.35[a] |
| I-151 | H | H | Cl | O | cyclopropyl | 1 | CH₂ | 2,4,5-trimethylphenyl | 3.39[a] |
| I-152 | H | H | Cl | O | cyclopropyl | 1 | CH₂ | 2,4-dimethylphenyl | 3.09[a] |
| I-153 | H | H | Cl | O | cyclopropyl | 1 | CH₂ | 2-ethylphenyl | 3.04[a] |
| I-154 | H | H | Cl | O | cyclopropyl | 1 | CH₂ | 2,6-dimethylphenyl | 3.02[a] |
| I-155 | tert-butoxycarbonyl | H | I | O | H | 1 | CH₂ | cyclohexyl | |
| I-156 | H | H | Me | O | H | 1 | CH₂ | 4-(difluoromethyl)phenyl | 1.66[a] |
| I-157 | H | H | Me | O | H | 1 | CH₂ | 2-(difluoromethyl)phenyl | 1.74[a] |
| I-158 | H | H | I | O | H | 1 | CH₂ | cyclohexyl | |
| I-159 | H | H | Me | O | H | 1 | CH₂ | 4-chloro-2-fluorophenyl | 2.00[a] |
| I-160 | tert-butoxycarbonyl | H | I | O | H | 1 | CH₂ | 2,4-difluorophenyl | |
| I-161 | tert-butoxycarbonyl | H | I | O | H | 1 | CH₂ | 4-fluorophenyl | |
| I-162 | benzyl | benzyl | H | O | H | 1 | CH₂ | 3-chloro-5- | 4.98[a] |
| | | | | | | | | (trifluoromethyl)pyridin-2-yl | |
| I-163 | H | H | Cl | O | H | 1 | CH₂ | 3,4-difluoro-2-methylphenyl | 2.23[a] |
| I-164 | H | H | Cl | O | H | 1 | CH₂ | 2-fluoro-4-methylphenyl | 2.18[a] |
| I-165 | H | H | H | O | H | 1 | CH₂ | 2,3,6-trifluorophenyl | 1.51[a] |
| I-166 | H | H | H | O | H | 1 | CH₂ | 3,4-difluorophenyl | 1.54[a] |
| I-167 | acetyl | H | Cl | O | H | 1 | CH₂ | cyclohexyl | 2.14[a] |
| I-168 | benzoyl | H | Cl | O | H | 1 | CH₂ | cyclohexyl | 3.19[a] |
| I-169 | H | H | I | O | H | 1 | CH₂ | 4-fluorophenyl | |
| I-170 | butyryl | H | Cl | O | H | 1 | CH₂ | cyclohexyl | 2.84[a] |
| I-171 | H | H | H | O | H | 1 | CH₂ | 2-fluoro-4-methoxyphenyl | 1.48[a] |
| I-172 | 2,2- | H | Cl | O | H | 1 | CH₂ | cyclohexyl | 3.18[a] |
| | dimethylpropanoyl | | | | | | | | |
| I-173 | H | H | Cl | O | H | 1 | CH₂ | 1-ethyl-3-methyl-1H-pyrazol-4-yl | |
| I-174 | H | tert-butoxycarbonyl | I | O | H | 0 | — | cyclohexyl | |
| I-175 | tert-butoxycarbonyl | H | I | O | H | 1 | CH₂ | tetrahydro-2H-pyran-4-yl | |
| I-176 | tert-butoxycarbonyl | H | I | O | H | 1 | CH₂ | tetrahydro-2H-pyran-2-yl | |
| I-177 | tert-butoxycarbonyl | H | I | O | H | 1 | CH₂ | 1-methylcyclohexyl | |
| I-178 | tert-butoxycarbonyl | H | I | O | H | 1 | CH₂ | 3-(trifluoromethyl)cyclohexyl | |
| I-179 | H | H | H | O | H | 1 | CH₂ | 4-chloro-2-fluorophenyl | 1.82[a] |
| I-180 | H | H | H | O | H | 1 | CH₂ | 2,4,6-trifluorophenyl | 1.50[a] |
| I-181 | H | H | Cl | O | H | 1 | CH₂ | 4-chloro-2-fluorophenyl | 2.40[a] |
| I-182 | 4-fluorobenzoyl | H | Cl | O | H | 1 | CH₂ | cyclohexyl | 3.30[a]; |
| | | | | | | | | | 3.30[c] |
| I-183 | H | H | Cl | O | cyclopropyl | 1 | CH₂ | phenyl | 2.41[a] |
| I-184 | H | H | Cl | O | cyclopropyl | 1 | CH₂ | 2-ethyl-6-methylphenyl | 3.35[a] |
| I-185 | methylsulfonyl | methylsulfonyl | Cl | O | H | 1 | CH₂ | 2-fluorophenyl | 2.80[a] |
| I-186 | H | H | I | O | H | 1 | CH₂ | 2,4-difluorophenyl | |
| I-187 | tert-butoxycarbonyl | H | I | O | H | 1 | CH₂ | 4-chlorophenyl | |
| I-188 | tert-butoxycarbonyl | H | I | O | H | 1 | CH₂ | phenyl | |
| I-189 | tert-butoxycarbonyl | H | I | O | H | 1 | CH₂ | cycloheptyl | |
| I-190 | tert-butoxycarbonyl | H | I | O | H | 1 | CH₂ | 4-(trifluoromethyl)cyclohexyl | |
| I-191 | tert-butoxycarbonyl | H | I | O | H | 1 | CH₂ | cyclopentyl | |
| I-192 | H | H | I | O | H | 1 | CH₂ | 4-chlorophenyl | |
| I-193 | H | H | I | O | H | 1 | CH₂ | 4-(trifluoromethyl)cyclohexyl | |
| I-194 | H | H | I | O | H | 1 | CH₂ | cyclopentyl | |
| I-195 | H | H | I | O | H | 1 | CH₂ | cycloheptyl | |
| I-196 | H | H | I | O | H | 1 | CH₂ | phenyl | |
| I-197 | tert-butoxycarbonyl | H | (trimethylsilyl)ethynyl | O | H | 1 | CH₂ | cyclohexyl | |
| I-198 | H | H | (trimethylsilyl)ethynyl | O | H | 1 | CH₂ | cyclohexyl | |
| I-199 | tert-butoxycarbonyl | H | H | O | H | 1 | CH₂ | cyclohexyl | |
| I-200 | benzyl | benzyl | Br | O | H | 1 | CH₂ | 3-chloro-5- | 6.06[a] |
| | | | | | | | | (trifluoromethyl)pyridin-2-yl | |
| I-201 | H | H | Cl | O | H | 0 | — | rel-[(1R,2R)-2,6-dimethyl-2,3- | |
| | | | | | | | | dihydro-1H-inden-1-yl] | |
| I-202 | H | H | Cl | O | H | 1 | CH₂ | 4-cyano-2-fluorophenyl | 1.64[a] |
| I-203 | H | H | I | O | H | 1 | CH₂ | 1-ethyl-3-methyl-1H-pyrazol-4-yl | |
| I-204 | H | H | Cl | O | H | 1 | CH₂ | piperidin-4-yl | |
| I-205 | tert-butoxycarbonyl | H | I | O | H | 1 | CH₂ | 1-(tert-butoxycarbonyl)piperidin-2- | |
| | | | | | | | | yl | |
| I-206 | tert-butoxycarbonyl | H | I | O | H | 1 | CH₂ | 1-(tert-butoxycarbonyl)piperidin-4- | |
| | | | | | | | | yl | |
| I-207 | tert-butoxycarbonyl | H | I | O | H | 1 | CH₂ | 1-ethyl-3-methyl-1H-pyrazol-4-yl | |
| I-208 | H | H | Cl | O | cyclopropyl | 1 | CH₂ | 2,6-dichlorophenyl | 3.02[a] |
| I-209 | H | H | Cl | O | cyclopropyl | 1 | CH₂ | 2,4-dichlorophenyl | 3.37[a] |
| I-210 | H | H | Cl | O | cyclopropyl | 1 | CH₂ | 2-chloro-6-fluorophenyl | 2.73[a] |
| I-211 | H | H | Cl | O | cyclobutyl | 1 | CH₂ | 2,4-dichlorophenyl | 3.85[a] |
| I-212 | H | H | Cl | O | cyclopropylmethyl | 1 | CH₂ | 2,4-dichlorophenyl | 3.74[a] |
| I-213 | H | H | Cl | O | iPr | 1 | CH₂ | 2,4-dichlorophenyl | 3.71[a] |
| I-214 | H | H | Cl | O | 2,2-dimethyl- | 1 | CH₂ | 2,4-dichlorophenyl | 4.12[a] |
| | | | | | cyclopropyl | | | | |
| I-215 | H | H | Cl | O | cyclopropyl | 1 | CH₂ | 2-chloro-6-(trifluoromethyl)phenyl | 3.19[a] |
| I-216 | H | H | Cl | O | cyclopropyl | 1 | CH₂ | 3-chloro-5-(trifluoromethyl)phenyl | 3.53[a] |
| I-217 | H | H | I | O | H | 1 | CH₂ | 3-(trifluoromethyl)cyclohexyl | |
| I-218 | H | H | Cl | O | H | 1 | CH₂ | 1-(tert-butoxycarbonyl)piperidin-2- | |
| | | | | | | | | yl | |
| I-219 | H | H | Cl | O | H | 1 | CH₂ | 1-(tert-butoxycarbonyl)piperidin-4- | |
| | | | | | | | | yl | |
| I-220 | 4-methoxybenzyl | H | Cl | O | H | 1 | CH₂ | 4-fluoro-2-(trifluoromethyl)phenyl | 4.18[a] |
| I-221 | 4-methoxybenzyl | H | Cl | O | H | 1 | CH₂ | 4-phenoxyphenyl | 4.41[a] |
| I-222 | 4-methoxybenzyl | H | Cl | O | H | 1 | CH₂ | 4-(4-fluorophenoxy)phenyl | 4.44[a] |
| I-223 | 4-methoxybenzyl | H | Cl | O | H | 1 | CH₂ | 4-fluoro-3-phenoxyphenyl | 4.34[a] |
| I-224 | 4-methoxybenzyl | H | Cl | O | H | 1 | CH₂ | 3-fluoro-4-methoxyphenyl | 3.44[a] |
| I-225 | 4-methoxybenzyl | H | Cl | O | H | 1 | CH₂ | 4-chloro-2-fluorophenyl | 4.03[a] |
| I-226 | 4-methoxybenzyl | H | Cl | O | H | 1 | CH₂ | 2,4-difluorophenyl | |
| I-227 | H | H | Cl | O | H | 1 | CH₂ | 4-phenoxyphenyl | 2.94[a] |
| I-228 | chloroacetyl | H | Cl | O | H | 1 | CH₂ | cyclohexyl | |
| I-229 | bromoacetyl | H | Cl | O | H | 1 | CH₂ | cyclohexyl | |
| I-230 | fluoroacetyl | H | Cl | O | H | 1 | CH₂ | cyclohexyl | |
| I-231 | methoxy(oxo)acetyl | H | Cl | O | H | 1 | CH₂ | cyclohexyl | |
| I-232 | 3-methoxy-3- | H | Cl | O | H | 1 | CH₂ | cyclohexyl | |
| | oxopropanoyl | | | | | | | | |
| I-233 | methoxyacetyl | H | Cl | O | H | 1 | CH₂ | cyclohexyl | |
| I-234 | methoxycarbonyl | H | Cl | O | H | 1 | CH₂ | cyclohexyl | |
| I-235 | trifluoroacetyl | H | Cl | O | H | 1 | CH₂ | cyclohexyl | |
| I-236 | 4-methoxybenzyl | H | Cl | O | H | 1 | CH₂ | 2-fluoro-3-methoxyphenyl | 3.46[a] |
| I-237 | H | H | Cl | O | H | 1 | CH₂ | 4-fluoro-3-phenoxyphenyl | 2.90[a] |
| I-238 | H | H | Cl | O | H | 1 | CH₂ | 4-(4-fluorophenoxy)phenyl | 3.00[a] |
| I-239 | methoxycarbonyl | H | Cl | O | H | 1 | CH₂ | 2-fluorophenyl | 1.98[a] |
| I-240 | H | H | Cl | O | H | 1 | CH₂ | 3-fluoro-4-methoxyphenyl | 1.91[a] |
| I-241 | tert-butoxycarbonyl | tert-butoxycarbonyl | 3- | O | H | 1 | CH₂ | cyclohexyl | |
| | | | (trifluoromethyl)phenyl | | | | | | |
| I-242 | 4-methoxybenzyl | H | Cl | O | H | 1 | CH₂ | 4-chlorophenyl | 3.99[a] |
| I-243 | H | H | Cl | O | H | 1 | CH₂ | 3,3-dimethylcyclobutyl | 2.49[a] |
| I-244 | 4-methoxybenzyl | H | Cl | O | H | 1 | CH₂ | 3,3-dimethylcyclobutyl | |
| I-245 | H | H | I | O | H | 1 | CH₂ | 1-methylcyclohexyl | |
| I-246 | H | H | I | O | H | 1 | CH₂ | tetrahydro-2H-pyran-2-yl | |
| I-247 | H | H | I | O | H | 1 | CH₂ | tetrahydro-2H-pyran-4-yl | |
| I-248 | fluoroacetyl | H | Cl | O | H | 1 | CH₂ | 2,4-difluorophenyl | |
| I-249 | chloroacetyl | H | Cl | O | H | 1 | CH₂ | 2,4-difluorophenyl | |
| I-250 | 3-methoxy-3- | H | Cl | O | H | 1 | CH₂ | 2,4-difluorophenyl | |
| | oxopropanoyl | | | | | | | | |
| I-251 | bromoacetyl | H | Cl | O | H | 1 | CH₂ | 2,4-difluorophenyl | |
| I-252 | methoxy(oxo)acetyl | H | Cl | O | H | 1 | CH₂ | 2,4-difluorophenyl | |
| I-253 | methoxyacetyl | H | Cl | O | H | 1 | CH₂ | 2,4-difluorophenyl | |
| I-254 | methoxyacetyl | methoxyacetyl | Cl | O | H | 1 | CH₂ | cyclohexyl | |
| I-255 | H | H | Cl | O | H | 1 | CH₂ | cyclobutyl | |
| I-256 | H | H | 3- | O | H | 1 | CH₂ | cyclohexyl | |
| | | | (trifluoromethyl)phenyl | | | | | | |
| I-257 | H | H | hydroxy(phenyl)methyl | O | H | 1 | CH₂ | cyclohexyl | |
| I-258 | tert-butoxycarbonyl | H | vinyl | O | H | 1 | CH₂ | 2,4-difluorophenyl | |
| I-259 | tert-butoxycarbonyl | H | vinyl | O | H | 1 | CH₂ | cyclohexyl | |
| I-260 | 2-furoyl | 2-furoyl | Cl | O | H | 1 | CH₂ | cyclohexyl | |
| I-261 | dichloroacetyl | H | Cl | O | H | 1 | CH₂ | cyclohexyl | |
| I-262 | 4-methoxy-4- | H | Cl | O | H | 1 | CH₂ | cyclohexyl | |
| | oxobutanoyl | | | | | | | | |
| I-263 | benzyl | benzyl | difluoromethyl | O | H | 1 | (S)—CHMe | cyclohexyl | 6.20[a] |
| I-264 | benzyl | benzyl | cyclopropyl | O | H | 1 | (S)—CHMe | cyclohexyl | 6.20[a] |
| I-265 | tert-butoxycarbonyl | tert-butoxycarbonyl | —SMe | O | H | 1 | CH₂ | cyclohexyl | |
| I-266 | tert-butoxycarbonyl | tert-butoxycarbonyl | —SMe | O | H | 1 | CH₂ | 2,4-difluorophenyl | |
| I-267 | H | 4-methoxybenzyl | Cl | O | H | 0 | — | 2-phenylcyclohexyl | 4.69[a] |
| I-268 | 4-methoxybenzyl | H | Cl | O | H | 1 | CH₂ | 3-chloro-5- | 4.29[a] |
| | | | | | | | | (trifluoromethyl)pyridin-2-yl | |
| I-269 | H | 4-methoxybenzyl | Cl | O | H | 0 | — | 2-phenylcyclopentyl | 4.41[a] |
| I-270 | H | H | difluoromethyl | O | H | 1 | (S)—CHMe | cyclohexyl | 2.96[a] |
| I-271 | H | H | cyclopropyl | O | H | 1 | (S)—CHMe | cyclohexyl | 2.68[a] |
| I-272 | H | H | —SMe | O | H | 1 | CH₂ | 2,4-difluorophenyl | |
| I-273 | H | H | vinyl | O | H | 1 | CH₂ | cyclohexyl | |
| I-274 | H | H | vinyl | O | H | 1 | CH₂ | 2,4-difluorophenyl | |
| I-275 | H | H | —SMe | O | H | 1 | CH₂ | cyclohexyl | |
| I-276 | H | H | Cl | O | 2-furoyl | 1 | CH₂ | 2,4-difluorophenyl | |
| I-277 | H | H | Cl | O | 2-thienylcarbonyl | 1 | CH₂ | 2,4-difluorophenyl | |
| I-278 | H | H | Cl | O | 3-furoyl | 1 | CH₂ | 2,4-difluorophenyl | |
| I-279 | 3-thienylcarbonyl | H | Cl | O | H | 1 | CH₂ | 2,4-difluorophenyl | |
| I-280 | trifluoroacetyl | H | Cl | O | H | 1 | CH₂ | 2,4-difluorophenyl | 2.64[a] |
| I-281 | H | H | H | O | H | 1 | CH₂ | 3-chlorophenyl | 1.78[a] |
| I-282 | H | H | H | O | H | 1 | CH₂ | 2-chlorophenyl | 1.69[a] |
| I-283 | benzyl | benzyl | H | O | H | 1 | CH₂ | 3-chlorophenyl | 4.71[a] |
| I-284 | benzyl | benzyl | H | O | H | 1 | CH₂ | 2-chlorophenyl | 4.67[a] |
| I-285 | benzyl | benzyl | H | O | H | 1 | CH₂ | 3,5-difluorophenyl | 4.53[a] |
| I-286 | H | H | Cl | O | H | 1 | CH₂ | 3-chloro-5- | 2.58[a] |
| | | | | | | | | (trifluoromethyl)pyridin-2-yl | |
| I-287 | H | H | Cl | O | H | 0 | — | 2-phenylcyclohexyl | 3.02[a] |
| I-288 | H | H | Cl | O | H | 0 | — | 2-phenylcyclopentyl | 2.71[a] |
| I-289 | difluoroacetyl | H | Cl | O | H | 1 | CH₂ | 2,4-difluorophenyl | |
| I-290 | difluoroacetyl | H | Cl | O | H | 1 | CH₂ | cyclohexyl | |
| I-291 | acryloyl | H | Cl | O | H | 1 | CH₂ | cyclohexyl | |
| I-292 | tert-butoxycarbonyl | H | —SPh | O | H | 1 | CH₂ | cyclohexyl | |
| I-293 | tert-butoxycarbonyl | H | —SPh | O | H | 1 | CH₂ | 2,4-difluorophenyl | |
| I-294 | H | H | —SPh | O | H | 1 | CH₂ | cyclohexyl | |
| I-295 | H | H | —SPh | O | H | 1 | CH₂ | 2,4-difluorophenyl | |
| I-296 | cyclopropylcarbonyl | H | Cl | O | H | 1 | CH₂ | 3-chloro-5- | 2.71[a] |
| | | | | | | | | (trifluoromethyl)pyridin-2-yl | |
| I-297 | H | cyclopropylcarbonyl | Cl | O | H | 0 | — | 2-phenylcyclopentyl | 2.86[a] |
| I-298 | tert-butoxycarbonyl | tert-butoxycarbonyl | trifluoromethyl | O | H | 1 | CH₂ | cyclohexyl | |
| I-299 | tert-butoxycarbonyl | tert-butoxycarbonyl | trifluoromethyl | O | H | 1 | CH₂ | 2,4-difluorophenyl | |
| I-300 | H | H | trifluoromethyl | O | H | 1 | CH₂ | cyclohexyl | |
| I-301 | H | H | trifluoromethyl | O | H | 1 | CH₂ | 2,4-difluorophenyl | |
| I-302 | H | cyclopropylcarbonyl | Cl | O | H | 0 | — | 2-phenylcyclohexyl | 3.19[a] |
| I-303 | H | H | H | O | H | 1 | CH₂ | 3,5-difluorophenyl | 4.55[a] |
| I-304 | H | H | H | O | H | 1 | CH₂ | 3-fluorophenyl | 1.46[a] |
| I-305 | H | H | H | O | H | 1 | CH₂ | 2-fluorophenyl | 1.41[a] |
| I-306 | H | H | H | O | H | 1 | CH₂ | 2,4-dichlorophenyl | 2.11[a] |
| I-307 | H | H | H | O | H | 1 | CH₂ | 3,5-dichlorophenyl | 2.18[a] |
| I-308 | H | H | H | O | H | 1 | CH₂ | 2,3-dichlorophenyl | 2.02[a] |
| I-309 | benzyl | benzyl | H | O | H | 1 | CH₂ | 4-fluorophenyl | 4.36[a] |
| I-310 | benzyl | benzyl | H | O | H | 1 | CH₂ | 3-fluorophenyl | 4.40[a] |
| I-311 | benzyl | benzyl | H | O | H | 1 | CH₂ | 2-fluorophenyl | 4.38[a] |
| I-312 | benzyl | benzyl | H | O | H | 1 | CH₂ | 3,5-dichlorophenyl | 2.90[a] |
| I-313 | benzyl | benzyl | H | O | H | 1 | CH₂ | 2,3-dichlorophenyl | 5.01[a] |
| I-314 | benzyl | benzyl | H | O | H | 1 | CH₂ | 4-chlorophenyl | 4.85[a] |
| I-315 | tert-butoxycarbonyl | H | ethynyl | O | H | 1 | CH₂ | 2,4-difluorophenyl | |
| I-316 | H | H | ethynyl | O | H | 1 | CH₂ | cyclohexyl | |
| I-317 | benzyl | benzyl | 4-fluorophenyl | O | H | 1 | (S)—CHMe | cyclohexyl | 6.63[a] |
| I-318 | benzyl | benzyl | 4-fluorophenyl | O | H | 1 | CH₂ | cyclohexyl | 6.27[a] |
| I-319 | benzyl | benzyl | 4-fluorophenyl | O | H | 0 | — | cyclohexyl | 5.98[a] |
| I-320 | benzyl | benzyl | 4-fluorophenyl | O | H | 1 | CH₂ | 2-fluorophenyl | 5.45[a] |
| I-321 | benzyl | benzyl | 4-fluorophenyl | O | H | 1 | CH₂ | 4-chlorophenyl | 5.72[a] |
| I-322 | benzyl | benzyl | H | O | H | 1 | CH₂ | 2,4-difluorophenyl | 4.45[a] |
| I-323 | benzyl | benzyl | H | O | H | 1 | CH₂ | 2,4-dichlorophenyl | 5.17[a] |
| I-324 | H | H | Cl | O | H | 1 | CH₂ | 4-fluoro-2-(trifluoromethyl)phenyl | 2.61[a] |
| I-325 | H | H | Cl | O | H | 1 | CH₂ | 2-fluoro-3-methoxyphenyl | 1.91[a] |
| I-326 | H | H | Cl | O | H | 1 | CH₂ | 2,5-dimethylphenyl | 2.50[a] |
| I-327 | H | H | Cl | O | H | 1 | CH₂ | 5-chloro-2-(trifluoromethyl)phenyl | 2.88[a] |
| I-328 | H | H | Cl | O | H | 1 | CH₂ | 2,4,5-trimethylphenyl | 2.78[a] |
| I-329 | H | H | Cl | O | H | 1 | CH₂ | 2-tert-butyl-5-methylphenyl | 3.46[a] |
| I-330 | tert-butoxycarbonyl | tert-butoxycarbonyl | 4- | O | H | 1 | CH₂ | cyclohexyl | |
| | | | (trifluoromethyl)phenyl | | | | | | |
| I-331 | tert-butoxycarbonyl | tert-butoxycarbonyl | 4- | O | H | 1 | CH₂ | 2,4-difluorophenyl | |
| | | | (trifluoromethyl)phenyl | | | | | | |
| I-332 | H | H | 4-fluorophenyl | O | H | 1 | CH₂ | cyclohexyl | 2.96[a] |
| I-333 | H | H | 4-fluorophenyl | O | H | 1 | (S)—CHMe | cyclohexyl | 3.29[a] |
| I-334 | H | H | 4-fluorophenyl | O | H | 0 | — | cyclohexyl | 2.66[a] |
| I-335 | H | H | 4-fluorophenyl | O | H | 1 | CH₂ | 2-fluorophenyl | 2.43[a] |
| I-336 | H | H | 4-fluorophenyl | O | H | 1 | CH₂ | 4-chlorophenyl | 2.73[a] |
| I-337 | H | H | Cl | O | H | 1 | CH₂ | cyclopropyl | 1.30[a] |
| I-338 | tert-butoxycarbonyl | H | ethynyl | O | H | 1 | CH₂ | cyclohexyl | |
| I-339 | H | H | ethynyl | O | H | 1 | CH₂ | 2,4-difluorophenyl | |
| I-340 | tert-butoxycarbonyl | tert-butoxycarbonyl | phenyl | O | H | 1 | CH₂ | cyclohexyl | |
| I-341 | H | H | allyl | O | H | 1 | CH₂ | 2,4-difluorophenyl | |
| I-342 | tert-butoxycarbonyl | tert-butoxycarbonyl | allyl | O | H | 1 | CH₂ | cyclohexyl | |
| I-343 | H | H | allyl | O | H | 1 | CH₂ | cyclohexyl | |
| I-344 | tert-butoxycarbonyl | tert-butoxycarbonyl | allyl | O | H | 1 | CH₂ | 2,4-difluorophenyl | |
| I-345 | H | H | phenyl | O | H | 1 | CH₂ | cyclohexyl | |
| I-346 | H | trifluoroacetyl | Cl | O | H | 1 | (R)—CHMe | cyclohexyl | |
| I-347 | H | trifluoroacetyl | Cl | O | H | 1 | (S)—CHMe | cyclohexyl | |
| I-348 | H | trifluoroacetyl | Cl | O | H | 1 | CHMe | cyclohexyl | |
| I-349 | H | H | Cl | O | H | 1 | CH₂ | 4-fluoro-3-methylphenyl | 2.28[a] |
| I-350 | 4-methoxybenzyl | H | Cl | O | H | 1 | CH₂ | 4-fluoro-3-methylphenyl | 3.89[a] |
| I-351 | 2- | H | Cl | O | H | 1 | CH₂ | 2,4-difluorophenyl | |
| | (trifluoromethyl)benzoyl | | | | | | | | |
| I-352 | H | H | Cl | O | 3,5- | 1 | CH₂ | 2,4-difluorophenyl | |
| | | | | | difluorobenzoyl | | | | |
| I-353 | H | H | Cl | O | 4- | 1 | CH₂ | 2,4-difluorophenyl | |
| | | | | | (trifluoromethyl)benzoyl | | | | |
| I-354 | H | H | Cl | O | 3,4- | 1 | CH₂ | 2,4-difluorophenyl | |
| | | | | | difluorobenzoyl | | | | |
| I-355 | H | H | Cl | O | 3- | 1 | CH₂ | 2,4-difluorophenyl | |
| | | | | | (trifluoromethyl)benzoyl | | | | |
| I-356 | H | H | —SEt | O | H | 1 | CH₂ | cyclohexyl | |
| I-357 | H | H | —SEt | O | H | 1 | CH₂ | 2,4-difluorophenyl | |
| I-358 | H | H | cyclopropyl | O | H | 1 | CH₂ | 4-chlorophenyl | 2.21[a] |
| I-359 | H | H | cyclopropyl | O | H | 1 | CH₂ | cyclohexyl | 2.37[a] |
| I-360 | H | H | cyclopropyl | O | H | 0 | — | cyclohexyl | 2.00[a] |
| I-361 | H | H | cyclopropyl | O | H | 1 | CH₂ | 2-fluorophenyl | 1.88[a] |
| I-362 | H | H | Cl | O | cyclopropyl | 1 | CH₂ | 2,5-difluorophenyl | 2.57[a] |
| I-363 | H | H | Cl | O | cyclopropyl | 1 | CH₂ | 2-fluoro-5-methylphenyl | 2.80[a] |
| I-364 | H | H | Cl | O | cyclopropyl | 1 | CH₂ | 5-chloro-2-fluorophenyl | 2.86[a] |
| I-365 | H | H | Cl | O | cyclopropyl | 1 | CH₂ | 2-bromo-5-chlorophenyl | 3.29[a] |
| I-366 | 4-methoxybenzyl | H | Cl | O | H | 1 | CH₂ | 4-bromophenyl | 4.01[a] |
| I-367 | 2,2,3,3,3- | H | Cl | O | H | 1 | CH₂ | cyclohexyl | |
| | pentafluoropropanoyl | | | | | | | | |
| I-368 | 2,3-difluorobenzoyl | H | Cl | O | H | 1 | CH₂ | 2,4-difluorophenyl | |
| I-369 | 2,2,3,3,3- | H | Cl | O | H | 1 | CH₂ | 2,4-difluorophenyl | |
| | pentafluoropropanoyl | | | | | | | | |
| I-370 | H | H | Et | O | H | 1 | CH₂ | cyclohexyl | |
| I-371 | tert-butoxycarbonyl | H | Et | O | H | 1 | CH₂ | cyclohexyl | |
| I-372 | H | H | Cl | O | H | 1 | CH₂ | 4-bromophenyl | 2.39[a] |
| I-373 | H | H | trifluoromethyl | O | H | 1 | CH₂ | tetrahydro-2H-pyran-2-yl | |
| I-374 | H | H | trifluoromethyl | O | H | 1 | CH₂ | 2-fluorophenyl | |
| I-375 | H | H | trifluoromethyl | O | H | 1 | (R)—CHMe | cyclohexyl | |
| I-376 | 4-methoxybenzyl | H | Cl | O | H | 1 | CH₂ | 4-chloro-2-methylphenyl | 4.20[a] |
| I-377 | H | H | I | O | H | 1 | CH₂ | 2-fluorophenyl | |
| I-378 | H | H | I | O | H | 1 | (R)—CHMe | cyclohexyl | |
| I-379 | 2,2,3,3,4,4,4- | H | Cl | O | H | 1 | CH₂ | 2,4-difluorophenyl | |
| | heptafluorobutanoyl | | | | | | | | |
| I-380 | 2-bromopropanoyl | H | Cl | O | H | 1 | CH₂ | 2,4-difluorophenyl | |
| I-381 | 2,2,3,3,4,4,4- | H | Cl | O | H | 1 | CH₂ | cyclohexyl | |
| | heptafluorobutanoyl | | | | | | | | |
| I-382 | 2-bromopropanoyl | H | Cl | O | H | 1 | CH₂ | cyclohexyl | |
| I-383 | 3,3,3- | H | Cl | O | H | 1 | CH₂ | 2,4-difluorophenyl | |
| | trifluoropropanoyl | | | | | | | | |
| I-384 | 3,3,3- | H | Cl | O | H | 1 | CH₂ | cyclohexyl | |
| | trifluoropropanoyl | | | | | | | | |
| I-385 | dichloroacetyl | H | Cl | O | H | 1 | CH₂ | 2,4-difluorophenyl | |
| I-386 | 4-methoxy-4- | H | Cl | O | H | 1 | CH₂ | 2,4-difluorophenyl | |
| | oxobutanoyl | | | | | | | | |
| I-387 | bromoacetyl | bromoacetyl | Cl | O | H | 1 | CH₂ | cyclohexyl | |
| I-388 | acryloyl | acryloyl | Cl | O | H | 1 | CH₂ | 2,4-difluorophenyl | |
| I-389 | H | H | Cl | O | H | 1 | CH₂ | 4-chloro-2-methylphenyl | 2.60[a] |
| I-390 | 4-methoxybenzyl | H | Cl | O | H | 1 | CH₂ | 4-chloro-3-methoxyphenyl | 3.76[a] |
| I-391 | 4-methoxybenzyl | H | Cl | O | H | 1 | CH₂ | 5-methyl-2-(1- | 4.92[a] |
| | | | | | | | | methylcyclopropyl)phenyl | |
| I-392 | 4-methoxybenzyl | H | Cl | O | H | 1 | CH₂ | 2-isopropyl-5-methylphenyl | 4.80[a] |
| I-393 | 4-methoxybenzyl | H | Cl | O | H | 1 | CH₂ | 2,5-dichlorophenyl | 4.34[a] |
| I-394 | benzyl | benzyl | difluoromethyl | O | H | 1 | CH₂ | cyclohexyl | 5.88[a] |
| I-395 | tert-butoxycarbonyl | H | Et | O | H | 1 | CH₂ | 2,4-difluorophenyl | |
| I-396 | H | H | trimethylsilyl | O | H | 1 | CH₂ | 2,4-difluorophenyl | |
| I-397 | H | H | trimethylsilyl | O | H | 1 | CH₂ | cyclohexyl | |
| I-398 | trifluoroacetyl | H | H | O | H | 1 | CH₂ | 2,4-difluorophenyl | 2.58[a] |
| I-399 | H | H | Et | O | H | 1 | CH₂ | 2,4-difluorophenyl | |
| I-400 | 2-chloropropanoyl | H | Cl | O | H | 1 | CH₂ | 2,4-difluorophenyl | |
| I-401 | 2-chloropropanoyl | H | Cl | O | H | 1 | CH₂ | cyclohexyl | |
| I-402 | [5-methyl-3- | H | Cl | O | H | 1 | CH₂ | cyclohexyl | |
| | (trifluoromethyl)-1H- | | | | | | | | |
| | pyrazol-1-yl]acetyl | | | | | | | | |
| I-403 | [5-methyl-3- | H | Cl | O | H | 1 | CH₂ | 2,4-difluorophenyl | |
| | (trifluoromethyl)-1H- | | | | | | | | |
| | pyrazol-1-yl]acetyl | | | | | | | | |
| I-404 | (1- | (1-fluoro- | Cl | O | H | 1 | CH₂ | cyclohexyl | |
| | fluorocyclopropyl)carbonyl | cyclopropyl)carbonyl | | | | | | | |
| I-405 | (1-chloro- | (1-chloro- | Cl | O | H | 1 | CH₂ | cyclohexyl | |
| | cyclopropyl)carbonyl | cyclopropyl)carbonyl | | | | | | | |
| I-406 | 4-methoxybenzyl | H | Cl | O | H | 1 | CH₂ | 3-fluorophenyl | |
| I-407 | H | H | Cl | O | (1-chloro- | 1 | CH₂ | 2,4-difluorophenyl | |
| | | | | | cyclopropyl)carbonyl | | | | |
| I-408 | chloro(fluoro)acetyl | H | Cl | O | H | 1 | CH₂ | 2,4-difluorophenyl | |
| I-409 | chloro(fluoro)acetyl | H | Cl | O | H | 1 | CH₂ | cyclohexyl | |
| I-410 | 4-methoxybenzyl | H | Cl | O | H | 1 | CH₂ | 2,4,6-trifluorophenyl | |
| I-411 | 4-methoxybenzyl | H | Cl | O | H | 1 | CH₂ | 3.4-difluorophenyl | |
| I-412 | H | H | Cl | O | (1- | 1 | CH₂ | 2.4-difluorophenyl | |
| | | | | | fluorocyclopropyl)carbonyl | | | | |
| I-413 | 4-methoxybenzyl | H | Cl | O | H | 1 | CH₂ | 2,3,4-trifluorophenyl | |
| I-414 | H | H | Cl | O | H | 1 | CH₂ | 5-methyl-2-(1- | 3.29[a] |
| | | | | | | | | methylcyclopropyl)phenyl | |
| I-415 | H | H | Cl | O | H | 1 | CH₂ | 2-isopropy1-5-methylphenyl | 3.15[a] |
| I-416 | H | H | Cl | O | H | 1 | CH₂ | 2.5-dichlorophenyl | 2.71[a] |
| I-417 | H | H | difluoromethyl | O | H | 1 | CH₂ | cyclohexyl | 2.66[a] |
| I-418 | H | H | Cl | S | H | 1 | (S)—CHMe | cyclohexyl | 3.85[a] |
| I-419 | tert-butoxycarbonyl | H | 1-hydroxyethyl | O | H | 1 | CH₂ | cyclohexyl | |
| I-420 | H | H | 1-hydroxyethyl | O | H | 1 | CH₂ | cyclohexyl | |
| I-421 | H | H | Cl | O | cyclohexylmethyl | 1 | CH₂ | H | |
| I-422 | tert-butoxycarbonyl | tert-butoxycarbonyl | I | O | H | 1 | CH₂ | cyclohexyl | |
| I-423 | H | H | Et | O | H | 1 | (S)—CHMe | cyclohexyl | |
| I-424 | H | H | Et | O | H | 1 | CH₂ | tetrahydro-2H-pyran-2-yl | |
| I-425 | H | H | trifluoromethyl | O | H | 1 | (S)—CHMe | cyclohexyl | |
| I-426 | 4-methoxybenzyl | H | Cl | O | H | 1 | CH₂ | 2,4-difluoro-3-methoxyphenyl | 3.69[a] |
| I-427 | H | 2,2,2-trifluoroethyl | Cl | O | H | 1 | (S)—CHMe | cyclohexyl | 4.11[a] |
| I-428 | H | cyclopropyl | Cl | S | H | 1 | (S)—CHMe | cyclohexyl | 4.84[a] |
| I-429 | H | 2,2,2-trifluoroethyl | Cl | S | H | 1 | (S)—CHMe | cyclohexyl | 5.14[a] |
| I-430 | formyl | H | H | O | H | 1 | CH₂ | 2,4-difluorophenyl | 1.79[a] |
| I-431 | 4-methoxybenzyl | H | Cl | S | H | 1 | CH₂ | 2,4-difluorophenyl | |
| I-432 | (1- | H | Cl | O | H | 1 | CH₂ | cyclohexyl | |
| | chlorocyclopropyl)carbonyl | | | | | | | | |
| I-433 | (1- | H | Cl | O | H | 1 | CH₂ | 2,4-difluorophenyl | |
| | chlorocyclopropyl)carbonyl | | | | | | | | |
| I-434 | H | H | ethynyl | O | H | 1 | CH₂ | tetrahydro-2H-pyran-2-yl | |
| I-435 | 4-methoxybenzyl | H | Cl | O | H | 1 | CH₂ | 2,3,5-trifluorophenyl | |
| I-436 | 4-methoxybenzyl | H | Cl | O | H | 1 | CH₂ | 2,3,6-trifluorophenyl | |
| I-437 | 4-methoxybenzyl | H | Cl | O | H | 1 | CH₂ | 4-fluorophenyl | |
| I-438 | 4-methoxybenzyl | H | Cl | O | H | 1 | CH₂ | 2,6-difluorophenyl | |
| I-439 | 4-methoxybenzyl | H | Cl | O | H | 1 | CH₂ | 2,3-difluorophenyl | |
| I-440 | 4-methoxybenzyl | H | Cl | O | H | 1 | CH₂ | 2,5-difluorophenyl | |
| I-441 | 4-methoxybenzyl | H | Cl | O | H | 1 | CH₂ | 3-chlorophenyl | |
| I-442 | 4-methoxybenzyl | H | Cl | O | H | 1 | CH₂ | 3,5-difluorophenyl | |
| I-443 | 4-methoxybenzyl | H | Cl | O | H | 1 | CH₂ | 2,4-dichlorophenyl | |
| I-444 | H | H | I | O | H | 1 | (S)—CHMe | cyclohexyl | |
| I-445 | tert-butoxycarbonyl | tert-butoxycarbonyl | I | O | H | 1 | (S)—CHMe | cyclohexyl | |
| I-446 | H | tert-butoxycarbonyl | I | O | H | 1 | (S)—CHMe | cyclohexyl | |
| I-447 | 4-methoxybenzyl | H | Cl | O | H | 1 | CH₂ | 3-chloro-4-fluorophenyl | |
| I-448 | H | H | ethynyl | O | H | 1 | CH₂ | 2,4,6-trifluorophenyl | |
| I-449 | trifluoroacetyl | H | Cl | S | H | 1 | CH₂ | cyclohexyl | |
| I-450 | pentanoyl | H | Cl | O | H | 1 | CH₂ | cyclohexyl | 3.20[c] |
| I-451 | fluoroacetyl | H | Cl | S | H | 1 | CH₂ | cyclohexyl | |
| I-452 | 2-chloro-2,3,3,3- | H | Cl | O | H | 1 | CH₂ | cyclohexyl | |
| | tetrafluoropropanoyl | | | | | | | | |
| I-453 | difluoroacetyl | H | Cl | O | H | 1 | CH₂ | 3,4-difluorophenyl | |
| I-454 | (1- | H | Cl | O | H | 1 | CH₂ | cyclohexyl | |
| | fluorocyclopropyl)carbonyl | | | | | | | | |
| I-455 | H | H | pyridin-4-yl | O | H | 1 | CH₂ | cyclohexyl | |
| I-456 | trifluoroacetyl | H | Cl | O | H | 1 | CH₂ | 3,4-difluorophenyl | |
| I-457 | fluoroacetyl | H | Cl | O | H | 1 | CH₂ | 3-fluorophenyl | |
| I-458 | trifluoroacetyl | H | Cl | O | H | 1 | CH₂ | 2,4,6-trifluorophenyl | |
| I-459 | difluoroacetyl | H | Cl | O | H | 1 | CH₂ | 2,4,6-trifluorophenyl | |
| I-460 | acryloyl | acryloyl | Cl | O | H | 1 | CH₂ | cyclohexyl | |
| I-461 | 4-methoxybenzyl | H | Cl | O | H | 1 | CH₂ | 2-chlorophenyl | |
| I-462 | 4-methoxybenzyl | H | Cl | O | H | 1 | CH₂ | 3,4-dichlorophenyl | |
| I-463 | H | H | Cl | O | H | 1 | CH₂ | 3-chloro-4-fluorophenyl | |
| I-464 | H | H | Cl | O | H | 1 | CH₂ | 2,4-difluoro-3-methoxyphenyl | 2.12[a] |
| I-465 | H | H | Cl | O | H | 1 | CH₂ | 2-chloro-3-methoxyphenyl | 2.23[a] |
| I-466 | trifluoroacetyl | H | Cl | O | H | 1 | CH₂ | 2,3,6-trifluorophenyl | |
| I-467 | formyl | H | Cl | O | H | 1 | CH₂ | 2,4-difluorophenyl | 1.89[a] |
| I-468 | difluoroacetyl | H | Cl | O | H | 1 | CH₂ | 4-fluorophenyl | |
| I-469 | difluoroacetyl | H | Cl | O | H | 1 | CH₂ | 2,3,6-trifluorophenyl | |
| I-470 | difluoroacetyl | H | Cl | O | H | 1 | CH₂ | 2,3,4-trifluorophenyl | |
| I-471 | chloroacetyl | H | Cl | O | H | 1 | CH₂ | 2,4,6-trifluorophenyl | |
| I-472 | methoxyacetyl | H | Cl | O | H | 1 | CH₂ | 2,4,6-trifluorophenyl | |
| I-473 | trifluoroacetyl | H | Cl | O | H | 1 | CH₂ | 2,3,4-trifluorophenyl | |
| I-474 | fluoroacetyl | H | Cl | O | H | 1 | CH₂ | 2,4,6-trifluorophenyl | |
| I-475 | 2-chloro-2,3,3,3- | H | Cl | O | H | 1 | CH₂ | 2,4-difluorophenyl | |
| | tetrafluoropropanoyl | | | | | | | | |
| I-476 | 3-chloro-2,2,3,3- | H | Cl | O | H | 1 | CH₂ | cyclohexyl | |
| | tetrafluoropropanoyl | | | | | | | | |
| I-477 | 5-chloro- | H | Cl | O | H | 1 | CH₂ | 2,4-difluorophenyl | |
| | 2,2,3,3,4,4,5,5- | | | | | | | | |
| | octafluoropentanoyl | | | | | | | | |
| I-478 | 3-chloro-2,2,3,3- | H | Cl | O | H | 1 | CH₂ | 2,4-difluorophenyl | |
| | tetrafluoropropanoyl | | | | | | | | |
| I-479 | 5-chloro- | H | Cl | O | H | 1 | CH₂ | cyclohexyl | |
| | 2,2,3,3,4,4,5,5- | | | | | | | | |
| | octafluoropentanoyl | | | | | | | | |
| I-480 | fluoroacetyl | fluoroacetyl | Cl | S | H | 1 | CH₂ | 2,4-difluorophenyl | |
| I-481 | fluoroacetyl | H | Cl | S | H | 1 | CH₂ | 2,4-difluorophenyl | |
| I-482 | trifluoroacetyl | H | Cl | O | H | 1 | CH₂ | 4-fluorophenyl | |
| I-483 | difluoroacetyl | H | Cl | O | H | 1 | CH₂ | 2,3,5-trifluorophenyl | |
| I-484 | pentanoyl | H | Cl | O | H | 1 | CH₂ | 2,4-difluorophenyl | |
| I-485 | fluoroacetyl | H | Cl | O | H | 1 | CH₂ | 2,3,6-trifluorophenyl | |
| I-486 | chloroacetyl | H | Cl | O | H | 1 | CH₂ | 2,3,6-trifluorophenyl | |
| I-487 | methoxyacetyl | H | Cl | O | H | 1 | CH₂ | 2,3,6-trifluorophenyl | |
| I-488 | H | H | ethynyl | O | H | 1 | (S)—CHMe | cyclohexyl | |
| I-489 | H | H | ethynyl | O | H | 1 | CH₂ | 4-fluorophenyl | |
| I-490 | H | H | ethynyl | O | H | 1 | CH₂ | 3-fluorophenyl | |
| I-491 | H | H | ethynyl | O | H | 1 | CH₂ | 2,3-difluorophenyl | |
| I-492 | tert-butoxycarbonyl | H | Cl | O | H | 1 | CH₂ | 2,4-difluorophenyl | |
| I-493 | H | H | Cl | O | H | 1 | CH₂ | 3,4-dichlorophenyl | |
| I-494 | tert-butoxycarbonyl | H | prop-1-yn-1-yl | O | H | 1 | CH₂ | cyclohexyl | |
| I-495 | H | H | prop-1-yn-1-yl | O | H | 1 | CH₂ | cyclohexyl | |
| I-496 | H | H | bromoethynyl | O | H | 1 | CH₂ | cyclohexyl | |
| I-497 | methoxyacetyl | H | Cl | O | H | 1 | CH₂ | 3,5-difluorophenyl | |
| I-498 | difluoroacetyl | H | Cl | O | H | 1 | CH₂ | 2,4-dichlorophenyl | |
| I-499 | trifluoroacetyl | H | Cl | O | H | 1 | CH₂ | 3-chloro-4-fluorophenyl | |
| I-500 | trifluoroacetyl | H | Cl | O | H | 1 | CH₂ | 2,4-dichlorophenyl | |
| I-501 | chloroacetyl | H | Cl | O | H | 1 | CH₂ | 3,5-difluorophenyl | |
| I-502 | difluoroacetyl | H | Cl | O | H | 1 | CH₂ | 2,6-difluorophenyl | |
| I-503 | trifluoroacetyl | H | Cl | O | H | 1 | CH₂ | 2,6-difluorophenyl | |
| I-504 | fluoroacetyl | H | Cl | O | H | 1 | CH₂ | 4-fluorophenyl | |
| I-505 | chloroacetyl | H | Cl | O | H | 1 | CH₂ | 2,6-difluorophenyl | |
| I-506 | fluoroacetyl | H | Cl | O | H | 1 | CH₂ | 2,6-difluorophenyl | |
| I-507 | chloroacetyl | H | Cl | O | H | 1 | CH₂ | 4-fluorophenyl | |
| I-508 | difluoroacetyl | H | Cl | O | H | 1 | CH₂ | 2,3-difluorophenyl | |
| I-509 | trifluoroacetyl | H | Cl | O | H | 1 | CH₂ | 3-chlorophenyl | |
| I-510 | trifluoroacetyl | H | Cl | O | H | 1 | CH₂ | 2,5-difluorophenyl | |
| I-511 | methoxyacetyl | H | Cl | O | H | 1 | CH₂ | 2,6-difluorophenyl | |
| I-512 | chloroacetyl | H | Cl | O | H | 1 | CH₂ | 2,5-difluorophenyl | |
| I-513 | fluoroacetyl | H | Cl | O | H | 1 | CH₂ | 2,5-difluorophenyl | |
| I-514 | difluoroacetyl | H | Cl | O | H | 1 | CH₂ | 2-chlorophenyl | |
| I-515 | difluoroacetyl | H | Cl | O | H | 1 | CH₂ | 3-chlorophenyl | |
| I-516 | difluoroacetyl | H | Cl | O | H | 1 | CH₂ | 3,5-difluorophenyl | |
| I-517 | trifluoroacetyl | H | Cl | O | H | 1 | CH₂ | 3,5-difluorophenyl | |
| I-518 | trifluoroacetyl | H | Cl | O | H | 1 | CH₂ | 2-chlorophenyl | |
| I-519 | difluoroacetyl | H | Cl | O | H | 1 | CH₂ | 2,5-difluorophenyl | |
| I-520 | difluoroacetyl | H | Cl | O | H | 1 | CH₂ | 3-chloro-4-fluorophenyl | |
| I-521 | fluoroacetyl | H | Cl | O | H | 1 | CH₂ | 3,5-difluorophenyl | |
| I-522 | H | difluoroacetyl | Cl | O | H | 1 | (S)—CHMe | cyclohexyl | 3.06[a] |
| I-523 | H | H | I | O | H | 0 | — | cyclohexyl | |
| I-524 | H | H | I | O | H | 1 | CH₂ | tetrahydrofuran-2-yl | |
| I-525 | H | H | I | O | H | 1 | CH₂ | tetrahydrofuran-3-yl | |
| I-526 | H | H | I | O | H | 0 | — | cyclopentyl | |
| I-527 | H | H | ethynyl | O | H | 1 | CH₂ | 2,6-difluorophenyl | |
| I-528 | H | H | ethynyl | O | H | 1 | CH₂ | 2-fluorophenyl | |
| I-529 | H | H | ethynyl | O | H | 1 | CH₂ | 2,5-difluorophenyl | |
| I-530 | H | H | ethynyl | O | H | 1 | CH₂ | 3,4-difluorophenyl | |
| I-531 | H | H | ethynyl | O | H | 1 | CH₂ | 3,5-difluorophenyl | |
| I-532 | H | H | fluoro | O | H | 1 | CH₂ | 2,4-difluorophenyl | |
| I-533 | chloroacetyl | H | Cl | O | H | 1 | CH₂ | 2-fluorophenyl | |
| I-534 | chloroacetyl | H | Cl | O | H | 1 | CH₂ | 2,4-dichlorophenyl | |
| I-535 | fluoroacetyl | H | Cl | O | H | 1 | CH₂ | 2-fluorophenyl | |
| I-536 | difluoroacetyl | H | Cl | O | H | 1 | CH₂ | 2-fluorophenyl | |
| I-537 | trifluoroacetyl | H | Cl | O | H | 1 | CH₂ | 2-fluorophenyl | |
| I-538 | fluoroacetyl | H | Cl | O | H | 1 | CH₂ | 2,4-dichlorophenyl | |
| I-539 | H | H | Br | O | H | 1 | CH₂ | cyclohexyl | 2.51[a] |
| I-540 | benzyl | benzyl | H | O | H | 1 | CH₂ | cyclohexyl | 5.22[a] |
| I-541 | benzyl | benzyl | Br | O | H | 1 | CH₂ | cyclohexyl | 6.25[a] |
| I-542 | H | H | Cl | O | methoxy | 1 | CH₂ | 2,4-difluorophenyl | 2.50[a] |
| I-543 | H | H | Cl | O | methoxy | 1 | CH₂ | cyclohexyl | 3.04[a] |
| I-544 | H | H | ethynyl | O | H | 1 | CH₂ | 1-methylcyclohexyl | |
| I-545 | H | H | ethynyl | O | H | 1 | CH₂ | tetrahydro-2H-pyran-4-yl | |
| I-546 | H | 4-methoxybenzyl | Cl | O | H | 0 | — | tert-butyl | 3.56[a]; |
| | | | | | | | | | 3.47[b] |
| I-547 | H | 4-methoxybenzyl | Cl | O | H | 0 | — | 1,2,3,4-tetrahydronaphthalen-1-yl | 4.20[a]; |
| | | | | | | | | | 4.10[b] |
| I-548 | H | 4-methoxybenzyl | Cl | O | H | 0 | — | rel-[(1R,2R,4R)-1,7,7- | 5.00[a]; |
| | | | | | | | | trimethylbicyclo[2.2.1]hept-2-yl] | 4.87[b] |
| I-549 | H | H | ethynyl | O | H | 1 | CH₂ | cycloheptyl | |
| I-550 | H | H | ethynyl | O | H | 0 | — | cyclohexyl | |
| I-551 | H | H | ethynyl | O | H | 0 | — | cyclopentyl | |
| I-552 | H | H | ethynyl | O | H | 1 | CH₂ | cyclopentyl | |
| I-553 | H | H | fluoro | O | H | 1 | CH₂ | cyclohexyl | |
| I-554 | H | 4-methoxybenzyl | Cl | O | H | 0 | — | 8-methyl-8-azabicyclo[3.2.1]oct-3- | 1.38[a]; |
| | | | | | | | | yl | 2.36[b] |
| I-555 | H | H | prop-1-yn-1-yl | O | H | 1 | CH₂ | cyclopentyl | |
| I-556 | H | H | prop-1-yn-1-yl | O | H | 1 | CH₂ | cycloheptyl | |
| I-557 | H | H | prop-1-yn-1-yl | O | H | 1 | CH₂ | 2,4-difluorophenyl | |
| I-558 | H | H | prop-1-yn-1-yl | O | H | 1 | CH₂ | tetrahydro-2H-pyran-2-yl | |
| I-559 | H | H | 3- | O | H | 1 | CH₂ | cyclohexyl | |
| | | | methoxyprop-1-yn-1-yl | | | | | | |
| I-560 | H | H | 3- | O | H | 1 | CH₂ | 2,4-difluorophenyl | |
| | | | methoxyprop-1-yn-1-yl | | | | | | |
| I-561 | H | H | 3- | O | H | 1 | CH₂ | cyclopentyl | |
| | | | methoxyprop-1-yn-1-yl | | | | | | |
| I-562 | H | H | Cl | O | H | 0 | — | tert-butyl | 1.72[a]; |
| | | | | | | | | | 1.68[b] |
| I-563 | H | H | Cl | O | H | 0 | — | 1,2,3,4-tetrahydronaphthalen-1-yl | 2.50[a]; |
| | | | | | | | | | 2.42[b] |
| I-564 | H | H | Cl | O | H | 0 | — | rel-[(1R,2R,4R)-1,7,7- | 3.20[a]; |
| | | | | | | | | trimethylbicyclo[2.2.1]hept-2-yl] | 3.11[b] |
| I-565 | H | H | cyano | O | H | 1 | CH₂ | cyclohexyl | |
| I-566 | 4-methoxy-4- | 4-methoxy-4- | Cl | O | H | 1 | CH₂ | 2,4-difluorophenyl | |
| | oxobutanoyl | oxobutanoyl | | | | | | | |
| I-567 | 5-methoxy-5- | H | Cl | O | H | 1 | CH₂ | 2,4-difluorophenyl | |
| | oxopentanoyl | | | | | | | | |
| I-568 | tert-butoxycarbonyl | tert-butoxycarbonyl | Cl | O | H | 1 | CH₂ | cyclohexyl | |
| I-569 | benzoyl | H | Cl | O | H | 1 | CH₂ | 4-chlorophenyl | 2.97[a] |
| I-570 | acetyl | H | Cl | O | H | 1 | CH₂ | 4-chlorophenyl | 2.03[a] |
| I-571 | acetyl | acetyl | Cl | O | H | 1 | CH₂ | 4-chlorophenyl | 2.99[a] |
| I-572 | H | 4-methoxybenzyl | Cl | O | H | 0 | — | rel-[(1R,4S)-1,7,7- | 5.02[a]; |
| | | | | | | | | trimethylbicyclo[2.2.1]hept-2-yl] | 4.87[b] |
| I-573 | H | H | prop-1-yn-1-yl | O | H | 1 | CH₂ | 2-fluorophenyl | |
| I-574 | difluoroacetyl | H | Cl | O | H | 1 | CH₂ | 4-chlorophenyl | 2.51[a] |
| I-575 | H | H | prop-1-yn-1-yl | O | H | 1 | (S)—CHMe | cyclohexyl | |
| I-576 | H | H | prop-1-yn-1-yl | O | H | 1 | CH₂ | tetrahydro-2H-pyran-4-yl | |
| I-577 | H | H | prop-1-yn-1-yl | O | H | 1 | CH₂ | 2,4,6-trifluorophenyl | |
| I-578 | 2,2,2-trifluoroethyl | H | Cl | O | H | 1 | CH₂ | 4-chlorophenyl | 3.44[a] |
| I-579 | methoxycarbonyl | H | Cl | O | H | 1 | CH₂ | 4-chlorophenyl | 2.29[a] |
| I-580 | cyclopropyl | H | Cl | O | H | 1 | CH₂ | 4-chlorophenyl | 3.07[a] |
| I-581 | prop-2-yn-1-yl | H | Cl | O | H | 1 | CH₂ | 4-chlorophenyl | 2.97[a] |
| I-582 | benzoyl | benzoyl | Cl | O | H | 1 | CH₂ | 4-chlorophenyl | 4.39[a] |
| I-583 | H | H | Me | O | H | 1 | CH₂ | 2,4,6-trifluorophenyl | 1.69[a] |
| I-584 | H | H | Me | O | H | 1 | CH₂ | 3,4,5-trifluorophenyl | 1.92[a] |
| I-585 | H | H | Me | O | H | 1 | CH₂ | 2,3,5-trifluorophenyl | 1.82[a] |
| I-586 | 4-methoxybenzyl | H | Cl | O | H | 1 | CH₂ | rel-[(1R,2S,5S)-6,6- | 5.06[a]; |
| | | | | | | | | dimethylbicyclo[3.1.1]hept-2-yl] | 4.93[b] |
| I-587 | H | H | Me | O | H | 1 | CH₂ | 2-chloro-6-fluorophenyl | 1.61[a] |
| I-588 | H | H | Me | O | H | 1 | CH₂ | 2,4,5-trifluorophenyl | 1.64[a] |
| I-589 | H | H | 3- | O | H | 1 | CH₂ | cycloheptyl | |
| | | | methoxyprop-1-yn-1-yl | | | | | | |
| I-590 | H | H | 3- | O | H | 1 | CH₂ | tetrahydro-2H-pyran-2-yl | |
| | | | methoxyprop-1-yn-1-yl | | | | | | |
| I-591 | H | H | 3- | O | H | 1 | (S)—CHMe | cyclohexyl | |
| | | | methoxyprop-1-yn-1-yl | | | | | | |
| I-592 | H | H | 3- | O | H | 1 | CH₂ | 2,4,6-trifluorophenyl | |
| | | | methoxyprop-1-yn-1-yl | | | | | | |
| I-593 | H | H | 3- | O | H | 1 | CH₂ | 2-fluorophenyl | |
| | | | methoxyprop-1-yn-1-yl | | | | | | |
| I-594 | H | H | Cl | O | H | 0 | — | rel-[(1R,4R)-1,7,7- | 3.21[a]; |
| | | | | | | | | trimethylbicyclo[2.2.1]hept-2-yl] | 3.12[b] |
| I-595 | H | 4-methoxybenzyl | Cl | O | H | 0 | — | rel-[(1R,2S,5R)-2-isopropyl-5- | 5.48[a]; |
| | | | | | | | | methylcyclohexyl] | 5.41[b] |
| I-596 | H | H | Me | O | H | 1 | CH₂ | 2,3,6-trifluorophenyl | 1.49[a] |
| I-597 | H | H | Me | O | H | 1 | CH₂ | 2,3,4-trifluorophenyl | 1.68[a] |
| I-598 | H | H | Cl | O | amino | 1 | CH₂ | cyclohexyl | |
| I-599 | H | H | 3- | O | H | 1 | CH₂ | cyclohexyl | |
| | | | acetoxyprop-1-yn-1-yl | | | | | | |
| I-600 | 4-methoxybenzyl | H | cyclopropyl | O | H | 1 | CH₂ | 4-cyclopropylphenyl | 3.94[a] |
| I-601 | H | propionyl | Cl | O | H | 1 | (S)—CHMe | cyclohexyl | 2.82[a] |
| I-602 | H | H | Cl | O | H | 1 | CH₂ | bicyclo[2.2.1]hept-2-yl | |
| I-603 | H | H | Cl | O | H | 1 | CH₂ | 4-tert-butylcyclohexyl | |
| I-604 | 2-furoyl | H | Cl | O | H | 1 | CH₂ | cyclohexyl | |
| I-605 | H | H | cyano | O | H | 1 | CH₂ | 2,4-difluorophenyl | |
| I-606 | H | H | cyclopropyl | O | H | 1 | CH₂ | 4-cyclopropylphenyl | 2.48[a] |
| I-607 | H | H | Cl | O | H | 0 | — | 8-methyl-8-azabicyclo[3.2.1]oct-3- | −0.20[c] |
| | | | | | | | | yl | |
| I-608 | H | H | Cl | O | H | 0 | — | rel-[(1R,2S,5R)-2-isopropyl-5- | 3.74[a]; |
| | | | | | | | | methylcyclohexyl] | 3.61[b] |
| I-609 | trifluoroacetyl | H | ethynyl | O | H | 1 | CH₂ | cyclohexyl | 3.05[a] |
| I-610 | H | H | ethynyl | O | H | 1 | CH₂ | 2,4,5-trifluorophenyl | |
| I-611 | H | H | ethynyl | O | H | 1 | CH₂ | 2,3,4-trifluorophenyl | |
| I-612 | H | H | ethynyl | O | H | 1 | CH₂ | 3,4,5-trifluorophenyl | |
| I-613 | H | H | ethynyl | O | H | 1 | CH₂ | 1-hydroxycyclohexyl | |
| I-614 | H | H | ethynyl | O | H | 1 | CH₂ | 1-fluorocyclohexyl | |
| I-615 | H | H | Cl | O | H | 1 | CH₂ | 1-fluorocyclohexyl | 2.17[a] |
| I-616 | H | H | cyano | O | H | 1 | (S)—CHMe | cyclohexyl | |
| I-617 | H | H | Cl | O | propionyl | 1 | CH₂ | cyclohexyl | |
| I-618 | H | H | 3- | O | H | 1 | CH₂ | cyclohexyl | 1.90[c] |
| | | | hydroxyprop-1-yn-1-yl | | | | | | |
| I-619 | H | H | Cl | O | 4-fluorobenzoyl | 1 | CH₂ | cyclohexyl | |
| I-620 | 2-thienylcarbonyl | H | Cl | O | H | 1 | CH₂ | cyclohexyl | |
| I-621 | H | H | Cl | O | H | 0 | — | rel-(1R,4aR,8aR)- | 3.39[a] |
| | | | | | | | | decahydronaphthalen-1-yl | |
| I-622 | H | H | Cl | O | H | 0 | — | rel-(1R,4aS,8aR)- | 3.48[a] |
| | | | | | | | | decahydronaphthalen-1-yl | |
| I-623 | 2,4-difluorobenzoyl | H | Cl | O | H | 1 | CH₂ | cyclohexyl | |
| I-624 | 3-fluorobenzoyl | H | Cl | O | H | 1 | CH₂ | cyclohexyl | |
| I-625 | 2-fluorobenzoyl | H | Cl | O | H | 1 | CH₂ | cyclohexyl | |
| I-626 | cyclohexylcarbonyl | H | Cl | O | H | 1 | CH₂ | 2,4-difluorophenyl | |
| I-627 | 2-fluorobenzoyl | H | Cl | O | H | 1 | CH₂ | 2,4-difluorophenyl | |
| I-628 | 4-methoxybenzyl | H | Cl | O | H | 1 | CH₂ | 2,3,4,6-tetrafluorophenyl | |
| I-629 | H | H | Cl | O | butyryl | 1 | CH₂ | cyclohexyl | |
| I-630 | H | cyclopropyl | Cl | O | H | 0 | — | decahydronaphthalen-1-yl | 4.44 + 4.32 + |
| | | | | | | | | | 4.54 + 4.27[a] |
| I-631 | H | 2,2,2-trifluoroethyl | 2,2,2- | O | H | 0 | — | rel-(1R,4aR,8aR)- | 4.82[a] |
| | | | trifluoroethanamino | | | | | decahydronaphthalen-1-yl | |
| I-632 | H | cyclopropyl | H | O | H | 0 | — | rel-(1R,4aR,8aR)- | 3.42 + 3.37[a] |
| | | | | | | | | decahydronaphthalen-1-yl | |
| I-633 | difluoroacetyl | H | ethynyl | O | H | 1 | CH₂ | cyclohexyl | |
| I-634 | 2,2,3,3,3- | H | ethynyl | O | H | 1 | CH₂ | cyclohexyl | |
| | pentafluoropropanoyl | | | | | | | | |
| I-635 | H | chloro(fluoro)acetyl | ethynyl | O | H | 1 | (S)—CHMe | cyclohexyl | |
| I-636 | H | 2-bromopropanoyl | ethynyl | O | H | 1 | (S)—CHMe | cyclohexyl | |
| I-637 | H | 2,2,3,3,3- | ethynyl | O | H | 1 | (S)—CHMe | cyclohexyl | |
| | | pentafluoropropanoyl | | | | | | | |
| I-638 | H | difluoroacetyl | ethynyl | O | H | 1 | (S)—CHMe | cyclohexyl | |
| I-639 | H | trifluoroacetyl | ethynyl | O | H | 1 | (S)—CHMe | cyclohexyl | |
| I-640 | 2-bromopropanoyl | H | ethynyl | O | H | 1 | CH₂ | cyclohexyl | |
| I-641 | 2,2,3,3,3- | H | ethynyl | O | H | 1 | CH₂ | 2,4-difluorophenyl | |
| | pentafluoropropanoyl | | | | | | | | |
| I-642 | trifluoroacetyl | H | ethynyl | O | H | 1 | CH₂ | 2,4-difluorophenyl | |
| I-643 | difluoroacetyl | H | ethynyl | O | H | 1 | CH₂ | 2,4-difluorophenyl | |
| I-644 | chloro(fluoro)acetyl | H | ethynyl | O | H | 1 | CH₂ | cyclohexyl | |
| I-645 | H | H | Cl | O | H | 1 | CH₂ | 2,3,4,6-tetrafluorophenyl | |
| I-646 | H | H | ethynyl | O | H | 1 | CH₂ | 1-hydroxycyclopentyl | |
| I-647 | H | H | Cl | O | H | 1 | CH₂ | 2-hydroxycyclohexyl | |
| I-648 | H | H | ethynyl | O | H | 1 | CH₂ | 2-hydroxycyclohexyl | |
| I-649 | H | H | Cl | O | H | 1 | CH₂ | 1-hydroxycyclopentyl | |
| I-650 | H | H | Cl | O | acetyl | 1 | CH₂ | cyclohexyl | |
| I-651 | 2,2,3,3- | H | Cl | O | H | 1 | CH₂ | 2,4-difluorophenyl | |
| | tetrafluoropropanoyl | | | | | | | | |
| I-652 | 5-methoxy-5- | H | Cl | O | H | 1 | CH₂ | cyclohexyl | |
| | oxopentanoyl | | | | | | | | |
| I-653 | 2,2,3,3- | H | Cl | O | H | 1 | CH₂ | cyclohexyl | |
| | tetrafluoropropanoyl | | | | | | | | |
| I-654 | trifluoroacetyl | H | cyano | O | H | 1 | CH₂ | 2,4-difluorophenyl | |
| I-655 | difluoroacetyl | H | cyano | O | H | 1 | CH₂ | 2,4-difluorophenyl | |
| I-656 | H | trifluoroacetyl | cyano | O | H | 1 | (S)—CHMe | cyclohexyl | |
| I-657 | H | difluoroacetyl | cyano | O | H | 1 | (S)—CHMe | cyclohexyl | |
| I-658 | H | H | Cl | O | H | 1 | CH₂ | rel-[(1R,3S)-3-hydroxycyclopentyl] | 0.72[a] |
| I-659 | H | H | methoxy | O | H | 1 | CH₂ | cyclohexyl | |
| I-660 | H | H | methoxy | O | H | 1 | CH₂ | 2,4-difluorophenyl | |
| I-661 | H | H | methoxy | O | H | 1 | (S)—CHMe | cyclohexyl | |
| I-662 | H | H | Cl | O | H | 0 | — | rel-[(1R,2S)-2-methylcyclohexyl] | |
| I-663 | H | H | Cl | O | H | 0 | — | rel-[(1R,2R)-2-methylcyclohexyl] | |
| I-664 | H | H | Cl | O | H | 0 | — | 2-methylcyclohexyl | |
| I-665 | H | H | Cl | O | H | 1 | CH₂ | 2,2-dichlorocyclopropyl | |
| I-666 | cyanoacetyl | H | Cl | O | H | 1 | CH₂ | 2,4-difluorophenyl | |
| I-667 | difluoroacetyl | H | Cl | S | H | 1 | CH₂ | cyclohexyl | |
| I-668 | 2,2- | H | Cl | S | H | 1 | CH₂ | cyclohexyl | |
| | difluoroethanethioyl | | | | | | | | |
| I-669 | 2,2,3,3,4,4,4- | H | Cl | S | H | 1 | CH₂ | cyclohexyl | |
| | heptafluorobutanoyl | | | | | | | | |
| I-670 | 4-methoxy-4- | H | Cl | S | H | 1 | CH₂ | cyclohexyl | |
| | oxobutanoyl | | | | | | | | |
| I-671 | trichloroacetyl | H | Cl | O | H | 1 | CH₂ | cyclohexyl | |
| I-672 | trichloroacetyl | H | Cl | O | H | 1 | CH₂ | 2,4-difluorophenyl | |
| I-673 | 2,2,3,3,3- | H | Cl | S | H | 1 | CH₂ | cyclohexyl | |
| | pentafluoropropanoyl | | | | | | | | |
| I-674 | 4-methoxybenzyl | H | Cl | O | H | 1 | CH₂ | 5-methyl-2-furyl | 3.27[a] |
| I-675 | 4-methoxybenzyl | H | Cl | O | H | 1 | CH₂ | 4-methylcyclohexyl | 4.56[a] |
| I-676 | 3-hydroxypropyl | H | Cl | O | H | 1 | CH₂ | cyclohexyl | 2.46[a] |
| I-677 | H | H | Cl | O | H | 1 | CH₂ | 2,4,5-trifluorophenyl | 2.03[a] |
| I-678 | H | 4-methoxybenzyl | Cl | O | H | 0 | — | 1-cyclobutyl-3-phenylpropyl | 5.00[a]; |
| | | | | | | | | | 4.86[b] |
| I-679 | H | 4-methoxybenzyl | Cl | O | H | 0 | — | 1-cyclohexyl-3-methoxy-3- | 4.17[a]; |
| | | | | | | | | oxopropyl | 4.05[b] |
| I-680 | H | 4-methoxybenzyl | Cl | O | H | 0 | — | cyclohexyl(phenyl)methyl | 5.17[a]; |
| | | | | | | | | | 4.99[b] |
| I-681 | H | 4-methoxybenzyl | Cl | O | H | 1 | CHMe | tetrahydrofuran-2-yl | 3.10[a]; |
| | | | | | | | | | 3.02[b] |
| I-682 | 3-{[tert- | H | Cl | O | H | 1 | CH₂ | cyclohexyl | |
| | butyl(dimethyl)silyl] | | | | | | | | |
| | oxy}propyl | | | | | | | | |
| I-683 | cyanoacetyl | H | Cl | O | H | 1 | CH₂ | cyclohexyl | |
| I-684 | pentanoyl | pentanoyl | Cl | O | H | 1 | CH₂ | cyclohexyl | |
| I-685 | 2,2,3,3,3- | H | Cl | S | H | 1 | CH₂ | 2,4-difluorophenyl | |
| | pentafluoropropanoyl | | | | | | | | |
| I-686 | 3-methoxy-3- | H | Cl | S | H | 1 | CH₂ | 2,4-difluorophenyl | |
| | oxopropanoyl | | | | | | | | |
| I-687 | 2,2,3,3,4,4,4- | H | Cl | S | H | 1 | CH₂ | 2,4-difluorophenyl | |
| | heptafluorobutanoyl | | | | | | | | |
| I-688 | 2,2- | H | Cl | S | H | 1 | CH₂ | 2,4-difluorophenyl | |
| | difluoroethanethioyl | | | | | | | | |
| I-689 | difluoroacetyl | H | Cl | S | H | 1 | CH₂ | 2,4-difluorophenyl | |
| I-690 | chloro(fluoro)acetyl | H | Cl | S | H | 1 | CH₂ | cyclohexyl | |
| I-691 | dichloroacetyl | H | Cl | S | H | 1 | CH₂ | cyclohexyl | |
| I-692 | H | H | Cl | O | H | 1 | CH₂ | rel-[(1R,2S)-2-methylcyclohexyl] | |
| I-693 | H | H | Cl | O | H | 1 | CH₂ | rel-[(1R,2R)-2-methylcyclohexyl] | |
| I-694 | H | H | methoxy | O | H | 0 | — | 2-methylcyclohexyl | |
| I-695 | H | H | methoxy | O | H | 0 | — | rel-[(1R,2S)-2-methylcyclohexyl] | |
| I-696 | H | H | methoxy | O | H | 0 | — | rel-[(1R,2R)-2-methylcyclohexyl] | |
| I-697 | H | H | Cl | O | H | 0 | — | rel-[(1R,2R)-2-chlorocyclohexyl] | |
| I-698 | H | H | ethoxy | O | H | 1 | CH₂ | cyclohexyl | |
| I-699 | H | H | ethoxy | O | H | 1 | CH₂ | 2,4-difluorophenyl | |
| I-700 | H | H | Cl | O | H | 1 | CH₂ | 4,4-dimethylcyclohexyl | |
| I-701 | H | H | Cl | O | H | 1 | CH₂ | rel-[(1R,2R,5R)-6,6- | 3.28[a]; |
| | | | | | | | | dimethylbicyclo[3.1.1]hept-2-yl] | 3.19[b] |
| I-702 | H | H | Cl | O | H | 0 | — | 1-cyclobutyl-3-phenylpropyl | 3.37[a]; |
| | | | | | | | | | 3.29[b] |
| I-703 | H | H | Cl | O | H | 0 | — | 1-cyclohexyl-3-methoxy-3- | 2.48[a]; |
| | | | | | | | | oxopropyl | 2.41[b] |
| I-704 | H | H | Cl | O | H | 0 | — | cyclohexyl(phenyl)methyl | 3.50[a]; |
| | | | | | | | | | 3.43[b] |
| I-705 | H | H | Cl | O | H | 1 | CHMe | tetrahydrofuran-2-yl | 1.32[a]; |
| | | | | | | | | | 1.33[b] |
| I-706 | H | H | Me | O | H | 1 | CH₂ | 2-hydroxycyclopentyl | 0.79[a] |
| I-707 | H | H | Me | O | H | 1 | CH₂ | 2-hydroxycyclopentyl | 0.82[a] |
| I-708 | acetyl | H | Cl | O | H | 1 | CH₂ | 2,4-difluorophenyl | |
| I-709 | methoxyacetyl | H | Cl | S | H | 1 | CH₂ | 2,4-difluorophenyl | |
| I-710 | 2- | H | Cl | S | H | 1 | CH₂ | 2,4-difluorophenyl | |
| | methoxyethanethioyl | | | | | | | | |
| I-711 | tert-butoxycarbonyl | tert-butoxycarbonyl | Br | O | H | 1 | CH₂ | cyclohexyl | |
| I-712 | tert-butoxycarbonyl | H | Br | O | H | 1 | CH₂ | cyclohexyl | |
| I-713 | tert-butoxycarbonyl | tert-butoxycarbonyl | Br | O | H | 1 | CH₂ | 2,4-difluorophenyl | |
| I-714 | tert-butoxycarbonyl | H | Br | O | H | 1 | CH₂ | 2,4-difluorophenyl | |
| I-715 | 3-oxopropyl | H | Cl | O | H | 1 | CH₂ | cyclohexyl | 2.83[a] |
| I-716 | H | H | Cl | O | H | 1 | CH₂ | 3-iodophenyl | 2.51[a] |
| I-717 | H | H | Cl | O | H | 1 | CH₂ | rel-[(1R,3S)-3-methylcyclohexyl] | |
| I-718 | H | H | Cl | O | H | 1 | CH₂ | rel-[(1R,3R)-3-methylcyclohexyl] | |
| I-719 | H | H | Cl | O | H | 1 | CH₂ | 3-methylcyclohexyl | |
| I-720 | H | H | Cl | O | H | 1 | CH₂ | 3,5-difluoropyridin-2-yl | |
| I-721 | 2-carboxyethyl | H | Cl | O | H | 1 | CH₂ | cyclohexyl | 2.42[a] |
| I-722 | H | H | Br | O | H | 1 | CH₂ | 2,4-difluorophenyl | |
| I-723 | 4-methoxybenzyl | H | Cl | O | H | 1 | CH₂ | 2-chloro-4,5-difluorophenyl | |
| I-724 | H | H | ethoxy | O | H | 1 | CH₂ | 2,4,6-trifluorophenyl | 2.09[a] |
| I-725 | H | H | ethoxy | O | H | 1 | CH₂ | cyclopentyl | 2.18[a] |
| I-726 | H | H | ethoxy | O | H | 1 | CH₂ | 1-hydroxycyclohexyl | 1.55[a] |
| I-727 | H | H | ethoxy | O | H | 0 | — | cyclohexyl | 2.14[a] |
| I-728 | H | H | ethoxy | O | H | 1 | CH₂ | cycloheptyl | 2.83[a] |
| I-729 | H | H | ethoxy | O | H | 1 | CH₂ | tetrahydro-2H-pyran-2-yl | 1.66[a] |
| I-730 | H | H | ethoxy | O | H | 1 | CH₂ | 2-fluorophenyl | 1.93[a] |
| I-731 | H | H | ethoxy | O | H | 1 | CH₂ | 4-chlorophenyl | 2.26[a] |
| I-732 | H | H | methoxy | O | H | 1 | CH₂ | 2,4,6-trifluorophenyl | 1.82[a] |
| I-733 | H | H | methoxy | O | H | 1 | CH₂ | 2-fluorophenyl | 1.64[a] |
| I-734 | H | H | methoxy | O | H | 1 | CH₂ | tetrahydro-2H-pyran-2-yl | 1.31[a] |
| I-735 | H | H | methoxy | O | H | 1 | CH₂ | cycloheptyl | 2.50[a] |
| I-736 | H | H | methoxy | O | H | 1 | CH₂ | cyclopentyl | 1.87[a] |
| I-737 | H | H | methoxy | O | H | 1 | CH₂ | 4-chlorophenyl | 1.98[a] |
| I-738 | H | H | Cl | O | H | 1 | CH₂ | 4-methylcyclohexyl | 2.75[a] |
| I-739 | 4-methoxybenzyl | H | Cl | O | H | 1 | CH₂ | bicyclo[2.2.1]hept-1-yl | 4.29[a] |
| I-740 | acetyl | acetyl | Cl | O | H | 1 | CH₂ | 2,4,6-trifluorophenyl | |
| I-741 | acetyl | H | Cl | O | H | 1 | CH₂ | 2,4,6-trifluorophenyl | |
| I-742 | hexanoyl | H | Cl | O | H | 1 | CH₂ | cyclohexyl | |
| I-743 | hexanoyl | hexanoyl | Cl | O | H | 1 | CH₂ | cyclohexyl | |
| I-744 | cyanoacetyl | H | Cl | S | H | 1 | CH₂ | cyclohexyl | |
| I-745 | butyryl | H | Cl | O | H | 1 | CH₂ | 2,4-difluorophenyl | |
| I-746 | hexanoyl | H | Cl | O | H | 1 | CH₂ | 2,4-difluorophenyl | |
| I-747 | fluoroacetyl | 4-methoxybenzyl | Cl | O | H | 1 | CH₂ | 2,4,6-trifluorophenyl | |
| I-748 | chloroacetyl | 4-methoxybenzyl | Cl | O | H | 1 | CH₂ | 2,4,6-trifluorophenyl | |
| I-749 | 2-chloro-2,3,3,3- | 2-chloro-2,3,3,3- | Cl | O | H | 1 | CH₂ | cyclohexyl | |
| | tetrafluoropropanoyl | tetrafluoropropanoyl | | | | | | | |
| I-750 | propionyl | H | Cl | O | H | 1 | CH₂ | 2,4,6-trifluorophenyl | |
| I-751 | propionyl | propionyl | Cl | O | H | 1 | CH₂ | 2,4,6-trifluorophenyl | |
| I-752 | hexanoyl | H | Cl | O | H | 1 | CH₂ | 2,4,6-trifluorophenyl | |
| I-753 | hexanoyl | hexanoyl | Cl | O | H | 1 | CH₂ | 2,4,6-trifluorophenyl | |
| I-754 | hexanoyl | H | Cl | O | H | 1 | CH₂ | 4-fluorophenyl | |
| I-755 | pentanoyl | H | Cl | O | H | 1 | CH₂ | 2,4,6-trifluorophenyl | |
| I-756 | acetyl | H | Cl | O | H | 1 | CH₂ | 4-fluorophenyl | |
| I-757 | acetyl | acetyl | Cl | O | H | 1 | CH₂ | 4-fluorophenyl | |
| I-758 | propionyl | H | Cl | O | H | 1 | CH₂ | 4-fluorophenyl | |
| I-759 | propionyl | propionyl | Cl | O | H | 1 | CH₂ | 4-fluorophenyl | |
| I-760 | difluoroacetyl | H | Br | O | H | 1 | CH₂ | cyclohexyl | |
| I-761 | trifluoroacetyl | H | Br | O | H | 1 | CH₂ | cyclohexyl | |
| I-762 | H | H | ethynyl | O | H | 0 | — | rel-[(1R,2S)-2-chlorocyclohexyl] | |
| I-763 | H | H | ethynyl | O | H | 0 | — | rel-[(1R,2R)-2-hydroxycyclohexyl] | |
| I-764 | H | H | ethynyl | O | H | 1 | CH₂ | 4,4-dimethylcyclohexyl | |
| I-765 | H | H | Cl | O | H | 1 | CH₂ | 2-chloro-4,5-difluorophenyl | |
| I-766 | propionyl | H | Cl | O | H | 1 | CH₂ | 2,4-difluorophenyl | |
| I-767 | butyryl | H | Cl | O | H | 1 | CH₂ | 2,4,6-trifluorophenyl | |
| I-768 | tert-butoxycarbonyl | H | Br | O | H | 1 | CH₂ | 2,4,6-trifluorophenyl | |
| I-769 | H | H | Br | O | H | 1 | CH₂ | 2,4,6-trifluorophenyl | |
| I-770 | H | tert-butoxycarbonyl | Br | O | H | 0 | — | cyclopentyl | |
| I-771 | H | H | Br | O | H | 0 | — | cyclopentyl | |
| I-772 | H | H | Cl | O | H | 1 | CH₂ | 2-hydroxycyclopentyl | 1.09[a] |
| I-773 | H | H | Cl | O | H | 1 | CH₂ | 2-hydroxycyclopentyl | 0.88[a] |
| I-774 | H | H | H | O | H | 1 | CH₂ | 2-hydroxycyclopentyl | 0.63[a] |
| I-775 | H | H | H | O | H | 1 | CH₂ | 3-hydroxycyclopentyl | 0.36[a] |
| I-776 | 2-cyanopropanoyl | H | Cl | O | H | 1 | CH₂ | cyclohexyl | |
| I-777 | 2-cyanopropanoyl | H | Cl | O | H | 1 | CH₂ | 2,4-difluorophenyl | |
| I-778 | butyryl | H | Cl | O | H | 1 | CH₂ | 4-fluorophenyl | |
| I-779 | pentanoyl | pentanoyl | Cl | O | H | 1 | CH₂ | 4-fluorophenyl | |
| I-780 | pentanoyl | H | Cl | O | H | 1 | CH₂ | 4-fluorophenyl | |
| I-781 | propionyl | H | Br | O | H | 1 | CH₂ | cyclohexyl | |
| I-782 | H | H | Cl | O | H | 1 | CH₂ | 4-fluoro-2-methoxyphenyl | 2.02[a] |
| I-783 | H | H | Cl | O | H | 1 | CH₂ | 4-cyanophenyl | 1.44[a] |
| I-784 | H | H | Cl | O | H | 1 | CH₂ | 4-fluoro-2-methylphenyl | 2.09[a] |
| I-785 | H | H | ethynyl | O | H | 1 | CH₂ | rel-[(1R,3S)-3-methylcyclohexyl] | |
| I-786 | H | H | ethynyl | O | H | 1 | CH₂ | rel-[(1R,3R)-3-methylcyclohexyl] | |
| I-787 | H | H | ethynyl | O | H | 1 | CH₂ | 3-methylcyclohexyl | |
| I-788 | H | H | ethynyl | O | H | 1 | CH₂ | rel-[(1R,2S)-2-methylcyclohexyl] | |
| I-789 | H | H | ethynyl | O | H | 1 | CH₂ | rel-[(1R,2R)-2-methylcyclohexyl] | |
| I-790 | H | H | ethynyl | O | H | 1 | CH₂ | 2-methylcyclohexyl | |
| I-791 | H | H | ethynyl | O | H | 0 | — | rel-[(1R,2S)-2-methylcyclohexyl] | |
| I-792 | H | H | ethynyl | O | H | 0 | — | rel-[(1R,2R)-2-methylcyclohexyl] | |
| I-793 | H | H | ethynyl | O | H | 0 | — | 2-methylcyclohexyl | |
| I-794 | H | H | ethynyl | O | H | 0 | — | 3-(ethoxycarbonyl)cyclopentyl | |
| I-795 | H | H | Br | O | H | 1 | CH₂ | cyclopentyl | |
| I-796 | H | H | Cl | O | H | 0 | — | 3-(ethoxycarbonyl)cyclopentyl | |
| I-797 | H | H | hydroxy | O | H | 1 | CH₂ | cyclohexyl | 2.12[a] |
| I-798 | H | H | ethynyl | O | H | 0 | — | trans-4-(ethoxycarbonyl)cyclohexyl | |
| I-799 | H | H | ethynyl | O | H | 0 | — | cis-4-(ethoxycarbonyl)cyclohexyl | |
| I-800 | H | H | ethynyl | O | H | 0 | — | 4-(ethoxycarbonyl)cyclohexyl | |
| I-801 | H | H | ethynyl | O | H | 0 | — | 3-carboxycyclopentyl | |
| I-802 | H | H | Br | O | H | 1 | CH₂ | rel-[(1R,3R)-3-methylcyclohexyl] | |
| I-803 | H | H | Br | O | H | 1 | CH₂ | rel-[(1R,3S)-3-methylcyclohexyl] | |
| I-804 | H | H | Br | O | H | 1 | CH₂ | 3-methylcyclohexyl | |
| I-805 | H | H | Cl | O | H | 1 | CH—CN | cyclohexyl | |
| I-806 | 3-chloro-2,2,3,3- | 3-chloro-2,2,3,3- | Cl | O | H | 1 | CH₂ | cyclohexyl | |
| | tetrafluoropropanoyl | tetrafluoropropanoyl | | | | | | | |
| I-807 | cyanoacetyl | H | Cl | S | H | 1 | CH₂ | 2,4-difluorophenyl | |
| I-808 | H | H | Cl | O | H | 0 | — | rel-(1R,8aS)-decahydronaphthalen- | 3.33[a] |
| | | | | | | | | 1-yl | |
| I-809 | difluoroacetyl | H | Cl | O | H | 1 | CH₂ | 4-fluoro-2-methylphenyl | 2.28[a] |
| I-810 | propionyl | propionyl | Cl | O | H | 1 | CH₂ | 4-cyanophenyl | 2.89[a] |
| I-811 | propionyl | H | Cl | O | H | 1 | CH₂ | 4-cyanophenyl | 1.54[a] |
| I-812 | trifluoroacetyl | H | Cl | O | H | 1 | CH₂ | 4-cyanophenyl | 2.12[a] |
| I-813 | difluoroacetyl | H | Cl | O | H | 1 | CH₂ | 4-cyanophenyl | 1.68[a] |
| I-814 | H | H | Cl | O | H | 1 | CH₂ | 5-methyl-2-furyl | 1.66[a] |
| I-815 | propionyl | H | Cl | O | H | 1 | CH₂ | 4-fluoro-2-methoxyphenyl | 2.10[a] |
| I-816 | trifluoroacetyl | H | Cl | O | H | 1 | CH₂ | 4-fluoro-2-methylphenyl | 2.72[a] |
| I-817 | propionyl | H | Cl | O | H | 1 | CH₂ | 4-fluoro-2-methylphenyl | 2.15[a] |
| I-818 | H | H | Br | O | H | 1 | (S)—CHMe | cyclohexyl | 2.84[a] |
| I-819 | trifluoroacetyl | H | Cl | O | H | 1 | CH₂ | 4-fluoro-2-methoxyphenyl | 2.72[a] |
| I-820 | difluoroacetyl | H | Cl | O | H | 1 | CH₂ | 4-fluoro-2-methoxyphenyl | 2.23[a] |
| I-821 | H | H | Cl | O | H | 1 | CH₂ | rel-[(1R,2R)-2-chlorocyclohexyl] | 2.44[a] |
| I-822 | H | H | Cl | O | H | 1 | CH₂ | bicyclo[2.2.1]hept-1-yl | 2.53[a] |
| I-823 | 4-methoxybenzyl | H | Cl | O | H | 1 | CH₂ | 1-methoxy-4-methylcyclohexyl | 4.33[a] |
| I-824 | 4-methoxybenzyl | H | Cl | O | H | 1 | CH₂ | 1-ethylcyclohexyl | 4.89[a] |
| I-825 | 4-methoxybenzyl | H | Cl | O | H | 1 | CH₂ | 1,4-dimethylcyclohexyl | 4.84[a] |
| I-826 | H | H | Cl | O | H | 1 | CH₂ | 1-fluorocyclopentyl | 1.81[a] |
| I-827 | H | H | Cl | O | H | 1 | CH₂ | 1-fluorocyclobutyl | 1.50[a] |
| I-828 | H | H | Br | O | H | 1 | CH₂ | 1-fluorocyclohexyl | 2.15[a] |
| I-829 | H | H | Br | O | H | 1 | CH₂ | 1-fluorocyclopentyl | 1.83[a] |
| I-830 | H | H | Br | O | H | 1 | CH₂ | 2,3,4-trifluorophenyl | |
| I-831 | H | H | Br | O | H | 1 | CH₂ | 4-fluorophenyl | 1.82[a] |
| I-832 | H | H | Br | O | H | 1 | CH₂ | 2-fluorophenyl | 1.79[a] |
| I-833 | H | H | Br | O | H | 1 | CH—CN | cyclohexyl | 2.21[a] |
| I-834 | iPr | H | Cl | O | H | 1 | CH₂ | 1-ethylcyclohexyl | 5.03[a] |
| I-835 | H | H | Cl | O | H | 1 | CH₂ | 1,4-dimethylcyclohexyl | 3.17[a] |
| I-836 | H | H | Cl | O | H | 1 | CH₂ | 1-ethylcyclohexyl | 3.20[a] |
| I-837 | H | H | Cl | O | H | 1 | CH₂ | 1-methoxy-4-methylcyclohexyl | 2.58[a] |
| I-838 | H | H | Br | O | H | 0 | — | rel-[(1R,2R)-2-methylcyclohexyl] | 2.34[a] |
| I-839 | H | H | Br | O | H | 0 | — | rel-[(1R,2R)-2-methylcyclohexyl] | 2.49[a] |
| I-840 | H | H | Br | O | H | 1 | CH₂ | 4-(trifluoromethyl)cyclohexyl | 2.68[a] |
| I-841 | H | H | Br | O | H | 0 | — | cyclohexyl | 2.11[a] |
| I-842 | H | H | Br | O | H | 1 | CH₂ | 3,3-dimethylcyclobutyl | 2.60[a] |
| I-843 | H | H | Cl | O | H | 0 | — | trans-4-(ethoxycarbonyl)cyclohexyl | 1.92[a] |
| I-844 | H | H | Cl | O | H | 0 | — | cis-4-(ethoxycarbonyl)cyclohexyl | 2.02[a] |
| I-845 | acetyl | H | Cl | O | H | 1 | CH₂ | 4-fluoro-2-methylphenyl | |
| I-846 | acetyl | acetyl | Cl | O | H | 1 | CH₂ | 4-cyanophenyl | 2.12[a] |
| I-847 | acetyl | H | Cl | O | H | 1 | CH₂ | 4-cyanophenyl | 1.24[a] |
| I-848 | H | H | Cl | O | H | 0 | — | 4-(ethoxycarbonyl)cyclohexyl | |
| I-849 | H | H | Br | O | H | 0 | — | 2-methylcyclohexyl | 2.38 + 2.52[a] |
| I-850 | H | H | Cl | O | H | 1 | CH₂ | cyclohexylmethyl | 2.92[a] |
| I-851 | H | H | Cl | O | H | 1 | CH₂ | benzyl | 1.98[a] |
| I-852 | H | H | Cl | O | H | 0 | — | phenyl | 1.83[a] |
| I-853 | H | H | H | O | H | 1 | CH₂ | cyclohexylmethyl | 2.23[a] |
| I-854 | H | H | H | O | H | 1 | CH₂ | benzyl | 1.52[a] |
| I-855 | H | H | H | O | H | 0 | — | phenyl | 1.41[a] |
| I-856 | H | 4-methoxybenzyl | Cl | O | H | 0 | — | 2,4-difluorophenyl | 3.83[a] |
| I-857 | H | H | Cl | O | H | 0 | — | 4-fluorophenyl | 2.04[a] |
| I-858 | H | 4-methoxybenzyl | Cl | O | H | 0 | — | 4-fluorophenyl | 3.76[a] |
| I-859 | H | 4-methoxybenzyl | Cl | O | cyclopropyl | 1 | CH₂ | 2,2,6-trimethylcyclohexyl | 6.09[a] |
| I-860 | H | 4-methoxybenzyl | Cl | O | cyclopropyl | 0 | — | 2,2-dimethylcyclohexyl | 5.31[a] |
| I-861 | H | 4-methoxybenzyl | Cl | O | H | 0 | — | 3-fluorophenyl | 3.85[a] |
| I-862 | H | 4-methoxybenzyl | Cl | O | H | 0 | — | 3,5-difluorophenyl | 4.15[a] |
| I-863 | H | H | Cl | O | cyclopropyl | 0 | — | 2,2-dimethylcyclohexyl | 3.30[a] |
| I-864 | H | H | Cl | O | H | 0 | — | 2,4-difluorophenyl | 2.07[a] |
| I-865 | H | H | Cl | O | H | 0 | — | 3-fluorophenyl | 2.07[a] |
| I-866 | H | H | Cl | O | H | 0 | — | 3,5-difluorophenyl | 2.54[a] |
| I-867 | H | 4-methoxybenzyl | Cl | O | H | 0 | — | 2,4-dichlorophenyl | 5.22[a] |
| I-868 | H | 4-methoxybenzyl | Cl | O | H | 0 | — | 4-chlorophenyl | 4.21[a] |
| I-869 | H | 4-methoxybenzyl | Cl | O | H | 0 | — | 1,1,3-trimethyl-1,3-dihydro-2- | 3.96[a] |
| | | | | | | | | benzofuran-4-yl | |
| I-870 | H | 4-methoxybenzyl | Cl | O | cyclopropyl | 1 | CH₂ | mesityl | 4.94[a] |
| I-871 | H | 4-methoxybenzyl | Cl | O | cyclopropyl | 1 | CH₂ | 2-isopropylphenyl | 4.71[a] |
| I-872 | H | 4-methoxybenzyl | Cl | O | cyclopropyl | 1 | CH₂ | 3,5-dimethylphenyl | 4.90[a] |
| I-873 | H | 4-methoxybenzyl | Cl | O | cyclopropyl | 1 | CHMe | phenyl | 4.17[a] |
| I-874 | H | 4-methoxybenzyl | Cl | O | cyclopropyl | 1 | CHMe | 2-ethylphenyl | 5.14[a] |
| I-875 | H | 4-methoxybenzyl | Cl | O | cyclopropyl | 0 | — | 1-phenylpropyl | 4.85[a] |
| I-876 | H | 4-methoxybenzyl | Cl | O | cyclopropyl | 1 | CH₂ | 2,5-dimethylphenyl | 4.77[a] |
| I-877 | H | 4-methoxybenzyl | Cl | O | H | 1 | CH₂ | 2-chlorobenzyl | 4.07[a] |
| I-878 | H | H | Cl | O | H | 0 | — | 4-chlorophenyl | 2.51[a] |
| I-879 | H | H | Cl | O | H | 0 | — | 1,1,3-trimethyl-1,3-dihydro-2- | 2.28[a] |
| | | | | | | | | benzofuran-4-yl | |
| I-880 | H | 4-methoxybenzyl | Cl | O | H | 0 | — | 1,1,3-trimethyl-2,3-dihydro-1H- | 5.42[a] |
| | | | | | | | | inden-4-yl | |
| I-881 | H | 4-methoxybenzyl | Cl | O | H | 0 | — | 2-cyanophenyl | 3.62[a] |
| I-882 | H | 4-methoxybenzyl | Cl | O | H | 0 | — | 3-chloro-4-methylphenyl | 4.63[a] |
| I-883 | H | 4-methoxybenzyl | Cl | O | H | 0 | — | methylsulfonyl | 1.86[a] |
| I-884 | H | H | Cl | O | H | 1 | CH₂ | H | 0.32[a] |
| I-885 | H | H | Cl | O | H | 0 | — | 1,1,3-trimethyl-2,3-dihydro-1H- | 3.62[a] |
| | | | | | | | | inden-4-yl | |
| I-886 | H | H | Cl | O | cyclopropyl | 1 | CHMe | phenyl | 2.73[a] |
| I-887 | H | H | Cl | O | cyclopropyl | 1 | CHMe | 2-ethylphenyl | 3.37[a] |
| I-888 | H | H | Cl | O | cyclopropyl | 0 | — | 1-phenylpropyl | 3.11[a] |
| I-889 | H | 4-methoxybenzyl | Cl | O | cyclopropyl | 1 | CH₂ | 2,4,5-trimethylphenyl | 5.11[a] |
| I-890 | H | 4-methoxybenzyl | Cl | O | cyclopropyl | 1 | CH₂ | 2-ethylphenyl | 4.74[a] |
| I-891 | H | 4-methoxybenzyl | Cl | O | cyclopropyl | 1 | CH₂ | 2,6-dimethylphenyl | 4.82[a] |
| I-892 | H | 4-methoxybenzyl | Cl | O | cyclopropyl | 1 | CH₂ | 2,4-dimethylphenyl | 4.80[a] |
| I-893 | H | 4-methoxybenzyl | Cl | O | cyclopropyl | 0 | — | cyclohexyl | 4.67[a] |
| I-894 | H | 4-methoxybenzyl | Cl | O | cyclopropyl | 1 | CHMe | sec-butyl | 4.85[a] |
| I-895 | H | H | Cl | O | H | 1 | CH₂ | 2-chlorobenzyl | 2.41[a] |
| I-896 | H | H | Cl | O | H | 0 | — | | −0.16[a] |
| I-897 | H | 4-methoxybenzyl | Cl | O | cyclopropyl | 1 | CH₂ | phenyl | 4.14[a] |
| I-898 | H | 4-methoxybenzyl | Cl | O | cyclopropyl | 1 | CH₂ | 2-ethyl-6-methylphenyl | 5.17[a] |
| I-899 | H | 4-methoxybenzyl | Cl | O | cyclopropyl | 0 | — | 1-phenylbutyl | 5.25[a] |
| I-900 | H | 4-methoxybenzyl | Cl | O | cyclopropyl | 1 | CH₂ | iPr | 4.09[a] |
| I-901 | H | H | Cl | O | cyclopropyl | 1 | CHMe | sec-butyl | 3.00[a] |
| I-902 | H | H | Cl | O | cyclopropyl | 1 | CH₂ | iPr | 2.25[a] |
| I-903 | benzyl | benzyl | H | O | H | 0 | — | 2,4-dichlorophenyl | 5.74[a] |
| I-904 | benzyl | benzyl | H | O | H | 1 | CH₂ | [3-chloro-5- | 5.03[a] |
| | | | | | | | | (trifluoromethyl)pyridin-2- | |
| | | | | | | | | yl]methyl | |
| I-905 | benzyl | benzyl | H | O | H | 0 | — | 2-cyanophenyl | 4.26[a] |
| I-906 | benzyl | benzyl | H | O | H | 0 | — | 3-chloro-4-methylphenyl | 5.68[a] |
| I-907 | benzyl | benzyl | H | O | cyclopropyl | 1 | CH₂ | mesityl | 6.25[a] |
| I-908 | H | H | Cl | O | cyclopropyl | 0 | — | 1-phenylbutyl | 3.48[a] |
| I-909 | H | H | H | O | cyclopropyl | 1 | CH₂ | 2-chloro-6-fluorophenyl | 2.28[a] |
| I-910 | benzyl | benzyl | H | O | cyclopropyl | 1 | CH₂ | phenyl | 5.36[a] |
| I-911 | benzyl | benzyl | H | O | cyclopropyl | 1 | CH₂ | 2-chloro-6-fluorophenyl | 5.54[a] |
| I-912 | benzyl | benzyl | H | O | cyclopropyl | 1 | CHMe | 3,5-dichlorophenyl | 6.54[a] |
| I-913 | H | H | Cl | S | H | 1 | CH₂ | cyclohexyl | 3.37[a] |
| I-914 | H | H | Cl | O | H | 0 | — | 2-methylphenyl | 2.08[a] |
| I-915 | H | H | Cl | O | H | 0 | — | 3-methylphenyl | 2.28[a] |
| I-916 | H | H | Cl | O | H | 0 | — | 4-methylphenyl | 2.27[a] |
| I-917 | H | H | Cl | O | cyclopropyl | 0 | — | 4-fluorophenyl | 2.21[a] |
| I-918 | H | 4-methoxybenzyl | Cl | O | cyclopropyl | 0 | — | 3,5-difluorophenyl | 4.10[a] |
| I-919 | H | H | H | O | H | 1 | CH₂ | [3-chloro-5- | 2.01[a] |
| | | | | | | | | (trifluoromethyl)pyridin-2- | |
| | | | | | | | | yl]methyl | |
| I-920 | H | H | H | O | H | 0 | — | 3-chloro-4-methylphenyl | 2.35[a] |
| I-921 | H | 4-methoxybenzyl | Cl | O | cyclopropyl | 0 | — | 2,4-difluorophenyl | 4.02[a] |
| I-922 | H | 4-methoxybenzyl | Cl | O | cyclopropyl | 0 | — | 4-fluorophenyl | 3.85[a] |
| I-923 | H | H | Cl | O | H | 0 | — | 2,4-dimethylphenyl | 2.44[a] |
| I-924 | H | H | Cl | O | H | 0 | — | 2,6-dimethylphenyl | 2.05[a] |
| I-925 | H | H | Cl | O | H | 0 | — | 2,3-dimethylphenyl | 2.27[a] |
| I-926 | H | H | Cl | O | H | 0 | — | 2,3-dihydro-1H-inden-4-yl | 2.75[a] |
| I-927 | H | H | Cl | O | H | 0 | — | 1-naphthyl | 2.39[a] |
| I-928 | H | 4-methoxybenzyl | Cl | O | cyclopropyl | 1 | CHMe | 3,5-dichlorophenyl | 5.36[a] |
| I-929 | H | 4-methoxybenzyl | Cl | O | cyclopropyl | 1 | CH₂ | 2,6-dichlorophenyl | 4.80[a] |
| I-930 | H | 4-methoxybenzyl | Cl | O | cyclopropyl | 1 | CH₂ | 2,4-dichlorophenyl | 5.08[a] |
| I-931 | H | 4-methoxybenzyl | Cl | O | cyclopropyl | 1 | CHMe | 2,4-dichlorophenyl | 5.22[a] |
| I-932 | H | 4-methoxybenzyl | Cl | O | cyclopropyl | 0 | — | 1-(2,4-dichlorophenyl)propyl | 5.62[a] |
| I-933 | H | 4-methoxybenzyl | Cl | O | cyclopropyl | 1 | CH₂ | 2-chloro-6-fluorophenyl | 4.44[a] |
| I-934 | H | 4-methoxybenzyl | Cl | O | cyclobutyl | 1 | CH₂ | 2,4-dichlorophenyl | 5.56[a] |
| I-935 | H | 4-methoxybenzyl | Cl | O | 2,4- | 1 | CH₂ | cyclopropyl | 5.45[a] |
| | | | | | dichlorobenzyl | | | | |
| I-936 | H | 4-methoxybenzyl | Cl | O | cyclopropyl | 0 | — | 1-(3,5-dichlorophenyl)propyl | 5.81[a] |
| I-937 | H | H | H | O | iPr | 1 | CH₂ | 2,4-dichlorophenyl | 3.08[a] |
| I-938 | benzyl | benzyl | H | O | cyclopropyl | 1 | CHMe | sec-butyl | 5.97[a] |
| I-939 | benzyl | benzyl | H | O | cyclopropyl | 1 | CH₂ | 2,6-dichlorophenyl | 5.90[a] |
| I-940 | benzyl | benzyl | H | O | cyclopropyl | 1 | CH₂ | 2,4-dichlorophenyl | 6.30[a] |
| I-941 | benzyl | benzyl | H | O | iPr | 1 | CH₂ | 2,4-dichlorophenyl | 6.48[a] |
| I-942 | benzyl | benzyl | H | O | 2,2- | 1 | CH₂ | 2,4-dichlorophenyl | 6.79[a] |
| | | | | | dimethylcyclopropyl | | | | |
| I-943 | benzyl | benzyl | H | O | cyclopropyl | 1 | CH₂ | 3-chloro-5-(trifluoromethyl)phenyl | 6.16[a] |
| I-944 | H | 4-methoxybenzyl | Cl | O | iPr | 1 | CH₂ | 2,4-dichlorophenyl | 5.39[a] |
| I-945 | H | 4-methoxybenzyl | Cl | O | 2,2- | 1 | CH₂ | 2,4-dichlorophenyl | 5.87[a] |
| | | | | | dimethylcyclopropyl | | | | |
| I-946 | H | 4-methoxybenzyl | Cl | O | cyclopropyl | 1 | CH₂ | 2-chloro-6-(trifluoromethyl)phenyl | 4.91[a] |
| I-947 | H | 4-methoxybenzyl | Cl | O | cyclopropyl | 1 | CH₂ | 3-chloro-5-(trifluoromethyl)phenyl | 5.11[a] |
| I-948 | H | 4-methoxybenzyl | Cl | O | cyclopropyl | 1 | CHMe | 3-(trifluoromethyl)phenyl | 4.91[a] |
| I-949 | H | 4-methoxybenzyl | Cl | O | cyclopropyl | 1 | CHMe | 2-(trifluoromethyl)phenyl | 4.83[a] |
| I-950 | benzyl | benzyl | Br | O | H | 1 | CH₂ | [3-chloro-5- | 5.97[a] |
| | | | | | | | | (trifluoromethyl)pyridin-2- | |
| | | | | | | | | yl]methyl | |
| I-951 | benzyl | benzyl | Br | O | H | 0 | — | 3-chloro-4-methylphenyl | 6.35[a] |
| I-952 | H | H | Cl | O | cyclopropyl | 1 | CHMe | 3,5-dichlorophenyl | 3.70[a] |
| I-953 | H | H | Cl | O | cyclopropyl | 1 | CHMe | 2,4-dichlorophenyl | 3.50[a] |
| I-954 | H | H | Cl | O | cyclopropyl | 0 | — | 1-(2,4-dichlorophenyl)propyl | 3.90[a] |
| I-955 | H | H | Cl | O | cyclopropyl | 0 | — | 1-(3,5-dichlorophenyl)propyl | 4.17[a] |
| I-956 | H | H | Cl | O | cyclopropyl | 1 | CHMe | 3-(trifluoromethyl)phenyl | 3.29[a] |
| I-957 | H | H | Cl | O | cyclopropyl | 1 | CHMe | 2-(trifluoromethyl)phenyl | 3.13[a] |
| I-958 | H | H | Cl | O | cyclopropyl | 0 | — | 2,4-difluorophenyl | 2.39[a] |
| I-959 | H | H | Cl | O | cyclopropyl | 0 | — | 3,5-difluorophenyl | 2.48[a] |
| I-960 | benzyl | benzyl | Br | O | H | 0 | — | 2,4-dichlorophenyl | 7.15[a] |
| I-961 | H | 4-methoxybenzyl | Cl | O | H | 1 | CH₂ | 4-fluorobenzyl | 3.72[a] |
| I-962 | H | 4-methoxybenzyl | Cl | O | H | 1 | CH₂ | 4-chlorobenzyl | 4.08[a] |
| I-963 | H | 4-methoxybenzyl | Cl | O | H | 1 | CH₂ | 2-fluorobenzyl | 3.74[a] |
| I-964 | H | H | Br | O | H | 1 | CH₂ | [3-chloro-5- | 2.55[a] |
| | | | | | | | | (trifluoromethyl)pyridin-2- | |
| | | | | | | | | yl]methyl | |
| I-965 | H | H | Br | O | H | 0 | — | 3-chloro-4-methylphenyl | 2.88[a] |
| I-966 | H | H | Cl | O | H | 1 | CH₂ | 4-fluorobenzyl | 2.13[a] |
| I-967 | H | H | H | O | cyclopropyl | 1 | CH₂ | 2,4-dichlorophenyl | 2.90[a] |
| I-968 | H | H | H | O | cyclopropyl | 1 | CH₂ | 2,6-dichlorophenyl | 2.53[a] |
| I-969 | H | H | H | O | 2,2- | 1 | CH₂ | 2,4-dichlorophenyl | 3.48[a] |
| | | | | | dimethylcyclopropyl | | | | |
| I-970 | H | H | H | O | cyclopropyl | 1 | CH₂ | 3-chloro-5-(trifluoromethyl)phenyl | 3.11[a] |
| I-971 | H | H | Cl | O | H | 1 | CH₂ | 4-chlorobenzyl | 2.46[a] |
| I-972 | H | H | Cl | O | H | 1 | CH₂ | 2-fluorobenzyl | 2.11[a] |
| I-973 | H | benzoyl | Cl | O | iPr | 1 | CHMe | Me | |
| I-974 | H | 2,2- | Cl | O | iPr | 1 | CHMe | Me | 2.80[a] |
| | | dimethylpropanoyl | | | | | | | |
| I-975 | acetyl | acetyl | Cl | O | iPr | 1 | CHMe | Me | 2.77[a] |
| I-976 | H | acetyl | Cl | O | iPr | 1 | CHMe | Me | |
| I-977 | H | H | H | O | cyclopropyl | 1 | CH₂ | mesityl | 2.86[a] |
| I-978 | H | H | H | O | cyclopropyl | 1 | CH₂ | phenyl | 2.02[a] |
| I-979 | H | H | H | O | cyclopropyl | 1 | CHMe | 3,5-dichlorophenyl | 3.19[a] |
| I-980 | H | H | Cl | O | cyclopropyl | 0 | — | cyclohexyl | 2.78[a] |
| I-981 | H | H | Cl | O | H | 1 | CH₂ | ethynyl | |
| I-982 | H | 4-methoxybenzyl | Cl | O | H | 0 | — | cyclohexylcarbonyl | 3.83[a] |
| I-983 | benzyl | benzyl | 4-fluorophenyl | O | H | 1 | CH₂ | benzyl | 5.68[a] |
| I-984 | H | H | H | O | H | 1 | CH₂ | sec-butyl | 1.44[a] |
| I-985 | H | H | H | O | H | 1 | CH₂ | iPr | 1.07[a] |
| I-986 | H | 4-methoxybenzyl | Cl | O | cyclopropyl | 1 | CH₂ | 2-fluoro-5-methylphenyl | 4.46[a] |
| I-987 | H | 4-methoxybenzyl | Cl | O | cyclopropyl | 1 | CH₂ | 2,5-difluorophenyl | 4.15[a] |
| I-988 | H | 4-methoxybenzyl | Cl | O | cyclopropyl | 1 | CH₂ | 5-chloro-2-fluorophenyl | 4.49[a] |
| I-989 | H | 4-methoxybenzyl | Cl | O | cyclopropyl | 1 | CH₂ | 2-bromo-5-chlorophenyl | 4.98[a] |
| I-990 | benzyl | benzyl | H | O | cyclopropyl | 1 | CH₂ | 2-fluoro-5-methylphenyl | 5.56[a] |
| I-991 | benzyl | benzyl | H | O | cyclopropyl | 1 | CH₂ | 2,5-difluorophenyl | 5.25[a] |
| I-992 | benzyl | benzyl | H | O | cyclopropyl | 1 | CH₂ | 5-chloro-2-fluorophenyl | 5.59[a] |
| I-993 | benzyl | benzyl | H | O | cyclopropyl | 1 | CH₂ | 2-bromo-5-chlorophenyl | 6.11[a] |
| I-994 | H | H | 4-fluorophenyl | O | H | 1 | CH₂ | benzyl | 2.59[a] |
| I-995 | H | H | H | O | cyclopropyl | 1 | CH₂ | 2-fluoro-5-methylphenyl | 2.44[a] |
| I-996 | H | benzyl | H | O | cyclopropyl | 1 | CH₂ | 2-fluoro-5-methylphenyl | 4.01[a] |
| I-997 | H | H | H | O | cyclopropyl | 1 | CH₂ | 2,5-difluorophenyl | 2.18[a] |
| I-998 | H | benzyl | H | O | cyclopropyl | 1 | CH₂ | 2,5-difluorophenyl | 3.74[a] |
| I-999 | H | H | H | O | cyclopropyl | 1 | CH₂ | 5-chloro-2-fluorophenyl | 2.49[a] |
| I-1000 | H | benzyl | H | O | cyclopropyl | 1 | CH₂ | 5-chloro-2-fluorophenyl | 4.06[a] |
| I-1001 | H | H | H | O | cyclopropyl | 1 | CH₂ | 2-bromo-5-chlorophenyl | 2.84[a] |
| I-1002 | H | benzyl | H | O | cyclopropyl | 1 | CH₂ | 2-bromo-5-chlorophenyl | 4.51[a] |
| I-1003 | H | 2,3-difluorobenzoyl | Cl | O | 2,3-difluorobenzoyl | 1 | CH₂ | 2,4-difluorophenyl | |
| I-1004 | H | 2,6-difluorobenzoyl | Cl | O | 2,6-difluorobenzoyl | 1 | CH₂ | 2,4-difluorophenyl | |
| I-1005 | H | H | trifluoromethyl | O | Me | 1 | CH₂ | cyclohexyl | |
| I-1006 | (1- | (1- | Cl | O | (1- | 1 | CH₂ | 2,4-difluorophenyl | |
| | fluorocyclopropyl)carbonyl | fluorocyclopropyl)carbonyl | | | fluorocyclopropyl)carbonyl | | | | |
| I-1007 | H | (1- | Cl | O | (1- | 1 | CH₂ | 2,4-difluorophenyl | |
| | | fluorocyclopropyl)carbonyl | | | fluorocyclopropyl)carbonyl | | | | |
| I-1008 | H | H | I | O | cyclohexylmethyl | 1 | CH₂ | H | |
| I-1009 | H | H | Et | O | Me | 1 | CH₂ | cyclohexyl | |
| I-1010 | tert-butoxycarbonyl | tert-butoxycarbonyl | I | O | Me | 1 | CH₂ | cyclohexyl | |
| I-1011 | H | tert-butoxycarbonyl | Cl | O | Me | 1 | CH₂ | cyclohexyl | |
| I-1012 | H | 2,4- | Cl | O | cyclopropyl | 1 | CH₂ | 2,4-difluorophenyl | 4.46[a] |
| | | dimethoxybenzyl | | | | | | | |
| I-1013 | H | (1- | Cl | O | (1- | 1 | CH₂ | 2,4-difluorophenyl | |
| | | chlorocyclo- | | | chlorocyclopropyl)carbonyl | | | | |
| | | propyl)carbonyl | | | | | | | |
| I-1014 | H | H | Cl | O | H | 1 | CH₂ | sec-butyl | 1.98[a] |
| I-1015 | H | H | Cl | S | H | 1 | CH₂ | 2,4,6-trifluorophenyl | 2.86[a] |
| I-1016 | H | H | Cl | S | H | 1 | CH₂ | 2,4-difluorophenyl | 2.74[a] |
| I-1017 | H | (1- | Cl | O | (1- | 1 | CH₂ | cyclohexyl | |
| | | fluorocyclopropyl)carbonyl | | | fluorocyclopropyl)carbonyl | | | | |
| I-1018 | H | H | Cl | O | H | 1 | CH₂ | iPr | 1.55[a] |
| I-1019 | H | H | Cl | S | H | 1 | CH₂ | 3,4-difluorophenyl | |
| I-1020 | H | H | Cl | S | H | 1 | CH₂ | 4-fluorophenyl | |
| I-1021 | H | H | Cl | S | H | 1 | CH₂ | 2-fluorophenyl | 2.73[a] |
| I-1022 | H | H | Cl | S | H | 1 | CH₂ | sec-butyl | 2.87[a] |
| I-1023 | H | H | Cl | S | H | 1 | CH₂ | iPr | |
| I-1024 | H | 4-methoxybenzyl | Cl | O | tert-butoxycarbonyl | 1 | CH₂ | cyclohexyl | |
| I-1025 | tert-butoxycarbonyl | 4-methoxybenzyl | Cl | O | tert-butoxycarbonyl | 1 | CH₂ | cyclohexyl | |
| I-1026 | H | H | Cl | S | H | 1 | CH₂ | 4-chlorophenyl | 3.12[a] |
| I-1027 | H | 4-methoxybenzyl | Cl | O | amino | 1 | CH₂ | cyclohexyl | |
| I-1028 | H | 2-furoyl | Cl | O | 2-furoyl | 1 | CH₂ | cyclohexyl | |
| I-1029 | H | 2-thienylcarbonyl | Cl | O | 2-thienylcarbonyl | 1 | CH₂ | cyclohexyl | |
| I-1030 | H | 4-fluorobenzoyl | Cl | O | 4-fluorobenzoyl | 1 | CH₂ | cyclohexyl | |
| I-1031 | H | 4-methoxybenzyl | Cl | O | H | 0 | — | phenoxy | 3.17[a] |
| I-1032 | 2-bromopropanoyl | 2-bromopropanoyl | Cl | O | H | 1 | CH₂ | cyclohexyl | |
| I-1033 | H | H | Cl | O | H | 0 | — | phenoxy | 1.53[a] |
| I-1034 | H | H | Cl | O | H | 1 | CH₂ | 2-cyclohexylethyl | |
| I-1035 | H | H | Cl | O | H | 1 | CH₂ | 2-cyclopentylethyl | |
| I-1036 | H | H | Cl | O | H | 0 | — | cyclohexylcarbonyl | 2.06[a] |
| I-1037 | H | H | Cl | O | H | 0 | — | 2,3-dimethylcyclohexyl | 2.71 + 2.84 + |
| | | | | | | | | | 2.88[a] |
| I-1038 | H | H | Cl | O | H | 0 | — | cyclohexanoxy | |
| I-1039 | H | 4-methoxybenzyl | Cl | O | H | 1 | CH₂ | methoxymethyl | 2.47[a] |
| I-1040 | H | 4-methoxybenzyl | Cl | O | H | 1 | CH₂ | (methylsulfanyl)methyl | 2.90[a] |
| I-1041 | H | 4-methoxybenzyl | Cl | O | H | 1 | CH₂ | vinyl | 2.77[a] |
| I-1042 | H | H | Cl | O | H | 1 | CH₂ | rel-[(1R,2R,3R)-2,3- | 3.14[a] |
| | | | | | | | | dimethylcyclohexyl] | |
| I-1043 | H | H | Br | O | H | 1 | CH₂ | rel-[(1R,2R)-2-methylcyclohexyl] | 2.83[a] |
| I-1044 | H | H | Br | O | H | 1 | CH₂ | rel-[(1R,2S)-2-methylcyclohexyl] | 2.90[a] |
| I-1045 | H | H | Br | O | H | 1 | CH₂ | 2-methylcyclohexyl | |
| I-1046 | H | H | Cl | O | H | 0 | — | rel-[(1R,2S,3R)-2,3- | |
| | | | | | | | | dimethylcyclohexyl] | |
| I-1047 | H | H | Cl | O | H | 0 | — | rel-[(1R,2R,3R)-2,3- | 2.77[a] |
| | | | | | | | | dimethylcyclohexyl] | |
| I-1048 | H | H | Cl | O | H | 0 | — | rel-[(1R,2R,3S)-2,3- | |
| | | | | | | | | dimethylcyclohexyl] | |
| I-1049 | H | H | Cl | O | H | 1 | CH₂ | pyridin-2-yl | 0.34[a] |
| I-1050 | H | H | Cl | O | H | 1 | CH₂ | 3-fluoropyridin-4-yl | 0.81[a] |
| I-1051 | H | H | Cl | O | H | 1 | CH₂ | 5-fluoropyridin-2-yl | 1.19[a] |
| I-1052 | H | H | Cl | O | H | 1 | CH₂ | 2-fluoropyridin-3-yl | 1.04[a] |
| I-1053 | H | H | Cl | O | H | 1 | CH₂ | 1-chlorocyclohexyl | 2.62[a] |
| I-1054 | H | H | Cl | O | H | 1 | CH₂ | 4-iodophenyl | 2.54[a] |
| I-1055 | H | H | Cl | O | H | 1 | CH₂ | 4-bromo-2-fluorophenyl | 2.47[a] |
| I-1056 | H | H | Cl | O | H | 0 | — | rel-[(1R,2S)-2-bromocyclohexyl] | 2.47[a] |
| I-1057 | H | H | 1-chlorovinyl | O | H | 1 | CH₂ | cyclohexyl | 2.67[a] |
| I-1058 | H | H | Cl | O | H | 1 | CH₂ | 1-bromocyclohexyl | 2.75[a] |
| I-1059 | H | 4-methoxybenzyl | Cl | O | methoxy | 1 | CH₂ | H | 2.90[a] |
| I-1060 | H | difluoroacetyl | fluoro | O | H | 1 | CH₂ | cyclohexyl | |
| I-1061 | H | trichloroacetyl | fluoro | O | H | 1 | CH₂ | cyclohexyl | |
| I-1062 | H | 2,2,3,3- | fluoro | O | H | 1 | CH₂ | cyclohexyl | |
| | | tetrafluoropropanoyl | | | | | | | |
| I-1063 | H | chloroacetyl | fluoro | O | H | 1 | CH₂ | cyclohexyl | |
| I-1064 | H | trifluoroacetyl | fluoro | O | H | 1 | CH₂ | cyclohexyl | |
| I-1065 | H | propionyl | fluoro | O | H | 1 | CH₂ | cyclohexyl | |
| I-1066 | H | H | Cl | O | H | 0 | — | rel-(1R,4aR,8aS)- | 3.31[a] |
| | | | | | | | | decahydronaphthalen-1-yl | |
| I-1067 | H | H | Cl | O | H | 0 | — | rel-(1R,4aS,8aS)- | 3.33[a] |
| | | | | | | | | decahydronaphthalen-1-yl | |
| I-1068 | H | H | Cl | O | H | 0 | — | rel-(1R,4aS,8aS)- | 3.33[a] |
| | | | | | | | | decahydronaphthalen-1-yl | |
| I-1069 | H | H | Cl | O | H | 0 | — | rel-(1R,4aR,8aR)- | 3.48 + 3.41[a] |
| | | | | | | | | decahydronaphthalen-1-yl | |
| I-1070 | H | H | Cl | O | H | 0 | — | rel-(1R,4aS,8aR)- | 3.41 + 3.48[a] |
| | | | | | | | | decahydronaphthalen-1-yl | |
| I-1071 | H | H | Cl | O | H | 0 | — | rel-(1R,4aS,8aR)- | 3.48 + 3.39[a] |
| | | | | | | | | decahydronaphthalen-1-yl | |
| I-1072 | H | H | Cl | O | H | 0 | — | rel-(1R,4aR,8aR)- | 3.41[a] |
| | | | | | | | | decahydronaphthalen-1-yl | |
| I-1073 | H | 4-methoxybenzyl | H | O | H | 1 | (S)—CHMe | cyclohexyl | 3.59[a] |
| I-1074 | H | H | fluoro | O | H | 1 | CH₂ | 2,4,5-trifluorophenyl | |
| I-1075 | H | H | fluoro | O | H | 1 | CH₂ | 2,4,6-trifluorophenyl | |
| I-1076 | trichloroacetyl | trichloroacetyl | fluoro | O | H | 1 | CH₂ | cyclohexyl | |
| I-1077 | propionyl | propionyl | fluoro | O | H | 1 | CH₂ | cyclohexyl | |
| I-1078 | H | H | Cl | O | H | 1 | CH₂ | 4-(tert-butoxycarbonyl-2- | 2.01[a] |
| | | | | | | | | hydroxycyclopentyl | |
| I-1079 | H | 4-methoxybenzyl | Cl | O | H | 0 | — | 1-cyclohexyl-2-phenylethyl | 5.31[a]; |
| | | | | | | | | | 5.10[b] |
| I-1080 | H | H | Cl | O | H | 1 | CH₂ | 4,4-difluorocyclohexyl | 1.85[a] |
| I-1081 | H | H | Cl | O | H | 1 | CH₂ | cycloheptylmethyl | |
| I-1082 | H | H | Cl | O | H | 1 | CH₂ | 2,2-dimethyl-1,3-dioxolan-4-yl | 1.12[a] |
| I-1083 | H | H | Cl | O | H | 0 | — | 4-(trifluoromethyl)phenolate | 2.26[a] |
| I-1084 | H | H | Cl | O | H | 0 | — | 3-fluorophenolate | 1.71[a] |
| I-1085 | H | 4-methoxybenzyl | Cl | O | H | 1 | CH₂ | 2-methoxyethyl | 2.67[a] |
| I-1086 | H | H | Cl | O | H | 1 | CH₂ | 2-methoxyethyl | 0.82[a] |
| I-1087 | H | H | Cl | O | H | 1 | CH₂ | 1,3-dioxolan-2-yl | 0.59[a] |
| I-1088 | H | H | Cl | O | H | 1 | CH₂ | vinyl | 0.89[a] |
| I-1089 | H | H | Cl | O | H | 1 | CH₂ | methoxymethyl | 0.59[a] |
| I-1090 | H | H | Cl | O | H | 0 | — | 2,4-difluorobenzoyl | 1.71[a] |
| I-1091 | H | H | Br | O | H | 0 | — | rel-(1R,4aR,8aR)- | 3.37[a] |
| | | | | | | | | decahydronaphthalen-1-yl | |
| I-1092 | H | H | Br | O | H | 0 | — | rel-(1R,4aR,8aR)- | 3.37[a] |
| | | | | | | | | decahydronaphthalen-1-yl | |
| I-1093 | H | H | Cl | O | H | 1 | CHMe | 2-methoxyphenyl | 2.36[a] |
| I-1094 | H | H | Cl | O | H | 0 | — | 2-methyl-1-phenylpropyl | 2.71[a] |
| I-1095 | H | H | Cl | O | H | 0 | — | 1-cyclohexyl-2-phenylethyl | 3.64[a]; |
| | | | | | | | | | 3.55[b] |
| I-1096 | H | H | Cl | O | H | 1 | CH₂ | cyclopentylmethyl | 3.00[a] |
| I-1097 | H | H | Cl | O | H | 1 | CH₂ | 4-fluorocyclohex-3-en-1-yl | 1.98[a] |
| I-1098 | H | H | Cl | O | H | 1 | CH₂ | 2,4-difluorobenzyl | 2.29[a] |
| I-1099 | H | 4-methoxybenzyl | Cl | O | H | 1 | CHMe | 2-methoxyphenyl | 4.06[a] |
| I-1100 | H | 4-methoxybenzyl | Cl | O | H | 0 | — | 2-methyl-1-phenylpropyl | 4.37[a] |
| I-1101 | H | H | Cl | O | H | 1 | CH₂ | (methylsulfanyl)methyl | 1.14[a] |
| I-1102 | H | 4-methoxybenzyl | Cl | O | H | 1 | CH₂ | 3-hydroxy-4-methoxyphenyl | 2.71[a] |
| I-1103 | H | 4-methoxybenzyl | Cl | O | H | 1 | CH₂ | pyrimidin-4-yl | 2.13[a] |
| I-1104 | H | H | Cl | O | H | 0 | — | 4-fluorophenolate | 1.57[a] |
| I-1105 | H | 2,2,3,3,3- | fluoro | O | H | 1 | CH₂ | 2,4,5-trifluorophenyl | |
| | | pentafluoropropanoyl | | | | | | | |
| I-1106 | H | trichloroacetyl | fluoro | O | H | 1 | CH₂ | 2,4-difluorophenyl | |
| I-1107 | H | difluoroacetyl | fluoro | O | H | 1 | CH₂ | 2,4-difluorophenyl | |
| I-1108 | acetyl | acetyl | fluoro | O | H | 1 | CH₂ | 2,4,6-trifluorophenyl | |
| I-1109 | H | acetyl | fluoro | O | H | 1 | CH₂ | 2,4,6-trifluorophenyl | |
| I-1110 | H | difluoroacetyl | fluoro | O | H | 1 | CH₂ | 2,4,6-trifluorophenyl | |
| I-1111 | H | 2,2,3,3,3- | fluoro | O | H | 1 | CH₂ | 2,4-difluorophenyl | |
| | | pentafluoropropanoyl | | | | | | | |
| I-1112 | H | 2,2,3,3- | fluoro | O | H | 1 | CH₂ | 2,4,5-trifluorophenyl | |
| | | tetrafluoropropanoyl | | | | | | | |
| I-1113 | H | 2,2,3,3- | fluoro | O | H | 1 | CH₂ | 2,4,6-trifluorophenyl | |
| | | tetrafluoropropanoyl | | | | | | | |
| I-1114 | H | 2,2,3,3,3- | fluoro | O | H | 1 | CH₂ | 2,4,6-trifluorophenyl | |
| | | pentafluoropropanoyl | | | | | | | |
| I-1115 | H | 2,2,3,3- | fluoro | O | H | 1 | CH₂ | 2,4-difluorophenyl | |
| | | tetrafluoropropanoyl | | | | | | | |
| I-1116 | H | trichloroacetyl | fluoro | O | H | 1 | CH₂ | 2,4,5-trifluorophenyl | |
| I-1117 | H | methoxyacetyl | fluoro | O | H | 1 | CH₂ | cyclohexyl | |
| I-1118 | H | acetyl | fluoro | O | H | 1 | CH₂ | 2,4-difluorophenyl | |
| I-1119 | acetyl | acetyl | fluoro | O | H | 1 | CH₂ | 2,4-difluorophenyl | |
| I-1120 | H | trichloroacetyl | fluoro | O | H | 1 | CH₂ | 2,4,6-trifluorophenyl | |
| I-1121 | H | difluoroacetyl | fluoro | O | H | 1 | CH₂ | 2,4,5-trifluorophenyl | |
| I-1122 | H | acetyl | fluoro | O | H | 1 | CH₂ | 2,4,5-trifluorophenyl | |
| I-1123 | acetyl | acetyl | fluoro | O | H | 1 | CH₂ | 2,4,5-trifluorophenyl | |
| I-1124 | H | H | Cl | O | H | 1 | CH₂ | 2-hydroxy-4- | 0.99[a] |
| | | | | | | | | (methoxycarbonyl)cyclopentyl | |
| I-1125 | cyclopropylcarbonyl | cyclopropylcarbonyl | Cl | O | H | 0 | — | rel-(1R,4aR,8aR)- | 5.28[a] |
| | | | | | | | | decahydronaphthalen-1-yl | |
| I-1126 | H | cyclopropylcarbonyl | Cl | O | H | 0 | — | rel-(1R,4aR,8aR)- | 3.51[a] |
| | | | | | | | | decahydronaphthalen-1-yl | |
| I-1127 | cyclopropylcarbonyl | cyclopropylcarbonyl | Cl | O | H | 1 | CH₂ | 2-fluorophenyl | 3.51[a] |
| I-1128 | H | isobutyryl | Cl | O | H | 0 | — | rel-(1R,4aR,8aR)- | 3.73[a] |
| | | | | | | | | decahydronaphthalen-1-yl | |
| I-1129 | H | propionyl | Cl | O | H | 0 | — | rel-(1R,4aR,8aR)- | 3.35[a] |
| | | | | | | | | decahydronaphthalen-1-yl | |
| I-1130 | H | H | Cl | S | H | 0 | — | rel-(1R,4aR,8aR)- | 4.46[a] |
| | | | | | | | | decahydronaphthalen-1-yl | |
| I-1131 | H | H | Cl | O | H | 1 | CH₂ | rel-[(1R,2S)-2-bromocyclohexyl] | 2.32[a] |
| I-1132 | H | H | Cl | O | H | 1 | CH₂ | rel-[(1R,2S)-2-bromocyclohexyl] | 2.46[a] |
| I-1133 | H | 4-methoxybenzyl | Cl | O | H | 1 | CH₂ | rel-[(1R,2S)-2-bromocyclohexyl] | |
| I-1134 | H | 4-methoxybenzyl | Cl | O | H | 1 | CH₂ | rel-[(1R,2S)-2-bromocyclohexyl] | |
| I-1135 | H | H | fluoro | O | H | 1 | CH₂ | 4-fluorophenyl | |
| I-1136 | H | H | fluoro | O | H | 1 | CH₂ | 4-chlorophenyl | |
| I-1137 | H | 2,2,3,3- | fluoro | O | H | 1 | CH₂ | 4-chlorophenyl | |
| | | tetrafluoropropanoyl | | | | | | | |
| I-1138 | H | trichloroacetyl | fluoro | O | H | 1 | CH₂ | 4-fluorophenyl | |
| I-1139 | H | 2,2,3,3- | fluoro | O | H | 1 | CH₂ | 4-fluorophenyl | |
| | | tetrafluoropropanoyl | | | | | | | |
| I-1140 | H | 2,2,3,3,3- | fluoro | O | H | 1 | CH₂ | 4-chlorophenyl | |
| | | pentafluoropropanoyl | | | | | | | |
| I-1141 | H | trichloroacetyl | fluoro | O | H | 1 | CH₂ | 4-chlorophenyl | |
| I-1142 | H | 2,2,3,3,3- | fluoro | O | H | 1 | CH₂ | 4-fluorophenyl | |
| | | pentafluoropropanoyl | | | | | | | |
| I-1143 | H | acetyl | fluoro | O | H | 1 | CH₂ | 4-chlorophenyl | |
| I-1144 | acetyl | acetyl | fluoro | O | H | 1 | CH₂ | 4-chlorophenyl | |
| I-1145 | H | difluoroacetyl | fluoro | O | H | 1 | CH₂ | 4-chlorophenyl | |
| I-1146 | H | acetyl | fluoro | O | H | 1 | CH₂ | 4-fluorophenyl | |
| I-1147 | H | H | Cl | O | H | 1 | CH₂ | 4-cyanocyclohexyl | 1.34[a] |
| I-1148 | H | H | Cl | O | H | 1 | CH₂ | isopropoxymethyl | 1.41[a] |
| I-1149 | H | 3,3,3- | fluoro | O | H | 1 | CH₂ | 4-chlorophenyl | |
| | | trifluoropropanoyl | | | | | | | |
| I-1150 | H | difluoroacetyl | fluoro | O | H | 1 | CH₂ | 4-fluorophenyl | |
| I-1151 | H | 3,3,3- | fluoro | O | H | 1 | CH₂ | 4-fluorophenyl | |
| | | trifluoropropanoyl | | | | | | | |
| I-1152 | H | 3,3,3- | fluoro | O | H | 1 | CH₂ | 2,4,6-trifluorophenyl | |
| | | trifluoropropanoyl | | | | | | | |
| I-1153 | H | 3,3,3- | fluoro | O | H | 1 | CH₂ | 2,4-difluorophenyl | |
| | | trifluoropropanoyl | | | | | | | |
| I-1154 | H | 3,3,3- | fluoro | O | H | 1 | CH₂ | 2,4,5-trifluorophenyl | |
| | | trifluoropropanoyl | | | | | | | |
| I-1155 | H | H | fluoro | O | H | 1 | CH₂ | 2,3,4-trifluorophenyl | |
| I-1156 | H | H | fluoro | O | H | 1 | CH₂ | cyclopentyl | |
| I-1157 | H | H | fluoro | O | H | 1 | CH₂ | cycloheptyl | |
| I-1158 | H | 4-methoxybenzyl | Cl | O | H | 1 | CHMe | cyclohexyl | 4.61[a] |
| I-1159 | H | H | Cl | O | H | 1 | CH₂ | prop-1-yn-1-yl | 0.98[a] |
| I-1160 | H | H | fluoro | O | H | 1 | CH₂ | tetrahydrofuran-2-yl | 0.72[a] |
| I-1161 | H | H | fluoro | O | H | 1 | CH₂ | tetrahydrofuran-3-yl | 0.49[a] |
| I-1162 | H | difluoroacetyl | fluoro | O | H | 1 | CH₂ | cyclopentyl | |
| I-1163 | H | propionyl | fluoro | O | H | 1 | CH₂ | 2,3,4-trifluorophenyl | |
| I-1164 | H | trifluoroacetyl | fluoro | O | H | 1 | CH₂ | 2,3,4-trifluorophenyl | |
| I-1165 | H | difluoroacetyl | fluoro | O | H | 1 | CH₂ | 2,3,4-trifluorophenyl | |
| I-1166 | H | trichloroacetyl | fluoro | O | H | 1 | CH₂ | 2,3,4-trifluorophenyl | |
| I-1167 | H | chloroacetyl | fluoro | O | H | 1 | CH₂ | 2,3,4-trifluorophenyl | |
| I-1168 | H | H | Cl | O | H | 1 | CH₂ | rel-[(1R,2R)-2-chlorocyclohexyl] | 2.34[a] |
| I-1169 | H | H | Cl | O | H | 1 | CH₂ | rel-[(1R,2S)-2-chlorocyclohexyl] | 2.34[a] |
| I-1170 | H | 4-methoxybenzyl | Cl | O | H | 0 | — | 1-cyclohexylpropyl | 4.96[a]; |
| | | | | | | | | | 4.83[b] |
| I-1171 | H | 4-methoxybenzyl | Cl | O | H | 1 | CH₂ | piperidin-3-yl | |
| I-1172 | H | 4-methoxybenzyl | Cl | O | H | 1 | CH₂ | 2-hydroxypyridin-3-yl | 2.00[a] |
| I-1173 | H | 4-methoxybenzyl | Cl | O | H | 0 | — | 2-methyl-1-(4-methylpyrimidin-5- | 2.84[a] |
| | | | | | | | | yl)propyl | |
| I-1174 | H | 4-methoxybenzyl | Cl | O | H | 0 | — | 1,1′-bi(cyclohexyl)-2-yl | 5.78[a] |
| I-1175 | H | 4-methoxybenzyl | Cl | O | H | 1 | CH₂ | 1,3,5-trimethyl-1H-pyrazol-4-yl | 2.34[a] |
| I-1176 | H | 4-methoxybenzyl | Cl | O | H | 1 | CHMe | 2,4-dimethyl-1,3-thiazol-5-yl | 2.73[a] |
| I-1177 | H | 4-methoxybenzyl | Cl | O | H | 1 | CHMe | 2-methylphenyl | 3.99[a] |
| I-1178 | H | 4-methoxybenzyl | Cl | O | H | 1 | CH₂ | 2-(difluoromethoxy)phenyl | 3.70[a] |
| I-1179 | H | 4-methoxybenzyl | Cl | O | H | 1 | CH₂ | 2,4-dimethoxyphenyl | 3.63[a] |
| I-1180 | H | 4-methoxybenzyl | Cl | O | H | 1 | CH₂ | 6-propylpyridin-2-yl | 2.59[a] |
| I-1181 | H | 4-methoxybenzyl | Cl | O | H | 1 | CHMe | cyclohex-3-en-1-yl | 4.18[a] |
| I-1182 | H | 4-methoxybenzyl | Cl | O | H | 0 | — | 1-cyano-2-methylcyclohexyl | 3.68[a] |
| I-1183 | H | 4-methoxybenzyl | Cl | O | H | 1 | CHMe | 2-(trifluoromethyl)phenyl | 4.18[a] |
| I-1184 | H | 4-methoxybenzyl | Cl | O | H | 0 | — | 2-isopropylcyclohexyl | 5.11[a] |
| I-1185 | H | 4-methoxybenzyl | Cl | O | H | 1 | CHMe | 3-(trifluoromethyl)phenyl | 4.18[a] |
| I-1186 | H | 4-methoxybenzyl | Cl | O | H | 0 | — | 1-(4-chlorophenyl)propyl | 4.41[a] |
| I-1187 | H | 4-methoxybenzyl | Cl | O | H | 1 | CHMe | 2-thienyl | 3.61[a] |
| I-1188 | H | 4-methoxybenzyl | Cl | O | H | 0 | — | 2-methyl-1-[4- | 3.85[a] |
| | | | | | | | | (trifluoromethyl)pyrimidin-5- | |
| | | | | | | | | yl]propyl | |
| I-1189 | H | 4-methoxybenzyl | Cl | O | H | 1 | CHMe | pyridin-4-yl | 1.54[a] |
| I-1190 | H | 4-methoxybenzyl | Cl | O | H | 0 | — | tetrahydro-2H-pyran-4-yl | 2.50[a] |
| I-1191 | H | 4-methoxybenzyl | Cl | O | H | 0 | — | 2,6-diethyl-4-methylcyclohexyl | 6.02[a] |
| I-1192 | H | 4-methoxybenzyl | Cl | O | H | 0 | — | 2-methyl-1-(2-methylphenyl)propyl | 4.69[a] |
| I-1193 | H | 4-methoxybenzyl | Cl | O | H | 1 | CH₂ | 5-ethylpyridin-2-yl | 2.34[a] |
| I-1194 | H | 4-methoxybenzyl | Cl | O | H | 1 | CH₂ | 2-(1H-pyrazol-1-yl)ethyl | 2.53[a] |
| I-1195 | H | 4-methoxybenzyl | Cl | O | H | 0 | — | cyclobutyl | 3.15[a] |
| I-1196 | H | 4-methoxybenzyl | Cl | O | H | 1 | CH₂ | tetrahydrofuran-3-ylmethyl | 2.59[a] |
| I-1197 | H | 4-methoxybenzyl | Cl | O | H | 1 | CHMe | pyridin-2-yl | 2.57[a] |
| I-1198 | H | 4-methoxybenzyl | Cl | O | H | 1 | CH₂ | 3-(difluoromethyl)-1-ethyl-1H- | 3.09[a] |
| | | | | | | | | pyrazol-4-yl | |
| I-1199 | H | 4-methoxybenzyl | Cl | O | H | 1 | CH₂ | 4-tert-butylphenyl | 4.69[a] |
| I-1200 | H | 4-methoxybenzyl | Cl | O | H | 1 | CH₂ | 4-methyltetrahydrofuran-3-yl | 2.68[a] |
| I-1201 | H | 4-methoxybenzyl | Cl | O | H | 1 | CH₂ | 4-methyl-3-thienyl | 3.61[a] |
| I-1202 | H | 4-methoxybenzyl | Cl | O | H | 0 | — | 1-(1-ethyl-3-methyl-1H-pyrazol-4- | 3.02[a] |
| | | | | | | | | yl)propyl | |
| I-1203 | H | 4-methoxybenzyl | Cl | O | H | 1 | CH₂ | 4-methyl-1,3-thiazol-2-yl | 2.80[a] |
| I-1204 | H | 4-methoxybenzyl | Cl | O | H | 1 | CH₂ | 2-methoxypyridin-4-yl | 2.78[a] |
| I-1205 | H | 4-methoxybenzyl | Cl | O | H | 1 | CH₂ | 1-ethyl-1H-pyrazol-4-yl | 2.48[a] |
| I-1206 | H | 4-methoxybenzyl | Cl | O | H | 0 | — | 3,3,5-trimethylcyclohexyl | 5.00[a] |
| I-1207 | H | 4-methoxybenzyl | Cl | O | H | 1 | CHMe | 4-isopropylphenyl | 4.69[a] |
| I-1208 | H | 4-methoxybenzyl | Cl | O | H | 1 | CH₂ | 3,4-dihydro-1H-isochromen-1-yl | 3.72[a] |
| I-1209 | H | 4-methoxybenzyl | Cl | O | H | 0 | — | 1-ethynyleyclohexyl | 3.99[a] |
| I-1210 | H | 4-methoxybenzyl | Cl | O | H | 0 | — | 2,3-dimethylcyclohexyl | 4.46[a] |
| I-1211 | H | 4-methoxybenzyl | Cl | O | H | 1 | CH₂ | 1-methyl-1H-pyrrol-2-yl | 3.15[a] |
| I-1212 | H | 4-methoxybenzyl | Cl | O | H | 1 | CHMe | 2-fluorophenyl | 3.79[a] |
| I-1213 | H | 4-methoxybenzyl | Cl | O | H | 1 | CH₂ | 1-methyl-1H-imidazol-5-yl | 1.35[a] |
| I-1214 | H | 4-methoxybenzyl | Cl | O | H | 1 | CH₂ | 1-ethyl-3,5-dimethyl-1H-pyrazol-4- | 2.57[a] |
| | | | | | | | | yl | |
| I-1215 | H | 4-methoxybenzyl | Cl | O | H | 0 | — | 2-ethylcyclohexyl | 4.49[a] |
| I-1216 | H | 4-methoxybenzyl | Cl | O | H | 0 | — | decahydronaphthalen-2-yl | 5.24[a] |
| I-1217 | H | 4-methoxybenzyl | Cl | O | H | 1 | CH₂ | 2-methyl-1,3-thiazol-4-yl | 2.69[a] |
| I-1218 | H | 4-methoxybenzyl | Cl | O | H | 0 | — | 3,5-bis(trifluoromethyl)cyclohexyl | 4.30[a] |
| I-1219 | H | 4-methoxybenzyl | Cl | O | H | 0 | — | 2,6-diisopropylcyclohexyl | 6.32[a] |
| I-1220 | H | 4-methoxybenzyl | Cl | O | H | 0 | — | 1-cyano-2-methylcyclopentyl | 3.41[a] |
| I-1221 | H | H | Cl | O | H | 0 | — | 1-cyclohexylpropyl | 3.12[a]; |
| | | | | | | | | | 3.04[b] |
| I-1222 | propionyl | propionyl | fluoro | O | H | 1 | CH₂ | cycloheptyl | |
| I-1223 | H | propionyl | fluoro | O | H | 1 | CH₂ | cycloheptyl | |
| I-1224 | H | 3,3,3- | fluoro | O | H | 1 | CH₂ | 2,3,4-trifluorophenyl | |
| | | trifluoropropanoyl | | | | | | | |
| I-1225 | acetyl | acetyl | fluoro | O | H | 1 | CH₂ | 2,3,4-trifluorophenyl | |
| I-1226 | H | 2,2,3,3,3- | fluoro | O | H | 1 | CH₂ | 2,3,4-trifluorophenyl | |
| | | pentafluoropropanoyl | | | | | | | |
| I-1227 | H | methoxyacetyl | fluoro | O | H | 1 | CH₂ | 2,3,4-trifluorophenyl | |
| I-1228 | H | H | Cl | O | H | 1 | CH₂ | (dimethylamino)methyl | |
| I-1229 | H | 4-methoxybenzyl | Cl | O | H | 1 | CH₂ | 4-(difluoromethoxy)phenyl | 3.63[a] |
| I-1230 | H | 4-methoxybenzyl | Cl | O | H | 1 | CH₂ | 2,5-dimethoxyphenyl | 3.59[a] |
| I-1231 | H | 4-methoxybenzyl | Cl | O | H | 1 | CH₂ | pyrazin-2-yl | 2.26[a] |
| I-1232 | H | 4-methoxybenzyl | Cl | O | H | 1 | —C(Me)₂— | 3-(trifluoromethyl)phenyl | 4.44[a] |
| I-1233 | H | 4-methoxybenzyl | Cl | O | H | 1 | CH₂ | 3-(trifluoromethoxy)phenyl | 4.13[a] |
| I-1234 | H | 4-methoxybenzyl | Cl | O | H | 1 | CHMe | 3-methoxyphenyl | 3.68[a] |
| I-1235 | H | 4-methoxybenzyl | Cl | O | H | 1 | CH₂ | 2-methoxypyridin-3-yl | 3.13[a] |
| I-1236 | H | 4-methoxybenzyl | Cl | O | H | 1 | CH₂ | 2-methyltetrahydrofuran-2-yl | 3.06[a] |
| I-1237 | H | 4-methoxybenzyl | Cl | O | H | 1 | CH₂ | 1-ethyl-5-methyl-1H-pyrazol-4-yl | 2.59[a] |
| I-1238 | H | 4-methoxybenzyl | Cl | O | H | 1 | CH₂ | 1-methyl-1H-pyrazol-3-yl | 2.39[a] |
| I-1239 | H | 4-methoxybenzyl | Cl | O | H | 1 | CHMe | 2-furylmethyl | 3.55[a] |
| I-1240 | H | 4-methoxybenzyl | Cl | O | H | 1 | CH₂ | 3-chloro-4-methoxyphenyl | 3.68[a] |
| I-1241 | H | 4-methoxybenzyl | Cl | O | H | 1 | CH₂ | 2-thienyl | 3.31[a] |
| I-1242 | H | 4-methoxybenzyl | Cl | O | H | 1 | CH₂ | 4-(trifluoromethoxy)phenyl | 4.15[a] |
| I-1243 | H | 4-methoxybenzyl | Cl | O | H | 1 | CH₂ | 3-chloro-2-methylphenyl | 4.20[a] |
| I-1244 | H | 4-methoxybenzyl | Cl | O | H | 1 | CH₂ | 2-(1H-imidazol-1-yl)ethyl | 1.36[a] |
| I-1245 | H | 4-methoxybenzyl | Cl | O | H | 0 | — | cycloheptyl | 4.27[a] |
| I-1246 | H | 4-methoxybenzyl | Cl | O | H | 1 | CH₂ | 2-chloro-5-(trifluoromethyl)phenyl | 4.37[a] |
| I-1247 | H | 4-methoxybenzyl | Cl | O | H | 1 | CH₂ | 2-ethoxyphenyl | 4.08[a] |
| I-1248 | H | 4-methoxybenzyl | Cl | O | H | 0 | — | 2,3-dihydro-1H-inden-2-yl | 3.90[a] |
| I-1249 | H | 4-methoxybenzyl | Cl | O | H | 1 | CH₂ | 4-chloro-3-(trifluoromethyl)phenyl | 4.34[a] |
| I-1250 | H | 4-methoxybenzyl | Cl | O | H | 1 | CHMe | 1-chlorocyclopropyl | 3.90[a] |
| I-1251 | H | 4-methoxybenzyl | Cl | O | H | 0 | — | 2-methyl-1-(pyrimidin-5-yl)propyl | 2.73[a] |
| I-1252 | H | 4-methoxybenzyl | Cl | O | H | 0 | — | 4-tert-butylcyclohexyl | 5.51[a] |
| I-1253 | H | 4-methoxybenzyl | Cl | O | H | 1 | CHMe | pyridin-3-yl | 1.63[a] |
| I-1254 | H | 4-methoxybenzyl | Cl | O | H | 0 | — | 3-ethyl-3,5,5-trimethylcyclohexyl | 5.72[a] |
| I-1255 | H | 4-methoxybenzyl | Cl | O | H | 1 | CH₂ | pyridin-2-ylmethyl | 1.52[a] |
| I-1256 | H | 4-methoxybenzyl | Cl | O | H | 1 | CHMe | 2,4-dichlorophenyl | 4.61[a] |
| I-1257 | H | 4-methoxybenzyl | Cl | O | H | 1 | CH₂ | 1-isopropyl-3-methyl-1H-pyrazol- | 2.82[a] |
| | | | | | | | | 4-yl | |
| I-1258 | H | 4-methoxybenzyl | Cl | O | H | 1 | CH₂ | 1-(2,2,2-trifluoroethyl)-1H-pyrazol- | 2.84[a] |
| | | | | | | | | 4-yl | |
| I-1259 | H | 4-methoxybenzyl | Cl | O | H | 1 | CH₂ | 3-methyl-1-pentyl-1H-pyrazol-4-yl | 3.57[a] |
| I-1260 | H | 4-methoxybenzyl | Cl | O | H | 1 | CH₂ | 1-butyl-3-methyl-1H-pyrazol-4-yl | 3.21[a] |
| I-1261 | H | 4-methoxybenzyl | Cl | O | H | 1 | CHMe | 3-thienyl | 3.61[a] |
| I-1262 | H | 4-methoxybenzyl | Cl | O | H | 1 | CH₂ | 1-isobutyl-5-methyl-1H-pyrazol-4- | 3.21[a] |
| | | | | | | | | yl | |
| I-1263 | H | 4-methoxybenzyl | Cl | O | H | 1 | CH₂ | 2-(2-methyl-1H-imidazol-1-yl)ethyl | 1.44[a] |
| I-1264 | H | 4-methoxybenzyl | Cl | O | H | 1 | CH₂ | 3-fluoro-5-(trifluoromethyl)phenyl | 4.15[a] |
| I-1265 | H | 4-methoxybenzyl | Cl | O | H | 0 | — | 1-phenylpropyl | 4.03[a] |
| I-1266 | H | 4-methoxybenzyl | Cl | O | H | 1 | CH₂ | cyclohex-1-en-1-ylmethyl | 4.44[a] |
| I-1267 | H | 4-methoxybenzyl | Cl | O | H | 1 | —C(Me)₂— | 4-methyl-1,3-thiazol-2-yl | 3.63[a] |
| I-1268 | H | 4-methoxybenzyl | Cl | O | H | 1 | CH₂ | 1-naphthyl | 4.11[a] |
| I-1269 | H | 4-methoxybenzyl | Cl | O | H | 1 | CH₂ | 2-thienylmethyl | 3.55[a] |
| I-1270 | H | 4-methoxybenzyl | Cl | O | H | 1 | CH₂ | 5-methyl-1,2,4-oxadiazol-3-yl | 2.47[a] |
| I-1271 | H | 4-methoxybenzyl | Cl | O | H | 1 | CH₂ | 3-methoxypyridin-2-yl | 2.70[a] |
| I-1272 | H | 4-methoxybenzyl | Cl | O | H | 1 | CH₂ | 4-fluoro-3-(trifluoromethyl)phenyl | 4.06[a] |
| I-1273 | H | 4-methoxybenzyl | Cl | O | H | 1 | CHMe | 4-bromophenyl | 4.23[a] |
| I-1274 | H | 4-methoxybenzyl | Cl | O | H | 1 | CH₂ | 6-isopropylpyridin-2-yl | 2.94[a] |
| I-1275 | H | 4-methoxybenzyl | Cl | O | H | 1 | CHMe | 1-naphthyl | 4.32[a] |
| I-1276 | H | 4-methoxybenzyl | Cl | O | H | 1 | CHMe | 4-(trifluoromethyl)phenyl | 4.23[a] |
| I-1277 | H | H | fluoro | O | H | 1 | CH₂ | 3-fluorophenyl | |
| I-1278 | H | H | fluoro | O | H | 1 | CH₂ | 2,3-difluorophenyl | |
| I-1279 | H | H | fluoro | O | H | 1 | CH₂ | 2,5-difluorophenyl | |
| I-1280 | H | H | fluoro | O | H | 1 | CH₂ | 3,4-difluorophenyl | |
| I-1281 | H | H | fluoro | O | H | 1 | CH₂ | 2,3,6-trifluorophenyl | |
| I-1282 | H | H | fluoro | O | H | 1 | CH₂ | 2-fluorophenyl | |
| I-1283 | H | H | fluoro | O | H | 1 | CH₂ | 3,5-difluorophenyl | |
| I-1284 | H | H | fluoro | O | H | 1 | CH₂ | 2,6-difluorophenyl | |
| I-1285 | H | H | fluoro | O | H | 1 | CH₂ | 3,4,5-trifluorophenyl | |
| I-1286 | tert-butoxycarbonyl | 4-methoxybenzyl | Cl | O | methoxy | 1 | CH₂ | H | 3.75[a] |
| I-1287 | H | H | fluoro | O | H | 1 | CH₂ | rel-[(1R,2R)-2-chlorocyclohexyl] | 2.21[a]; |
| | | | | | | | | | 2.20[c] |
| I-1288 | H | H | Cl | O | H | 1 | CH₂ | 2-cyanophenyl | 1.43[a] |
| I-1289 | H | H | Cl | O | H | 1 | CH₂ | 3-cyanophenyl | 1.41[a] |
| I-1290 | H | H | Cl | O | H | 1 | CH₂ | rel-[(1R,2S,3R)-2,3- | |
| | | | | | | | | dimethylcyclohexyl] | |
| I-1291 | H | H | Cl | O | H | 1 | CH₂ | rel-[(1R,2R,3R)-2,3- | 3.10[a] |
| | | | | | | | | dimethylcyclohexyl] | |
| I-1292 | H | 2-bromopropanoyl | fluoro | O | H | 1 | CH₂ | cyclohexyl | |
| I-1293 | H | H | I | O | H | 1 | CH₂ | rel-[(1R,2R)-2-chlorocyclohexyl] | 2.50[a] |
| I-1294 | H | H | Cl | O | H | 1 | CH₂ | 2,2-difluorocyclohexyl | 2.07[a] |
| I-1295 | H | tert-butoxycarbonyl | fluoro | O | H | 1 | CH₂ | 2-chlorocyclohexyl | |
| I-1296 | H | H | fluoro | O | H | 1 | CH₂ | 4-fluorocyclohex-3-en-1-yl | 1.69[a] |
| I-1297 | H | H | fluoro | O | H | 1 | CH₂ | 4,4-difluorocyclohexyl | 1.66[a] |
| I-1298 | H | H | fluoro | O | H | 0 | — | cyclopentyl | |
| I-1299 | H | H | fluoro | O | H | 1 | CH₂ | 2-methylcyclohexyl | |
| I-1300 | H | bromoacetyl | fluoro | O | H | 1 | CH₂ | cyclohexyl | |
| I-1301 | H | H | fluoro | O | H | 1 | CH₂ | 2,3-dimethylcyclohexyl | |
| I-1302 | H | H | fluoro | O | H | 0 | — | cyclohexyl | |
| I-1303 | H | H | fluoro | O | H | 1 | (R)—CHMe | cyclohexyl | |
| I-1304 | H | H | fluoro | O | H | 1 | (S)—CHMe | cyclohexyl | |
| I-1305 | H | H | fluoro | O | H | 1 | CH₂ | 2,3,5-trifluorophenyl | |
| I-1306 | H | H | Cl | O | H | 1 | CH₂ | rel-[(1R,2R)-2-fluorocyclohexyl] | 1.97[a] |
| I-1307 | H | H | Cl | O | H | 1 | CH₂ | 3-(ethoxycarbonyl)phenyl | 2.03[a] |
| I-1308 | H | 4-methoxybenzyl | Cl | O | H | 0 | — | 7-tert-butyl-1,2,3,4- | 5.41[a] |
| | | | | | | | | tetrahydronaphthalen-2-yl | |
| I-1309 | H | 4-methoxybenzyl | Cl | O | H | 1 | CH₂ | 5-chloropyrazin-2-yl | 2.98[a] |
| I-1310 | H | 4-methoxybenzyl | Cl | O | H | 0 | — | 5,6,7,8-tetrahydroisoquinolin-8-yl | 1.67[a] |
| I-1311 | H | 4-methoxybenzyl | Cl | O | H | 0 | — | 6-tert-butyl-1,2,3,4- | 5.46[a] |
| | | | | | | | | tetrahydronaphthalen-1-yl | |
| I-1312 | H | 4-methoxybenzyl | Cl | O | H | 1 | CH₂ | 5-(difluoromethyl)-1-ethyl-1H- | 3.09[a] |
| | | | | | | | | pyrazol-4-yl | |
| I-1313 | H | 4-methoxybenzyl | Cl | O | H | 1 | CH₂ | 3,5-bis(trifluoromethyl)phenyl | 4.46[a] |
| I-1314 | H | 4-methoxybenzyl | Cl | O | H | 1 | CH₂ | 3-methylpyridin-4-yl | 1.52[a] |
| I-1315 | H | 4-methoxybenzyl | Cl | O | H | 1 | CH₂ | 1-(2,2-difluoroethyl)-1H-pyrazol-4- | 2.58[a] |
| | | | | | | | | yl | |
| I-1316 | H | 4-methoxybenzyl | Cl | O | H | 1 | CH₂ | 2-methoxypyrimidin-4-yl | 2.56[a] |
| I-1317 | H | 4-methoxybenzyl | Cl | O | H | 1 | CHMe | 1,3-thiazol-2-yl | 2.82[a] |
| I-1318 | H | 4-methoxybenzyl | Cl | O | H | 1 | CH₂ | 5-methoxypyridin-2-yl | 2.45[a] |
| I-1319 | H | 4-methoxybenzyl | Cl | O | H | 1 | CH₂ | 6-methoxypyridin-2-yl | 3.41[a] |
| I-1320 | H | 4-methoxybenzyl | Cl | O | H | 1 | CH₂ | 1,5-dimethyl-1H-pyrrol-2-yl | 3.39[a] |
| I-1321 | H | 4-methoxybenzyl | Cl | O | H | 1 | CH₂ | 4-methoxy-3- | 3.85[a] |
| | | | | | | | | (trifluoromethyl)phenyl | |
| I-1322 | H | 4-methoxybenzyl | Cl | O | H | 1 | —C(CH₂)₂— | 2-bromophenyl | 4.40[a] |
| I-1323 | H | 4-methoxybenzyl | Cl | O | H | 1 | CH₂ | 1,3-thiazol-5-yl | 2.39[a] |
| I-1324 | H | 4-methoxybenzyl | Cl | O | H | 0 | — | 1-(1H-imidazol-1-yl)-3,3- | 1.82[a] |
| | | | | | | | | dimethylbutan-2-yl | |
| I-1325 | H | 4-methoxybenzyl | Cl | O | H | 0 | — | 2,6,6-trimethyl-4,5,6,7-tetrahydro- | 4.74[a] |
| | | | | | | | | 1-benzofuran-4-yl | |
| I-1326 | H | 4-methoxybenzyl | Cl | O | H | 1 | CH—CN | 3,4-difluorophenyl | 3.61[a] |
| I-1327 | H | 4-methoxybenzyl | Cl | O | H | 1 | —C(CH₂)₂— | 2-chlorophenyl | 4.24[a] |
| I-1328 | H | 4-methoxybenzyl | Cl | O | H | 1 | CH₂ | 3,5-dimethoxyphenyl | 3.41[a] |
| I-1329 | H | 4-methoxybenzyl | Cl | O | H | 1 | —C(Me)₂— | 1,2-oxazol-3-yl | 3.04[a] |
| I-1330 | H | 4-methoxybenzyl | Cl | O | H | 1 | CH₂ | 4-methoxypyridin-3-yl | 1.49[a] |
| I-1331 | H | 4-methoxybenzyl | Cl | O | H | 0 | — | 5-tert-butyl-2,3-dihydro-1H-inden- | 5.22[a] |
| | | | | | | | | 1-yl | |
| I-1332 | H | 4-methoxybenzyl | Cl | O | H | 1 | CHMe | 3-isopropyl-1,2,4-oxadiazol-5-yl | 3.46[a] |
| I-1333 | H | 4-methoxybenzyl | Cl | O | H | 1 | CH₂ | 1,5-diethyl-1H-pyrazol-4-yl | 2.82[a] |
| I-1334 | H | 4-methoxybenzyl | Cl | O | H | 0 | — | 6,7,8,9-tetrahydro-5H- | 4.40[a] |
| | | | | | | | | benzo[7]annulen-5-yl | |
| I-1335 | H | 4-methoxybenzyl | Cl | O | H | 1 | CH—CN | 2,3-dichlorophenyl | 4.05[a] |
| I-1336 | H | 4-methoxybenzyl | Cl | O | H | 0 | — | 3-methyltetrahydro-2H-pyran-4-yl | 2.80[a] |
| I-1337 | H | 4-methoxybenzyl | Cl | O | H | 1 | CHMe | 4,5-dihydro-1,2-oxazol-3-yl | 2.56[a] |
| I-1338 | H | 4-methoxybenzyl | Cl | O | H | 1 | CHMe | 1,2-oxazol-5-yl | 2.73[a] |
| I-1339 | H | 4-methoxybenzyl | Cl | O | H | 1 | CH₂ | 1-cyclopentyl-3-methyl-1H- | 3.23[a] |
| | | | | | | | | pyrazol-4-yl | |
| I-1340 | H | 4-methoxybenzyl | Cl | O | H | 1 | CH₂ | 5-chloro-1-benzothiophen-3-yl | 4.37[a] |
| I-1341 | H | 4-methoxybenzyl | Cl | O | H | 1 | CH₂ | 1,5-dimethyl-1H-pyrazol-4-yl | 2.33[a] |
| I-1342 | H | 4-methoxybenzyl | Cl | O | H | 1 | CH₂ | 5-methoxypyridin-3-yl | 1.89[a] |
| I-1343 | H | 4-methoxybenzyl | Cl | O | H | 1 | CHMe | 6-chloropyridin-3-yl | 3.17[a] |
| I-1344 | H | 4-methoxybenzyl | Cl | O | H | 1 | CH₂ | 3,4,5-trimethoxyphenyl | 3.08[a] |
| I-1345 | H | 4-methoxybenzyl | Cl | O | H | 1 | CH₂ | 6-methylpyridin-2-yl | 1.78[a] |
| I-1346 | H | 4-methoxybenzyl | Cl | O | H | 0 | — | octahydropentalen-1-yl | 4.35[a] |
| I-1347 | H | 4-methoxybenzyl | Cl | O | H | 0 | — | 1,1′-bi(cyclohexyl)-4-yl | 6.25[a] |
| I-1348 | H | 4-methoxybenzyl | Cl | O | H | 1 | CHMe | 3,4-dimethoxyphenyl | 3.25[a] |
| I-1349 | H | 4-methoxybenzyl | Cl | O | H | 1 | CHMe | 1-ethyl-3-methyl-1H-pyrazol-4-yl | 2.73[a] |
| I-1350 | H | cyanoacetyl | fluoro | O | H | 1 | CH₂ | cyclohexyl | |
| I-1351 | H | H | fluoro | O | H | 0 | — | 2-chlorocyclohexyl | |
| I-1352 | H | 4-methoxybenzyl | Cl | O | H | 1 | CH₂ | 2-(methylsulfanyl)ethyl | |
| I-1353 | H | 4-methoxybenzyl | Cl | O | H | 0 | — | prop-2-en-1-oxy | 2.62[a] |
| I-1354 | H | H | Cl | O | H | 1 | CH₂ | 1-methyl-1H-pyrrol-2-yl | |
| I-1355 | H | H | Cl | O | H | 1 | CH₂ | 1-methyl-1H-imidazol-5-yl | |
| I-1356 | H | H | fluoro | O | H | 0 | — | rel-[(1R,2S)-2-methylcyclohexyl] | 2.11[a] |
| I-1357 | H | H | fluoro | O | H | 0 | — | rel-[(1R,2R)-2-methylcyclohexyl] | 2.21[a] |
| I-1358 | H | H | fluoro | O | H | 1 | CH₂ | 1-bromocyclohexyl | 2.56[a] |
| I-1359 | H | H | ethynyl | O | H | 1 | CH₂ | rel-[(1R,2R)-2-fluorocyclohexyl] | 2.05[a] |
| I-1360 | H | 4-methoxybenzyl | Cl | O | H | 1 | CH₂ | 2-bromo-4,5-difluorophenyl | 4.12[a]; |
| | | | | | | | | | 4.00[b] |
| I-1361 | H | H | Cl | O | H | 0 | — | 4-methyl-1,2,5-oxadiazol-3-yl | 1.27[a] |
| I-1362 | H | H | Cl | O | H | 1 | CH₂ | 2-methyl-1,3-thiazol-4-yl | 1.14[a] |
| I-1363 | H | H | Cl | O | H | 1 | CH₂ | 2-hydroxypyridin-3-yl | 0.51[a] |
| I-1364 | H | H | Cl | O | H | 1 | CH₂ | 1-ethyl-3,5-dimethyl-1H-pyrazol-4- | 1.11[a] |
| | | | | | | | | yl | |
| I-1365 | H | H | Cl | O | H | 0 | — | cyclobutyl | 1.42[a] |
| I-1366 | H | H | Cl | O | H | 1 | CH₂ | 2,4-dimethoxyphenyl | 2.08[a] |
| I-1367 | H | H | Cl | O | H | 0 | — | 1-cyano-2-methylcyclohexyl | 2.08[a] |
| I-1368 | H | H | Cl | O | H | 1 | CH₂ | 4-methyl-1,3-thiazol-2-yl | 1.24[a] |
| I-1369 | H | H | Cl | O | H | 1 | CH₂ | 4-methyltetrahydrofuran-3-yl | 1.16 + 1.11[a] |
| I-1370 | H | H | Cl | O | H | 1 | CHMe | 3-(trifluoromethyl)phenyl | 2.66[a] |
| I-1371 | H | H | Cl | O | H | 0 | — | 2-isopropylcyclohexyl | 3.09 + 3.29[a] |
| I-1372 | H | H | Cl | O | H | 1 | CHMe | 2-(trifluoromethyl)phenyl | 2.64[a] |
| I-1373 | H | H | Cl | O | H | 1 | CH₂ | 1,3,5-trimethyl-1H-pyrazol-4-yl | 0.87[a] |
| I-1374 | H | H | Cl | O | H | 0 | — | 1,1′-bi(cyclohexyl)-2-yl | 4.05 + 4.30[a] |
| I-1375 | H | H | Cl | O | H | 0 | — | 2-methyl-1-(4-methylpyrimidin-5- | 1.43[a] |
| | | | | | | | | yl)propyl | |
| I-1376 | H | H | Cl | O | H | 0 | — | 1-(4-chlorophenyl)propyl | 2.82[a] |
| I-1377 | H | H | Cl | O | H | 1 | CH₂ | 2-methoxypyridin-4-yl | 1.14[a] |
| I-1378 | H | H | Cl | O | H | 1 | CH₂ | 1-ethyl-1H-pyrazol-4-yl | 1.01[a] |
| I-1379 | H | H | Cl | O | H | 1 | CHMe | cyclohex-3-en-1-yl | 2.48[a] |
| I-1380 | H | H | Cl | O | H | 1 | CH₂ | 6-propylpyridin-2-yl | 0.91[a] |
| I-1381 | H | H | Cl | O | H | 0 | — | 1-(1-ethyl-3-methyl-1H-pyrazol-4- | 1.56[a] |
| | | | | | | | | yl)propyl | |
| I-1382 | H | H | Cl | O | H | 0 | — | 3,3,5-trimethylcyclohexyl | 3.25[a] |
| I-1383 | H | H | Cl | O | H | 1 | CH₂ | 4-methyl-3-thienyl | 2.01[a] |
| I-1384 | H | H | Cl | O | H | 0 | — | 2,6-diisopropylcyclohexyl | 4.46 + 4.67 + |
| | | | | | | | | | 4.56[a] |
| I-1385 | H | H | Cl | O | H | 0 | — | 1-cyano-2-methylcyclopentyl | 1.84[a] |
| I-1386 | H | H | Cl | O | H | 0 | — | 2-ethylcyclohexyl | 2.71[a] |
| I-1387 | H | H | Cl | O | H | 0 | — | decahydronaphthalen-2-yl | 3.48 + 3.37[a] |
| I-1388 | H | H | Cl | O | H | 0 | — | 1-ethynyleyclohexyl | 2.30[a] |
| I-1389 | H | H | Cl | O | H | 0 | — | 3,5-bis(trifluoromethyl)cyclohexyl | 2.82 + 2.92[a] |
| I-1390 | H | H | Cl | O | H | 1 | CHMe | 2-fluorophenyl | 2.17[a] |
| I-1391 | H | H | Cl | O | H | 0 | — | 2-methyl-1-(2-methylphenyl)propyl | 3.06[a] |
| I-1392 | H | H | Cl | O | H | 1 | CHMe | 4-isopropylphenyl | 3.09[a] |
| I-1393 | H | H | Cl | O | H | 0 | — | 2,6-diethyl-4-methylcyclohexyl | 4.15 + 3.94 + |
| | | | | | | | | | 4.33 + 4.26[a] |
| I-1394 | H | H | Cl | O | H | 0 | — | 2-methyl-1-[4- | 2.32[a] |
| | | | | | | | | (trifluoromethyl)pyrimidin-5- | |
| | | | | | | | | yl]propyl | |
| I-1395 | H | H | Cl | O | H | 1 | CH₂ | 5-ethylpyridin-2-yl | 0.71[a] |
| I-1396 | H | H | Cl | O | H | 0 | — | tetrahydro-2H-pyran-4-yl | 0.81[a] |
| I-1397 | H | H | Cl | O | H | 1 | CH₂ | 4-tert-butylphenyl | 3.11[a] |
| I-1398 | H | H | Cl | O | H | 1 | CHMe | 2-thienyl | 1.98[a] |
| I-1399 | H | H | Cl | O | H | 1 | CHMe | pyridin-4-yl | |
| I-1400 | H | H | Cl | O | H | 1 | CH₂ | 3-(difluoromethyl)-1-ethyl-1H- | 1.59[a] |
| | | | | | | | | pyrazol-4-yl | |
| I-1401 | H | H | Cl | O | H | 1 | CHMe | pyridin-2-yl | 0.61[a] |
| I-1402 | H | H | Cl | O | H | 1 | CH₂ | 2-(1H-pyrazol-1-yl)ethyl | 1.05[a] |
| I-1403 | H | H | Cl | O | H | 1 | CH₂ | tetrahydrofuran-3-ylmethyl | 1.03[a] |
| I-1404 | H | H | Cl | O | H | 1 | CHMe | 2,4-dimethyl-1,3-thiazol-5-yl | 1.14[a] |
| I-1405 | H | H | Cl | O | H | 1 | CHMe | 2-methylphenyl | 2.37[a] |
| I-1406 | H | H | Cl | O | H | 1 | CH₂ | 2-(difluoromethoxy)phenyl | 2.19[a] |
| I-1407 | H | H | H | O | H | 0 | — | 1-cyclohexylpropyl | 2.36[a]; |
| | | | | | | | | | 2.30[b] |
| I-1408 | H | H | fluoro | O | H | 0 | — | decahydronaphthalen-1-yl | |
| I-1409 | H | H | fluoro | O | H | 1 | CH₂ | bicyclo[2.2.1]hept-2-yl | 2.23[a] |
| I-1410 | H | H | Cl | O | H | 1 | CH₂ | rel-[(1R,3R)-3- | 2.18[a] |
| | | | | | | | | (ethoxycarbonyl)cyclohexyl] | |
| I-1411 | H | H | fluoro | O | H | 1 | CH₂ | rel-[(1R,2R)-2-fluorocyclohexyl] | 1.74[a] |
| I-1412 | H | H | fluoro | O | H | 1 | CH₂ | rel-[(1R,2R)-2-methylcyclohexyl] | 2.47[a] |
| I-1413 | H | H | fluoro | O | H | 1 | CH₂ | rel-[(1R,2S)-2-methylcyclohexyl] | 2.53[a] |
| I-1414 | H | H | fluoro | O | H | 1 | CH₂ | 2,2-difluorocyclohexyl | 1.88[a] |
| I-1415 | H | 4-methoxybenzyl | Cl | O | H | 1 | CH₂ | rel-[(1R,3R)-3- | 3.79[a] |
| | | | | | | | | (ethoxycarbonyl)cyclohexyl] | |
| I-1416 | H | H | ethynyl | O | H | 1 | CH₂ | rel-[(1R,2R)-2-chlorocyclohexyl] | 2.37[a] |
| I-1417 | H | H | Cl | O | H | 1 | CH₂ | 2-(ethoxycarbonyl)cyclohexyl | 2.40[a] |
| I-1418 | H | H | fluoro | O | H | 1 | CH₂ | rel-[(1R,2R)-2-bromocyclohexyl] | 2.29[a] |
| I-1419 | H | H | fluoro | O | H | 1 | CH₂ | rel-[(1R,2S)-2-bromocyclohexyl] | 2.23[a] |
| I-1420 | H | 2-cyanopropanoyl | fluoro | O | H | 1 | CH₂ | cyclohexyl | |
| I-1421 | trifluoroacetyl | trifluoroacetyl | fluoro | O | H | 1 | CH₂ | cyclohexyl | |
| I-1422 | H | H | Cl | O | H | 1 | CH₂ | trans-4-chlorocyclohexyl | 2.13[a] |
| I-1423 | H | H | Cl | O | H | 1 | CH₂ | rel-[(1R,3S)-3-chlorocyclohexyl] | 2.15[a] |
| I-1424 | H | H | Cl | O | H | 1 | CH₂ | 2-(methylsulfanyl)ethyl | 1.39[a] |
| I-1425 | H | H | Cl | O | H | 1 | CHMe | 4-(trifluoromethyl)phenyl | 2.73[a] |
| I-1426 | H | H | Cl | O | H | 1 | CHMe | 1-naphthyl | 2.73[a] |
| I-1427 | H | H | Cl | O | H | 1 | CH₂ | 6-isopropylpyridin-2-yl | 1.08[a] |
| I-1428 | H | H | Cl | O | H | 1 | CHMe | 4-bromophenyl | 2.61[a] |
| I-1429 | H | H | Cl | O | H | 1 | CH₂ | 4-fluoro-3-(trifluoromethyl)phenyl | 2.54[a] |
| I-1430 | H | H | Cl | O | H | 1 | CH₂ | 2-methyltetrahydrofuran-2-yl | 1.34[a] |
| I-1431 | H | H | Cl | O | H | 1 | CH₂ | 1-ethyl-5-methyl-1H-pyrazol-4-yl | 1.16[a] |
| I-1432 | H | H | Cl | O | H | 1 | CH₂ | 1-methyl-1H-pyrazol-3-yl | 0.82[a] |
| I-1433 | H | H | Cl | O | H | 1 | CHMe | 2-furylmethyl | 1.92[a] |
| I-1434 | H | H | Cl | O | H | 1 | CH₂ | 3-chloro-4-methoxyphenyl | 2.14[a] |
| I-1435 | H | H | Cl | O | H | 1 | CH₂ | 2-thienyl | 1.70[a] |
| I-1436 | H | H | I | O | H | 1 | CH₂ | 2,4,5-trifluorophenyl | 2.13[a] |
| I-1437 | H | H | Cl | O | H | 1 | CH₂ | 3-methoxypyridin-2-yl | 0.79[a] |
| I-1438 | H | H | Cl | O | H | 1 | CH₂ | 4-(trifluoromethoxy)phenyl | 2.63[a] |
| I-1439 | H | H | Cl | O | H | 1 | CH₂ | 4-(difluoromethoxy)phenyl | 2.17[a] |
| I-1440 | H | H | Cl | O | H | 1 | CH₂ | 2,5-dimethoxyphenyl | 2.03[a] |
| I-1441 | H | H | Cl | O | H | 1 | CH₂ | pyrazin-2-yl | 0.60[a] |
| I-1442 | H | H | Cl | O | H | 1 | —C(Me)₂— | 3-(trifluoromethyl)phenyl | 2.92[a] |
| I-1443 | H | H | Cl | O | H | 1 | CH₂ | 3-(trifluoromethoxy)phenyl | 2.61[a] |
| I-1444 | H | H | Cl | O | H | 1 | CHMe | 3-methoxyphenyl | 2.10[a] |
| I-1445 | H | H | Cl | O | H | 1 | CH₂ | 4-methoxyphenyl | 1.85[a] |
| I-1446 | H | H | Cl | O | H | 1 | CH₂ | 2-methoxypyridin-3-yl | 1.49[a] |
| I-1447 | H | H | Cl | O | H | 1 | CHMe | 1-chlorocyclopropyl | 2.11[a] |
| I-1448 | H | H | Cl | O | H | 0 | — | 2-methyl-1-(pyrimidin-5-yl)propyl | 1.29[a] |
| I-1449 | H | H | Cl | O | H | 1 | CHMe | pyridin-3-yl | |
| I-1450 | H | H | Cl | O | H | 1 | CHMe | 2,4-dichlorophenyl | 3.00[a] |
| I-1451 | H | H | Cl | O | H | 1 | CH₂ | pyridin-2-ylmethyl | |
| I-1452 | H | H | Cl | O | H | 1 | CH₂ | 3-chloro-2-methylphenyl | 2.58[a] |
| I-1453 | H | H | Cl | O | H | 0 | — | cycloheptyl | 2.47[a] |
| I-1454 | H | H | Cl | O | H | 1 | CH₂ | 2-(1H-imidazol-1-yl)ethyl | |
| I-1455 | H | H | Cl | O | H | 1 | CH₂ | 2-ethoxyphenyl | 2.44[a] |
| I-1456 | H | H | Cl | O | H | 1 | CH₂ | 2-chloro-5-(trifluoromethyl)phenyl | 2.80[a] |
| I-1457 | H | H | Cl | O | H | 1 | CH₂ | 4-chloro-3-(trifluoromethyl)phenyl | 2.82[a] |
| I-1458 | H | H | Cl | O | H | 0 | — | 2,3-dihydro-1H-inden-2-yl | 2.25[a] |
| I-1459 | H | H | Cl | O | H | 0 | — | 1-phenylpropyl | 2.40[a] |
| I-1460 | H | H | Cl | O | H | 1 | CH₂ | cyclohex-1-en-1-ylmethyl | 2.68[a] |
| I-1461 | H | H | Cl | O | H | 1 | CH₂ | 2-(2-methyl-1H-imidazol-1-yl)ethyl | |
| I-1462 | H | H | Cl | O | H | 1 | CH₂ | 3-fluoro-5-(trifluoromethyl)phenyl | 2.63[a] |
| I-1463 | H | H | Cl | O | H | 1 | CH₂ | 1-naphthyl | 2.51[a] |
| I-1464 | H | H | Cl | O | H | 1 | CH₂ | 2-thienylmethyl | 1.94[a] |
| I-1465 | H | H | Cl | O | H | 1 | —C(Me)₂— | 4-methyl-1,3-thiazol-2-yl | 1.82[a] |
| I-1466 | H | H | Cl | O | H | 1 | CH₂ | 1-(2,2,2-trifluoroethyl)-1H-pyrazol- | 1.39[a] |
| | | | | | | | | 4-yl | |
| I-1467 | H | H | Cl | O | H | 1 | CH₂ | 3-methyl-1-pentyl-1H-pyrazol-4-yl | 2.04[a] |
| I-1468 | H | H | Cl | O | H | 1 | CH₂ | 1-isopropyl-3-methyl-1H-pyrazol- | 1.34[a] |
| | | | | | | | | 4-yl | |
| I-1469 | H | H | Cl | O | H | 1 | CHMe | 3-thienyl | 1.97[a] |
| I-1470 | H | H | Cl | O | H | 1 | CH₂ | 1-isobutyl-5-methyl-1H-pyrazol-4- | 1.73[a] |
| | | | | | | | | yl | |
| I-1471 | H | H | Cl | O | H | 1 | CH₂ | 1-butyl-3-methyl-1H-pyrazol-4-yl | 1.72[a] |
| I-1472 | H | H | Cl | O | H | 1 | CH₂ | 5-methyl-1,2,4-oxadiazol-3-yl | 0.77[a] |
| I-1473 | H | H | Cl | O | H | 1 | CH₂ | rel-[(1R,2S)-2-carboxycyclohexyl] | 1.37[a] |
| I-1474 | H | 4-methoxybenzyl | Cl | O | H | 1 | CH₂ | cis-4-(ethoxycarbonyl)cyclohexyl | 3.67[a] |
| I-1475 | H | 4-methoxybenzyl | Cl | O | H | 1 | CH₂ | rel-[(1R,2S)-2-carboxycyclohexyl] | 2.83[a] |
| I-1476 | H | H | Cl | O | H | 1 | CH₂ | cis-4-(ethoxycarbonyl)cyclohexyl | 2.07[a] |
| I-1477 | H | H | Cl | O | H | 1 | CH₂ | 5-chloropyrazin-2-yl | 1.37[a] |
| I-1478 | H | H | Cl | O | H | 1 | CH₂ | 3-methylpyridin-4-yl | |
| I-1479 | H | H | Cl | O | H | 1 | CH₂ | 1,3-thiazol-5-yl | 0.82[a] |
| I-1480 | H | H | Cl | O | H | 0 | — | 5,6,7,8-tetrahydroisoquinolin-8-yl | 0.39[a] |
| I-1481 | H | H | Cl | O | H | 1 | CH₂ | 6-methoxypyridin-2-yl | 1.78[a] |
| I-1482 | H | H | Cl | O | H | 1 | CH₂ | 4-methoxy-3- | 2.51[a] |
| | | | | | | | | (trifluoromethyl)phenyl | |
| I-1483 | H | H | Cl | O | H | 1 | CH₂ | 1-cyclopentyl-3-methyl-1H- | 1.80[a] |
| | | | | | | | | pyrazol-4-yl | |
| I-1484 | H | H | Cl | O | H | 0 | — | 3-methyltetrahydro-2H-pyran-4-yl | 1.13 + 1.19[a] |
| I-1485 | H | H | Cl | O | H | 1 | CHMe | 1,2-oxazol-5-yl | 1.13[a] |
| I-1486 | H | H | Cl | O | H | 1 | CH₂ | 2-methoxypyrimidin-4-yl | 1.07[a] |
| I-1487 | H | H | Cl | O | H | 1 | CH₂ | 6-methylpyridin-2-yl | |
| I-1488 | H | H | Cl | O | H | 1 | CHMe | 1,3-thiazol-2-yl | 1.26[a] |
| I-1489 | H | H | Cl | O | H | 1 | CH₂ | 5-methoxypyridin-2-yl | 0.82[a] |
| I-1490 | H | H | Cl | O | H | 1 | CH₂ | 5-chloro-1-benzothiophen-3-yl | 2.88[a] |
| I-1491 | H | H | Cl | O | H | 1 | CHMe | 6-chloropyridin-3-yl | 1.65[a] |
| I-1492 | H | H | Cl | O | H | 1 | CHMe | 3-isopropyl-1,2,4-oxadiazol-5-yl | 1.86[a] |
| I-1493 | H | H | Cl | O | H | 1 | CH₂ | 1,5-dimethyl-1H-pyrazol-4-yl | 0.94[a] |
| I-1494 | H | H | Cl | O | H | 1 | CH₂ | 4-methoxypyridin-3-yl | |
| I-1495 | H | H | Cl | O | H | 1 | CHMe | 1-ethyl-3-methyl-1H-pyrazol-4-yl | 1.35[a] |
| I-1496 | H | H | Cl | O | H | 1 | —C(Me)₂— | 1,2-oxazol-3-yl | 1.37[a] |
| I-1497 | H | H | Cl | O | H | 0 | — | 6,7,8,9-tetrahydro-5H- | 2.82[a] |
| | | | | | | | | benzo[7]annulen-5-yl | |
| I-1498 | H | H | Cl | O | H | 1 | CH₂ | 1,5-diethyl-1H-pyrazol-4-yl | 1.47[a] |
| I-1499 | H | H | Cl | O | H | 1 | CH₂ | 1-(2,2-difluoroethyl)-1H-pyrazol-4- | 1.14[a] |
| | | | | | | | | yl | |
| I-1500 | H | H | Cl | O | H | 1 | —C(CH₂)₂— | 2-chlorophenyl | 2.62[a] |
| I-1501 | H | H | Cl | O | H | 1 | CH₂ | 3,5-dimethoxyphenyl | 1.97[a] |
| I-1502 | H | H | Cl | O | H | 0 | — | 6-tert-butyl-1,2,3,4- | 3.96[a] |
| | | | | | | | | tetrahydronaphthalen-1-yl | |
| I-1503 | H | H | Cl | O | H | 1 | CH₂ | 5-methoxypyridin-3-yl | 0.26[a] |
| I-1504 | H | H | Cl | O | H | 1 | CHMe | 4,5-dihydro-1,2-oxazol-3-yl | 0.86[a] |
| I-1505 | H | H | Cl | O | H | 1 | —C(CH₂)₂— | 2-bromophenyl | 2.76[a] |
| I-1506 | H | H | Cl | O | H | 1 | CH₂ | 3,5-bis(trifluoromethyl)phenyl | 3.13[a] |
| I-1507 | H | H | Cl | O | H | 1 | CH₂ | 5-(difluoromethyl)-1-ethyl-1H- | 1.68[a] |
| | | | | | | | | pyrazol-4-yl | |
| I-1508 | H | H | Cl | O | H | 0 | — | octahydropentalen-1-yl | 2.66[a] |
| I-1509 | H | H | Cl | O | H | 0 | — | 1,1′-bi(cyclohexyl)-4-yl | 4.49[a] |
| I-1510 | H | H | Cl | O | H | 1 | CHMe | 3,4-dimethoxyphenyl | 1.83[a] |
| I-1511 | H | H | Cl | O | H | 1 | CH₂ | 3,4,5-trimethoxyphenyl | 1.68[a] |
| I-1512 | H | trifluoroacetyl | fluoro | O | H | 1 | CH₂ | 2,4,5-trifluorophenyl | |
| I-1513 | H | propionyl | fluoro | O | H | 1 | CH₂ | 2,4,5-trifluorophenyl | |
| I-1514 | H | tert-butoxycarbonyl | fluoro | O | H | 1 | CH₂ | rel-[(1R,2R)-2-chlorocyclohexyl] | 3.41[a] |
| I-1515 | H | tert-butoxycarbonyl | fluoro | O | H | 1 | CH₂ | rel-[(1R,2R)-2-chlorocyclohexyl] | 3.41[a] |
| I-1516 | H | H | I | O | H | 1 | CH₂ | 2,3,4-trifluorophenyl | 2.18[a] |
| I-1517 | H | 4-methoxybenzyl | Cl | O | H | 1 | CH₂ | rel-[(1R,2S)-2- | 4.14[a] |
| | | | | | | | | (ethoxycarbonyl)cyclohexyl] | |
| I-1518 | tert-butoxycarbonyl | tert-butoxycarbonyl | fluoro | O | H | 1 | CH₂ | rel-[(1R,2R)-2-fluorocyclohexyl] | |
| I-1519 | H | tert-butoxycarbonyl | fluoro | O | H | 1 | CH₂ | rel-[(1R,2R)-2-fluorocyclohexyl] | 3.05[a] |
| I-1520 | H | H | Cl | O | H | 1 | CH₂ | cis-4-carboxycyclohexyl | 1.12[a] |
| I-1521 | H | H | Cl | O | H | 1 | CH₂ | rel-[(1R,2R)-2- | 2.41[a] |
| | | | | | | | | (ethoxycarbonyl)cyclohexyl] | |
| I-1522 | H | 4-methoxybenzyl | Cl | O | H | 1 | CH₂ | cis-4-carboxycyclohexyl | 2.56[a] |
| I-1523 | H | 2-chloro-2,3,3,3- | fluoro | O | H | 1 | CH₂ | cyclohexyl | |
| | | tetrafluoropropanoyl | | | | | | | |
| I-1524 | H | H | Cl | O | H | 1 | CH₂ | rel-[(1R,3R)-3-cyanocyclohexyl] | 1.45[a] |
| I-1525 | H | H | fluoro | O | H | 1 | CH₂ | rel-[(1R,2R)-2-chlorocyclohexyl] | 2.16[a] |
| I-1526 | H | H | I | O | H | 1 | CH₂ | 4-methoxyphenyl | 1.69[a] |
| I-1527 | H | H | Cl | O | H | 1 | CH₂ | rel-[(1R,2S)-2-cyanocyclohexyl] | 1.66[a] |
| I-1528 | H | H | fluoro | O | H | 1 | CH₂ | tetrahydro-2H-pyran-2-yl | 1.22[a] |
| I-1529 | H | H | Cl | O | H | 1 | CH—CN | 2,3-dichlorophenyl | 2.58[a] |
| I-1530 | H | H | Cl | O | H | 0 | — | 1-(1H-imidazol-1-yl)-3,3- | 0.82[a] |
| | | | | | | | | dimethylbutan-2-yl | |
| I-1531 | H | H | Cl | O | H | 0 | — | 2,6,6-trimethyl-4,5,6,7-tetrahydro- | 3.16[a] |
| | | | | | | | | 1-benzofuran-4-yl | |
| I-1532 | H | H | Cl | O | H | 0 | — | 4-tert-butylcyclohexyl | 3.69[a] |
| I-1533 | H | H | Cl | O | H | 0 | — | 5-tert-butyl-2,3-dihydro-1H-inden- | 3.70[a] |
| | | | | | | | | 1-yl | |
| I-1534 | H | H | Cl | O | H | 0 | — | 7-tert-butyl-1,2,3,4- | 3.94[a] |
| | | | | | | | | tetrahydronaphthalen-2-yl | |
| I-1535 | H | H | Cl | O | H | 0 | — | 3-ethyl-3,5,5-trimethylcyclohexyl | 4.01[a] |
| I-1536 | H | H | fluoro | O | H | 1 | CH₂ | rel-[(1R,2R)-2-chlorocyclohexyl] | 2.16[a] |
| I-1537 | H | difluoroacetyl | fluoro | O | H | 1 | CH₂ | rel-[(1R,2R)-2-chlorocyclohexyl] | |
| I-1538 | H | trifluoroacetyl | fluoro | O | H | 1 | CH₂ | rel-[(1R,2R)-2-chlorocyclohexyl] | |
| I-1539 | H | H | amino | O | H | 1 | CH₂ | cyclohexyl | 1.37[a] |
| I-1540 | H | H | I | O | H | 1 | CH₂ | rel-[(1R,2S)-2-methylcyclohexyl] | 2.73[a] |
| I-1541 | H | H | I | O | H | 1 | CH₂ | rel-[(1R,2S)-2-methylcyclohexyl] | 2.67[a] |
| I-1542 | H | H | Cl | O | H | 1 | CH₂ | 2-bromo-4,5-difluorophenyl | 4.47[a]; |
| | | | | | | | | | 2.42[b] |
| I-1543 | H | trifluoroacetyl | fluoro | O | H | 1 | CH₂ | rel-[(1R,2R)-2-chlorocyclohexyl] | |
| I-1544 | H | 2-chloro-2,3,3,3- | fluoro | O | H | 1 | CH₂ | 2,4,5-trifluorophenyl | |
| | | tetrafluoropropanoyl | | | | | | | |
| I-1545 | H | 2,2,3,3,3- | fluoro | O | H | 1 | CH₂ | cyclohexyl | |
| | | pentafluoropropanoyl | | | | | | | |
| I-1546 | H | 2,2,3,3,3- | fluoro | O | H | 1 | CH₂ | rel-[(1R,2R)-2-chlorocyclohexyl] | |
| | | pentafluoropropanoyl | | | | | | | |
| I-1547 | H | 2-chloro-2,3,3,3- | fluoro | O | H | 1 | CH₂ | rel-[(1R,2R)-2-chlorocyclohexyl] | |
| | | tetrafluoropropanoyl | | | | | | | |
| I-1548 | H | 4-methoxybenzyl | Cl | O | H | 0 | — | spiro[2.5]oct-1-yl | 4.39[a] |
| I-1549 | H | H | Cl | O | H | 0 | — | spiro[2.5]oct-1-yl | 2.54[a] |
| I-1550 | H | 4-methoxybenzyl | Cl | O | H | 0 | — | rel-[(1R,2S)-2-methylcyclohexyl] | 4.18[a] |
| I-1551 | H | 4-methoxybenzyl | Cl | O | H | 0 | — | rel-[(1R,2R)-2-methylcyclohexyl] | 4.39[a] |
| I-1552 | H | 4-methoxybenzyl | Cl | O | H | 0 | — | 2-methylcyclohexyl | 4.32 + 4.13[a] |
| I-1553 | H | H | Cl | O | H | 1 | CH₂ | 2-bromophenyl | 1.88[a] |
| I-1554 | H | H | Cl | O | H | 1 | CH₂ | 2-(trifluoromethyl)cyclohexyl | 2.51[a] |
| I-1555 | H | 4-methoxybenzyl | Cl | O | H | 1 | CH₂ | 4-cyanophenyl | 3.23[a] |
| I-1556 | H | 4-methoxybenzyl | Cl | O | 4- | 1 | CH₂ | 2,4-difluorophenyl | 5.14[a] |
| | | | | | (trifluoromethyl)benzoyl | | | | |
| I-1557 | H | 2,2,3,3,3- | fluoro | O | H | 1 | CH₂ | rel-[(1R,2R)-2-chlorocyclohexyl] | |
| | | pentafluoropropanoyl | | | | | | | |
| I-1558 | H | methoxyacetyl | fluoro | O | H | 1 | CH₂ | rel-[(1R,2R)-2-chlorocyclohexyl] | |
| I-1559 | H | 2-chloro-2,3,3,3- | fluoro | O | H | 1 | CH₂ | rel-[(1R,2R)-2-chlorocyclohexyl] | |
| | | tetrafluoropropanoyl | | | | | | | |
| I-1560 | H | propionyl | fluoro | O | H | 1 | CH₂ | rel-[(1R,2R)-2-chlorocyclohexyl] | |
| I-1561 | H | propionyl | fluoro | O | H | 1 | CH₂ | rel-[(1R,2R)-2-chlorocyclohexyl] | |
| I-1562 | H | methoxyacetyl | fluoro | O | H | 1 | CH₂ | rel-[(1R,2R)-2-chlorocyclohexyl] | |
| I-1563 | H | H | Cl | O | H | 0 | — | 3-methylcyclohexyl | 2.37[a] |
| I-1564 | H | H | I | O | H | 0 | — | 2-methylcyclohexyl | 2.14[a] |
| I-1565 | H | H | I | O | H | 0 | — | rel-[(1R,2R)-2-chlorocyclohexyl] | 2.12[a] |
| I-1566 | H | H | Cl | O | H | 1 | CH₂ | rel-[(1R,2S)-2- | 2.63[a] |
| | | | | | | | | (trifluoromethyl)cyclohexyl] | |
| I-1567 | H | H | Cl | O | H | 1 | CH₂ | rel-[(1R,2R)-2- | 2.69[a] |
| | | | | | | | | (trifluoromethyl)cyclohexyl] | |
| I-1568 | H | H | Cl | O | H | 1 | CH₂ | 3-hydroxy-4-methoxyphenyl | 1.03[a] |
| I-1569 | H | H | Cl | O | H | 1 | CHMe | cyclohexyl | 2.84[a] |
| I-1570 | H | methoxyacetyl | fluoro | O | H | 1 | CH₂ | 2,4,5-trifluorophenyl | |
| I-1571 | H | tert-butoxycarbonyl | Cl | O | H | 1 | CH₂ | rel-[(1R,2R)-2-chlorocyclohexyl] | 3.53[a] |
| I-1572 | H | tert-butoxycarbonyl | fluoro | O | H | 1 | CH₂ | rel-[(1R,2R)-2-chlorocyclohexyl] | 3.34[a] |
| I-1573 | H | cyclohexylcarbonyl | fluoro | O | H | 1 | CH₂ | 2,4,5-trifluorophenyl | |
| I-1574 | H | cyclohexylcarbonyl | fluoro | O | H | 1 | CH₂ | cyclohexyl | |
| I-1575 | H | 2,4-difluorobenzoyl | fluoro | O | H | 1 | CH₂ | cyclohexyl | |
| I-1576 | H | H | Cl | O | H | 1 | CH₂ | trans-4-methylcyclohexyl | |
| I-1577 | H | H | Cl | O | H | 1 | CH₂ | cis-4-methylcyclohexyl | |
| I-1578 | H | tert-butoxycarbonyl | Cl | O | H | 1 | CH₂ | rel-[(1R,2R)-2-chlorocyclohexyl] | 3.52[a] |
| I-1579 | H | H | Cl | O | H | 1 | CH₂ | cis-4-chlorocyclohexyl | 2.20[a] |
| I-1580 | H | H | Cl | O | H | 1 | CH₂ | rel-[(1R,3R)-3-chlorocyclohexyl] | 2.23[a] |
| I-1581 | H | tert-butoxycarbonyl | Cl | O | H | 1 | CH₂ | rel-[(1R,2R)-2-chlorocyclohexyl] | 3.56[a] |
| I-1582 | H | methoxy(oxo)acetyl | fluoro | O | H | 1 | CH₂ | 2,4,5-trifluorophenyl | |
| I-1583 | H | chloroacetyl | fluoro | O | H | 1 | CH₂ | 2,4,5-trifluorophenyl | |
| I-1584 | H | 3-methoxy-3- | fluoro | O | H | 1 | CH₂ | 2,4,5-trifluorophenyl | |
| | | oxopropanoyl | | | | | | | |
| I-1585 | H | isobutyryl | fluoro | O | H | 1 | CH₂ | 2,4,5-trifluorophenyl | |
| I-1586 | H | butyryl | fluoro | O | H | 1 | CH₂ | 2,4,5-trifluorophenyl | |
| I-1587 | H | 4-methoxy-4- | fluoro | O | H | 1 | CH₂ | 2,4,5-trifluorophenyl | |
| | | oxobutanoyl | | | | | | | |
| I-1588 | isobutyryl | isobutyryl | fluoro | O | H | 1 | CH₂ | 2,4,5-trifluorophenyl | |

Specific preferred compounds of the formula (VI) are shown in Table 2.

TABLE 2
Preferred compounds of the formula (VI)

| Ex N^{o} | NR⁴R⁵ | N[R²](A)yR¹ | LogP |
|---|---|---|---|
| II-001 | 1,1-diphenylmethanimino | cyclohexanamino | 5.13^{[a]} |
| II-002 | 1,1-diphenylmethanimino | 1-cyclohexylmethanamino | 5.65^{[a]} |
| II-003 | 1,1-diphenylmethanimino | (S)-1-cyclohexylethanamino | 5.93^{[a]} |
| II-004 | 1,1-diphenylmethanimino | 1-(2-chlorophenyl)methanamino | 4.89^{[a]} |
| II-005 | 1,1-diphenylmethanimino | 1-(tetrahydrofuran-2- | 3.83^{[a]} |
| | | yl)methanamino | |
| II-006 | 1,1-diphenylmethanimino | 1-(tetrahydro-2H-pyran-4- | 3.60^{[a]} |
| | | yl)methanamino | |
| II-007 | 1,1-diphenylmethanimino | 1-(tetrahydro-2H-pyran-3- | 3.71^{[a]} |
| | | yl)methanamino | |
| II-008 | 1,1-diphenylmethanimino | 1-(tetrahydro-2H-pyran-2- | 4.55^{[a]} |
| | | yl)methanamino | |
| II-009 | benzenesulfonamido | (S)-1-cyclohexylethanamino | 3.75^{[a]} |
| II-010 | 1-(4-methoxyphenyl)methanamino | 1-(2-chlorophenyl)propan-2- | 4.33^{[a]} |
| | | amino | |
| II-011 | amino | 1-(2-chlorophenyl)propan-2- | 2.64^{[a]} |
| | | amino | |
| II-012 | N-(phenylsulfonyl)benzenesulfonamido | 1-(2-fluorophenyl)methanamino | 4.15^{[a]} |
| II-013 | 1,1-diphenylmethanimino | tert-butyl 4-[amino- | |
| | | methyl]piperidine-1-carboxylate | |
| II-014 | 2-(methylsulfanyl)acetamido | 1-cyclohexylmethanamino | |
| II-015 | 2-(methylsulfanyl)acetamido | 1-(2,4- | |
| | | difluorophenyl)methanamino | |
| II-016 | N,N-dimethylimidoformamido | 1-cyclohexylmethanamino | |
| II-017 | amino | 6-methoxy-3,4-dihydroquinolin- | 2.11^{[a]}; 2.06^{[b]} |
| | | 1(2H)-yl | |
| II-018 | amino | 7-(trifluoromethyl)-3,4- | 2.78^{[a]}; 2.71^{[b]} |
| | | dihydroquinolin-1(2H)-yl | |
| II-019 | amino | 6-fluoro-3,4-dihydroisoquinolin- | 2.09^{[a]}; 2.05^{[b]} |
| | | 2(1H)-yl | |
| II-020 | amino | 3,4-dihydroisoquinolin-2(1H)-yl | 2.02^{[a]}; 1.98^{[b]} |
| II-021 | amino | 8-methyl-3,4-dihydroquinolin- | 2.43^{[a]}; 2.36^{[b]} |
| | | 1(2H)-yl | |
| II-022 | amino | 5,8-dichloro-3,4- | 2.88^{[a]}; 2.82^{[b]} |
| | | dihydroisoquinolin-2(1H)-yl | |
| II-023 | 1-(4-methoxyphenyl)methanamino | 1-(4-chlorophenyl)propan-2- | 4.32^{[a]} |
| | | amino | |
| II-024 | 1-(pyrrolidin-1-yl)methanimino | (S)-1-cyclohexylethanamino | 2.73^{[a]} |
| II-025 | N,N-dimethylimidoformamido | (S)-1-cyclohexylethanamino | 3.13^{[a]} |
| II-026 | amino | 6-chloro-3,4-dihydroquinolin- | 2.66^{[a]}; 2.60^{[b]} |
| | | 1(2H)-yl | |
| II-027 | amino | 1-(4-chlorophenyl)propan-2- | 2.68^{[a]} |
| | | amino | |
| II-028 | N,N-dimethylimidoformamido | 1-(2,4- | |
| | | difluorophenyl)methanamino | |
| II-029 | amino | 2,3-dihydro-1H-indol-1-yl | 2.08^{[a]}; 2.03^{[b]} |
| II-030 | 1-(pyrrolidin-1-yl)methanimino | 1-(4-chlorophenyl)methanamino | 2.17^{[a]} |
| II-031 | N,N-dimethylimidoformamido | 1-(4-chlorophenyl)methanamino | 2.41^{[a]} |
| II-032 | —N═S(Me)₂ | 1-cyclohexylmethanamino | 1.82^{[a]} |
| II-033 | —N═S(—CH₂—CH₂—CH₂—CH₂—) | 1-cyclohexylmethanamino | 2.15^{[a]} |
| II-034 | —NH—SCF₃ | 1-cyclohexylmethanamino | |
| II-035 | amino | octahydro-2H-isoindol-2-yl | |
| II-036 | 1-(pyrrolidin-1-yl)methanimino | rel-[rel-(1R,4aS,8aS)- | 3.52^{[a]} |
| | | decahydronaphthalen-1-amino] | |
| II-037 | 1-(pyrrolidin-1-yl)methanimino | rel-[rel-(1R,4aR,8aS)- | 3.83^{[a]} |
| | | decahydronaphthalen-1-amino] | |
| II-038 | N,N-dimethylimidoformamido | rel-[rel-(1R,4aR,8aS)- | 3.94^{[a]} |
| | | decahydronaphthalen-1-amino] | |
| II-039 | N,N-dimethylimidoformamido | rel-[rel-(1R,4aR,8aS)- | 4.19^{[a]} |
| | | decahydronaphthalen-1-amino] | |
| II-040 | amino | rel-(4aR,8aS)- | |
| | | octahydroisoquinolin-2(1H)-yl | |
| II-041 | —N═S(Me)₂ | 1-(2,4- | 1.38^{[a]} |
| | | difluorophenyl)methanamino | |
| II-042 | —N═S(—CH₂—CH₂—CH₂—CH₂—) | 1-(2,4- | 1.64^{[a]} |
| | | difluorophenyl)methanamino | |
| II-043 | —N═S(Me)₂ | 1-(2,4,6- | 1.42^{[a]} |
| | | trifluorophenyl)methanamino | |
| II-044 | —N═S(—CH₂—CH₂—CH₂—CH₂—) | 1-(2,4,6 | 1.66^{[a]} |
| | | trifluorophenyl)methanamino | |
| II-045 | —N═S(—CH₂—CH₂—CH₂—CH₂—) | 1-(3,4,5- | 1.96^{[a]} |
| | | trifluorophenyl)methanamino | |
| II-046 | —N═S(Me)₂ | 1-(3,4,5- | 1.68^{[a]} |
| | | trifluorophenyl)methanamino | |
| II-047 | —N═S(Me)₂ | 1-(2,3,4- | 1.57^{[a]} |
| | | trifluorophenyl)methanamino | |
| II-048 | —N═S(—CH₂—CH₂—CH₂—CH₂—) | 1-(2,3,4- | 1.83^{[a]} |
| | | trifluorophenyl)methanamino | |
| II-049 | —N═S(O)(Me)₂ | 1-cyclohexylmethanamino | 2.47^{[a]} |
| II-050 | —N═S(Me)-4-tolyl | 1-cyclohexylmethanamino | 3.51^{[a]} |
| II-051 | —NH—SO₂—NH—Me | 1-cyclohexylmethanamino | 2.38^{[a]} |
| II-052 | —N═S(Et)₂ | 1-cyclohexylmethanamino | 2.00^{[a]} |
| II-062 | 1-(4-methoxyphenyl)methanamino | 7-(trifluoromethyl)-3,4- | 4.37^{[a]}; 4.25^{[b]} |
| | | dihydroquinolin-1(2H)-yl | |
| II-063 | 1-(4-methoxyphenyl)methanamino | 6-methoxy-3,4-dihydroquinolin- | 3.72^{[a]} |
| | | 1(2H)-yl | |
| II-064 | 1-(4-methoxyphenyl)methanamino | 3,4-dihydroisoquinolin-2(1H)-yl | 3.68^{[a]} |
| II-065 | 1-(4-methoxyphenyl)methanamino | 6-fluoro-3,4-dihydroisoquinolin- | 3.73^{[a]} |
| | | 2(1H)-yl | |
| II-066 | 1-(4-methoxyphenyl)methanamino | 6-chloro-3,4-dihydroquinolin- | 4.37^{[a]} |
| | | 1(2H)-yl | |
| II-067 | 1-(4-methoxyphenyl)methanamino | 8-methyl-3,4-dihydroquinolin- | 4.24^{[a]}; 4.14^{[b]} |
| | | 1(2H)-yl | |
| II-068 | 1-(4-methoxyphenyl)methanamino | 5,8-dichloro-3,4- | 4.66^{[a]}; 4.55^{[b]} |
| | | dihydroisoquinolin-2(1H)-yl | |
| II-069 | 1-(4-methoxyphenyl)methanamino | 2,3-dihydro-1H-indol-1-yl | 3.83^{[a]}; 3.76^{[b]} |
| II-083 | 1,1-diphenylmethanimino | 2-cyclohexylethanamino | 5.92^{[a]} |
| II-084 | —N═S(CH₂—Ph)₂ | 1-cyclohexylmethanamino | 4.38^{[a]} |
| II-085 | —N═S(Me)—2-chlorobenzyl | 1-cyclohexylmethanamino | 3.61^{[a]} |
| II-086 | —N═S(Et)—CH₂—CH₂—CH₃ | 1-cyclohexylmethanamino | 1.90^{[c]} |
| II-087 | —N═S(CH₂—CH₂—CH₃)—CH₂—CH₂—CH₃ | 1-cyclohexylmethanamino | |
| II-088 | —N═S(—CH₂—CH₂—CH₂—CH₂—CH₂) | 1-cyclohexylmethanamino | 2.60^{[a]} |
| II-089 | —N═S(CH3)—CH₂—CH₂—CH₃ | 1-cyclohexylmethanamino | 2.23^{[a]} |
| II-090 | —N═S(—CH₂—CH₂—O—CH₂—CH₂) | 1-cyclohexylmethanamino | 2.49^{[a]} |
| II-091 | —N═S(Me)—Et | 1-cyclohexylmethanamino | 1.94^{[a]} |
| II-092 | —N═S(Ph)₂ | 1-cyclohexylmethanamino | 4.77^{[a]} |
| II-095 | 1,1-diphenylmethanimino | 2-phenylethanamino | 4.69^{[a]} |
| II-096 | 1,1-diphenylmethanimino | 1-cyclohexyl-N-(4- | |
| | | methoxybenzyl)methanamino | |
| II-097 | 1-(4-methoxyphenyl)methanamino | 3,4-dihydroquinolin-1(2H)-yl | 3.84^{[a]} |
| II-098 | 1-(4-methoxyphenyl)methanamino | 5-chloro-3,4-dihydroisoquinolin- | 4.20^{[a]} |
| | | 2(1H)-yl | |
| II-099 | 1-(4-methoxyphenyl)methanamino | 6-methyl-3,4-dihydroquinolin- | 4.24^{[a]}; 4.15^{[b]} |
| | | 1(2H)-yl | |
| II-100 | 1-(4-methoxyphenyl)methanamino | 2-methyl-3,4-dihydroquinolin- | 4.14^{[a]}; 4.09^{[b]} |
| | | 1(2H)-yl | |
| II-101 | amino | 2-methyl-3,4-dihydroquinolin- | 2.48^{[a]}; 2.39^{[b]} |
| | | 1(2H)-yl | |
| II-102 | 1-(4-methoxyphenyl)methanamino | pyrrolidin-1-yl | 2.72^{[a]}; 2.66^{[b]} |
| II-106 | 1-(4-methoxyphenyl)methanamino | amino | 1.94^{[a]} |
| II-107 | 1,1-diphenylmethanimino | anilinato | 4.62^{[a]} |
| II-108 | 1-(4-methoxyphenyl)methanamino | piperidin-1-yl | 3.22^{[a]}; 3.16^{[b]} |

Specific preferred compounds of the formula (VI-a) are shown in Table 2a.

TABLE 2a
Preferred compounds of the formula (VI-a)

| Ex N^{o} | NR⁴R⁵ | N[R²](A)yR¹ | LogP |
|---|---|---|---|
| II-053 | —N═S(Me)₂ | 1-cyclohexylmethanamino | 1.53^{[a]} |
| II-054 | —N═S(Me)-4-tolyl | 1-cyclohexylmethanamino | 3.10^{[a]} |
| II-055 | —N═S(—CH₂—CH₂—CH₂—CH₂—) | 1-cyclohexylmethanamino | 1.72^{[a]} |
| II-056 | N,N-dimethylimidoformamido | 1-cyclohexylmethanamino | |
| II-057 | —N═S(Me)₂ | 1-(2,4- | 1.30^{[a]} |
| | | difluorophenyl)methanamino | |
| II-058 | —N═S(Me)-4-tolyl | 1-(2,4- | 2.74^{[a]} |
| | | difluorophenyl)methanamino | |
| II-059 | —N═S(—CH₂—CH₂—CH₂—CH₂—) | 1-(2,4- | 1.57^{[a]} |
| | | difluorophenyl)methanamino | |
| II-060 | N,N-dimethylimidoformamido | 1-(2,4- | |
| | | difluorophenyl)methanamino | |
| II-061 | —N═S(Me)₂ | 1-cyclopentylmethanamino | 0.32^{[a]} |
| II-070 | —N═S(Me)₂ | 1-(tetrahydrofuran-2- | 0.44^{[a]} |
| | | yl)methanamino | |
| II-071 | —N═S(Me)₂ | 1-(tetrahydrofuran-3- | 0.26^{[a]} |
| | | yl)methanamino | |
| II-072 | —N═S(Me)₂ | 1-(2,4,5- | 1.43^{[a]} |
| | | trifluorophenyl)methanamino | |
| II-073 | —N═S(Me)-4-tolyl | 1-(2,4,5- | 2.87^{[a]} |
| | | trifluorophenyl)methanamino | |
| II-074 | —N═S(—CH₂—CH₂—CH₂—CH₂—) | 1-(2,4,5- | 1.53^{[a]} |
| | | trifluorophenyl)methanamino | |
| II-075 | N,N-dimethylimidoformamido | 1-(2,4,5- | |
| | | trifluorophenyl)methanamino | |
| II-076 | —N═S(Me)₂ | 1-(2,3,4- | 1.39^{[a]} |
| | | trifluorophenyl)methanamino | |
| II-077 | —N═S(Me)-4-tolyl | 1-(2,3,4- | 2.73^{[a]} |
| | | trifluorophenyl)methanamino | |
| II-078 | —N═S(—CH₂—CH₂—CH₂—CH₂—) | 1-(2,3,4- | 1.55^{[a]} |
| | | trifluorophenyl)methanamino | |
| II-079 | —N═S(Me)₂ | 1-(2,4,6- | 1.40^{[a]} |
| | | trifluorophenyl)methanamino | |
| II-080 | —N═S(Me)-4-tolyl | 1-(2,4,6- | 2.63^{[a]} |
| | | trifluorophenyl)methanamino | |
| II-081 | —N═S(—CH₂—CH₂—CH₂—CH₂—) | 1-(2,4,6- | 1.39^{[a]} |
| | | trifluorophenyl)methanamino | |
| II-082 | N,N-dimethylimidoformamido | 1-(2,4,6- | |
| | | trifluorophenyl)methanamino | |
| II-093 | —N═S(Et)—CH₂—CH₂—CH₃ | 1-cyclohexylmethanamino | 2.07^{[a]} |
| II-094 | —N═S(Et)—CH₂—CH₂—CH₃ | 1-(2,4- | 1.77^{[a]} |
| | | difluorophenyl)methanamino | |
| II-103 | —N═S(Et)—CH₂—CH₂—CH₃ | 1-(2,4,5- | 2.10^{[a]} |
| | | trifluorophenyl)methanamino | |
| II-104 | —N═S(Et)—CH₂—CH₂—CH₃ | 1-(2,3,4- | 1.96^{[a]} |
| | | trifluorophenyl)methanamino | |
| II-105 | —N═S(Et)—CH₂—CH₂—CH₃ | 1-(2,4,6- | 1.92^{[a]} |
| | | trifluorophenyl)methanamino | |

Preferably, the one or more compounds of the formulae (G), (I), (II), (III), (IV), (V), (VI), (VI-a) and (VII), each as defined above, and the salts thereof, are used in the context of the present invention, wherein the structural elements in the formulae (G), (I), (II), (III), (IV), (V), (VI), (VI-a) and (VII), each have, independently from one another, the meaning as defined above in the context of the meaning as defined above in one of the preferred, more preferred, or particularly preferred embodiments.

The present invention also provides processes for preparing the compounds of the general formula (G) and/or their salts. This includes processes which can be carried out analogously to known methods.

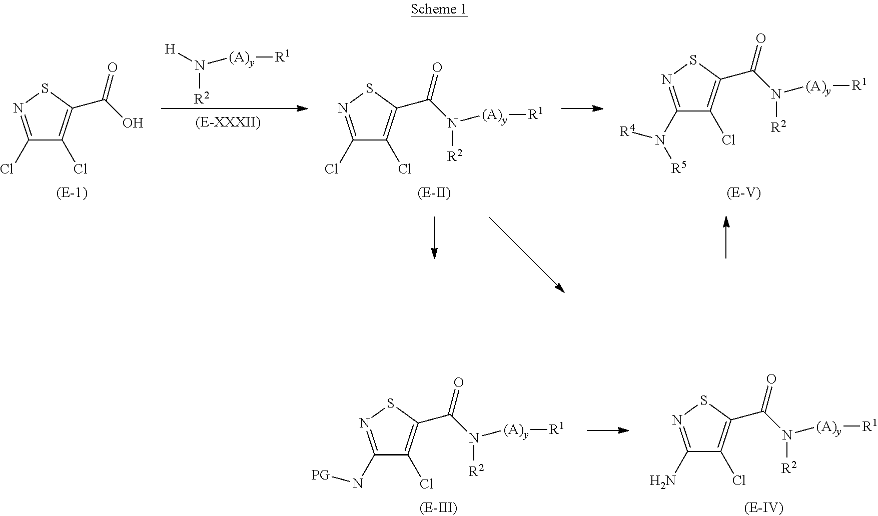

Compounds according to the invention of structure type (E-V) may, for example, can originate from compounds of type (E-IV), which bear an amino group in the 3-position of the isothiazole ring. For this purpose, alkylation, acylation or sulphonylation reactions may, inter alia, be used, in which (E-IV) in a solvent is reacted, for example, with a suitable alkyl halide, alkyl sulphonate, acyl halide, acid anhydride or sulphonyl halide. Compounds of type (E-V) are, in individual cases, also directly accessible from intermediates of type (E-II), by reacting (E-II) directly with a suitable amine of the general structure R⁴—NH—R⁵, wherein, if appropriate, a transition metal catalyst or precatalyst, possibly in combination with a suitable ligand and a base, for example K₃PO₄, Cs₂CO₃ or other bases, is required. Such reactions are usually conducted in a solvent commonly used in organic chemistry, for example dimethylformamide, toluene or other solvents, and at elevated temperature, for example between 50° C. and 200° C.

Compounds of structure type (E-IV) can be prepared either directly from the compounds (E-II) or via structure type (E-III). In the first case, (E-II) is reacted with ammonia in a solvent at elevated temperatures, wherein a suitable catalyst system may be used. In the second case, a suitable ammonia surrogate, which includes a protecting group which can be cleaved, is used in place of ammonia. Such a surrogate may be, for example, benzophenone imine or 4-methoxybenzylamine or other surrogates. The reaction must generally also be conducted with the aid of a catalyst composed of a transition metal complex and optionally one or more additional ligands. Suitable transition metal complexes are, for example, Pd(PPh₃)₄, Pd₂dba₃, PdCl₂(PPh₃)₂, etc., and suitable ligands are, for example, XantPhos [4,5-bis(diphenylphosphino)-9,9-dimethylxanthene], Mor-DalPhos [di(1-adamantyl)-2-morpholinophenylphosphine], BrettPhos [2-(dicyclohexylphosphino)3,6-dimethoxy-2′,4′,6′-triisopropyl-1,1′-biphenyl], etc.. These catalyst systems, besides a multitude of other systems and the reaction conditions required for the reactions, are described in detail in the literature, for example, in D. Surry, S. Buchwald Chem. Sci., 2011, 2, 27.

The conditions for the conversion of structure type (E-III) to structure type (E-IV) depend on the nature of the protecting group PG to be cleaved. Generally used in this connection and preferred in this context, however, are acids such as dilute mineral acids or organic acids (HCl in MeOH, trifluoroacetic acid (TFA), if PG is, for example, diphenylmethylene; 4-methoxybenzyl; benzyl, etc.) or oxidative reagents (such as dichlorodicyanoquinone, if the protecting group PG is, for example, 4-methoxybenzyl, etc.).

The synthesis of (E-II) is effected via amide bond formation between the commercially available acid (E-I) and a suitable amine (E-XXXII). In this context, a very large number of possible reaction procedures are described in the literature, for example, in V. Pattabiraman, J. Bode, Nature Vol.: 480 (2011) Issue: 7378, pp. 471-479 and literature cited therein. A very large number of the amines (E-XXXII) are commercially available.

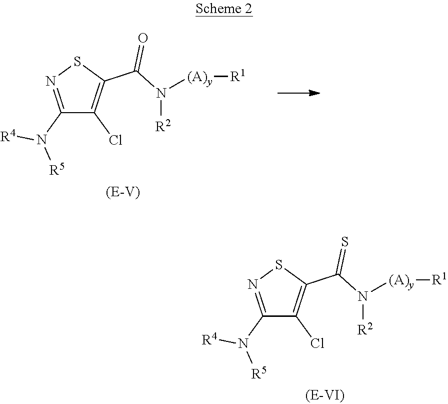

The resulting amide compounds of type (E-V), for example, can be converted in a further step into the corresponding thioamides (E-VI) as shown in Scheme 2, by reaction with a sulphur-transferring reagent (thionation agent) such as, for example, P4510 or Lawesson's reagent [2,4-Bis(4-methoxyphenyl)-1,3,2,4-dithiadiphosphetane-2,4-disulfide]. For this purpose, (E-V) is stirred with equimolar amounts or an excess of Lawesson's reagent in an inert solvent such as, for example, toluene or xylene at temperatures between 80° C. and 200° C. In an analogous manner, the resulting compounds of structure types (E-III), (E-IV), (E-XX), (E-XXIII), (E-XXVIII) can be converted to the corresponding thioamides.

Scheme 1 describes synthetic routes to the target compounds according to the invention, in which amide formation initially takes place and then an amino substituent is introduced into the 3-position of the isothiazole ring. The sequence of these transformations may also be changed, which results in the synthetic route shown in Scheme 3.

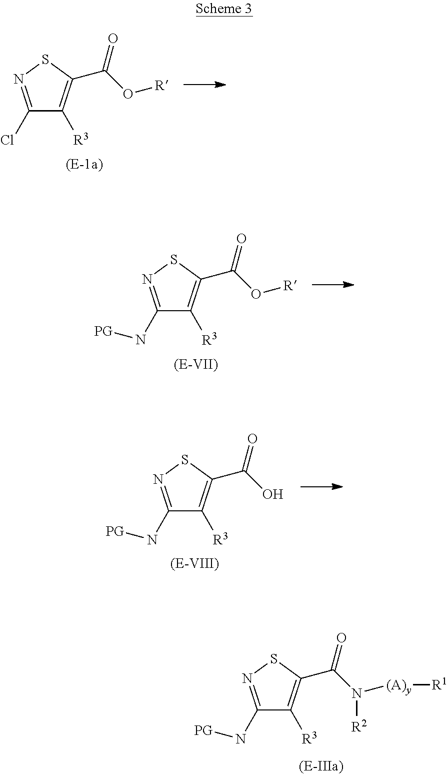

Compound (E-Ia) can be obtained by esterification using a conventional method from the corresponding carboxylic acid (for example compound (E-I)), wherein R′ may be (C₁-C₆)-alkyl, (C₁-C₆)-haloalkyl, (C₃-C₈)-cycloalkyl, (C₃-C₈)-cycloalkyl-(C₁-C₄)-alkyl or optionally substituted phenyl. The structure (E-VII) is then reacted with a suitable ammonium surrogate, comprising a cleavable protecting group PG, in an analogous manner to the reaction of (E-II) to give (E-III) described in Scheme 1. The following step is the cleavage of the ester group, which may be conveniently carried out under basic conditions, for example, by using an inorganic base such as NaOH or KOH in solvents such as MeOH, tetrahydrofuran (THF), water or other solvents, or mixtures of these solvents. The resulting acid of structure type (E-VIII) is reacted with a suitable amine (E-XXXII) to give the amide (E-IIIa), wherein again one of the many amide forming reactions described in the literature can be used.

The compounds (E-III), (E-IV) or (E-V) may in turn be used as starting points for derivatizations leading to further compounds according to the invention. For instance, the chlorine atom can be removed under reductive conditions for example, in order to afford compounds bearing a hydrogen atom in the 4-position of the isothiazole ring. This reaction may be achieved using a heterogeneous catalyst such as, for example, Pd on activated carbon in a hydrogen atmosphere at pressures between 1 bar and 50 bar in solvents such as ethyl acetate, ethanol, THF, etc.

An alternative strategy to the synthetic routes shown in Schemes 1 and 3, which is particularly suitable for the synthesis of a multitude of compounds according to the invention having different R³ substituents, is shown in Scheme 4. Compounds with R³ =iodo allow an easy access to various 4-substituted isothiazole derivatives of formula (G).

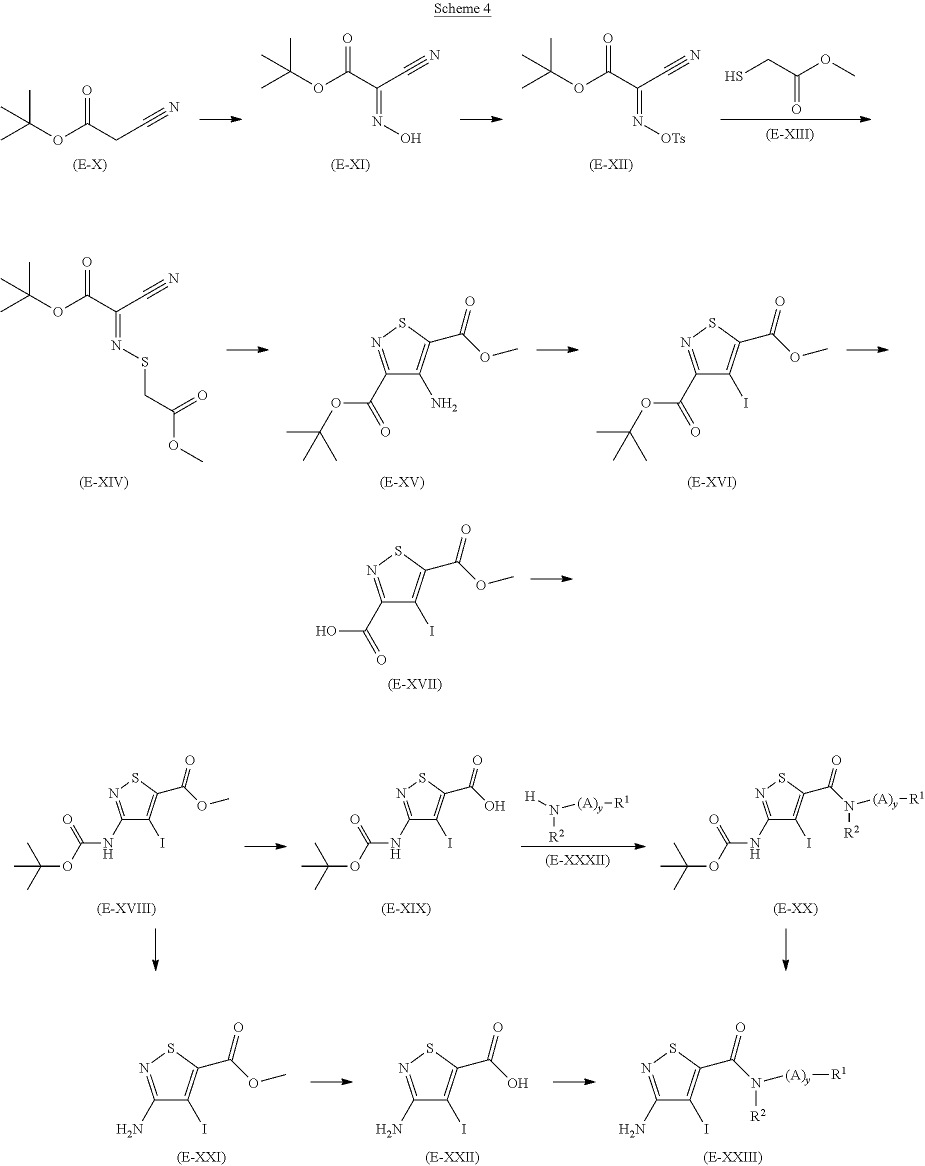

The main intermediates for a broad range of derivatizations of the 4-position of the isothiazole ring (R³) may be, for example, structures of type (E-XX) or (E-XXIII), which may be obtained from the corresponding acids (E-XIX) and (E-XXII) by the common amidation reactions with suitable amines (E-XXXII), as described above. The acids in turn are available from the corresponding esters (E-XVIII) and (E-XXI) by basic ester cleavage, for example, with the aid of inorganic bases such as NaOH or LiOH or other bases in aqueous solvents or solvent mixtures.

The intermediate (E-XVIII) can be obtained, for example, from the acid (E-XVII) by Hoffman degradation, Curtius or Schmidt rearrangement or by a related reaction, wherein the tertiary butyl carbamate, which is readily isolatable, is directly obtained using a suitable reaction procedure (t-BuOH as solvent or solvent constituent). This tertiary butyl carbamate (E-XVIII) may be cleaved to the free amine (E-XXI) by treatment with acid, such as, for example, trifluoroacetic acid or dilute mineral acid.

The required acid (E-XVII) may be obtained, for example, from the tertiary butyl ester (E-XVI) by the action of acid, such as, for example, trifluoroacetic acid or dilute mineral acid. The latter may be obtained from the amino compound (E-XV) by the Sandmeyer reaction or related reactions. For instance, (E-XV) may be reacted, for example, with an alkyl nitrite, such as isoamyl nitrite, and iodine in an inert solvent, such as acetonitrile, at temperatures between 20° C. and 150° C.

The amino compound (E-XV) may be synthesized from the compound (E-XIV) by cyclization, by firstly treating the latter with a weak base, for example triethylamine or other organic bases, and directly after with ethanolic HCl.

The starting compound (E-XIV) required for the cyclization can be readily prepared in three steps from the cyanoacetic ester (E-X). For this purpose, (E-X) is initially reacted with NaNO₂ in aqueous acetic acid, which forms the oxime (E-XI), which may be converted in a second step to the para-tolylsulphonate. For this purpose, (E-XI) is stirred with a suitable sulphonylating reagent, for example para-tolylsulphonyl chloride, and an organic base, for example pyridine. The resulting tosylate (E-XII) is reacted in the third step with the thioglycolate (E-XIII), forming a N—S bond, to give the cyclization precursor (E-XIV). This reaction generally takes place in a commonly used organic solvent such as ethanol, with the aid of an organic base such as pyridine.

The compounds (E-XVIII), (E-XX), and (E-XXIII) described in Scheme 4 may also be starting materials for further compounds according to the present invention, in particular having diverse R³ substituents. Reactions that may be used for this purpose, among others, are shown in Schemes 5, 6 and 7.

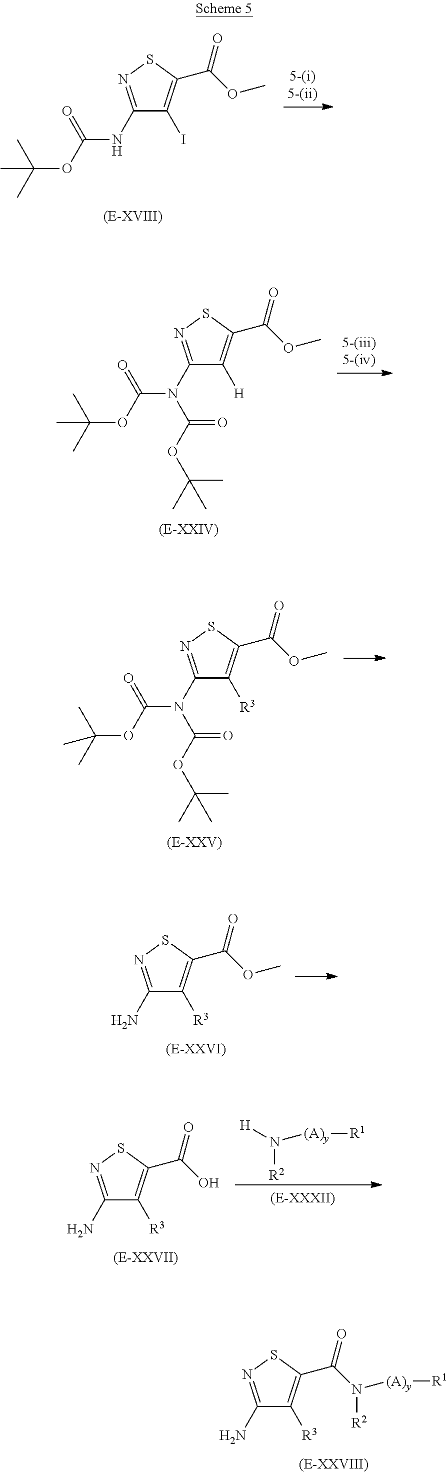

As shown in Scheme 5, starting from compound (E-XVIII), in a first step 5-(i) the iodine atom is removed, which may be accomplished by the action of zinc dust in acidic medium, for example by means of acetic acid as solvent. Subsequently, in step 5-(ii), a second BOC (tert.-butyloxycarbonyl) group is introduced on the nitrogen bound to the 3-position of the isothiazole, which yields compound (E-XXIV). This compound can be deprotonated in the 4-position of the isothiazole ring in step 5-(iii) using a suitable organometallic base such as TMPZnClLiCl (Knochel et al. Angew. Chem. Int. Ed. 2011, 50, 9794-9824), such that a heterocyclic organometallic compound is formed, which may then be reacted in a cross-coupling reaction in step 5-(iv) to afford the compound (E-XXV), wherein in R³ is not hydrogen. If the cross-coupling reaction is carried out with R³-Hal, then Hal can be chlorine, bromine or iodine.

The cross-coupling reaction in step 5-(iv) is generally carried out with the aid of a transition metal catalyst or transition metal precatalyst (Pd₂dba₃, PdCl₂(PPh₃)₂, etc.) and a suitable complex-forming ligand (PPh₃, P(o-furyl)₃, etc.) in a suitable solvent (THF, toluene, etc.), generally at temperatures in the range of 25° C. and 120° C.

The further steps to the target compounds are firstly cleavage of the BOC groups under acidic conditions (e.g. trifluoroacetic acid) to give (E-XXVI), secondly cleavage of the methyl ester (E-XXVII) under basic conditions (e.g. NaOH in a mixture of methanol and water), and thirdly amide bond formation with amines (E-XXXII) to give the compounds (E-XXVIII).

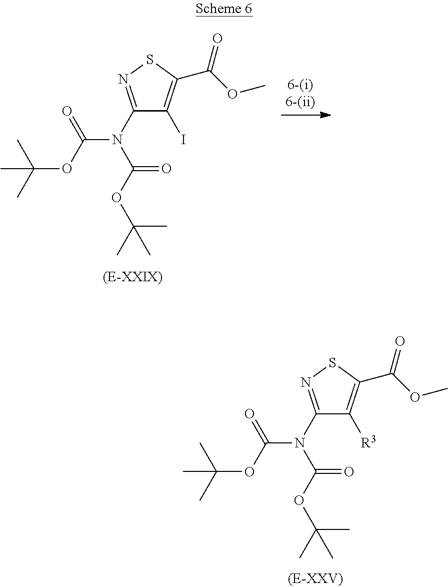

Scheme 6 shows how 4-iodothiazoles, such as (E-XXIX), can be converted into compounds (E-XXV), wherein R³ is not iodine. 4-Iodothiazoles can be converted directly to a metallized isothiazoles via a metal-halogen exchange. For this purpose, in step 6-(i) the double tertiary butyl carbamate protected compound (E-XXIX) is metallized in the 4-position, e.g. reacted with a suitable organometallic compound, a Grignard compound for example, in an inert solvent, generally at temperatures below −50° C. The isothiazole compound metallized in the 4-position thus obtained, in addition to the cross-coupling reaction described above, can also be directly subjected to reaction with a sufficiently reactive electrophilic agent in step 6-(ii). In such a case, no catalyst is required. The electrophilic reaction partners used may be, for example, alkyl halides such as methyl iodide, isopropyl iodide, or alkenyl halides such as allyl bromide, or alkynyl halides such as propargyl bromide or any substituted arylalkyl halide such as benzyl bromide or amides such as, for example, dimethylformamide or other carbonyl compounds such as acetone, propionaldehyde or ethyl formate, etc. or also disulphide compounds such as, for example, dimethyl disulphide. A product of structural formula (E-XXV) is obtained in all cases as a result of the reactions.

However, 4-iodoisothiazoles may also participate directly in transition metal-catalyzed cross-coupling reactions, without intermediate generation of isothiazolyl metal species, whereby a multiplicity of different residues R³ residues are also accessible. Reactions which may be used in this context are, for example, the Suzuki-Miyaura reaction (reaction with arylboronic acids or heteroarylboronic acids) or the Sonogashira reaction (reaction with terminal alkynes) or numerous variants of these two reactions.

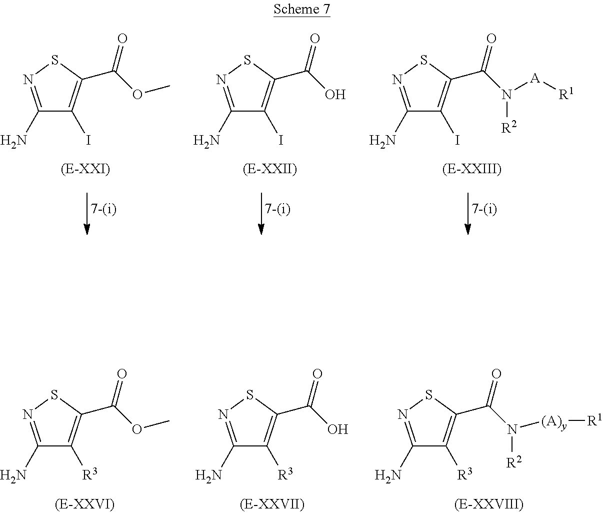

In Scheme 7, the conversion of compounds respective (E-XXI), (E-XXII) or (E-XXIII) to the respective compounds (E-XXVI), (E-XXVII) or (E-XXVIII) in step 7-(i) is shown. Depending on the desired R³ residue in compounds (E-XXVI), (E-XXVII) or (E-XXVIII), wherein in each case R³ is not iodine, different catalysts and reaction conditions in step 7-(i) may be used, e.g. when using cross-coupling reactions. Reaction conditions suitable in step 7-(i) are described in the literature and summarized in recent reviews: Chinchilla et al. Chem. Soc. Rev. 2011, 40, 5084-5121; Suzuki et al. Chem. Rev. 1995, 95, 2457-2483; Science of Synthesis, Cross Coupling and Heck-Type Reactions, 2013, Volumes 1 to 3, Editor: G. A. Molander (Volume 1), M. Larhed (Volume 2), J. P. Wolfe (Volume 3), Georg Thieme Verlag, Stuttgart, N.Y.

It may be advantageous in some cases to introduce the residue R² independently of the residue R¹ as shown in Scheme 8. For this purpose, a compound of type (E-XXX) is initially prepared by one of the routes described above, which is then converted to compound (E-XXXI), wherein R² is not hydrogen. By this conversion compound (E-XXX) can be, for example, alkylated, acylated or sulphonylated.

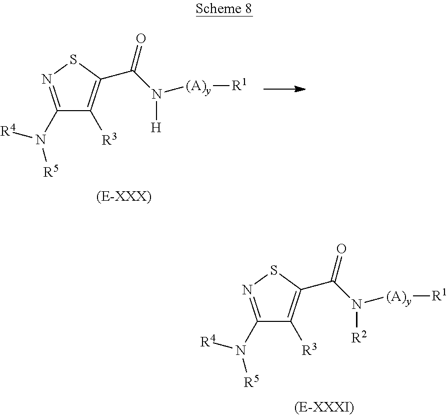

It is evident that the compounds (E-III), (E-IIIa), (E-IV), (E-V), (E-VI), (E-XXIII), (E-XXVIII), and (E-XXXI), prepared by the methods described herein, may themselves in turn be starting points for further chemical reactions, which can lead to additional compounds according to the invention. This applies in particular to reactions which afford modifications or structural variations in the residues R¹, R², R³, R⁴ and/or R⁵.

The present invention further relates to a process for preparing a compound of the formulae (G) as defined hereinabove, and/or a salt thereof, characterized in that
(a) a compound of formula (G) wherein W is oxygen is obtained in a chemical synthesis comprising the step of reacting a compound of the formula (E-II)

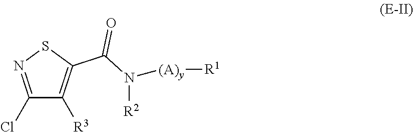

in which R¹, R², R³, A and y each have the meaning as defined in formula (G), and wherein R³ preferably represents a halogen atom, in particular a chlorine atom or a fluorine atom,
with HNR⁴R⁵, wherein R⁴ and R⁵ each have the meaning as defined in formula (G),
or
(b) a compound of formula (G) wherein W is oxygen is obtained in a chemical synthesis comprising the step of reacting a compound of the formula (E-VIII) or of the formula (E-XXVII)

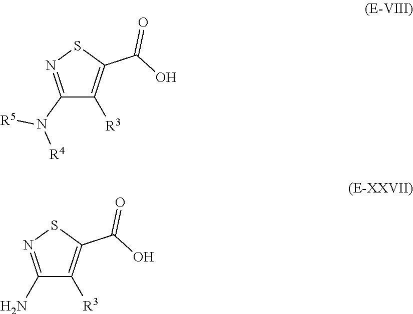

in which R³ has the meaning as defined in formula (G), preferably R³ represents hydrogen or a halogen atom (in case of a halogen atom, preferably a chlorine atom, a bromine atom or an iodine atom), and wherein R⁴ and R⁵ each have the meaning as defined in formula (G), and preferably R⁴ and/or R⁵ represent a protecting group,
with a compound of formula (E-XXXII)

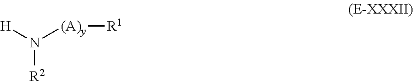

wherein y, A, R¹ and R² each have the meaning as defined in formula (G),
or
(c) a compound of formula (G), wherein W is sulphur is obtained in a chemical synthesis comprising the step of reacting a compound of the formula (E-V)

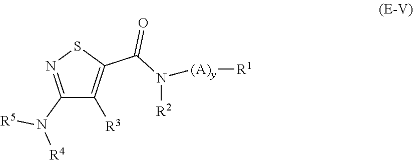

in which R¹, R², R³, R⁴, R⁵ A and y each have the meaning as defined in formula (G), and wherein R³ preferably represents a halogen atom, in particular a chlorine atom,
with a thionation agent, preferably P₄S₁₀ or Lawesson's reagent.

Several intermediates were found to be particularly suitable in the processes for preparing a compound of the formulae (G) according to the present inventon as defined hereinabove, for example those described in the Schemes 1 to 8 above.

Therefore, in a further aspect, the present invention relates to a compound of the formula (Z-A), (Z-B) and/or a salt thereof,

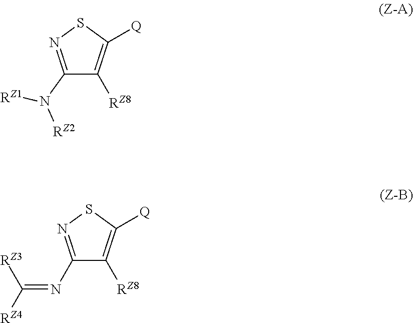

wherein
Q is hydrogen, CN, COCl, COF, CO₂H and salts thereof, CONR¹³R¹⁴, and CO₂R^{q}, wherein R^{q} is (C₁-C₉)-alkyl or (C₁-C₉)-haloalkyl,
R^{Z8} is selected from the group consisting of H, F, Cl, Br, I, CH₃, CH₂F, CHF₂ and CF₃,
R^{Z1} and R^{Z2} are each independently hydrogen, CN, CH₂aryl, X—C(═Y)—, wherein Y is NH, O or S and X is NH₂, OH, SH, (C₁-C₈)-alkyl, (C₁-C₈)-haloalkyl, (C₁-C₈)-alkoxy, (C₁-C₈)-haloalkoxy, (C₁-C₆)-alkylthio, HN(C₁-C₈)-alkyl, or aryl, wherein each aryl is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, and (C₁-C₄)-alkylsulphonyl,
R^{Z3} is hydrogen, (C₁-C₈)-alkyl, (C₁-C₈)-haloalkyl, (C₁-C₈)-alkoxy, (C₁-C₈)-haloalkoxy, (C₁-C₆)-alkylthio, or aryl, wherein aryl is unsubstituted or is substituted by one or more residues from the group consisting of halogen, nitro, hydroxyl, cyano, N¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, and (C₁-C₄)-alkylsulphonyl,
R^{Z4} is (C₁-C₈)-alkyl, (C₁-C₈)-haloalkyl, (C₁-C₈)-alkoxy, (C₁-C₈)-haloalkoxy, (C₁-C₆)-alkylthio, or aryl, wherein aryl is unsubstituted or is substituted by one or more residues from the group consisting of halogen, nitro, hydroxyl, cyano, NR¹³R¹, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, and (C₁-C₄)-alkylsulphonyl,
wherein R¹³ and R″ in each case each independently have the meaning as defined herein, preferably the meaning as defined in one of the preferred embodiments.

Preferably, the present invention relates to compounds of the formula (Z-A) or (Z-B) as defined above, and/or a salt thereof, wherein

Q is CN, COCl, COF, CO₂H and salts thereof, and CO₂R^{q}, wherein R^{q} is (C₁-C₆)-alkyl or (C₁-C₆)-haloalkyl,

R^{Z8} is selected from the group consisting of H, F, Cl, Br, I, CH₃, CH₂F, CHF₂ and CF₃,

R^{Z1} and R^{Z2} are each independently hydrogen, CN, CH₂phenyl, X—C(═Y)—, wherein Y is NH, O or S and X is NH₂, OH, SH, (C₁-C₆)-alkyl, (C₁-C₆)-haloalkyl, (C₁-C₆)-alkoxy, (C₁-C₆)-haloalkoxy, (C₁-C₄)-alkylthio, HN(C₁-C₆)-alkyl, or phenyl, wherein each phenyl is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, and (C₁-C₄)-haloalkoxy,

R^{Z3} is hydrogen, (C₁-C₆)-alkyl, (C₁-C₆)-haloalkyl, (C₁-C₆)-alkoxy, (C₁-C₆)-haloalkoxy , (C₁-C₄)-alkylthio, or phenyl, wherein phenyl is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, and (C₁-C₄)-alkylsulphonyl,

R^{Z4} is (C₁-C₆)-alkyl, (C₁-C₆)-haloalkyl, (C₁-C₆)-alkoxy, (C₁-C₆)-haloalkoxy, (C₁-C₄)-alkylthio, or phenyl, wherein phenyl is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, and (C₁-C₄)-alkylsulphonyl, wherein R¹³ and R¹⁴ in each case each independently have the meaning as defined herein, preferably the meaning as defined in one of the preferred embodiments.

More preferably, the present invention relates to compounds of the formula (Z-A) or (Z-B) as defined above, and/or a salt thereof, wherein

Q is CN, COCl, COF, CO₂H and salts thereof, and CO₂R^{q}, wherein R^{q} is (C₁-C₆)-alkyl,

R^{Z8} is selected from the group consisting of H, F, Cl, Br, I, CH₃, CH₂F, CHF₂ and CF₃,

R^{Z1} and R^{Z2} are each independently hydrogen, CN, CH₂phenyl, X—C(═Y)—, wherein Y is NH, O or S and X is NH₂, OH, SH, (C₁-C₆)-alkyl, (C₁-C₆)-haloalkyl, (C₁-C₆)-alkoxy, (C₁-C₄)-alkylthio, HN(C₁-C₆)-alkyl, or phenyl, wherein each phenyl is unsubstituted or is substituted by one or more residues from the group consisting of halogen, hydroxyl, cyano, (C₁-C₄)-alkyl, (C₁-C₄)-haloalkyl, and (C₁-C₄)-alkoxy,

R^{Z3} is hydrogen, (C₁-C₆)-alkyl, (C₁-C₆)-haloalkyl, (C₁-C₆)-alkoxy, (C₁-C₆)-haloalkoxy, (C₁-C₄)-alkylthio, or phenyl, wherein phenyl is unsubstituted or is substituted by one or more residues from the group consisting of halogen, hydroxyl, cyano, (C₁-C₄)-alkyl, (C₁-C₄)-haloalkyl, (C₁-C₄)-alkoxy, and (C₁-C₄)-haloalkoxy,

R^{Z4} is (C₁-C₆)-alkyl, (C₁-C₆)-haloalkyl, (C₁-C₆)-alkoxy, (C₁-C₆)-haloalkoxy, (C₁-C₄)-alkylthio, or phenyl, wherein phenyl is unsubstituted or is substituted by one or more residues from the group consisting of halogen, hydroxyl, cyano, (C₁-C₄)-alkyl, (C₁-C₄)-haloalkyl, (C₁-C₄)-alkoxy, and (C₁-C₄)-haloalkoxy.

Even more preferably, the present invention relates to compounds of the formula (Z-A) or (Z-B) as defined above, and/or a salt thereof, wherein

Q is CN, COCl, COF, CO₂H and salts thereof, and CO₂R^{q}, wherein R^{q} is (C₁-C₄)-alkyl,

R^{Z8} is selected from the group consisting of H, F, Cl, Br, I, CH₃, CH₂F, CHF₂ and CF₃,

R^{Z1} and R^{Z2} are each independently hydrogen, CN, CH₂phenyl, X—C(═Y)—, wherein Y is NH, O or S and X is NH₂, OH, SH, (C₁-C₆)-alkyl, HN(C₁-C₆)-alkyl, or phenyl, wherein each phenyl is unsubstituted or is substituted by one or more residues from the group consisting of halogen, hydroxyl, cyano, (C₁-C₄)-alkyl, (C₁-C₄)-haloalkyl, and (C₁-C₄)-alkoxy,

R^{Z3} is hydrogen, (C₁-C₆)-alkyl or phenyl, wherein phenyl is unsubstituted or is substituted by one or more residues from the group consisting of halogen, hydroxyl, cyano, (C₁-C₄)-alkyl, (C₁-C₄)-haloalkyl, (C₁-C₄)-alkoxy, and (C₁-C₄)-haloalkoxy,

R^{Z4} is (C₁-C₆)-alkyl or phenyl, wherein phenyl is unsubstituted or is substituted by one or more residues from the group consisting of halogen, hydroxyl, cyano, (C₁-C₄)-alkyl, (C₁-C₄)-haloalkyl, (C₁-C₄)-alkoxy, and (C₁-C₄)-haloalkoxy.

Preferred compounds of formulae (E-II), (E-VII), (E-VIII), (E-XVIII), (E-XXI), (Z-A) and (Z-B) which are particularly useful as intermediates in the processeses for preparing the compounds of the formulae (G) according to the present invention as defined hereinabove are those mentioned in Tables 3 and 4 hereinafter.

The present invention particularly preferably relates to a compound of the formula (Z-1), (Z-2), (Z-3), (Z-4), (Z-5), (Z-6) and/or a salt thereof,

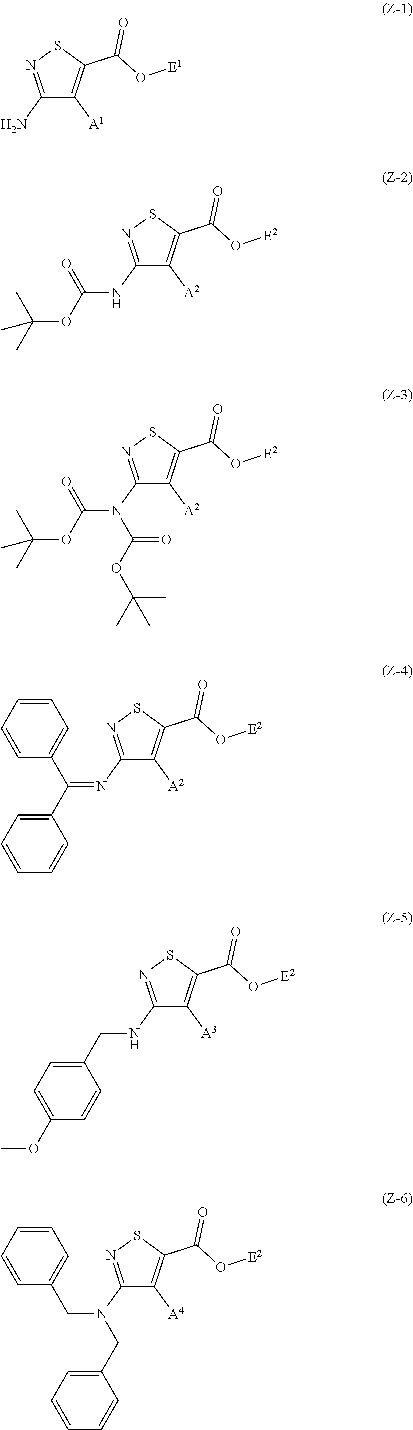

wherein

A¹ is selected from the group consisting of H, F, Cl, Br and I,

A² is selected from the group consisting of F, Cl, Br and I,

A³ is H or Cl,

A⁴ is H or Br,

E¹ is selected from the group consisting of H, methyl, ethyl and iso-propyl,

E² is selected from the group consisting of H, methyl, ethyl, iso-propyl, and tert.-butyl.

Depending on the type of reaction and the reaction conditions used, the skilled person will select suitable organic solvents, such as:
aliphatic hydrocarbons such as pentane, hexane, cyclohexane or petroleum ether; aromatic hydrocarbons such as toluene, o-, m- or p-xylene,
halogenated hydrocarbons such as methylene chloride, chloroform or chlorobenzene,
ethers, such as diethyl ether, diisopropyl ether, tert-butyl methyl ether, dioxane, anisole and tetrahydrofuran (THF),
nitriles such as acetonitrile or propionitrile,
ketones such as acetone, methyl ethyl ketone, diethyl ketone and tert-butyl methyl ketone,
alcohols such as methanol, ethanol, n-propanol, isopropanol, n-butanol and tert-butanol, and also
dimethyl sulphoxide, dimethylformamide, dimethylacetamide, sulpholane,
mixtures of the organic solvents mentioned.

If the compounds described in the context of the present invention, in particular the intermediates and compounds (G) of the present invention, are obtained as solids, the purification can also be carried out by recrystallization or digestion.

The following acids are generally suitable for preparing the acid addition salts of the compounds of the formula (G): hydrohalic acids, such as hydrochloric acid or hydrobromic acid, furthermore phosphoric acid, nitric acid, sulphuric acid, mono- or bifunctional carboxylic acids and hydroxycarboxylic acids, such as acetic acid, maleic acid, succinic acid, fumaric acid, tartaric acid, citric acid, salicylic acid, sorbic acid, or lactic acid, and also sulphonic acids, such as p-toluenesulphonic acid and 1,5-naphthalenedisulphonic acid. The acid addition compounds of the formula (G) can be obtained in a simple manner by the customary methods for forming salts, for example by dissolving a compound of the formula (G) in a suitable organic solvent, such as, for example, methanol, acetone, methylene chloride or benzene, and adding the acid at temperatures of from 0 to 100° C., and they can be isolated in a known manner, for example by filtration, and, if appropriate, purified by washing with an inert organic solvent.

The base addition salts of the compounds of the formula (G) are preferably prepared in inert polar solvents, such as, for example, water, methanol or acetone, at temperatures of from 0 to 100° C. Examples of bases which are suitable for the preparation of the salts according to the invention are alkali metal carbonates, such as potassium carbonate, alkali metal hydroxides and alkaline earth metal hydroxides, for example NaOH or KOH, alkali metal hydrides and alkaline earth metal hydrides, for example NaH, alkali metal alkoxides and alkaline earth metal alkoxides, for example sodium methoxide or potassium tert-butoxide, or ammonia, ethanolamine or quaternary ammonium hydroxide.

What is meant by the “inert solvents” referred to in the above process variants are in each case solvents which are inert under the respective reaction conditions.

Collections of compounds of the formula (G) which can be synthesized by the aforementioned process can also be prepared in a parallel manner, it being possible for this to take place in a manual, partly automated or completely automated manner In this connection, it is possible to automate the reaction procedure, the work-up or the purification of the products and/or intermediates. Overall, this is understood as meaning a procedure as described, for example, by S. H. DeWitt in “Annual Reports in Combinatorial Chemistry and Molecular Diversity: Automated Synthesis”, Volume 1, Verlag Escom, 1997, pages 69 to 77.

For the parallelized reaction procedure and workup it is possible to use a range of commercially available instruments, of the kind offered by, for example, the companies Stem Corporation, Woodrolfe Road, Tollesbury, Essex, CM9 8SE, England, or H+P Labortechnik GmbH, Bruckmannring 28, 85764 OberschleiBheim, Germany. For the parallel purification of compounds (G) or of intermediates produced during the preparation, there are available, inter alia, chromatography apparatuses, for example from ISCO, Inc., 4700 Superior Street, Lincoln, Nebr. 68504, USA. The apparatuses listed allow a modular procedure in which the individual process steps are automated, but between the process steps manual operations have to be carried out. This can be circumvented by using partly or completely integrated automation systems in which the respective automation modules are operated, for example, by robots. Automation systems of this type can be acquired, for example, from Zymark Corporation, Zymark Center, Hopkinton, Mass. 01748, USA.

Besides the methods described here, the preparation of compounds of the formula (G) can take place completely or partially by solid-phase supported methods. For this purpose, individual intermediates or all intermediates in the synthesis or a synthesis adapted for the corresponding procedure are bonded to a synthesis resin. Solid-phase-supported synthesis methods are described extensively in the specialist literature, for example Barry A. Bunin in “The Combinatorial Index”, Academic Press, 1998.

The use of solid-phase-supported synthesis methods permits a number of protocols, which are known from the literature and which for their part may be performed manually or in an automated manner, to be carried out. For example, the “teabag method” (Houghten, U.S. Pat. No. 4,631,211; Houghten et al., Proc. Natl. Acad. Sci, 1985, 82, 5131-5135) in which products from IRORI, 11149 North Torrey Pines Road, La Jolla, Calif. 92037, USA, are employed, may be semiautomated. The automation of solid-phase-supported parallel syntheses is performed successfully, for example, by apparatuses from Argonaut Technologies, Inc., 887 Industrial Road, San Carlos, Calif. 94070, USA or MultiSynTech GmbH, Wullener Feld 4, 58454 Witten, Germany.

The preparation according to the processes described herein produces compounds of the formula (G) in the form of substance collections or libraries. Accordingly, the present invention also provides libraries of compounds of the formula (G) which comprise at least two compounds of the formula (G), and precursors thereof.

The compounds of the formula (G) used in the context of the present invention or according to the invention (and/or their salts) have excellent fungicidal efficacy, in particular against a broad spectrum of fungi and particularly against economically important fungal plant pathogens.

In the context of the present invention, “control” or “controlling” of harmful microorganisms such as phytopathogenic fungi means a reduction in infestation by harmful microorganisms, in particular phytopathogenic fungi, compared with the untreated plant measured as fungicidal efficacy. Preferably, a reduction of at least 25% (i.e. 25% or more), and more preferably a reduction of at least 50% (i.e. 50% or more) is achieved, in each case compared to the untreated plant. Even more preferably, the infestation by harmful microorganisms, in particular phytopathogenic fungi, is suppressed by 70-100% compared to the untreated plant. The infestation of the untreated plant in each case is defined as 100% infestation.

In the context of the present invention, the “control” or “controlling” of harmful microorganisms, in particular phytopathogenic fungi, may be curative, i.e. for treatment of already infected plants, or protective, i.e. for protection of plants which have not yet been infected.

More specifically, the present invention preferably relates to a method for controlling phytopathogenic harmful fungi, characterized in that one or more compounds of the formula (G) according to the present invention are applied to the phytopathogenic harmful fungi and/or their habitat.

The present invention therefore also relates to a method for controlling fungi and/or for controlling one or more plant diseases caused by fungal plant pathogens, characterized in that an effective amount of, preferably a fungicidally effective amount of
one or more compounds of the formula (G) and/or salts thereof as defined hereinabove, preferably in one of the preferred, more preferred or particularly preferred embodiments,
   or
a composition according to the present invention as defined hereinafter comprising one or more compounds of the formula (G) and/or salts thereof as defined hereinabove, preferably a composition as defined in one of the preferred, more preferred or particularly preferred embodiments,
   is applied to the fungi, the plant, to a portion of the plant and/or to plant seeds.

Suitable concentrations of the compounds of the formula (G) used according to the present invention for controlling fungi, preferably for controlling fungi in plants or plant seeds, are for example 125 ppm, 250 ppm, 500 ppm or 1000 ppm.

In a further aspect, the present invention relates to the use of one or more compounds of the formula (G) according to the present invention or of compositions according to the present invention for controlling fungi, preferably for controlling fungi in plants or plant seeds.

In a further aspect, the present invention preferably relates to the use of one or more compounds of the formula (G) according to the present invention or of compositions according to the present invention for controlling phytopathogenic harmful fungi.

The use according to the invention for controlling fungi and/or for controlling one or more plant diseases caused by fungal plant pathogens also includes the case in which the active compound of the formula (G) or its salt is not formed from a precursor substance (“prodrug”) until after application on the plant, in the plant or in the soil.

The compounds of the formula (G) to be used according to the invention or the compounds of the formula (G) according to the invention and/or their salts showed remarkable efficacy against various phytopathogenic harmful fungi, inter alia against species selected from the group consisting of Botrytis spp., Phytophthora spp., Puccinia spp., Pyrenophora spp., Septoria spp., Sphaerotheca spp., Uromyces spp., Alternaria spp., and Venturia spp.

In particular, the compounds of the formula (G) to be used according to the invention or the compounds of the formula (G) according to the invention and/or their salts showed remarkable efficacy against various phytopathogenic harmful fungi, inter alia against species selected from the group consisting of Botrytis spp., Phytophthora spp., Puccinia spp., Pyrenophora spp., Septoria spp., Sphaerotheca spp., and Uromyces spp.

The compounds of the formula (G) to be used according to the invention or the compounds of the formula (G) according to the invention and/or their salts allowed remarkable control of species selected from the group of Botrytis cinerea, Phytophthora infestans, Puccinia recondita, Pyrenophora teres, Septoria trifici, Sphaerotheca fuliginea, Uromyces appendiculatus, Alternaria solani and Venturia inaequalis.

In particular, the compounds of the formula (G) to be used according to the invention or the compounds of the formula (G) according to the invention and/or their salts allowed remarkable control of species selected from the group of Botrytis cinerea, Phytophthora infestans, Puccinia recondita, Pyrenophora teres, Septoria trifici, Sphaerotheca fuliginea, and Uromyces appendiculatus.

As described in more detail in the biological examples below, the compounds of the formula (G) according to the invention showed remarkable efficacy in controlling Botrytis cinerea (grey mould), Phytophthora infestans (tomato late blight), Puccinia recondita (brown rust on wheat), Pyrenophora teres (net blotch on barley), Septoria trifici (leaf spot on wheat), Sphaerotheca fuliginea (powdery mildew on cucurbits), Uromyces appendiculatus (bean rust), Alternaria solani on tomatoes, and Venturia inaequalis (apple scab on apples).

In particular, as described in more detail in the biological examples below, the compounds of the formula (G) according to the invention showed remarkable efficacy in controlling Botrytis cinerea (grey mould), Phytophthora infestans (tomato late blight), Puccinia recondita (brown rust on wheat), Pyrenophora teres (net blotch on barley), Septoria trifici (leaf spot on wheat), Sphaerotheca fuliginea (powdery mildew on cucurbits), and Uromyces appendiculatus (bean rust).

In a further aspect, the present invention preferably relates to the use of one or more compounds of the formula (G) according to the present invention or of compositions according to the present invention for treatment of transgenic plants, of seeds and of seed of transgenic plants.

Thus, in a further aspect, the present invention relates to a composition, characterized in that said composition comprises one or more compounds of the formula (G) and/or salts thereof as defined hereinabove, preferably in one of the preferred, more preferred or particularly preferred embodiments,

and one or more further substances selected from groups (i) and/or (ii):

(i) one or more further agrochemically active substances, preferably selected from the group consisting of further fungicides, insecticides, acaricides, nematicides, herbicides, safeners, fertilizers and/or plant growth regulators,

(ii) one or more formulation auxiliaries customary in crop protection, preferably said formulation auxiliaries are selected from agrochemically acceptable adjuvants, preferably selected from the group consisting of surfactants, liquid diluents and solid diluents.

Such a composition according to the present invention preferably comprises a biologically effective amount, preferably a fungicidally effective amount, one or more compounds of the formula (G) and/or salts thereof as defined hereinabove.

The compounds of the formula (G) and/or salts thereof can be formulated in various ways according to which biological and/or physicochemical parameters are required. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil- or water-based dispersions, oil-miscible solutions, capsule suspensions (CS), dusting products (DP), seed-dressing products, granules for broadcasting and soil application, granules (GR) in the form of microgranules, sprayable granules, coated granules and adsorption granules, water-dispersible granules (WG), water-soluble granules (SG), ULV formulations, microcapsules and waxes.

The compounds of the formula (G) and/or salts thereof can be employed as such or in the form of their preparations (formulations) combined with other pesticidally active compounds, such as, for example, insecticides, acaricides, nematicides, herbicides, further fungicides, safeners, fertilizers and/or growth regulators, for example as finished formulations or as tank mixes. The combination formulations can be prepared on the basis of the abovementioned formulations, while taking account of the physical properties and stabilities of the active compounds to be combined.

Isomers

Depending on the nature of the substituents, the compounds of the formula (G) may be in the form of geometric and/or optically active isomers or corresponding isomer mixtures in different compositions.

These stereoisomers are, for example, enantiomers, diastereomers, atropisomers or geometric isomers. Accordingly, the invention encompasses both pure stereoisomers and any mixture of these isomers.

Methods and Uses

The invention also relates to a method for controlling unwanted microorganisms, characterized in that the compounds of the formula (G) are applied to the microorganisms and/or in their habitat.

The invention further relates to seed which has been treated with at least one compound of the formula (G).

The invention also provides a method for protecting seed against unwanted microorganisms by using seed treated with at least one compound of the formula (G).

The compounds of the formula (G) have potent microbicidal activity and can be used for control of unwanted microorganisms, such as fungi and bacteria, in crop protection and in the protection of materials.

The compounds of the formula (G) have very good fungicidal properties and can be used in crop protection, for example for control of Plasmodiophoromycetes, Oomycetes, Chytridiomycetes, Zygomycetes, Ascomycetes, Basidiomycetes and Deuteromycetes.

Bactericides can be used in crop protection, for example, for control of Pseudomonadaceae, Rhizobiaceae, Enterobacteriaceae, Corynebacteriaceae and Streptomycetaceae.

The compounds of the formula (G) can be used for curative or protective control of phytopathogenic fungi. The invention therefore also relates to curative and protective methods for controlling phytopathogenic fungi by the use of the inventive active ingredients or compositions, which are applied to the seed, the plant or plant parts, the fruit or the soil in which the plants grow.

Plants

All plants and plant parts can be treated in accordance with the invention. Plants are understood here to mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the transgenic plants and including the plant cultivars which are protectable and non-protectable by plant breeders' rights. Plant parts are understood to mean all parts and organs of plants above and below the ground, such as shoot, leaf, flower and root, examples of which include leaves, needles, stalks, stems, flowers, fruit bodies, fruits and seeds, and also roots, tubers and rhizomes. The plant parts also include harvested material and vegetative and generative propagation material, for example cuttings, tubers, rhizomes, slips and seeds.

Plants which can be treated in accordance with the invention include the following: cotton, flax, grapevine, fruit, vegetables, such as Rosaceae sp. (for example pome fruits such as apples and pears, but also stone fruits such as apricots, cherries, almonds and peaches, and soft fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leek, onion), Papilionaceae sp. (for example peas); major crop plants, such as Gramineae sp. (for example maize, turf, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pak choi, kohlrabi, radishes, and oilseed rape, mustard, horseradish and cress), Fabacae sp. (for example bean, peanuts), Papilionaceae sp. (for example soya bean), Solanaceae sp. (for example potatoes), Chenopodiaceae sp. (for example sugar beet, fodder beet, swiss chard, beetroot); useful plants and ornamental plants for gardens and wooded areas; and genetically modified varieties of each of these plants.

Pathogens

Non-limiting examples of pathogens of fungal diseases which can be treated in accordance with the invention include:

diseases caused by powdery mildew pathogens, for example Blumeria species, for example Blumeria graminis; Podosphaera species, for example Podosphaera leucotricha; Sphaerotheca species, for example Sphaerotheca fuliginea; Uncinula species, for example Uncinula necator;

diseases caused by rust disease pathogens, for example Gymnosporangium species, for example Gymnosporangium sabinae; Hemileia species, for example Hemileia vastatrix; Phakopsora species, for example Phakopsora pachyrhizi or Phakopsora meibomiae; Puccinia species, for example Puccinia recondita, Puccinia graminis oder Puccinia striiformis; Uromyces species, for example Uromyces appendiculatus;

diseases caused by pathogens from the group of the Oomycetes, for example Albugo species, for example Albugo candida; Bremia species, for example Bremia lactucae; Peronospora species, for example Peronospora pisi or P. brassicae; Phytophthora species, for example Phytophthora infestans; Plasmopara species, for example Plasmopara viticola; Pseudoperonospora species, for example Pseudoperonospora humuli or Pseudoperonospora cubensis; Pythium species, for example Pythium ultimum;

leaf blotch diseases and leaf wilt diseases caused, for example, by Alternaria species, for example Alternaria solani; Cercospora species, for example Cercospora beticola; Cladiosporium species, for example Cladiosporium cucumerinum; Cochliobolus species, for example Cochliobolus sativus (conidial form: Drechslera, syn: Helminthosporium) or Cochliobolus miyabeanus; Colletotrichum species, for example Colletotrichum lindemuthanium; Cycloconium species, for example Cycloconium oleaginum; Diaporthe species, for example Diaporthe citri; Elsinoe species, for example Elsinoe fawcettii; Gloeosporium species, for example Gloeosporium laeticolor; Glomerella species, for example Glomerella cingulata; Guignardia species, for example Guignardia bidwelli; Leptosphaeria species, for example Leptosphaeria maculans; Magnaporthe species, for example Magnaporthe grisea; Microdochium species, for example Microdochium nivale; Mycosphaerella species, for example Mycosphaerella graminicola, Mycosphaerella arachidicola or Mycosphaerella fijiensis; Phaeosphaeria species, for example Phaeosphaeria nodorum; Pyrenophora species, for example Pyrenophora teres or Pyrenophora tritici repentis; Ramularia species, for example Ramularia collo-cygni or Ramularia areola; Rhynchosporium species, for example Rhynchosporium secalis; Septoria species, for example Septoria apii or Septoria lycopersici; Stagonospora species, for example Stagonospora nodorum; Typhula species, for example Typhula incarnata; Venturia species, for example Venturia inaequalis;

root and stem diseases caused, for example, by Corticium species, for example Corticium graminearum; Fusarium species, for example Fusarium oxysporum; Gaeumannomyces species, for example Gaeumannomyces graminis; Plasmodiophora species, for example Plasmodiophora brassicae; Rhizoctonia species, for example Rhizoctonia solani; Sarocladium species, for example Sarocladium oryzae; Sclerotium species, for example Sclerotium oryzae; Tapesia species, for example Tapesia acuformis; Thielaviopsis species, for example Thielaviopsis basicola;

ear and panicle diseases (including corn cobs) caused, for example, by Alternaria species, for example Alternaria spp.; Aspergillus species, for example Aspergillus flavus; Cladosporium species, for example Cladosporium cladosporioides; Claviceps species, for example Claviceps purpurea; Fusarium species, for example Fusarium culmorum; Gibberella species, for example Gibberella zeae; Monographella species, for example Monographella nivalis; Stagnospora species, for example Stagnospora nodorum;

diseases caused by smut fungi, for example Sphacelotheca species, for example Sphacelotheca reiliana; Tilletia species, for example Tilletia caries or Tilletia controversa; Urocystis species, for example Urocystis occulta; Ustilago species, for example Ustilago nuda;

fruit rot caused, for example, by Aspergillus species, for example Aspergillus flavus; Botrytis species, for example Botrytis cinerea; Penicillium species, for example Penicillium expansum or Penicillium purpurogenum; Rhizopus species, for example Rhizopus stolonifer; Sclerotinia species, for example Sclerotinia sclerotiorum; Verticilium species, for example Verticilium alboatrum; seed- and soil-borne rot and wilt diseases, and also diseases of seedlings, caused, for example, by Alternaria species, for example Alternaria brassicicola; Aphanomyces species, for example Aphanomyces euteiches; Ascochyta species, for example Ascochyta lentis; Aspergillus species, for example Aspergillus flavus; Cladosporium species, for example Cladosporium herbarum; Cochliobolus species, for example Cochliobolus sativus (conidial form: Drechslera, Bipolaris Syn: Helminthosporium); Colletotrichum species, for example Colletotrichum coccodes; Fusarium species, for example Fusarium culmorum; Gibberella species, for example Gibberella zeae; Macrophomina species, for example Macrophomina phaseolina; Microdochium species, for example Microdochium nivale; Monographella species, for example Monographella nivalis; Penicillium species, for example Penicillium expansum; Phoma species, for example Phoma lingam; Phomopsis species, for example Phomopsis sojae; Phytophthora species, for example Phytophthora cactorum; Pyrenophora species, for example Pyrenophora graminea; Pyricularia species, for example Pyricularia oryzae; Pythium species, for example Pythium ultimum; Rhizoctonia species, for example Rhizoctonia solani; Rhizopus species, for example Rhizopus oryzae; Sclerotium species, for example Sclerotium rolfsii; Septoria species, for example Septoria nodorum; Typhula species, for example Typhula incarnata; Verticillium species, for example Verticillium dahliae;

cancers, galls and witches' broom caused, for example, by Nectria species, for example Nectria galligena;

wilt diseases caused, for example, by Monilinia species, for example Monilinia laxa;

deformations of leaves, flowers and fruits caused, for example, by Exobasidium species, for example Exobasidium vexans; Taphrina species, for example Taphrina deformans;

degenerative diseases in woody plants, caused, for example, by Esca species, for example Phaeomoniella chlamydospora, Phaeoacremonium aleophilum or Fomitiporia mediterranea; Ganoderma species, for example Ganoderma boninense;

diseases of flowers and seeds caused, for example, by Botrytis species, for example Botrytis cinerea;

diseases of plant tubers caused, for example, by Rhizoctonia species, for example Rhizoctonia solani; Helminthosporium species, for example Helminthosporium solani;

diseases caused by bacterial pathogens, for example Xanthomonas species, for example Xanthomonas campestris pv. oryzae; Pseudomonas species, for example Pseudomonas syringae pv. lachrymans; Erwinia species, for example Erwinia amylovora.

Preference is given to controlling the following diseases of soya beans:

Fungal diseases on leaves, stems, pods and seeds caused, for example, by Alternaria leaf spot (Alternaria spec. atrans tenuissima), Anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

Mycotoxins

In addition, the compounds of the formula (G) can reduce the mycotoxin content in the harvested material and the foods and feeds prepared therefrom. Mycotoxins include particularly, but not exclusively, the following: deoxynivalenol (DON), nivalenol, 15-Ac-DON, 3-Ac-DON, T2- and HT2-toxin, fumonisins, zearalenon, moniliformin, fusarin, diaceotoxyscirpenol (DAS), beauvericin, enniatin, fusaroproliferin, fusarenol, ochratoxins, patulin, ergot alkaloids and aflatoxins which can be produced, for example, by the following fungi: Fusarium spec., such as F. acuminatum, F. asiaticum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides etc., and also by Aspergillus spec., such as A. flavus, A. parasiticus, A. nomius, A. ochraceus, A. clavatus, A. terreus, A. versicolor, Penicillium spec., such as P. verrucosum, P. viridicatum, P. citrinum, P. expansum, P. claviforme, P. roqueforti, Claviceps spec., such as C. purpurea, C. fusiformis, C. paspali, C. africana, Stachybotrys spec. and others.

Material Protection

The compounds of the formula (G) can also be used in the protection of materials, for protection of industrial materials against attack and destruction by phytopathogenic fungi.

In addition, the compounds of the formula (G) can be used as antifouling compositions, alone or in combinations with other active ingredients.

Industrial materials in the present context are understood to mean inanimate materials which have been prepared for use in industry. For example, industrial materials which are to be protected by inventive compositions from microbial alteration or destruction may be adhesives, glues, paper, wallpaper and board/cardboard, textiles, carpets, leather, wood, fibers and tissues, paints and plastic articles, cooling lubricants and other materials which can be infected with or destroyed by microorganisms. Parts of production plants and buildings, for example cooling-water circuits, cooling and heating systems and ventilation and air-conditioning units, which may be impaired by the proliferation of microorganisms may also be mentioned within the scope of the materials to be protected. Industrial materials within the scope of the present invention preferably include adhesives, sizes, paper and card, leather, wood, paints, cooling lubricants and heat transfer fluids, more preferably wood.

The compounds of the formula (G) may prevent adverse effects, such as rotting, decay, discoloration, decoloration or formation of mould.

In the case of treatment of wood the compounds of the formula (G) may also be used against fungal diseases liable to grow on or inside timber. The term “timber” means all types of species of wood, and all types of working of this wood intended for construction, for example solid wood, high-density wood, laminated wood, and plywood. The method for treating timber according to the invention mainly consists in contacting a composition according to the invention; this includes for example direct application, spraying, dipping, injection or any other suitable means.

In addition, the compounds of the formula (G) can be used to protect objects which come into contact with saltwater or brackish water, especially hulls, screens, nets, buildings, moorings and signalling systems, from fouling.

The compounds of the formula (G) can also be employed for protecting storage goods. Storage goods are understood to mean natural substances of vegetable or animal origin or processed products thereof which are of natural origin, and for which long-term protection is desired. Storage goods of vegetable origin, for example plants or plant parts, such as stems, leaves, tubers, seeds, fruits, grains, can be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The inventive compositions may prevent adverse effects, such as rotting, decay, discoloration, decoloration or formation of mould.

Microorganisms capable of degrading or altering the industrial materials include, for example, bacteria, fungi, yeasts, algae and slime organisms The compounds of the formula (G) preferably act against fungi, especially moulds, wood-discoloring and wood-destroying fungi (Ascomycetes, Basidiomycetes, Deuteromycetes and Zygomycetes), and against slime organisms and algae. Examples include microorganisms of the following genera: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Ophiostoma spp., Ceratocystis spp., Humicola spp., Petriella spp., Trichurus spp., Coriolus spp., Gloeophyllum spp., Pleurotus spp., Poria spp., Serpula spp. and Tyromyces spp., Cladosporium spp., Paecilomyces spp. Mucor spp., Escherichia, such as Escherichia coli; Pseudomonas, such as Pseudomonas aeruginosa; Staphylococcus, such as Staphylococcus aureus, Candida spp. and Saccharomyces spp., such as Saccharomyces cerevisae.

Formulations

The present invention further relates to a composition for controlling unwanted microorganisms, comprising at least one of the compounds of the formula (G). These are preferably fungicidal compositions which comprise agriculturally suitable auxiliaries, solvents, carriers, surfactants or extenders.

According to the invention, a carrier is a natural or synthetic, organic or inorganic substance with which the active ingredients are mixed or combined for better applicability, in particular for application to plants or plant parts or seed. The carrier, which may be solid or liquid, is generally inert and should be suitable for use in agriculture.

Useful solid carriers include: for example ammonium salts and natural rock flours, such as kaolins, clays, talc, chalk, quartz, attapulgite, montmorillonite or diatomaceous earth, and synthetic rock flours, such as finely divided silica, alumina and silicates; useful solid carriers for granules include: for example, crushed and fractionated natural rocks such as calcite, marble, pumice, sepiolite and dolomite, and also synthetic granules of inorganic and organic flours, and granules of organic material such as paper, sawdust, coconut shells, maize cobs and tobacco stalks; useful emulsifiers and/or foam-formers include: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and also protein hydroly sates; suitable dispersants are nonionic and/or ionic substances, for example from the classes of the alcohol-POE and/or -POP ethers, acid and/or POP POE esters, alkylaryl and/or POP POE ethers, fat and/or POP POE adducts, POE- and/or POP-polyol derivatives, POE- and/or POP-sorbitan or -sugar adducts, alkyl or aryl sulfates, alkyl- or arylsulfonates and alkyl or aryl phosphates or the corresponding PO-ether adducts. Additionally suitable are oligo- or polymers, for example those derived from vinylic monomers, from acrylic acid, from EO and/or PO alone or in combination with, for example, (poly)alcohols or (poly)amines. It is also possible to use lignin and its sulfonic acid derivatives, unmodified and modified celluloses, aromatic and/or aliphatic sulfonic acids and also their adducts with formaldehyde.

The active ingredients can be converted to the customary formulations, such as solutions, emulsions, wettable powders, water- and oil-based suspensions, powders, dusts, pastes, soluble powders, soluble granules, granules for broadcasting, suspoemulsion concentrates, natural products impregnated with active ingredient, synthetic substances impregnated with active ingredient, fertilizers and also microencapsulations in polymeric substances.

The active ingredients can be applied as such, in the form of their formulations or the use forms prepared therefrom, such as ready-to-use solutions, emulsions, water- or oil-based suspensions, powders, wettable powders, pastes, soluble powders, dusts, soluble granules, granules for broadcasting, suspoemulsion concentrates, natural products impregnated with active ingredient, synthetic substances impregnated with active ingredient, fertilizers and also microencapsulations in polymeric substances. Application is accomplished in a customary manner, for example by watering, spraying, atomizing, broadcasting, dusting, foaming, spreading-on and the like. It is also possible to deploy the active ingredients by the ultra-low volume method or to inject the active ingredient preparation/the active ingredient itself into the soil. It is also possible to treat the seed of the plants.

The formulations mentioned can be prepared in a manner known per se, for example by mixing the active ingredients with at least one customary extender, solvent or diluent, emulsifier, dispersant and/or binder or fixing agent, wetting agent, a water repellent, if appropriate siccatives and UV stabilizers and if appropriate dyes and pigments, antifoams, preservatives, secondary thickeners, stickers, gibberellins and also other processing auxiliaries.

The present invention includes not only formulations which are already ready for use and can be deployed with a suitable apparatus to the plant or the seed, but also commercial concentrates which have to be diluted with water prior to use.

The compounds of the formula (G) may be present as such or in their (commercial) formulations and in the use forms prepared from these formulations as a mixture with other (known) active ingredients, such as insecticides, attractants, sterilants, bactericides, acaricides, nematicides, fungicides, growth regulators, herbicides, fertilizers, safeners and/or semiochemicals.

The auxiliaries used may be those substances which are suitable for imparting particular properties to the composition itself or and/or to preparations derived therefrom (for example spray liquors, seed dressings), such as certain technical properties and/or also particular biological properties. Typical auxiliaries include:

extenders, solvents and carriers.

Suitable extenders are, for example, water, polar and nonpolar organic chemical liquids, for example from the classes of the aromatic and nonaromatic hydrocarbons (such as paraffins, alkylbenzenes, alkylnaphthalenes, chlorobenzenes), the alcohols and polyols (which may optionally also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulfones and sulfoxides (such as dimethyl sulfoxide).

Liquefied gaseous extenders or carriers are understood to mean liquids which are gaseous at standard temperature and under standard pressure, for example aerosol propellants such as halohydrocarbons, or else butane, propane, nitrogen and carbon dioxide.

In the formulations it is possible to use tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins and synthetic phospholipids. Further additives may be mineral and vegetable oils.

If the extender used is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, or else water.

Compositions comprising compounds of the formula (G) may additionally comprise further components, for example surfactants. Suitable surfactants are emulsifiers and/or foam formers, dispersants or wetting agents having ionic or nonionic properties, or mixtures of these surfactants. Examples thereof are salts of polyacrylic acid, salts of lignosulfonic acid, salts of phenolsulfonic acid or naphthalenesulfonic acid, polycondensates of ethylene oxide with fatty alcohols or with fatty acids or with fatty amines, substituted phenols (preferably alkylphenols or arylphenols), salts of sulfosuccinic esters, taurine derivatives (preferably alkyl taurates), phosphoric esters of polyethoxylated alcohols or phenols, fatty esters of polyols, and derivatives of the compounds containing sulfates, sulfonates and phosphates, for example alkylaryl polyglycol ethers, alkylsulfonates, alkyl sulfates, arylsulfonates, protein hydrolysates, lignosulfite waste liquors and methylcellulose. The presence of a surfactant is necessary if one of the active ingredients and/or one of the inert carriers is insoluble in water and when application is effected in water. The proportion of surfactants is between 5 and 40 per cent by weight of the inventive composition.

It is possible to use dyes such as inorganic pigments, for example iron oxide, titanium oxide and Prussian Blue, and organic dyes such as alizarin dyes, azo dyes and metal phthalocyanine dyes, and trace nutrients such as salts of iron, manganese, boron, copper, cobalt, molybdenum and zinc.

Further additives may be perfumes, mineral or vegetable, optionally modified oils, waxes and nutrients (including trace nutrients), such as salts of iron, manganese, boron, copper, cobalt, molybdenum and zinc.

Additional components may be stabilizers, such as cold stabilizers, preservatives, antioxidants, light stabilizers, or other agents which improve chemical and/or physical stability.

If appropriate, other additional components may also be present, for example protective colloids, binders, adhesives, thickeners, thixotropic substances, penetrants, stabilizers, sequestering agents, complex formers. In general, the active ingredients can be combined with any solid or liquid additive commonly used for formulation purposes.

The formulations contain generally between 0.05 and 99% by weight, 0.01 and 98% by weight, preferably between 0.1 and 95% by weight, more preferably between 0.5 and 90% of active ingredient, most preferably between 10 and 70 per cent by weight.

The formulations described above can be used for controlling unwanted microorganisms, in which the compositions comprising compounds of the formula (G) are applied to the microorganisms and/or in their habitat.

Mixtures

Compounds of the formula (G) can be used as such or in formulations thereof and can be mixed with known fungicides, bactericides, acaricides, nematicides or insecticides, in order thus to broaden, for example, the activity spectrum or to prevent development of resistance.

Useful mixing partners include, for example, known further fungicides, insecticides, acaricides, nematicides or else bactericides (see also “The Pesticide Manual”, 16th edition, November 2012, The British Crop Protection Council and the Royal Soc. of Chemistry).

A mixture with other known active ingredients, such as herbicides, or with fertilizers and growth regulators, safeners and/or semiochemicals, is also possible.

Seed Treatment

The invention furthermore includes a method for treating seed.

A further aspect of the present invention relates in particular to seeds (dormant, primed, pregerminated or even with emerged roots and leaves) treated with at least one of the compounds of the formula (G). The inventive seeds are used in methods for protection of seeds and emerged plants from the seeds from phytopathogenic harmful fungi. In these methods, seed treated with at least one inventive active ingredient is used.

The compounds of the formula (G) are also suitable for the treatment of seeds and young seedlings. A large part of the damage to crop plants caused by harmful organisms is triggered by the infection of the seeds before sowing or after germination of the plant. This phase is particularly critical since the roots and shoots of the growing plant are particularly sensitive, and even small damage may result in the death of the plant. Accordingly, there is great interest in protecting the seed and the germinating plant by using appropriate compositions.

It is also desirable to optimize the amount of the active ingredient used so as to provide the best possible protection for the seeds, the germinating plants and emerged seedlings from attack by phytopathogenic fungi, but without damaging the plants themselves by the active ingredient used. In particular, methods for the treatment of seed should also take into consideration the intrinsic phenotypes of transgenic plants in order to achieve optimum protection of the seed and the germinating plant with a minimum of crop protection compositions being employed.

The present invention therefore also relates to a method for protecting seeds, germinating plants and emerged seedlings against attack by animal pests and/or phytopathogenic harmful microorganisms by treating the seeds with an inventive composition. The invention also relates to the use of the compositions according to the invention for treating seeds for protecting the seeds, the germinating plants and emerged seedlings against animal pests and/or phytopathogenic microorganisms. The invention further relates to seeds which have been treated with an inventive composition for protection from animal pests and/or phytopathogenic microorganisms.

One of the advantages of the present invention is that the treatment of the seeds with these compositions not only protects the seed itself, but also the resulting plants after emergence, from animal pests and/or phytopathogenic harmful microorganisms. In this way, the immediate treatment of the crop at the time of sowing or shortly thereafter protect plants as well as seed treatment in prior to sowing. It is likewise considered to be advantageous that the inventive active ingredients or compositions can be used especially also for transgenic seed, in which case the plant which grows from this seed is capable of expressing a protein which acts against pests, herbicidal damage or abiotic stress. The treatment of such seeds with the inventive active ingredients or compositions, for example an insecticidal protein, can result in control of certain pests. Surprisingly, a further synergistic effect can be observed in this case, which additionally increases the effectiveness for protection against attack by pests, microorganisms, weeds or abiotic stress.

The compounds of the formula (G) are suitable for protection of seed of any plant variety which is used in agriculture, in the greenhouse, in forests or in horticulture. More particularly, the seed is that of cereals (such as wheat, barley, rye, millet and oats), oilseed rape, maize, cotton, soybeen, rice, potatoes, sunflower, beans, coffee, beet (e.g. sugar beet and fodder beet), peanut, vegetables (such as tomato, cucumber, onions and lettuce), lawns and ornamental plants. Of particular significance is the treatment of the seed of wheat, soybean, oilseed rape, maize and rice.

As also described below, the treatment of transgenic seed with the inventive active ingredients or compositions is of particular significance. This refers to the seed of plants containing at least one heterologous gene which allows the expression of a polypeptide or protein, e.g. having insecticidal properties. These heterologous genes in transgenic seeds may originate, for example, from microorganisms of the species Bacillus, Rhizobium, Pseudomonas, Serratia, Trichoderma, Clavibacter, Glomus or Gliocladium. These heterologous genes preferably originate from Bacillus sp., in which case the gene product is effective against the European corn borer and/or the Western corn rootworm. Particularly preferably, the heterologous genes originate from Bacillus thuringiensis.

In the context of the present invention, the inventive composition is applied to seeds either alone or in a suitable formulation. Preferably, the seed is treated in a state in which it is sufficiently stable for no damage to occur in the course of treatment. In general, seeds can be treated at any time between harvest and some time after sowing. It is customary to use seed which has been separated from the plant and freed from cobs, shells, stalks, coats, hairs or the flesh of the fruits. For example, it is possible to use seed which has been harvested, cleaned and dried down to a moisture content of less than 15% by weight. Alternatively, it is also possible to use seed which, after drying, for example, has been treated with water and then dried again, or seeds just after priming, or seeds stored in primed conditions or pre-germinated seeds, or seeds sown on nursery trays, tapes or paper.

When treating the seeds, it generally has to be ensured that the amount of the inventive composition applied to the seed and/or the amount of further additives is selected such that the germination of the seed is not impaired, or that the resulting plant is not damaged. This must be ensured particularly in the case of active ingredients which can exhibit phytotoxic effects at certain application rates.

The compounds of the formula (G) can be applied directly, i.e. without containing any other components and without having been diluted. In general, it is preferable to apply the compositions to the seed in the form of a suitable formulation. Suitable formulations and methods for seed treatment are known to those skilled in the art. The compounds of the formula (G) can be converted to the customary formulations relevant to on-seed applications, such as solutions, emulsions, suspensions, powders, foams, slurries or combined with other coating compositions for seed, such as film forming materials, pelleting materials, fine iron or other metal powders, granules, coating material for inactivated seeds, and also ULV formulations.

These formulations are prepared in a known manner, by mixing the active ingredients or active ingredient combinations with customary additives, for example customary extenders and solvents or diluents, dyes, wetting agents, dispersants, emulsifiers, antifoams, preservatives, secondary thickeners, adhesives, gibberellins, and also water.

Useful dyes which may be present in the seed dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed dressing formulations usable in accordance with the invention are all substances which promote wetting and which are conventionally used for the formulation of active agrochemical ingredients. Usable with preference are alkylnaphthalenesulfonates, such as diisopropyl- or diisobutylnaphthalenesulfonates.

Useful dispersants and/or emulsifiers which may be present in the seed dressing formulations usable in accordance with the invention are all nonionic, anionic and cationic dispersants conventionally used for the formulation of active agrochemical ingredients. Usable with preference are nonionic or anionic dispersants or mixtures of nonionic or anionic dispersants. Useful nonionic dispersants include especially ethylene oxide/propylene oxide block polymers, alkylphenol polyglycol ethers and tristryrylphenol polyglycol ether, and the phosphated or sulfated derivatives thereof. Suitable anionic dispersants are especially lignosulfonates, polyacrylic acid salts and arylsulfonate/formaldehyde condensates.

Antifoams which may be present in the seed dressing formulations usable in accordance with the invention are all foam-inhibiting substances conventionally used for the formulation of active agrochemical ingredients. Silicone antifoams and magnesium stearate can be used with preference.

Preservatives which may be present in the seed dressing formulations usable in accordance with the invention are all substances usable for such purposes in agrochemical compositions. Examples include dichlorophene and benzyl alcohol hemiformal.

Secondary thickeners which may be present in the seed dressing formulations usable in accordance with the invention are all substances usable for such purposes in agrochemical compositions. Preferred examples include cellulose derivatives, acrylic acid derivatives, xanthan, modified clays and finely divided silica.

Adhesives which may be present in the seed dressing formulations usable in accordance with the invention are all customary binders usable in seed dressing products. Preferred examples include polyvinylpyrrolidone, polyvinyl acetate, polyvinyl alcohol and tylose.

The formulations for on-seed applications usable in accordance with the invention can be used to treat a wide variety of different kinds of seed either directly or after prior dilution with water. For instance, the concentrates or the preparations obtainable therefrom by dilution with water can be used to dress the seed of cereals, such as wheat, barley, rye, oats, and triticale, and also seeds of maize, soybean, rice, oilseed rape, peas, beans, cotton, sunflowers, and beets, or else a wide variety of different vegetable seeds. The formulations usable in accordance with the invention, or the dilute preparations thereof, can also be used for seeds of transgenic plants. In this case, additional synergistic effects may also occur in interaction with the substances formed by expression.

For treatment of seeds with the formulations usable in accordance with the invention, or the preparations prepared therefrom by adding water, all mixing units usable customarily for on-seed applications are useful. Specifically, the procedure in on-seed applications is to place the seeds into a mixer, to add the particular desired amount of the formulations, either as such or after prior dilution with water, and to mix everything until all applied formulations are distributed homogeneously on the seeds. If appropriate, this is followed by a drying operation.

The application rate of the formulations usable in accordance with the invention can be varied within a relatively wide range. It is guided by the particular content of the active ingredients in the formulations and by the seeds. The application rate of each single active ingredient is generally between 0.001 and 15 g per kilogram of seed, preferably between 0.01 and 5 g per kilogram of seed.

GMO

As already mentioned above, it is possible to treat all plants and their parts in accordance with the invention. In a preferred embodiment, wild plant species and plant cultivars, or those obtained by conventional biological breeding methods, such as crossing or protoplast fusion, and also parts thereof, are treated. In a further preferred embodiment, transgenic plants and plant cultivars obtained by genetic engineering methods, if appropriate in combination with conventional methods (Genetically Modified Organisms), and parts thereof are treated. The terms “parts” or “parts of plants” or “plant parts” have been explained above. More preferably, plants of the plant cultivars which are commercially available or are in use are treated in accordance with the invention. Plant cultivars are understood to mean plants which have new properties (“traits”) and have been obtained by conventional breeding, by mutagenesis or by recombinant DNA techniques. They can be cultivars, varieties, bio- or genotypes.

The method of treatment according to the invention can be used in the treatment of genetically modified organisms (GMOs), e.g. plants or seeds. Genetically modified plants (or transgenic plants) are plants of which a heterologous gene has been stably integrated into genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology, RNA interference—RNAi technology or microRNA miRNA—technology). A heterologous gene that is located in the genome is also called a transgene. A transgene that is defined by its particular location in the plant genome is called a transformation or transgenic event.

Plants and plant cultivars which are preferably to be treated according to the invention include all plants which have genetic material which impart particularly advantageous, useful traits to these plants (whether obtained by breeding and/or biotechnological means).

Plants and plant cultivars which are also preferably to be treated according to the invention are resistant against one or more biotic stresses, i.e. said plants show a better defense against animal and microbial pests, such as against nematodes, insects, mites, phytopathogenic fungi, bacteria, viruses and/or viroids.

Plants and plant cultivars which may also be treated according to the invention are those plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance.

Plants and plant cultivars which may also be treated according to the invention, are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content and composition for example cotton or starch, protein content, oil content and composition, nutritional value, reduction in anti-nutritional compounds, improved processability and better storage stability.

Plants that may be treated according to the invention are hybrid plants that already express the characteristic of heterosis or hybrid vigor which results in generally higher yield, vigor, health and resistance towards biotic and abiotic stresses).

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance.

Plants or plant cultivars (obtained by plant biotechnology methods such as genetic engineering) which may also be treated according to the invention show altered quantity, quality and/or storage-stability of the harvested product and/or altered properties of specific ingredients of the harvested product.

Plants or plant cultivars (that can be obtained by plant biotechnology methods such as genetic engineering) which may also be treated according to the invention are plants, such as cotton plants, with altered fiber characteristics. Such plants can be obtained by genetic transformation, or by selection of plants contain a mutation imparting such altered fiber characteristics.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants contain a mutation imparting such altered oil profile characteristics.

Plants or plant cultivars (that can be obtained by plant biotechnology methods such as genetic engineering) which may also be treated according to the invention are plants, such as oilseed rape or related Brassica plants, with altered seed shattering characteristics. Such plants can be obtained by genetic transformation, or by selection of plants contain a mutation imparting such altered seed shattering characteristics and include plants such as oilseed rape plants with delayed or reduced seed shattering.

Plants or plant cultivars (that can be obtained by plant biotechnology methods such as genetic engineering) which may also be treated according to the invention are plants, such as Tobacco plants, with altered post-translational protein modification patterns.

Application Rates

When using the compounds of the formula (G) as fungicides, the application rates can be varied within a relatively wide range, depending on the kind of application. The application rate of the inventive active ingredients is
in the case of treatment of plant parts, for example leaves: from 0.1 to 10 000 g/ha, preferably from 10 to 1000 g/ha, more preferably from 50 to 300 g/ha (in the case of application by watering or dripping, it is even possible to reduce the application rate, especially when inert substrates such as rockwool or perlite are used);
in the case of seed treatment: from 0.1 to 200 g per 100 kg of seed, preferably from 1 to 150 g per 100 kg of seed, more preferably from 2.5 to 25 g per 100 kg of seed, even more preferably from 2.5 to 12.5 g per 100 kg of seed;
in the case of soil treatment: from 0.1 to 10 000 g/ha, preferably from 1 to 5000 g/ha.

These application rates are merely by way of example and are not limiting for the purposes of the invention.

The invention is illustrated by the examples below. However, the invention is not limited to the examples.

### EXAMPLES

In an exemplary manner, some synthesis examples of compounds of the general formula (G) are described below. In the examples, the amounts (including percentages) refer to the weight, unless especially stated otherwise.

The symbols “>” and “<” mean “greater than” and “smaller than”, respectively. The symbol “≥” means “greater than or equal to”, the symbol “≤” means “smaller than or equal to”.

If, in the context of the description and the examples, the terms “R” and “S” are given for the absolute configuration on a centre of chirality of the stereoisomers of the formula (G), this RS nomenclature follows, unless defined differently, the Cahn-Ingold-Prelog rule.

In the context of the present invention and in the Tables mentioning specific and preferred compounds according to the present invention, the following abbreviations may be used:
H=hydrogen
Me=methyl or CH₃
Et=ethyl
Pr=propyl
Bu=butyl
nAlkyl=n-alkyl, e.g. nPr=n-propyl
cAlkyl=cycloalkyl, e.g. cPr=cyclopropyl, cHexyl=cyclohexyl
iAlkyl=isooalkyl, e.g. iPr=isopropyl
tAlkyl=tertiary alkyl, e.g. tBu=tert-butyl
F, Cl, Br, I=fluorine, chlorine, bromine and iodine, respectively, in accordance with the conventional chemical atom symbol
MeO or OMe=methoxy
CN=cyano
NO₂=nitro
Ph=phenyl
diHal=diHal, e.g. diF=difluoro
triHal=triHal, e.g. triF=trifluoro
—CCH=ethynyl (—CCH)

The position of a substituent, e.g. at the phenyl ring in position 2, is stated as a prefix to the symbol or the abbreviation of the radical, for example
2-C₁₌₂-chloro
2-Me=2-methyl

Numerations of the substituent positions for di- or trisubstituted substitution patterns are analogously stated as a prefix, for example
2,3-Cl₂=2,3-dichloro (e.g. as substitution at the phenyl ring)
2,4-diF=2,4-difluoro (e.g. as substitution at the phenyl ring)
2,4-F₂=2,4-difluoro (e.g. as substitution at the phenyl ring)
2,4,6-triF=2,4,6-trifluoro (e.g. as substitution at the phenyl ring)
2-F-4-Cl=2-fluoro, 4-chloro (e.g. as substitution at the phenyl ring)
5-F-2-Me=5-fluoro, 2-methyl (e.g. as substitution at the phenyl ring)

Other abbreviations are to be understood analogously to the examples stated above.

In addition, the customary chemical symbols and formulae apply, such as, for example, CH₂ for methylene or CF₃ for trifluoromethyl or OH for hydroxyl.

Correspondingly, composite meanings are defined as composed of the abbreviations mentioned, for example
4-CF₃-cHexyl=4- trifluoromethyl-cyclohexyl

NMR-Peak Lists and LogP Values

1H-NMR data of selected examples are written in form of 1H-NMR-peak lists. To each signal peak are listed the δ-value in ppm and the signal intensity in round brackets. Between the δ-value signal intensity pairs are semicolons as delimiters.

The peak list of an example has therefore the form:

δ1 (intensityl); δ2 (intensity2); . . . ; δi (intensityi); . . . ; δn (intensityn)

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

For calibrating chemical shift for 1H spectra, tetramethylsilane and/or the chemical shift of the solvent was used, especially in the case of spectra measured in DMSO (Dimethyl sulfoxide). Therefore in NMR peak lists, tetramethylsilane peak can occur, but not necessarily.

The 1H-NMR peak lists are similar to classical 1H-NMR prints and contains therefore usually all peaks, which are listed at classical NMR-interpretation.

Additionally they can show like classical 1H-NMR prints signals of solvents, stereoisomers of the target compounds, which are also object of the invention, and/or peaks of impurities.

To show compound signals in the delta-range of solvents and/or water the usual peaks of solvents, for example peaks of DMSO in DMSO-D₆ and the peak of water are shown in our 1H-NMR peak lists and have usually on average a high intensity .

The peaks of stereoisomers of the target compounds and/or peaks of impurities have usually on average a lower intensity than the peaks of target compounds (for example with a purity >90%).

Such stereoisomers and/or impurities can be typical for the specific preparation process. Therefore their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical 1H-NMR interpretation.

Further details of NMR-data description with peak lists can be found in the publication “Citation of NMR Peaklist Data within Patent Applications” of the Research Disclosure Database Number 564025.

Measurement of LogP values was performed according to EEC directive 79/831 Annex V.A8 by HPLC (High Performance Liquid Chromatography) on reversed phase columns with the following methods:

^{[a]}LogP value and [a] LogP value is determined by measurement of LC-UV, in an acidic range, with 0.1% formic acid in water and acetonitrile as eluent (linear gradient from 10% acetonitrile to 95% acetonitrile).

^{[b]}LogP value and [b] LogP is determined by measurement of LC-UV, in a neutral range, with 0.001 molar ammonium acetate solution in water and acetonitrile as eluent (linear gradient from 10% acetonitrile to 95% acetonitrile).

^{[c]} LogP value and [c] LogP is determined by measurement of LC-UV, in an acidic range, with 0.1% phosphoric acid and acetonitrile as eluent (linear gradient from 10% acetonitrile to 95% acetonitrile).

Calibration was done with straight-chain alkan2-ones (with 3 to 16 carbon atoms) with known LogP values (measurement of LogP values using retention times with linear interpolation between successive alkanones). Lambda-max-values were determined using UV-spectra from 200 nm to 400 nm and the peak values of the chromatographic signals

The compounds according to the present invention, such as described in the Tables 1 to 4, are obtained according to or analogously to the following chemical synthesis examples.

### (A) Chemical Synthesis Examples

### 1. Synthesis of 3-amino-4-chloro-N-(cyclohexylmethypisothiazole-5-carboxamide

### 1.1. Synthesis of 3,4-dichloro-N-(cyclohexylmethypisothiazole-5-carboxamide

730 mg of 3,4-dichloroisothiazole-5-carboxylic acid (3.7 mmol) were dissolved in 10 ml of dichloromethane and a drop of dimethylformamide was added. 1.4 g of oxalyl chloride (11.1 mmol) were added dropwise at room temperature. After stirring for 1 h at room temperature, the solution was evaporated to dryness on a rotary evaporator. The residue was taken up in 3 ml of dichloromethane and slowly added dropwise to a solution of 626 mg of 1-cyclohexylmethanamine (5.5 mmol) and 746 mg of triethylamine (7 4 mmol) in 10 ml of dichloromethane. The mixture was stirred at room temperature for 1 h. The reaction mixture was then added to water and extracted repeatedly with dichloromethane. The concentrated extracts were dried over MgSO4, concentrated and purified by column chromatography. Yield: 1.05 g (97% of theory).

¹H-NMR (400 MHz, CDCl₃ δ, ppm) 6.86 (br, 1H), 3.34 (tr, 2H), 1.77 (m, 4H), 1.66 (m, 1H), 1.58 (m, 1H), 1.3-1.15 (m, 3H), 1.0 (m, 2H).

### 1.2. Synthesis of 4-chloro-N-(cyclohexylmethyl)-3-[(diphenylmethylene)amino]isothiazole-5-carboxamide

1.27 g of 3,4-dichloro-N-(cyclohexylmethyl)isothiazole-5-carboxamide (4 3 mmol) were dissolved in 6 ml of toluene. To this solution were added consecutively 1.87 g of benzophenone imine (10 mmol), 2.8 g of caesium carbonate (8.68 mmol), 102 mg of Xantphos (0.17 mmol) and 79 mg of tri(dibenzylideneacetone)dipalladium (Pd₂dba₃; 0.087 mmol). The reaction vessel with the resulting solution was then briefly evacuated and immediately filled with argon three times in succession. The mixture was then heated in an oil bath preheated to 100° C. for 24 h.

After cooling, the reaction solution was added to water, extracted repeatedly with ethyl acetate, dried, concentrated and purified by column chromatography. Yield: 253 mg (13% of theory).

¹H-NMR (400 MHz, CDCl₃ δ, ppm) 7.82 (d, 2H), 7.57-7.21 (m, 8H), 6.80 (br, 1H), 3.29 (tr, 2H), 1.76 (m, 4H), 1.67 (m, 1H), 1.58 (m, 1H), 1.3-1.15 (m, 3H), 1.04-0.95 (m, 2H).

### 1.3. Synthesis of 3-amino-4-chloro-N-(cyclohexylmethyl)isothiazole-5-carboxamide

184 mg of 4-chloro-N-(cyclohexylmethyl)-3-[(diphenylmethylene)amino]isothiazole-5-carboxamide were dissolved in 1.5 ml of tetrahydrofuran (THF) to which 0.5 ml of 6N hydrochloric acid was added and the mixture was stirred at room temperature until the reactant was completely consumed according to thin-layer chromatography. The reaction mixture was added to a little water and extracted with ethyl acetate. On drying the ethyl acetate phases, concentrating and chromatography, the desired product was obtained. Yield: 89 mg (77% of theory).

¹H-NMR (400 MHz, CDCl₃ δ, ppm) 6.68 (br, 1H), 4.73 (br, 2H), 3.32 (tr, 2H), 1.77 (m, 4H), 1.66 (m, 1H), 1.57 (m, 1H), 1.32-1.13 (m, 3H), 1.06-0.96 (m, 2H).

### 2. Synthesis of 3-amino-4-chloro-N-(3,4,5-trifluorobenzypisothiazole-5-carboxamide

### 2.1. Synthesis of ethyl 3,4-dichloroisothiazole-5-carboxylate

15 g of 3,4-dichloroisothiazole-5-carboxylic acid (75 7 mmol) were dissolved in 300 ml of ethanol and 8.4 ml of concentrated sulphuric acid were added. The mixture was stirred under reflux for 20 h. The reaction mixture was then concentrated to half the original volume, neutralized with saturated NaHCO₃, added to water and extracted with dichloromethane. The dichloromethane phases were dried and carefully concentrated on a rotary evaporator. Yield: 15.2 g (89% of theory).

¹H-NMR (400 MHz, CDCl₃ δ, ppm) 4.44 (q, 2H), 1.42 (tr, 3H).

### 2.2. Synthesis of ethyl 4-chloro-3-[(diphenylmethylene)amino]isothiazole-5-carboxylate

14.06 g of ethyl 3,4-dichloroisothiazole-5-carboxylate (62.19 mmol) were dissolved in 150 ml of toluene. To this solution were added consecutively 13.9 g of benzophenone imine (74.6 mmol), 40.5 g caesium carbonate (124.4 mmol), 1.44 g Xantphos (2.49 mmol) and 1.14 g tri(dibenzylidenaceton)dipalladium (Pd₂dba₃; 1.24 mmol). The reaction vessel with the resulting solution was then briefly evacuated and immediately filled with argon three times in succession. The mixture was then heated for 24 h in an oil bath which had been preheated to 100° C. After cooling, the reaction solution was filtered through a 2 cm thick layer of silica gel, which was rinsed repeatedly with dichloromethane. The filtrate was concentrated and the residue purified by column chromatography. Yield: 5.29 g (23% of theory).

¹H-NMR (400 MHz, CDCl₃ δ, ppm) 7.82 (d, 2H), 7.59-7.22 (m, 8H), 4.36 (q, 2H), 1.37 (tr, 3H).

### 2.3. Synthesis of ethyl 3-amino-4-chloroisothiazole-5-carboxylate

10.1 g of ethyl 4-chloro-3-[(diphenylmethylene)amino]isothiazole-5-carboxylate (27 2 mmol) were dissolved in 200 ml of tetrahydrofuran (THF) to which 12 ml of 6N hydrochloric acid was added and the mixture was stirred at room temperature until the reactant was completely consumed according to thin-layer chromatography. The reaction mixture was added to a little water and extracted with ethyl acetate. On drying the ethyl acetate phases, concentrating and chromatography, the desired product was obtained. Yield: 4.51 g (91% of theory).

¹H-NMR (400 MHz, CDCl₃ δ, ppm) 5.0-4.5 (br, 2H), 4.40 (q, 2H), 1.39 (tr, 3H).

### 2.4. Synthesis of 3-amino-4-chloroisothiazole-5-carboxylic acid

To 4.27 g of ethyl 3-amino-4-chloroisothiazole-5-carboxylate (20.6 mmol) in a mixture of 50 ml of ethanol and 50 ml of THF were added 26 ml of 2N sodium hydroxide solution and the mixture was stirred at room temperature for 1 h. The reaction mixture was then adjusted to pH 5 by careful addition of 2N hydrochloric acid, whereupon a portion of the product precipitated in the form of crystals. The crystals were filtered off under suction and the filtrate was extracted with ethyl acetate. The organic extracts were dried and concentrated, whereby a further batch of the product was obtained. Yield: 3.57 g (97% of theory).

¹H-NMR (400 MHz, DMSO 6, ppm) 14.05 (br, 1H), 6.58 (s, 2H).

### 2.5. Synthesis of 3-amino-4-chloro-N-(3,4,5-trifluorobenzyl)isothiazole-5-carboxamide

To 100 mg of 3-amino-4-chloroisothiazole-5-carboxylic acid (0.56 mmol) in 4 ml of dichloromethane were added 130 mg of 3,4,5-trifluorobenzylamine (0.78 mmol), 170 mg of triethylamine (1.68 mmol) and 0.83 ml of a 50 percent solution of n-propylphosphonic anhydride (T3P; 1.4 mmol) in THF and the mixture was stirred overnight at room temperature. The reaction mixture was then added to water and extracted repeatedly with ethyl acetate. The organic extracts were washed with saturated sodium chloride solution, dried and concentrated on a rotary evaporator. The residue was purified by column chromatography. Yield: 102 mg (57% of theory).

¹H-NMR (400 MHz, DMSO δ, ppm) 9.0 (tr,1H), 7.26 (m,2H), 6.56 (s,2H), 4.43 (d,2H).

### 3. Synthesis of 3-amino-4-chloro-N-(2,4-difluorobenzyl)isothiazole-5-carbothioamide

### 3.1. Synthesis of 3-amino-4-chloro-N-(2,4-difluorobenzyl)isothiazole-5-carboxamide

Analogously to the synthesis of compound 1-35 described above, 120 mg of 3-amino-4-chloroisothiazole-5-carboxylic acid (0.67 mmol) were reacted with 143 mg (1 mmol) of 2,4-difluorobenzylamine Yield: 188 mg (91% of theory).

¹H-NMR (400 MHz, CDCl₃ δ, ppm) 7.41 (m, 1H), 7.03 (br, 1H), 6.86 (m, 2H), 4.73 (br, 2H), 4.66 (d, 2H).

### 3.2. Synthesis of 3-amino-4-chloro-N-(2,4-difluorobenzyl)isothiazole-5-carbothioamide

83 mg of 3-amino-4-chloro-N-(2,4-difluorobenzyl)isothiazole-5-carboxamide (0.27 mmol) and 121 mg of 4-methoxyphenyldithiophosphonic anhydride (Lawesson's reagent; 0 3 mmol) in 2 ml of THF were stirred at room temperature for 6 h and at 50° C. for 1 h. After cooling, the mixture was added to water and extracted with dichloromethane. The dichloromethane phases were dried and concentrated. The residue was purified by column chromatography. Yield: 17.1 mg (20% of theory).

¹H-NMR (400 MHz, CDCl₃ δ, ppm) 8.67 (br, 1H), 7.48 (m, 1H), 6.90 (m, 2H), 5.02 (d, 2H).

### 4. Synthesis of 4-chloro-N-(cyclohexylmethyl)-3-(pentanoylamino)isothiazole-5-carboxamide

To 70 mg of 3-amino-4-chloro-N-(cyclohexylmethyl)isothiazole-5-carboxamide (0.25 mmol) in 3 ml of dichloromethane were added 52 mg of triethylamine (0.51 mmol), 4 mg of 4-dimethylaminopyridine and 62 mg of n-pentanoyl chloride (0.51 mmol) and the mixture was stirred for 3 h at room temperature. The mixture was then added to water and extracted with dichloromethane. The dichloromethane phases were dried and concentrated. The residue was purified by chromatography. Yield: 53 mg (57% of theory)

¹H-NMR (400 MHz, CDCl₃ δ, ppm) 7.68 (bs, 1H), 6.73 (bs, 1H), 3.33 (t, 2H), 2.63 (bs, 2H), 1.73 (m, 6H), 1.59 (m, 2H), 1.43 (m, 2H), 1.21 (m, 4H), 1.00 (m, 4H).

### 5. Synthesis of 4-chloro-N-(2,6-difluorobenzyl)-3-Ktrifluoroacetypaminolisothiazole-5-carboxamide

### 5.1. Synthesis of 3-amino-4-chloro-N-(2,6-difluorobenzyl)isothiazole-5-carboxamide

Analogously to the synthesis of compound 1-35 described above, 100 mg of 3-amino-4-chloroisothiazole-5-carboxylic acid (0.56 mmol) were reacted with 115 mg of 2,6-difluorobenzylamine (0.78 mmol). Yield: 125 mg (74% of theory).

¹H-NMR (400 MHz, CDCl₃ δ, ppm) 7.31 (m, 1H), 7.05 (bs, 1H), 6.94 (m, 2H), 4.76 (d, 2H), 4.72 (bs, 2H).

### 5.2. Synthesis of 4-chloro-N-(2,6-difluorobenzyl)-3-Ktrifluoroacetypaminolisothiazole-5-carboxamide

To 20 mg of 3-amino-4-chloro-N-(2,6-difluorobenzyl)isothiazole-5-carboxamide (0.06 mmol) in 1 ml of dichloromethane were added 13 mg of triethylamine (0.13 mmol), 2 mg of 4-dimethylaminopyridine and 28 mg of trifluoroacetic anhydride (0.13 mmol) and the mixture was stirred for 3 h at room temperature. The mixture was then added to water and extracted with dichloromethane. The dichloromethane phases were dried and concentrated and the residue was purified by chromatography. Yield: 13 mg (49% of theory).

¹H-NMR (400 MHz, CDCl₃ δ, ppm) 8.29 (bs, 1H), 7.31 (m, 1H), 7.09 (bs,1H), 6.94 (m, 2H), 4.78 (d, 2H).

6. Synthesis of 3-amino-4-chloro-N-[(1-chlorocyclopropyflcarbonyl]-N-(2,4-difluorobenzyl)isothiazole-5-carboxamide

To 80 mg of 3-amino-4-chloro-N-(2,4-difluorobenzyl)isothiazole-5-carboxamide (0.26 mmol) in 4 ml of dichloromethane were added 53 mg of triethylamine (0.52 mmol), 4 mg of 4-dimethylaminopyridine and 73 mg of 1-chlorocyclopropanecarbonyl chloride (0.52 mmol) and the mixture was stirred for 2 h at room temperature. The mixture was then added to water and extracted with dichloromethane. The dichloromethane phases were dried and concentrated. The residue obtained therefrom was purified by chromatography. Yield: 24 mg (22% of theory).

¹H-NMR (400 MHz, CDCl₃ δ, ppm) 7.37 (m, 1H), 6.83 (m, 2H), 5.06 (s, 2H), 4.82 (bs, 2H), 1.66 (m, 2H), 1.30 (m, 2H).

### 7. Synthesis of 3-amino-N-(cyclohe xy lmethyl)-4-ethynylisothiazole-5-carboxamide

### 7.1. Synthesis of methyl 3-amino-4-Ktrimethylsilyflethynyllisothiazole-5-carboxylate

To 650 mg (2.28 mmol) of methyl 3-amino-4-iodo-1,2-thiazole-5-carboxylate in 11 ml of DMF were added 43.6 mg (0.229 mmol) of CuI, 160 mg (0.229 mmol) of Pd(PPh₃)₂Cl₂ and 0.638 ml (5.58 mmol) of triethylamine and the mixture was stirred at room temperature for 5 min under protective gas (argon). 0.647 ml (4.58 mmol) of ethynyltrimethylsilane were added dropwise and then the mixture was stirred for 1 h at 100° C. The mixture was then concentrated on a rotary evaporator and the residue was treated with a saturated NH₄Cl solution and extracted with dichloromethane/heptane 1:9. The organic phase was dried over Na₂SO₄, filtered and concentrated by rotary evaporation. The residue was purified by chromatography. Yield: 486 mg (83% of theory)

¹H-NMR (400 MHz, CDCl₃ δ, ppm) 4.91 (br, 2H), 3.92 (s, 3H), 0.29 (s, 9H).

### 7.2. Synthesis of 3-amino-4-ethynylisothiazole-5-carboxylic acid

300 mg (1.18 mmol) of methyl 3-amino-4-[(trimethylsilyl)ethynyl]isothiazole-5-carboxylate were dissolved in 10 ml of THF/methanol 1:1. NaOH (3.54 mmol) dissolved in 2 ml of water was added dropwise. After stirring for 1 h at room temperature, the mixture was concentrated on a rotary evaporator. The residue was treated with 2M HCl and the mixture was extracted with ethyl acetate. The organic phase was dried over Na₂SO₄, filtered and concentrated by rotary evaporation. Yield: 197 mg (100%) of crude product.

### 7.3. Synthesis of 3-amino-N-(cyclohexylmethyl)-4-ethynylisothiazole-5-carboxamide

135 mg (0.8 mmol) of 3-amino-4-ethynylisothiazole-5-carboxylic acid, 0.26 ml (2.0 mmol) of cyclohexylmethylamine, 1.27 g (2.0 mmol, 50% in THF) of n-propylphosphonic anhydride (T3P) and 0.335 ml (2.4 mmol) of triethylamine were dissolved in 8 ml of THF and the mixture was stirred at 55° C. for 1.5 h. The mixture was then concentrated on a rotary evaporator, the residue then treated with 2M NaOH and extracted repeatedly with ethyl acetate. The organic extracts were dried with Na₂SO₄ and concentrated on a rotary evaporator. The residue was purified by column chromatography. Yield: 156 mg (74% of theory).

¹H-NMR (400 MHz, CDCl₃ δ, ppm) 7.12 (br, 1H), 4.83 (br, 2H), 3.77 (s, 1H), 3.32 (t, 2H), 1.80-1.56 (m, 6H), 1.31-1.15 (m, 3H), 1.06-1.00 (m, 2H).

### 8. Synthesis of 3-amino-N-(cyclohexylmethyl)-4-ethylisothiazole-5-carboxamide

### 8.1. Synthesis of methyl 3-[(tert-butoxycarbonyl)amino]-4-vinylisothiazole-5-carboxylate

1.10 g (2.86 mmol) of methyl 3-[(tert-butoxycarbonyl)amino]-4-iodoisothiazole-5-carboxylate, 460 mg (3.44 mmol) of potassium trifluoro(vinyl)borate and 0.6 ml (4.30 mmol) of triethylamine were dissolved in 7.7 ml of ethanol and the mixture was stirred for 5 min at room temperature under protective gas (argon). 25.4 mg (0.143 mmol) of PdCl₂ were added and the mixture was heated for 1 h at 100° C. in a microwave. The mixture was then concentrated and the residue extracted with NaHCO₃ and ethyl acetate, separated off, dried with Na₂SO₄ and concentrated by rotary evaporation. The residue was purified by column chromatography. Yield: 580 mg (71% of theory).

¹H-NMR (400 MHz, CDCl₃ δ, ppm) 7.16 (br, 1H), 7.10-7.02 (dd, 1H), 5.72-5.66 (m, 2H), 3.91 (s, 3H), 1.53 (s, 9H).

### 8.2. Synthesis of 3-[(tert-butoxycarbonyl)amino]-4-vinylisothiazole-5-carboxylic acid

680 mg (2.39 mmol) of methyl 3-[(-butoxycarbonyl)amino]-4-vinylisothiazole-5-carboxylate were dissolved in 35 ml of THF. 2.63 ml of a 2M NaOH solution were added dropwise. After stirring for 2 h at room temperature, the mixture was concentrated on a rotary evaporator. The residue was treated with 2M HC₁ and extracted with ethyl acetate. The organic phase was dried over Na₂SO₄, filtered and concentrated by rotary evaporation. Yield: 645 mg (100%) of crude product.

### 8.3. Synthesis of tert-butyl {5-[(cyclohexylmethyl)carbamoyl]-4-vinylisothiazol-3-yl}carbamate)

323 mg (1.20 mmol) of 3-[(tert-butoxycarbonyl)amino]-4-vinylisothiazole-5-carboxylic acid, 0.46 ml (3.59 mmol) of cyclohexylmethylamine, 950 mg (2.99 mmol, 50% in THF) of n-propylphosphonic anhydride (T3P) and 0.50 ml (3.59 mmol) of triethylamine were dissolved in 9 ml of THF and the mixture stirred at 55° C. for 1.5 h. The mixture was then concentrated on a rotary evaporator, the residue treated with 1M HCl and extracted repeatedly with dichloromethane. The organic extracts were dried with Na₂SO₄ and concentrated on a rotary evaporator. The residue was purified by column chromatography. Yield: 412 mg (94% of theory).

¹H-NMR (400 MHz, CDCl₃ δ, ppm) 6.94 (br, 1H), 6.83-6.76 (dd, 1H), 6.16 (br, 1H), 5.76-5.65 (m, 2H), 3.25 (t, 2H), 1.74-1.68 (m, 4H), 1.54-1.47 (m, 11H), 1.36-1.11 (m, 3H), 1.01-0.88 (m, 2H).

### 8.4. Synthesis of tert-butyl {5-[(cyclohexylmethyl)carbamoyl]-4-ethylisothiazol-3-yl}carbamate)

100 mg (0.274 mmol) of tert-butyl {5-[(cyclohexylmethyl)carbamoyl]-4-vinylisothiazol-3-yl}carbamate) were dissolved in 2.7 ml of methanol and 2.91 mg (0.027 mmol) of Pd/C (5%) were added. After stirring for 18 h at room temperature under hydrogen, the mixture was filtered and concentrated by rotary evaporation. Yield: 100 mg (99% of theory).

¹H-NMR (400 MHz, CDCl₃ δ, ppm) 6.75 (br, 1H), 5.87 (br, 1H), 3.27 (t,2H), 2.81 (q, 2H), 1.77-1.67 (m, 4H), 1.58-1.53 (m, 11H), 1.28-1.15 (m, 6H), 1.02-0.93 (m, 2H).

### 8.5. Synthesis of 3-amino-N-(cyclohexylmethyl)-4-ethylisothiazole-5-carboxamide

75 mg (0.204 mmol) of tert-butyl {5-[(cyclohexylmethyl)carbamoyl]-4-ethylisothiazol-3-yl}carbamate) and 0.204 ml (2.65 mmol) of TFA were together dissolved in 1 ml of dichloromethane and the mixture was stirred at room temperature for 30 min. The mixture was then concentrated on a rotary evaporator, the residue treated with 2M NaOH and extracted repeatedly with dichloromethane. The organic extracts were dried with Na₂SO₄ and concentrated on a rotary evaporator. The residue was purified by column chromatography.

Yield: 51 mg (93% of theory).

¹H-NMR (400 MHz, CDCl₃ δ, ppm) 5.82 (br, 1H), 4.52 (br, 2H), 3.25 (t, 2H), 2.74 (q, 2H), 1.76-1.53 (m, 6H), 1.27-1.15 (m, 6H), 1.02-0.88 (m, 2H).

### 9. Synthesis of 3-amino-4-bromo-N-cyclohexyl-1,2-thiazole-5-carboxamide

### 9.1. Synthesis of N,N-dibenzyl-1,2-thiazol-3-amine

To a stirred solution of N-benzyl-1,2-thiazol-3-amine (65.0 g, 1.0 eq.; prepared according to J. Org. Chem. 1979, 44(7) 1118-1124) in THF (1000 ml) at 0° C., NaH (2.0 eq.) was added and stirred for 30 minutes. Then Benzyl bromide (1.5 eq.) was added at same temperature and the reaction mixture was stirred at 50° C. for 12 hours. After completion of reaction, the reaction mixture was poured into ice water and extracted with ethyl acetate. The combined organic layers were washed with water, brine, dried over anhydrous sodium sulphate and the solvent was removed under reduced pressure to get crude product. This crude product was purified over silica gel (100-200 mesh) column chromatography by eluting with 10% EtOAc/petroleum ether to afford pure N,N-dibenzyl-1,2-thiazol-3-amine (65.0 g, 68% of theory).

### 9.2. Synthesis of 3-(dibenzylamino)-1,2-thiazole-5-carboxylic acid

To a stirred solution of N,N-dibenzyl-1,2-thiazol-3-amine (4×16.0 g, 1.0 eq.) in THF (200 ml) at −78° C., 1.6M nBuLi (1.0 eq. in Hexane) was added drop wisely. The resulting mixture was stirred for 30 minutes at same temperature. Then dry CO₂ gas was bubbled through the solution for 30 minutes at −78° C. After completion of reaction, the reaction mixture was quenched with 1N HCl solution and extracted with ethyl acetate. The combined organic layers were washed with water, brine, dried over anhydrous sodium sulphate and the solvent was removed under reduced pressure to get crude product. This crude product was purified over silica gel (100-200 mesh) column chromatography by eluting with 60% EtOAc/petroleum ether to afford pure 3-(dibenzylamino)-1,2-thiazole-5-carboxylic acid (20.5 g, 28% of theory).

### 9.3. Synthesis of ethyl 3-(dibenzylamino)-1,2-thiazole-5-carboxylate

To a solution of 3-(dibenzylamino)-1,2-thiazole-5-carboxylic acid (21.0 g, 1.0 eq.) in DMF (200 mL) at room temperature, potassium carbonate (2.0 eq.) was added and stirred for 30 minutes. Then ethyl iodide (5.0 eq.) was added to reaction mixture and stirring continued at the same temperature for five hours. After completion of reaction, the reaction mixture was poured into ice water and extracted with ethyl acetate. The combined organic layers were washed with water, brine, dried over anhydrous sodium sulphate and the solvent was removed under reduced pressure to get crude product. This crude product was purified over silica gel (100-200 mesh) column chromatography by eluting with 10% EtOAc/petroleum ether to afford pure ethyl 3-(dibenzylamino)-1,2-thiazole-5-carboxylate (18.0 g, 79% of theory).

### 9.4. Synthesis of ethyl 4-bromo-3-(dibenzylamino)-1,2-thiazole-5-carboxylate

A solution of ethyl 3-(dibenzylamino)-1,2-thiazole-5-carboxylate (11.6 g, 32.9 mmol) and N-bromosuccinimide (6.44 g, 36.2 mmol) in dry DMF (20 mL) was stirred in the dark at room temperature for 18 h. The mixture was diluted with water, washed with aq. NaHCO₃, water, extracted with ethyl acetate (3×50 mL). The combined organic layers were sequentially washed with sat. aq. Na₂SO₃, then with sat. aq. LiCl, then dried over MgSO₄ and concentrated in vacuo. The residue was purified by column chromatography. Yield: 9.35 g (63% of theory).

¹H-NMR (300 MHz, DMSO-d₆ 6, ppm) 7.40-7.20 (m, 10H), 4.52 (s, 4H), 4.36 (q, 2H), 1.33 (t, 3H).

### 9.5. Synthesis of 3-amino-4-bromo-1,2-thiazole-5-carboxylic acid

To a solution of ethyl 4-bromo-3-(dibenzylamino)-1,2-thiazole-5-carboxylate (4.12 g, 9.55 mmol) in toluene (5 mL) at 10° C. was added drowise trifluoromethanesulfonic acid (3.58 g, 23.8 mmol), then the resulting mixture was stirred at 110° C. for 3 h. After cooling down to room temperature, the mixture was diluted with water, and extracted with ethyl acetate (3×30 mL). The combined organic layers were sequentially washed with aq. NaHCO₃, then dried over MgSO₄ and concentrated in vacuo. The residue was suspended in dichloromethane, the resulting purple solid was filtered off to give 3-amino-4-bromo-1,2-thiazole-5-carboxylic acid. Yield: 1.69 g (75% of theory).

Melting point: 164.5° C.

¹H-NMR (400 MHz, DMSO-d₆ 6, ppm) 14.04 (br s, 1H), 6.52 (s, 2H).

### 9.6. Synthesis of 3-amino-4-bromo-N-cyclohexyl-1,2-thiazole-5-carboxamide

A solution of 3-amino-4-bromo-1,2-thiazole-5-carboxylic acid (150 mg, 0.63 mmol), cyclohexanamine (127 mg, 1.27 mmol), n-propylphosphonic anhydride (T3P) (50% in THF, 1220 mg, 1.91 mmol), and triethylamine (259 mg, 2.55 mmol) in 1,4-Dioxane (5 mL) was stirred at 55° C. for 2 h. The mixture was diluted with water, and extracted with ethyl acetate (3×20 mL). The combined organic layers were sequentially washed with aq. NaHCO₃, water, and brine, then dried over MgSO₄ and concentrated in vacuo. The residue was purified by column chromatography. Yield: 175 mg (90% of theory).

¹H-NMR (400 MHz, DMSO-d₆ 6, ppm) 8.37 (d, 1H), 6.43 (s, 2H), 3.80-3.60 (br m, 1H), 1.90-1.50 (m, 5H), 1.45-1.05 (m, 5H).

### 10. Synthesis of 3-amino-N-(cyclohexylmethyl)-4-(difluoromethyl)-1,2-thiazole-5-carboxamide

### 10.1. Synthesis of 3-(dibenzy lamino)-4-formyl-1,2-thiazole-5-carboxy lic acid

To a solution of 3-(dibenzylamino)isothiazole-5-carboxylic acid (3.00 g, 9.25 mmol) and 1,2-bis(dimethylamino)ethane (3.49 mL, 23.12 mmol) in dry THF (20 mL) was added dropwise at −78° C. a solution of n-butyllithium (2.5 M in hexanes, 9.25 mL, 23.12 mmol). After stirring for 1 h at −78° C., dry dimethylformamide (1.57 mL, 20.35 mmol) was added to the mixture. The mixture was allowed to slowly warm up to room temperature and further stirred for 3 h. The mixture was diluted with water and acidified with aq. HCl (1N) to pH 2, then extracted with ethyl acetate. The combined organic layers were dried over MgSO₄ and concentrated in vacuo. The crude residue was used as such in the next step. Yield: 3.25 g (37% of theory).

### 10.2. Synthesis of ethyl 3-(dibenzylamino)-4-formyl-1,2-thiazole-5-carboxylate

To a solution of crude 3-(dibenzylamino)-4-formyl-1,2-thiazole-5-carboxylic acid (3.25 g, 9.20 mmol) and iodoethane (7.19 g, 46.1 mmol) in dry DMF (12.5 mL) was added solid potassium carbonate (2.55 g, 18.4 mmol), and the resulting suspension was stirred at 50° C. for 4 h. The mixture was diluted with water, then extracted several times with ethyl acetate. The combined organic layers were dried over MgSO₄ and concentrated in vacuo. The residue was purified by column chromatography. Yield: 511 mg (11% of theory).

### 10.3. Synthesis of ethyl 3-(dibenzylamino)-4-(difluoromethyl)-1,2-thiazole-5-carboxylate

To a solution of ethyl 3-(dibenzylamino)-4-formyl-1,2-thiazole-5-carboxylate (1.38 g, 3.63 mmol) in dry dichloromethane (3 mL) was added Diethylaminosulfur trifluoride (DAST, 1.20 mL=9.07 mmol). The mixture was stirred at room temperature for 3 h, then poured onto an aqueous saturated solution of NaHCO₃. The mixture was extracted with dichloromethane, the combined organic layers dried over MgSO₄ and concentrated in vacuo. The residue was purified by column chromatography. Yield: 514 mg (32% of theory).

¹H-NMR (300 MHz, DMSO-d₆ 6, ppm) 7.51 (t, 1H), partially overlapping with 7.37-7.12 (m, 10H), 4.52 (s, 4H), 4.36 (q, 2H),1.32 (t, 3H).

### 10.4. Synthesis of 3-(dibenzylamino)-4-(difluoromethyl)-1,2-thiazole-5-carboxylic acid

To a solution of ethyl 3-(dibenzylamino)-4-(difluoromethyl)-1,2-thiazole-5-carboxylate (400 mg, 0.71 mmol) in Ethanol:THF=(1:1) (3 mL) was added aq. NaOH (1N, 6.40 mL=6.40 mmol). The mixture was stirred at room temperature for 2h, then acidified to pH 1-2 with aq. HCl (1N), then extracted with ethyl acetate. The combined organic layers were dried over MgSO₄ and concentrated in vacuo. Yield: 454 mg (90% of theory).

¹H-NMR (300 MHz, DMSO-d₆ 6, ppm) 14.6 (br. S, 1H) 7.59 (t, 1H), 7.37-7.17 (m, 10H), 4.49 (s, 4H).

### 10.5. Synthesis of N-(cyclohexylmethyl)-3-(dibenzylamino)-4-(difluoromethyl)-1,2-thiazole-5-carboxamide

A solution of 3-(dibenzylamino)-4-(difluoromethyl)-1,2-thiazole-5-carboxylic acid (514 mg, 0.71 mmol), 1-cyclohexylmethanamine (87 mg, 0.78 mmol), n-propylphosphonic anhydride (T3P) (50% in THF, 584 mg, 0.92 mmol), and triethylamine (93 mg, 0.92 mmol) in dichloromethane (5 mL) was stirred at room temperature for 14 h. The mixture was diluted with water, and extracted with dichloromethane. The combined organic layers were washed with water, then dried over MgSO₄ and concentrated in vacuo. The residue was purified by column chromatography. Yield: 303 mg (92% of theory).

¹H-NMR (300 MHz, DMSO-d₆ 6, ppm) 8.83 (t, 1H) 7.28 (t, 1H), overlapping with 7.35-7.15 (m, 10H), 4.48 (s, 4H), 3.12-3.00 (m, 2H), 1.73-1.40 (m, 6H), 1.33-1.00 (m, 3H), 1.00-0.81 (m, 2H).

### 10.6. Synthesis of 3-amino-N-(cyclohexylmethyl)-4-(difluoromethyl)-1,2-thiazole-5-carboxamide

A solution of N-(cyclohexylmethyl)-3-(dibenzylamino)-4-(difluoromethyl)-1,2-thiazole-5-carboxamide (303 mg, 0.65 mmol) and 2,3-Dichloro-5,6-dicyano-1,4-benzoquinone (732 mg, 3.23 mmol) in dichloromethane (5 mL) was stirred at room temperature for 12 h, then refluxed for 12 h, then stirred at room temperature again for 5 days. The resulting mixture was washed with aq. NaOH (1N), the organic layer was washed with brine, dried over MgSO₄ and concentrated in vacuo. The residue was purified by column chromatography. Yield: 88 mg (47% of theory).

¹H-NMR (300 MHz, DMSO-d₆ 6, ppm) 8.71 (t, 1H) 7.18 (t, 1H), 6.33 (s, 2H), 3.10-2.98 (m, 2H), 1.72-1.48 (m, 6H), 1.30-1.05 (m,3H), 1.00-0.80 (m, 2H).

### 11. Synthesis of 3-amino-4-cyclopropyl-N-cyclohexyl-1,2-thiazole-5-carboxamide

### 11.1. Synthesis of ethyl 4-cyclopropyl-3-(dibenzylamino)isothiazole-5-carboxylate

A solution of ethyl 4-bromo-3-(dibenzylamino)isothiazole-5-carboxylate (2.08 g, 4.82 mmol), cyclopropyl boronic acid (538 mg, 6.27 mmol), palladium(II) acetate (54.1 mg, 0.24 mmol), tricyclohexylphosphine (135 mg, 0.48 mmol) and potassium phosphate (3.58 g, 16.9 mmol) in Toluene:water 2:1 (37.5 mL) was degassed by bubbling a flow of argon through it for 10min, then stirred at 100° C. for 3 h. The mixture was diluted with water, then extracted with ethyl acetate. The combined organic layers were dried over MgSO₄ and concentrated in vacuo, to provide the crude product which was used as such for the next step. Yield: 2.15 g (68% of theory).

### 11.2. Synthesis of 4-cyclopropyl-3-(dibenzylamino)isothiazole-5-carboxylic acid

A solution of ethyl 4-cyclopropyl-3-(dibenzylamino)isothiazole-5-carboxylate (1.90 g, 4.84 mmol) and aqueous sodium hydroxide (1N, 24.2 mmol) in EtOH:THF (1:1) (28 mL) was stirred at room temperature for 2 h. The mixture was acidified with aqueous HCl (1N) to pH 1-2, then extracted with ethyl acetate. The combined organic layers were dried over MgSO₄ and concentrated in vacuo, to provide a mixture of acid and ester which was used as such for the next step. Yield: 837 mg (20% of theory).

### 11.3. Synthesis of N-(cyclohexylmethyl)-3-(dibenzylamino)-4-cyclopropyl-1,2-thiazole-5-carboxamide

A solution of 4-cyclopropyl-3-(dibenzylamino)isothiazole-5-carboxylic acid (360 mg, 0.65 mmol), cyclohexanamine (71 mg, 0.72 mmol), n-propylphosphonic anhydride (T3P) (50% in THF, 539 mg, 0.85 mmol), and triethylamine (86 mg, 0.85 mmol) in Dichloromethane (5 mL) was stirred at room temperature for 72 h. The mixture was diluted with water, and extracted with dichloromethane. The combined organic layers were washed with water, then dried over MgSO₄ and concentrated in vacuo. The residue was purified by column chromatography. Yield: 365 mg (38% of theory).

### 11.4. Synthesis of 3-amino-4-cyclopropyl-N-cyclohexyl-1,2-thiazole-5-carboxamide

A solution of N-(cyclohexylmethyl)-3-(dibenzylamino)-4-cyclopropyl-1,2-thiazole-5-carboxamide (365 mg, 0.25 mmol) and 2,3-Dichloro-5,6-dicyano-1,4-benzoquinone (279 mg, 1.23 mmol) in dichloromethane (5 mL) was stirred at room temperature for 12 h. The resulting mixture was washed with aq. NaOH (1N), the organic layer was dried over MgSO₄ and concentrated in vacuo. The residue was purified by column chromatography. Yield: 8 mg (12% of theory).

¹H-NMR (300 MHz, CDCl₃ δ, ppm) 6.37 (br, 1H), 4.73 (br, 2H), 4.05-3.85 (br m, 1H), 2.13-1.93 (m, 2H), 1.85-0.70 (m, 13H).

In the following preferred compounds useful as intermediates for the preparation of compounds of the formula (G) according to the present invention are described.

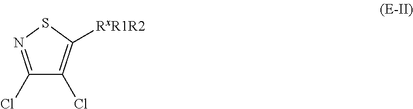

The residue R^{xR1R2} in the above formula corresponds to the group C(O)—N(R²)-(A)_{y}-R¹ in formula (E-H) in Scheme 1 shown above.

Preferred compounds of the formula (E-II) are those mentioned in the following Table 3.

TABLE 3
Preferred compounds of formula (E-II)

| Ex N^{o} | R^{xR1R2} | LogP |
|---|---|---|
| E-II-001 | (cyclohexylmethyl)carbamoyl | 4.11^{[a]} |
| E-II-002 | (1-(S)-cyclohexylethyl)carbamoyl | 4.56^{[a]} |
| E-II-003 | (2-chlorobenzyl)carbamoyl | 3.52^{[a]} |
| E-II-004 | (tetrahydro-2H-pyran-4-ylmethyl)carbamoyl | 2.05^{[a]} |
| E-II-005 | (tetrahydro-2H-pyran-3-ylmethyl)carbamoyl | 2.18^{[a]} |
| E-II-006 | (tetrahydrofuran-2-ylmethyl)carbamoyl | 2.20^{[a]} |
| E-II-007 | (tetrahydrofuran-3-ylmethyl)carbamoyl | 1.84^{[a]} |
| E-II-008 | (2-fluorobenzyl)carbamoyl | 3.23^{[a]} |
| E-II-009 | (2,4-difluorobenzyl)carbamoyl | |
| E-II-010 | (tetrahydro-2H-pyran-2-ylmethyl)carbamoyl | 2.87^{[a]} |
| E-II-011 | (2,5-difluorobenzyl)carbamoyl | 3.17^{[a]} |
| E-II-012 | decahydronaphthalen-1-ylcarbamoyl | 5.34^{[a]} |
| E-II-013 | (2-cyclohexylpropan-2-yl)carbamoyl | 5.39^{[a]} |
| E-II-014 | cyclopropyl(2,4-difluorobenzyl)carbamoyl | 4.01^{[a]} |
| E-II-015 | (2-tert-butyl-5-methylbenzyl)(cyclopropyl)carbamoyl | 5.65^{[a]} |
| E-II-016 | {[trans-4-(cyclopropylcarbamoyl)cyclohexyl]methyl}carbamoyl | 2.21^{[a]} |
| E-II-017 | rel-{[(1R,2S,4R)-1,7,7-trimethylbicyclo[2.2.1]hept-2-yl]carbamoyl} | 5.02^{[a]}; 4.96^{[b]} |
| E-II-018 | (8-methyl-8-azabicyclo[3.2.1]oct-3-yl)carbamoyl | 0.66^{[a]} |
| E-II-020 | 2,3-dihydro-1H-inden-1-ylcarbamoyl | 3.70^{[a]}; 3.63^{[b]} |
| E-II-021 | 1,2,3,4-tetrahydronaphthalen-1-ylcarbamoyl | 4.07^{[a]}; 3.98^{[b]} |
| E-II-022 | rel-{[(1R,4R)-1,7,7-trimethylbicyclo[2.2.1]hept-2-yl]carbamoyl} | 5.07^{[a]}; 4.94^{[b]} |
| E-II-023 | rel-({[(1R,2R,5R)-6,6-dimethylbicyclo[3.1.1]hept-2- | 5.13^{[a]}; 5.02^{[b]} |
| | yl]methyl}carbamoyl) | |
| E-II-024 | rel-{[(1R,2R,5S)-2-isopropyl-5-methylcyclohexyl]carbamoyl} | 5.72^{[a]}; 5.64^{[b]} |
| E-II-025 | rel-[(1R,4aS,8aS)-decahydronaphthalen-1-ylcarbamoyl] | 5.27^{[a]} |
| E-II-026 | rel-[(1R,4aR,8aS)-decahydronaphthalen-1-ylcarbamoyl] | 5.43^{[a]} |
| E-II-027 | rel-[(1R,8aR)-decahydronaphthalen-1-ylcarbamoyl] | 5.08 + 5.17^{[a]} |
| E-II-028 | (1-cyclohexylethyl)(methoxy)carbamoyl | 5.27^{[a]}; 5.09^{[b]} |
| E-II-029 | (1-cyclobutyl-3-phenylpropyl)carbamoyl | 5.04^{[a]}; 4.91^{[b]} |
| E-II-030 | (1-cyclohexyl-3-methoxy-3-oxopropyl)carbamoyl | 4.03^{[a]}; 3.95^{[b]} |
| E-II-031 | [cyclohexyl(phenyl)methyl]carbamoyl | 5.19^{[a]}; 5.05^{[b]} |
| E-II-032 | [1-(tetrahydrofuran-2-yl)ethyl]carbamoyl | 2.72^{[a]}; 2.69^{[b]} |
| E-II-033 | (2-cyclohexylethyl)carbamoyl | 4.40^{[a]} |
| E-II-034 | [1-(cyclohex-3-en-1-yl)-2-phenylethyl]carbamoyl | 4.87^{[a]}; 4.75^{[b]} |

Preferred compounds of the formula (E-VII-VIII) correspond to formulae (E-VII), (E-VIII), (E-XVIII), (E-XXI), (Z-A) and (Z-B):

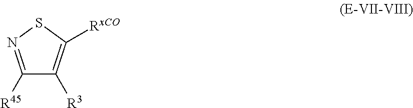

The residue R^{xCO} in the formula (E-VII-VIII) corresponds to the group CO₂H in formula (E-VII), to CO₂R′ in formula (E-VIII), and to CO₂Me (i.e. methoxycarbonyl) in formulae (E-XVIII) and (E-XXI), as shown in Schemes 3 and 4 shown above, respectively.

The residue R^{xCO} in the above formula corresponds to Q in formulae (Z-A) and (Z-B), respectively.

The residue R³ in the formula (E-VII-VIII) corresponds to R³ in the formula (G) according to the present invention.

The residue R⁴⁵ in the above formula corresponds to the group PG-N in formulae (E-VII) and (E-VIII) in Scheme 3, and additionally R⁴⁵ may be an amino group.

Compounds (E-XVIII) and (E-XXI) correspond to compounds (E-XVIII) and (E-XXI) shown in Scheme 4 above.

The residue R⁴⁵ in the above formula corresponds to NR^{Z1}R^{Z2} in formula (Z-A) and to N═CR^{Z3}R^{Z4} in formula (Z-B) in the formulae (Z-A) and (Z-B) shown above.

Preferred compounds of the formula (E-VII-VIII) are those mentioned in the following Table 4.

TABLE 4
Preferred compounds of formula (E-VII-VIII)

| Ex N^{o} | R⁴⁵ | R³ | R^{xCO} | LogP |
|---|---|---|---|---|
| E-VII- | amino | Cl | methoxycarbonyl | 1.42^{[a]} |
| 001 | | | | |
| E-VII- | 1-(4- | Cl | tert-butoxycarbonyl | 4.74^{[a]} |
| 002 | methoxyphenyl)methanamino | | | |
| E-VII- | amino | Cl | tert-butoxycarbonyl | 2.66^{[a]} |
| 003 | | | | |
| E-VII- | 1-(4- | Cl | isopropoxycarbonyl | 4.36^{[a]} |
| 004 | methoxyphenyl)methanamino | | | |
| E-VII- | amino | Cl | isopropoxycarbonyl | 2.17^{[a]}; 2.16^{[b]} |
| 005 | | | | |
| E-VII- | 1,1- | Cl | methoxycarbonyl | 4.40^{[a]} |
| 006 | diphenylmethanimino | | | |
| E-VII- | tert- | cyano | methoxycarbonyl | |
| 007 | butyloxycarbonylamino | | | |
| E-VII- | N-benzyl-1- | Br | ethoxycarbonyl | 5.80^{[a]} |
| 008 | phenylmethanamino | | | |
| E-VII- | tert- | H | methoxycarbonyl | |
| 009 | butyloxycarbonylamino | | | |
| E-VII- | 4-fluorobenzamido | Cl | ethoxycarbonyl | 2.62^{[a]} |
| 010 | | | | |
| E-VII- | bis(tert- | Cl | isopropoxycarbonyl | 5.00^{[a]} |
| 011 | butyloxycarbonyl)amino | | | |
| E-VII- | N-benzyl-1- | H | ethoxycarbonyl | 5.13^{[a]} |
| 012 | phenylmethanamino | | | |
| E-VII- | N-benzyl-1- | Cl | ethoxycarbonyl | 5.73^{[a]} |
| 013 | phenylmethanamino | | | |
| E-VII- | bis(tert- | I | methoxycarbonyl | |
| 014 | butyloxycarbonyl)amino | | | |
| E-VII- | tert- | benzenethioxy | methoxycarbonyl | |
| 015 | butyloxycarbonylamino | | | |
| E-VII- | bis(tert- | H | methoxycarbonyl | |
| 016 | butyloxycarbonyl)amino | | | |
| E-VII- | bis(tert- | 3-(trifluoromethyl)phenyl | methoxycarbonyl | |
| 017 | butyloxycarbonyl)amino | | | |
| E-VII- | 2,2- | Cl | ethoxycarbonyl | 2.48^{[a]} |
| 018 | dimethylpropanamido | | | |
| E-VII- | bis(tert- | Cl | ethoxycarbonyl | |
| 019 | butyloxycarbonyl)amino | | | |
| E-VII- | benzamido | Cl | ethoxycarbonyl | 2.45^{[a]} |
| 020 | | | | |
| E-VII- | N-benzoylbenzamido | Cl | ethoxycarbonyl | |
| 021 | | | | |
| E-VII- | N-acetylacetamido | Cl | ethoxycarbonyl | 2.47^{[a]} |
| 022 | | | | |
| E-VII- | N-benzyl-1- | difluoromethyl | ethoxycarbonyl | 5.68^{[a]} |
| 023 | phenylmethanamino | | | |
| E-VII- | N-benzyl-1- | cyclopropyl | ethoxycarbonyl | 5.91^{[a]} |
| 024 | phenylmethanamino | | | |
| E-VII- | tert- | vinyl | methoxycarbonyl | |
| 025 | butyloxycarbonylamino | | | |
| E-VII- | bis(tert- | cyano | methoxycarbonyl | |
| 026 | butyloxycarbonyl)amino | | | |
| E-VII- | bis(tert- | SMe | methoxycarbonyl | |
| 027 | butyloxycarbonyl)amino | | | |
| E-VII- | tert- | Cl | (cyclohexylmethoxy)carbonyl | |
| 028 | butyloxycarbonylamino | | | |
| E-VII- | tert- | (trimethylsilyl)ethynyl | methoxycarbonyl | |
| 029 | butyloxycarbonylamino | | | |
| E-VII- | amino | Cl | (cyclohexylmethoxy)carbonyl | |
| 030 | | | | |
| E-VII- | N-benzyl-1- | 4-fluorophenyl | ethoxycarbonyl | 6.11^{[a]} |
| 031 | phenylmethanamino | | | |
| E-VII- | amino | Cl | [(2,4- | |
| 032 | | | difluorobenzyl)oxy]carbonyl | |
| E-VII- | tert- | ethynyl | methoxycarbonyl | |
| 033 | butyloxycarbonylamino | | | |
| E-VII- | bis(tert- | trifluoromethyl | methoxycarbonyl | |
| 034 | butyloxycarbonyl)amino | | | |
| E-VII- | tert- | trifluoroacetyl | methoxycarbonyl | |
| 035 | butyloxycarbonylamino | | | |
| E-VII- | bis(tert- | trimethylsilyl | methoxycarbonyl | |
| 036 | butyloxycarbonyl)amino | | | |
| E-VII- | bis(tert- | allyl | methoxycarbonyl | |
| 037 | butyloxycarbonyl)amino | | | |
| E-VII- | tert- | allyl | methoxycarbonyl | |
| 038 | butyloxycarbonylamino | | | |
| E-VII- | tert- | prop-1-en-2-yl | methoxycarbonyl | |
| 039 | butyloxycarbonylamino | | | |
| E-VII- | bis(tert- | benzoyl | methoxycarbonyl | |
| 040 | butyloxycarbonyl)amino | | | |
| E-VII- | tert- | SEt | methoxycarbonyl | |
| 041 | butyloxycarbonylamino | | | |
| E-VII- | bis(tert- | phenyl | methoxycarbonyl | |
| 042 | butyloxycarbonyl)amino | | | |
| E-VII- | bis(tert- | 4-(trifluoromethyl)phenyl | methoxycarbonyl | |
| 043 | butyloxycarbonyl)amino | | | |
| E-VII- | amino | SEt | methoxycarbonyl | |
| 044 | | | | |
| E-VII- | amino | H | methoxycarbonyl | |
| 045 | | | | |
| E-VII- | amino | trifluoromethyl | methoxycarbonyl | |
| 046 | | | | |
| E-VII- | N-(2,4- | Cl | isopropoxycarbonyl | |
| 047 | difluorobenzoyl)-2,4- | | | |
| | difluorobenzamido | | | |
| E-VII- | 2,6- | Cl | isopropoxycarbonyl | |
| 048 | difluorobenzamido | | | |
| E-VII- | 2,4- | Cl | isopropoxycarbonyl | |
| 049 | difluorobenzamido | | | |
| E-VII- | bis(tert- | acetyl | methoxycarbonyl | |
| 050 | butyloxycarbonyl)amino | | | |
| E-VII- | tert- | Et | methoxycarbonyl | |
| 051 | butyloxycarbonylamino | | | |
| E-VII- | amino | Et | methoxycarbonyl | |
| 052 | | | | |
| E-VII- | amino | (trimethylsilyl)ethynyl | methoxycarbonyl | |
| 053 | | | | |
| E-VII- | tert- | pyridin-4-yl | methoxycarbonyl | |
| 054 | butyloxycarbonylamino | | | |
| E-VII- | tert- | prop-1-yn-1-yl | methoxycarbonyl | |
| 055 | butyloxycarbonylamino | | | |
| E-VII- | tert- | I | isopropoxycarbonyl | |
| 056 | butyloxycarbonylamino | | | |
| E-VII- | bis(tert- | I | isopropoxycarbonyl | |
| 057 | butyloxycarbonyl)amino | | | |
| E-VII- | amino | prop-1-yn-1-yl | methoxycarbonyl | |
| 058 | | | | |
| E-VII- | amino | F | isopropoxycarbonyl | |
| 059 | | | | |
| E-VII- | amino | Br | ethoxycarbonyl | 1.93^{[a]} |
| 060 | | | | |
| E-VII- | tert- | ethoxy | methoxycarbonyl | |
| 061 | butyloxycarbonylamino | | | |
| E-VII- | tert- | methoxy | methoxycarbonyl | |
| 062 | butyloxycarbonylamino | | | |
| E-VII- | 1-(4- | 1-(4- | isopropoxycarbonyl | 4.56^{[a]}; 4.51^{[b]} |
| 063 | methoxyphenyl)methanamino | methoxyphenyl)methanamino | | |
| E-VII- | amino | methoxy | methoxycarbonyl | |
| 064 | | | | |
| E-VII- | amino | ethoxy | methoxycarbonyl | |
| 065 | | | | |
| E-VII- | bis(tert- | Br | methoxycarbonyl | |
| 066 | butyloxycarbonyl)amino | | | |
| E-VII- | bis(tert- | F | isopropoxycarbonyl | |
| 067 | butyloxycarbonyl)amino | | | |
| E-VII- | tert- | F | isopropoxycarbonyl | |
| 068 | butyloxycarbonylamino | | | |
| E-VII- | 1-(4- | Cl | ethoxycarbonyl | 3.87^{[a]} |
| 069 | methoxyphenyl)methanamino | | | |
| E-VII- | 1-(4- | H | isopropoxycarbonyl | 3.63^{[a]} |
| 070 | methoxyphenyl)methanamino | | | |
| E-VII- | tert-butyl (4- | H | isopropoxycarbonyl | 5.78^{[a]} |
| 071 | methoxybenzyloxycarbonyl)amino | | | |
| E-VIII- | tert- | Cl | carboxy | 1.35^{[a]} |
| 001 | butyloxycarbonylamino | | | |
| E-VIII- | 1-(4- | Cl | carboxy | 2.21^{[a]} |
| 002 | methoxyphenyl)methanamino | | | |
| E-VIII- | N-benzyl-1- | H | carboxy | 3.35^{[a]} |
| 003 | phenylmethanamino | | | |
| E-VIII- | N-benzyl-1- | Br | carboxy | 3.83^{[a]} |
| 004 | phenylmethanamino | | | |
| E-VIII- | N-benzyl-1- | Cl | carboxy | 3.75^{[a]} |
| 005 | phenylmethanamino | | | |
| E-VIII- | N-benzyl-1- | formyl | carboxy | 3.21^{[a]} |
| 006 | phenylmethanamino | | | |
| E-VIII- | 4-fluorobenzamido | Cl | carboxy | 1.02^{[a]} |
| 007 | | | | |
| E-VIII- | N-benzyl-1- | cyclopropyl | carboxy | 4.11^{[a]} |
| 008 | phenylmethanamino | | | |
| E-VIII- | N-benzyl-1- | difluoromethyl | carboxy | 3.78^{[a]} |
| 009 | phenylmethanamino | | | |
| E-VIII- | N-benzyl-1- | 4-fluorophenyl | carboxy | 4.49^{[a]} |
| 010 | phenylmethanamino | | | |
| E-VIII- | bis(tert- | I | carboxy | |
| 011 | butyloxycarbonyl)amino | | | |
| E-VIII- | amino | Cl | carboxy | −0.13^{[a]} |
| 012 | | | | |
| E-VIII- | 2,6- | Cl | carboxy | |
| 013 | difluorobenzamido | | | |
| E-VIII- | amino | ethoxy | carboxy | |
| 014 | | | | |
| E-VIII- | amino | Br | carboxy | −0.07^{[a]} |
| 015 | | | | |
| E-VIII- | 1-(4- | H | carboxy | 1.87^{[a]} |
| 016 | methoxyphenyl)methanamino | | | |
| E- | tert- | I | methoxycarbonyl | |
| XVIII | butyloxycarbonylamino | | | |
| E-XXI | amino | I | methoxycarbonyl | |
| Z-A- | N-benzyl-1- | H | H | 4.13^{[a]} |
| 001 | phenylmethanamino | | | |
| Z-A- | 4-fluorobenzamido | Cl | H | 1.74^{[a]} |
| 002 | | | | |
| Z-A- | N-benzyl-1- | 4-fluorophenyl | H | 5.56^{[a]} |
| 003 | phenylmethanamino | | | |
| Z-A- | 2,2- | Cl | H | 1.55^{[a]} |
| 004 | dimethylpropanamido | | | |
| Z-A- | 1-(4- | Cl | H | 3.00^{[a]} |
| 005 | methoxyphenyl)methanamino | | | |
| Z-A- | tert-butyl (4- | Cl | H | 4.03^{[a]} |
| 006 | methoxybenzyloxycarbonyl)amino | | | |
| Z-A- | N-benzyl-1-(4- | Cl | H | 5.03^{[a]} |
| 007 | methoxyphenyl)methanamino | | | |
| Z-A- | 1-(4-methoxyphenyl)- | Cl | H | 5.48^{[a]} |
| 008 | N-(4- | | | |
| | methylbenzyl)methanamino | | | |
| Z-B- | 1,1- | Cl | H | |
| 001 | diphenylmethanimino | | | |

NMR Peak Lists

NMR peak lists for compounds according to formula (G) in the context of the present invention. The numbering refers to Tables 1, 2 and 2a above.

Lengthy table referenced here
US20190110476A1-20190418-T00001
Please refer to the end of the specification for access instructions.

### (B) Biological Examples

Example: In Vivo Preventive Test on Botrvtis cinerea (Grey Mould)

Solvent: 5% by volume of Dimethyl sulfoxide
10% by volume of Acetone

Emulsifier: 1 μl of Tween® 80 per mg of active ingredient

The active ingredients are made soluble and homogenized in a mixture of Dimethyl sulfoxide/Acetone/Tween® 80 and then diluted in water to the desired concentration.

The young plants of gherkin are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Botrytis cinerea spores. The contaminated gherkin plants are incubated for 4 to 5 days at 17° C. and at 90% relative humidity.

The test is evaluated 4 to 5 days after the inoculation. 0% means an efficacy which corresponds to that of the control plants while an efficacy of 100% means that no disease is observed.

In this test, the following compounds according to the invention showed efficacy of at least 70% at a concentration of 500 ppm of active ingredient: I-001; I-002; I-003; I-004; I-005; I-006; I-007; I-009; I-014; I-016; I-017; I-020; I-028; I-030; I-043; I-051; I-052; I-058; I-081; I-082; I-085; I-086; I-089; I-093; I-095; I-096; I-104; I-109; I-110; I-123; I-125; I-128; I-129; I-134; I-145; I-152; I-153; I-159; I-164; I-181; I-184; I-186; I-195; I-202; I-228; I-232; I-239; I-245; I-246; I-248; I-249; I-250; I-251; I-253; I-255; I-261; I-272; I-274; I-276; I-277; I-278; I-279; I-282; I-289; I-304; I-305; I-316; I-339; I-353; I-372; I-379; I-380; I-381; I-383; I-385; I-386; I-407; I-410; I-431; I-433; I-448; I-449; I-450; I-451; I-452; I-453; I-454; I-456; I-457; I-458; I-459; I-466; I-467; I-468; I-469; I-471; I-472; I-473; I-474; I-475; I-476; I-477; I-478; I-481; I-482; I-483; I-484; I-488; I-489; I-496; I-498; I-502; I-503; I-506; I-507; I-508; I-510; I-513; I-514; I-519; I-527; I-528; I-529; I-530; I-535; I-536; I-537; I-539; I-542; I-549; I-550; I-553; I-574; I-583; I-602; I-604; I-609; I-610; I-615; I-619; I-620; I-628; I-629; I-633; I-634; I-635; I-641; I-642; I-643; I-644; I-645; I-647; I-650; I-651; I-653; I-662; I-663; I-664; I-666; I-672; I-675; I-677; I-684; I-686; I-693; II-008; II-015; II-016; II-028; II-032; II-033; II-041; II-042.

Example: In Vivo Preventive Test on Phvtophthora infestans (Tomato Late Blight)

| | |
|---|---|
| Solvent: | 5% by volume of Dimethyl sulfoxide |
| | 10% by volume of Acetone |
| Emulsifier: | 1 μl of Tween ® 80 per mg of active ingredient |

The active ingredients are made soluble and homogenized in a mixture of Dimethyl sulfoxide/Acetone/Tween® 80 and then diluted in water to the desired concentration.

The young plants of tomato are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Phytophthora infestans spores. The contaminated tomato plants are incubated for 5 days at 16-18° C. and at 100% relative humidity.

The test is evaluated 5 days after the inoculation. 0% means an efficacy which corresponds to that of the control plants while an efficacy of 100% means that no disease is observed.

In this test, the following compounds according to the invention showed efficacy of at least 70% at a concentration of 500 ppm of active ingredient: I-003; I-009; I-011; I-012; I-013; I-015; I-016; I-020; I-021; I-023; I-024; I-028; I-030; I-032; I-035; I-036; I-037; I-038; I-040; I-041; I-043; I-044; I-045; I-050; I-051; I-052; I-053; I-055; I-057; I-058; I-059; I-061; I-062; I-063; I-065; I-066; I-068; I-069; I-070; I-071; I-074; I-077; I-082; I-085; I-086; I-087; I-090; I-091; I-093; I-094; I-095; I-097; I-100; I-101; I-105; I-106; I-108; I-110; I-114; I-120; I-122; I-123; I-124; I-126; I-127; I-128; I-129; I-130; I-132; I-133; I-135; I-137; I-140; I-141; I-142; I-143; I-144; I-145; I-147; I-148; I-149; I-159; I-160; I-163; I-165; I-166; I-169; I-172; I-177; I-179; I-180; I-181; I-186; I-192; I-193; I-195; I-196; I-198; I-199; I-202; I-217; I-229; I-232; I-239; I-240; I-243; I-245; I-247; I-251; I-258; I-260; I-261; I-262; I-270; I-271; I-272; I-275; I-278; I-281; I-282; I-291; I-300; I-301; I-303; I-304; I-305; I-306; I-315; I-324; I-332; I-337; I-338; I-341; I-343; I-349; I-353; I-355; I-356; I-357; I-360; I-361; I-368; I-370; I-377; I-382; I-383; I-384; I-388; I-389; I-396; I-397; I-398; I-399; I-403; I-405; I-406; I-417; I-418; I-420; I-421; I-423; I-424; I-427; I-428; I-429; I-431; I-435; I-437; I-440; I-444; I-450; I-461; I-463; I-466; I-467; I-469; I-479; I-484; I-487; I-490; I-491; I-492; I-498; I-499; I-500; I-501; I-503; I-509; I-514; I-515; I-518; I-520; I-523; I-524; I-525; I-526; I-529; I-534; I-538; I-542; I-543; I-545; I-550; I-555; I-556; I-560; I-563; I-564; I-565; I-566; I-570; I-575; I-583; I-584; I-585; I-589; I-591; I-594; I-596; I-597; I-601; I-605; I-616; I-618; I-619; I-620; I-621; I-622; I-623; I-630; I-632; I-633; I-634; I-635; I-636; I-637; I-638; I-640; I-644; I-645; I-655; I-656; I-657; I-661; I-662; I-663; I-664; I-666; I-667; I-668; I-669; I-670; I-673; I-676; I-684; I-687; I-688; I-694; I-695; I-696; I-697; I-703; I-705; II-008; II-009; II-016; II-024; II-025; II-028; II-031; II-035; II-036; II-038.

Example: in vivo preventive test on Puccinia recondita (brown rust on wheat)

| | |
|---|---|
| Solvent: | 5% by volume of Dimethyl sulfoxide |
| | 10% by volume of Acetone |
| Emulsifier: | 1 μl of Tween ® 80 per mg of active ingredient |

The active ingredients are made soluble and homogenized in a mixture of Dimethyl sulfoxide/Acetone/Tween® 80 and then diluted in water to the desired concentration.

The young plants of wheat are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Puccinia recondita spores. The contaminated wheat plants are incubated for 24 hours at 20° C. and at 100% relative humidity and then for 10 days at 20° C. and at 70-80% relative humidity.

The test is evaluated 11 days after the inoculation. 0% means an efficacy which corresponds to that of the control plants while an efficacy of 100% means that no disease is observed.

In this test, the following compounds according to the invention showed efficacy of at least 70% at a concentration of 500 ppm of active ingredient: I-001; I-003; I-009; I-016; I-020; I-025; I-054; I-073; I-074; I-085; I-091; I-094; I-101; I-134; I-163; I-164; I-166; I-168; I-195; I-243; I-250; I-260; I-261; I-291; I-339; I-341; I-343; I-352; I-353; I-354; I-379; I-381; I-382; I-385; I-386; I-400; I-408; I-409; I-489; I-495; I-496; I-523; I-524; I-525; I-527; I-528; I-530; I-531; I-534; I-536; I-537; I-538; I-544; I-545; I-549; I-551; I-552; I-571; I-589; I-592; I-597; I-602; I-604; I-614; I-615; I-624; I-633; I-634; I-638; I-639; I-641; I-643; I-645; I-647; I-648; I-649; I-651; I-652; I-653; I-659; I-663; I-664; I-665; I-666; I-667; I-669; I-670; I-671; I-673; I-685; I-687; I-688; I-691; I-692; I-693; II-001; II-008; II-028.

Example: In Vivo Preventive Test on Pyrenophora teres (Net Blotch on Barley)

| | |
|---|---|
| Solvent: | 5% by volume of Dimethyl sulfoxide |
| | 10% by volume of Acetone |
| Emulsifier: | 1 μl of Tween ® 80 per mg of active ingredient |

The active ingredients are made soluble and homogenized in a mixture of Dimethyl sulfoxide/Acetone/Tween® 80 and then diluted in water to the desired concentration.

The young plants of barley are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Pyrenophora teres spores. The contaminated barley plants are incubated for 48 hours at 20° C. and at 100% relative humidity and then for 12 days at 20° C. and at 70-80% relative humidity.

The test is evaluated 14 days after the inoculation. 0% means an efficacy which corresponds to that of the control plants while an efficacy of 100% means that no disease is observed.

In this test, the following compounds according to the invention showed efficacy of at least 70% at a concentration of 500 ppm of active ingredient: 1-002; I-003; I-004; I-005; I-008; I-009; I-020; I-029; I-031; I-045; I-085; I-109; I-123; I-133; I-134; I-158; I-181; I-182; I-184; I-186; I-193; I-195; I-228; I-248; I-251; I-260; I-339; I-353; I-354; I-369; I-400; I-409; I-448; I-450; I-452; I-458; I-459; I-460; I-466; I-468; I-471; I-472; I-474; I-476; I-477; I-482; I-484; I-485; I-488; I-489; I-495; I-502; I-503; I-506; I-544; I-555; I-556; I-557; I-565; I-566; I-567; I-574; I-604; I-609; I-611; I-614; I-615; I-619; I-620; I-623; I-624; I-625; I-633; I-640; I-644; I-651; I-684; I-689; I-690; I-700; II-002; II-041; II-042.

Example: In Vivo Preventive Test on Septoria tritici (Leaf Spot on Wheat)

| | |
|---|---|
| Solvent: | 5% by volume of Dimethyl sulfoxide |
| | 10% by volume of Acetone |
| Emulsifier: | 1 μl of Tween ® 80 per mg of active ingredient |

The active ingredients are made soluble and homogenized in a mixture of Dimethyl sulfoxide/Acetone/Tween® 80 and then diluted in water to the desired concentration.

The young plants of wheat are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Septoria tritici spores. The contaminated wheat plants are incubated for 72 hours at 18° C. and at 100% relative humidity and then for 21 days at 20° C. and at 90% relative humidity.

The test is evaluated 24 days after the inoculation. 0% means an efficacy which corresponds to that of the control plants while an efficacy of 100% means that no disease is observed.

In this test, the following compounds according to the invention showed efficacy of at least 70% at a concentration of 500 ppm of active ingredient: I-003; I-006; I-007; I-020; I-024; I-028; I-029; I-030; I-035; I-041; I-045; I-060; I-115; I-128; I-129; I-132; I-133; I-135; I-145; I-152; I-153; I-163; I-164; I-169; I-181; I-182; I-186; I-192; I-193; I-195; I-198; I-202; I-225; I-243; I-251; I-261; I-273; I-274; I-278; I-279; I-304; I-305; I-306; I-315; I-337; I-352; I-353; I-354; I-355; I-367; I-368; I-380; I-381; I-382; I-384; I-385; I-396; I-397; I-398; I-401; I-405; I-410; I-428; I-431; I-436; I-438; I-440; I-448; I-452; I-460; I-464; I-467; I-468; I-471; I-472; I-474; I-477; I-479; I-484; I-485; I-489; I-490; I-498; I-499; I-500; I-503; I-506; I-507; I-555; I-557; I-567; I-573; I-574; I-575; I-577; I-578; I-580; I-583; I-599; I-600; I-605; I-607; I-613; I-614; I-616; I-618; I-619; I-621; I-622; I-624; I-625; I-628; I-629; I-631; I-632; I-633; I-634; I-635; I-636; I-637; I-638; I-639; I-640; I-641; I-642; I-644; I-645; I-660; I-662; I-663; I-664; I-665; I-673; I-686; I-688; I-691; I-694; I-698; I-700; II-001; II-022; II-030; II-031; II-038; II-039; II-040.

Example: In Vivo Preventive Test on Sphaerotheca fuliginea (Powdery Mildew on Cucurbits)

| | |
|---|---|
| Solvent: | 5% by volume of Dimethyl sulfoxide |
| | 10% by volume of Acetone |
| Emulsifier: | 1 μl of Tween ® 80 per mg of active ingredient |

The active ingredients are made soluble and homogenized in a mixture of Dimethyl sulfoxide/Acetone/Tween® 80 and then diluted in water to the desired concentration.

The young plants of gherkin are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Sphaerotheca fuliginea spores. The contaminated gherkin plants are incubated for 72 hours at 18° C. and at 100% relative humidity and then for 12 days at 20° C. and at 70-80% relative humidity.

The test is evaluated 15 days after the inoculation. 0% means an efficacy which corresponds to that of the control plants while an efficacy of 100% means that no disease is observed.

In this test, the following compounds according to the invention showed efficacy of at least 70% at a concentration of 500 ppm of active ingredient: I-003; I-011; I-016; I-020; I-030; I-040; I-044; I-061; I-062; I-065; I-066; I-070; I-082; I-084; I-085; I-105; I-128; I-153; I-198; I-226; I-243; I-261; I-273; I-291; I-300; I-305; I-338; I-343; I-359; I-401; I-440; I-487; I-495; I-573; I-575; I-577; I-590; I-605; I-618; I-629; I-635; I-637; I-638; I-639; I-660; I-662; I-663; I-664; I-668; I-669; I-670; I-673; I-682; I-686; I-691; I-692; I-695; I-697; I-705; II-008; II-024; II-025; II-031.

Example: In Vivo Preventive Test on Uromyces appendiculatus (Bean Rust)

| | |
|---|---|
| Solvent: | 5% by volume of Dimethyl sulfoxide |
| | 10% by volume of Acetone |
| Emulsifier: | 1 μl of Tween ® 80 per mg of active ingredient |

The active ingredients are made soluble and homogenized in a mixture of Dimethyl sulfoxide/Acetone/Tween® 80 and then diluted in water to the desired concentration.

The young plants of bean are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Uromyces appendiculatus spores. The contaminated bean plants are incubated for 24 hours at 20° C. and at 100% relative humidity and then for 10 days at 20° C. and at 70-80% relative humidity.

The test is evaluated 11 days after the inoculation. 0% means an efficacy which corresponds to that of the control plants while an efficacy of 100% means that no disease is observed.

In this test, the following compounds according to the invention showed efficacy of at least 70% at a concentration of 500 ppm of active ingredient: I-003; I-004; I-017; I-020; I-029; I-089; I-095; I-101; I-125; I-134; I-158; I-164; I-261; I-316; I-339; I-400; I-404; I-407; I-559; I-565; I-566; I-567; I-604; I-609; I-610; I-611; I-612; I-614; I-652; I-667; I-669; I-671; I-672; I-677; I-684; II-028.

Example: In Vivo Preventive Test on Botrvtis cinerea (Grey Mould)

| | |
|---|---|
| Solvent: | 5% by volume of Dimethyl sulfoxide |
| | 10% by volume of Acetone |
| Emulsifier: | 1 μl of Tween ® 80 per mg of active ingredient |

The active ingredients are made soluble and homogenized in a mixture of Dimethyl sulfoxide/Acetone/Tween® 80 and then diluted in water to the desired concentration.

The young plants of gherkin are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Botrytis cinerea spores. The contaminated gherkin plants are incubated for 4 to 5 days at 17° C. and at 90% relative humidity.

The test is evaluated 4 to 5 days after the inoculation. 0% means an efficacy which corresponds to that of the control plants while an efficacy of 100% means that no disease is observed.

In this test the following compounds according to the invention showed efficacy between 70% and 79% at a concentration of 500 ppm of active ingredient: I-006; I-007; I-043; I-052; I-058; I-081; I-096; I-110; I-129; I-134; I-145; I-202; I-304; I-353; I-379; I-431; I-433; I-456; I-467; I-530; I-550; I-619; I-635; I-642; I-666; I-672; I-675; I-745; I-747; I-779; I-789; I-797; I-1080; I-1109; I-1112; I-1560; II-008; II-103.

In this test the following compounds according to the invention showed efficacy between 80% and 89% at a concentration of 500 ppm of active ingredient: I-095; I-104; I-123; I-152; I-159; I-272; I-274; I-316; I-452; I-475; I-488; I-549; I-574; I-604; I-609; I-615; I-620; I-629; I-644; I-650; I-651; I-684; I-693; I-776; I-781; I-787; I-830; I-879; I-913; I-1058; I-1062; I-1115; I-1120; I-1121; I-1160; I-1436; I-1512; I-1523; II-028; II-032; II-033; II-051; II-082.

In this test the following compounds according to the invention showed efficacy between 90% and 100% at a concentration of 500 ppm of active ingredient: I-001; I-002; I-003; I-004; I-005; I-009; I-014; I-016; I-017; I-020; I-028; I-030; I-051; I-082; I-085; I-086; I-089; I-093; I-109; I-125; I-128; I-153; I-164; I-170; I-181; I-184; I-186; I-195; I-228; I-232; I-239; I-245; I-246; I-248; I-249; I-250; I-251; I-253; I-255; I-261; I-276; I-277; I-278; I-279; I-282; I-289; I-305; I-339; I-372; I-380; I-381; I-383; I-385; I-386; I-407; I-410; I-448; I-449; I-450; I-451; I-453; I-454; I-457; I-458; I-459; I-466; I-468; I-469; I-471; I-472; I-473; I-474; I-476; I-477; I-478; I-481; I-482; I-483; I-484; I-489; I-496; I-498; I-502; I-503; I-506; I-507; I-508; I-510; I-513; I-514; I-519; I-527; I-528; I-529; I-535; I-536; I-537; I-539; I-542; I-553; I-583; I-602; I-610; I-628; I-633; I-634; I-641; I-643; I-645; I-647; I-653; I-662; I-663; I-664; I-677; I-686; I-708; I-710; I-715; I-717; I-719; I-720; I-722; I-732; I-740; I-741; I-752; I-753; I-754; I-755; I-758; I-759; I-760; I-761; I-762; I-766; I-767; I-769; I-777; I-783; I-812; I-813; I-821; I-826; I-827; I-828; I-829; I-831; I-832; I-838; I-839; I-841; I-849; I-901; I-984; I-1007; I-1014; I-1015; I-1016; I-1017; I-1018; I-1020; I-1021; I-1026; I-1037; I-1051; I-1055; I-1056; I-1063; I-1098; I-1105; I-1106; I-1107; I-1110; I-1113; I-1114; I-1116; I-1122; I-1123; I-1131; I-1132; I-1135; I-1136; I-1137; I-1138; I-1140; I-1141; I-1142; I-1145; I-1147; I-1152; I-1222; I-1227; I-1293; I-1294; I-1302; I-1305; I-1409; I-1411; I-1412; I-1413; I-1414; I-1416; I-1418; I-1419; I-1421; I-1422; I-1423; I-1453; I-1508; I-1525; I-1528; I-1536; I-1540; I-1541; I-1543; I-1544; I-1545; I-1548; I-1563; I-1569; II-015; II-016; II-041; II-042; II-044; II-058; II-061; II-094.

Example: In Vivo Preventive Test on Phytophthora infestans (Tomato Late Blight)

| | |
|---|---|
| Solvent: | 5% by volume of Dimethyl sulfoxide |
| | 10% by volume of Acetone |
| Emulsifier: | 1 μl of Tween ® 80 per mg of active ingredient |

The active ingredients are made soluble and homogenized in a mixture of Dimethyl sulfoxide/Acetone/Tween® 80 and then diluted in water to the desired concentration.

The young plants of tomato are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Phytophthora infestans spores. The contaminated tomato plants are incubated for 5 days at 16-18° C. and at 100% relative humidity.

The test is evaluated 5 days after the inoculation. 0% means an efficacy which corresponds to that of the control plants while an efficacy of 100% means that no disease is observed.

In this test the following compounds according to the invention showed efficacy between 70% and 79% at a concentration of 500 ppm of active ingredient: 1-032; I-068; I-098; I-100; I-101; I-106; I-199; I-261; I-278; I-360; I-420; I-463; I-524; I-529; I-545; I-550; I-591; I-633; I-655; I-666; I-697; I-705; I-728; I-742; I-753; I-1028; I-1085; I-1133; I-1141; I-1151; I-1307; I-1352; I-1448; I-1464; I-1561.

In this test the following compounds according to the invention showed efficacy between 80% and 89% at a concentration of 500 ppm of active ingredient: 1-053; I-093; I-149; I-195; I-332; I-338; I-389; I-406; I-424; I-435; I-487; I-555; I-618; I-623; I-634; I-645; I-684; I-712; I-736; I-743; I-754; I-1058; I-1153; I-1368; I-1382; I-1398; I-1450; I-1469; I-1485; I-1491; I-1499; I-1520; I-1524; I-1548; I-1550; I-1563; II-008.

In this test the following compounds according to the invention showed efficacy between 90% and 100% at a concentration of 500 ppm of active ingredient: I-003; I-009; I-011; I-012; I-013; I-015; I-016; I-020; I-021; I-023; I-024; I-028; I-030; I-035; I-036; I-037; I-038; I-040; I-041; I-043; I-044; I-045; I-050; I-051; I-052; I-055; I-057; I-058; I-059; I-061; I-062; I-063; I-065; I-066; I-069; I-070; I-071; I-074; I-077; I-079; I-082; I-085; I-086; I-087; I-090; I-091; I-094; I-095; I-097; I-105; I-108; I-110; I-114; I-120; I-122; I-123; I-124; I-126; I-127; I-128; I-129; I-130; I-132; I-133; I-135; I-137; I-140; I-141; I-142; I-143; I-144; I-145; I-147; I-148; I-159; I-160; I-163; I-165; I-166; I-169; I-172; I-177; I-179; I-180; I-181; I-186; I-192; I-193; I-196; I-198; I-202; I-217; I-229; I-232; I-239; I-240; I-243; I-245; I-247; I-251; I-258; I-260; I-262; I-270; I-271; I-272; I-275; I-281; I-282; I-291; I-300; I-301; I-303; I-304; I-305; I-306; I-315; I-324; I-337; I-341; I-343; I-349; I-353; I-355; I-356; I-357; I-361; I-368; I-370; I-377; I-382; I-383; I-384; I-388; I-396; I-397; I-398; I-399; I-403; I-405; I-410; I-417; I-418; I-421; I-423; I-427; I-428; I-429; I-431; I-437; I-440; I-444; I-450; I-461; I-466; I-467; I-469; I-479; I-484; I-490; I-491; I-492; I-498; I-499; I-500; I-501; I-503; I-509; I-514; I-515; I-518; I-520; I-523; I-525; I-526; I-534; I-538; I-542; I-543; I-556; I-560; I-563; I-564; I-565; I-566; I-570; I-575; I-583; I-584; I-585; I-589; I-594; I-596; I-597; I-601; I-605; I-616; I-619; I-620; I-621; I-622; I-630; I-632; I-635; I-636; I-637; I-638; I-640; I-644; I-656; I-657; I-658; I-661; I-662; I-663; I-664; I-667; I-668; I-669; I-670; I-673; I-676; I-683; I-687; I-688; I-694; I-695; I-696; I-703; I-710; I-713; I-714; I-715; I-717; I-718; I-719; I-721; I-723; I-724; I-725; I-726; I-727; I-732; I-733; I-734; I-737; I-744; I-746; I-755; I-762; I-765; I-767; I-771; I-774; I-782; I-783; I-784; I-785; I-786; I-787; I-791; I-792; I-793; I-797; I-802; I-803; I-804; I-805; I-807; I-808; I-809; I-810; I-811; I-813; I-814; I-815; I-817; I-818; I-819; I-820; I-832; I-833; I-836; I-837; I-838; I-839; I-841; I-842; I-845; I-846; I-847; I-849; I-850; I-857; I-895; I-901; I-913; I-984; I-985; I-1003; I-1014; I-1018; I-1020; I-1021; I-1022; I-1023; I-1026; I-1038; I-1049; I-1052; I-1053; I-1056; I-1067; I-1069; I-1086; I-1087; I-1091; I-1092; I-1094; I-1096; I-1098; I-1125; I-1126; I-1127; I-1128; I-1129; I-1130; I-1140; I-1148; I-1149; I-1152; I-1160; I-1161; I-1166; I-1167; I-1221; I-1278; I-1281; I-1282; I-1288; I-1289; I-1295; I-1351; I-1356; I-1367; I-1379; I-1383; I-1385; I-1386; I-1388; I-1390; I-1399; I-1403; I-1408; I-1410; I-1413; I-1417; I-1424; I-1435; I-1441; I-1445; I-1447; I-1453; I-1477; I-1478; I-1479; I-1484; I-1516; I-1521; I-1525; I-1539; I-1542; I-1551; I-1552; I-1553; I-1554; I-1564; I-1565; I-1566; I-1567; I-1569; II-009; II-016; II-024; II-025; II-028; II-031; II-035; II-036; II-038; II-051; II-058; II-060; II-070; II-071; II-082; II-084; II-085; II-094.

Example: In Vivo Preventive Test on Puccinia recondita (Brown Rust on Wheat)

| | |
|---|---|
| Solvent: | 5% by volume of Dimethyl sulfoxide |
| | 10% by volume of Acetone |
| Emulsifier: | 1 μl of Tween ® 80 per mg of active ingredient |

The active ingredients are made soluble and homogenized in a mixture of Dimethyl sulfoxide/Acetone/Tween® 80 and then diluted in water to the desired concentration.

The young plants of wheat are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Puccinia recondita spores. The contaminated wheat plants are incubated for 24 hours at 20° C. and at 100% relative humidity and then for 10 days at 20° C. and at 70-80% relative humidity.

The test is evaluated 11 days after the inoculation. 0% means an efficacy which corresponds to that of the control plants while an efficacy of 100% means that no disease is observed.

In this test the following compounds according to the invention showed efficacy between 70% and 79% at a concentration of 500 ppm of active ingredient: I-001; I-085; I-094; I-101; I-134; I-164; I-166; I-168; I-195; I-243; I-250; I-260; I-343; I-352; I-382; I-386; I-400; I-408; I-409; I-524; I-531; I-534; I-551; I-592; I-597; I-634; I-638; I-641; I-643; I-645; I-648; I-652; I-659; I-664; I-669; I-670; I-688; I-691; I-715; I-718; I-719; I-757; I-776; I-779; I-786; I-787; I-795; I-804; I-826; I-831; I-835; I-850; I-857; I-963; I-1055; I-1057; I-1058; I-1061; I-1072; I-1109; I-1114; I-1130; I-1277; I-1278; I-1279; I-1280; I-1282; I-1303; I-1358; I-1416; I-1436; I-1523; I-1563.

In this test the following compounds according to the invention showed efficacy between 80% and 89% at a concentration of 500 ppm of active ingredient: I-009; I-020; I-025; I-074; I-086; I-163; I-261; I-291; I-339; I-341; I-353; I-354; I-379; I-381; I-385; I-489; I-495; I-523; I-525; I-527; I-530; I-571; I-589; I-602; I-604; I-615; I-624; I-633; I-647; I-651; I-653; I-663; I-665; I-666; I-667; I-673; I-685; I-687; I-692; I-708; I-709; I-722; I-740; I-742; I-743; I-745; I-749; I-751; I-760; I-766; I-769; I-822; I-828; I-829; I-840; I-914; I-1046; I-1051; I-1062; I-1063; I-1075; I-1120; I-1125; I-1127; I-1129; I-1136; I-1138; I-1139; I-1141; I-1145; I-1147; I-1285; I-1548; II-001; II-008; II-028; II-044; II-082.

In this test the following compounds according to the invention showed efficacy between 90% and 100% at a concentration of 500 ppm of active ingredient: 1-003; I-016; I-054; I-073; I-091; I-170; I-496; I-528; I-536; I-537; I-538; I-544; I-545; I-549; I-552; I-614; I-639; I-649; I-671; I-693; I-720; I-761; I-1015; II-043; II-045; II-079; II-080; II-084.

Example: In Vivo Preventive Test on Pyrenophora teres (Net Blotch on Barley)

| | |
|---|---|
| Solvent: | 5% by volume of Dimethyl sulfoxide |
| | 10% by volume of Acetone |
| Emulsifier: | 1 μl of Tween ® 80 per mg of active ingredient |

The active ingredients are made soluble and homogenized in a mixture of Dimethyl sulfoxide/Acetone/Tween® 80 and then diluted in water to the desired concentration.

The young plants of barley are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Pyrenophora teres spores. The contaminated barley plants are incubated for 48 hours at 20° C. and at 100% relative humidity and then for 12 days at 20° C. and at 70-80% relative humidity.

The test is evaluated 14 days after the inoculation. 0% means an efficacy which corresponds to that of the control plants while an efficacy of 100% means that no disease is observed.

In this test the following compounds according to the invention showed efficacy between 70% and 79% at a concentration of 500 ppm of active ingredient: I-029; I-079; I-085; I-109; I-123; I-133; I-134; I-158; I-182; I-184; I-193; I-195; I-228; I-248; I-251; I-339; I-353; I-354; I-369; I-400; I-466; I-468; I-476; I-477; I-482; I-484; I-485; I-488; I-489; I-495; I-503; I-565; I-566; I-574; I-615; I-619; I-623; I-624; I-633; I-700; I-724; I-745; I-750; I-751; I-752; I-754; I-755; I-761; I-776; I-778; I-785; I-786; I-791; I-821; I-835; I-852; I-854; I-914; I-1007; I-1026; I-1061; I-1096; I-1115; I-1127; I-1145; I-1278; I-1279; I-1292; I-1300; I-1302; I-1303; I-1350; I-1390; I-1409; I-1418; I-1423; I-1549; II-002; II-041; II-042; II-045; II-061; II-079.

In this test the following compounds according to the invention showed efficacy between 80% and 89% at a concentration of 500 ppm of active ingredient: I-002; I-003; I-005; I-009; I-031; I-045; I-086; I-186; I-409; I-410; I-450; I-452; I-458; I-459; I-460; I-471; I-472; I-474; I-502; I-506; I-555; I-556; I-557; I-567; I-604; I-609; I-614; I-625; I-640; I-644; I-651; I-671; I-684; I-689; I-850; I-851; I-1029; I-1063; I-1075; I-1076; I-1097; I-1106; I-1107; I-1110; I-1111; I-1113; I-1120; I-1150; I-1155; I-1282; I-1284; I-1414; I-1421; I-1548; I-1564; II-043; II-082.

In this test the following compounds according to the invention showed efficacy between 90% and 100% at a concentration of 500 ppm of active ingredient: I-004; I-008; I-020; I-181; I-260; I-448; I-544; I-611; I-620; I-690; I-1015; I-1037; I-1074; I-1157.

Example: In Vivo Preventive Test on Septoria tritici (Leaf Spot on Wheat)

| | |
|---|---|
| Solvent: | 5% by volume of Dimethyl sulfoxide |
| | 10% by volume of Acetone |
| Emulsifier: | 1 μl of Tween ® 80 per mg of active ingredient |

The active ingredients are made soluble and homogenized in a mixture of Dimethyl sulfoxide/Acetone/Tween® 80 and then diluted in water to the desired concentration.

The young plants of wheat are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Septoria tritici spores. The contaminated wheat plants are incubated for 72 hours at 18° C. and at 100% relative humidity and then for 21 days at 20° C. and at 90% relative humidity.

The test is evaluated 24 days after the inoculation. 0% means an efficacy which corresponds to that of the control plants while an efficacy of 100% means that no disease is observed.

In this test the following compounds according to the invention showed efficacy between 70% and 79% at a concentration of 500 ppm of active ingredient: I-007; I-028; I-029; I-045; I-060; I-129; I-135; I-145; I-164; I-182; I-186; I-192; I-198; I-279; I-305; I-315; I-368; I-381; I-401; I-436; I-438; I-464; I-468; I-485; I-498; I-499; I-507; I-555; I-567; I-575; I-577; I-607; I-621; I-622; I-628; I-631; I-660; I-686; I-698; I-700; I-716; I-721; I-724; I-734; I-746; I-747; I-753; I-767; I-769; I-778; I-783; I-792; I-807; I-813; I-841; I-842; I-855; I-857; I-884; I-984; I-997; I-1003; I-1049; I-1052; I-1085; I-1086; I-1094; I-1125; I-1281; I-1282; I-1284; I-1288; I-1304; I-1353; I-1369; I-1408; I-1413; I-1474; I-1475; I-1517; I-1521; I-1550; I-1552; II-001; II-030; II-038; II-040.

In this test the following compounds according to the invention showed efficacy between 80% and 89% at a concentration of 500 ppm of active ingredient: I-003; I-006; I-024; I-030; I-035; I-041; I-115; I-128; I-132; I-133; I-153; I-169; I-193; I-195; I-243; I-251; I-261; I-273; I-274; I-278; I-304; I-306; I-337; I-384; I-398; I-428; I-440; I-452; I-460; I-472; I-474; I-477; I-484; I-490; I-503; I-506; I-573; I-599; I-600; I-616; I-619; I-625; I-629; I-632; I-633; I-634; I-635; I-639; I-665; I-673; I-691; I-694; I-741; I-762; I-780; I-793; I-846; I-850; I-901; I-1018; I-1029; I-1066; I-1072; I-1089; I-1124; I-1128; I-1129; I-1130; I-1141; I-1370; I-1384; I-1386; I-1453; I-1548; I-1551; I-1554; II-039; II-082.

In this test the following compounds according to the invention showed efficacy between 90% and 100% at a concentration of 500 ppm of active ingredient: I-020; I-086; I-098; I-152; I-163; I-181; I-202; I-225; I-352; I-353; I-354; I-355; I-367; I-380; I-382; I-385; I-396; I-397; I-405; I-410; I-431; I-448; I-467; I-471; I-479; I-489; I-500; I-557; I-574; I-578; I-580; I-583; I-605; I-613; I-614; I-618; I-624; I-636; I-637; I-638; I-640; I-641; I-642; I-644; I-645; I-662; I-663; I-664; I-688; I-718; I-719; I-749; I-752; I-786; I-787; I-810; I-812; I-847; I-1022; I-1055; I-1149; I-1151; I-1152; I-1357; I-1358; I-1359; I-1367; I-1379; I-1385; I-1388; I-1390; I-1493; I-1541; I-1563; I-1569; II-022; II-031.

Example: In Vivo Preventive Test on Sphaerotheca fuliginea (Powdery Mildew on Cucurbits)

| | |
|---|---|
| Solvent: | 5% by volume of Dimethyl sulfoxide |
| | 10% by volume of Acetone |
| Emulsifier: | 1 μl of Tween ® 80 per mg of active ingredient |

The active ingredients are made soluble and homogenized in a mixture of Dimethyl sulfoxide/Acetone/Tween® 80 and then diluted in water to the desired concentration.

The young plants of gherkin are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Sphaerotheca fuliginea spores. The contaminated gherkin plants are incubated for 72 hours at 18° C. and at 100% relative humidity and then for 12 days at 20° C. and at 70-80% relative humidity.

The test is evaluated 15 days after the inoculation. 0% means an efficacy which corresponds to that of the control plants while an efficacy of 100% means that no disease is observed.

In this test the following compounds according to the invention showed efficacy between 70% and 79% at a concentration of 500 ppm of active ingredient: I-085; I-243; I-273; I-343; I-359; I-487; I-590; I-605; I-618; I-635; I-658; I-662; I-686; I-695; I-705; I-812; I-813; I-834; I-841; I-901; I-1018; I-1298; I-1300; I-1473; I-1528; II-008.

In this test the following compounds according to the invention showed efficacy between 80% and 89% at a concentration of 500 ppm of active ingredient: I-011; I-016; I-044; I-061; I-066; I-070; I-079; I-082; I-305; I-577; I-683; I-743; I-744; I-785; I-808; I-816; I-823; I-1049; I-1278; II-024; II-025.

In this test the following compounds according to the invention showed efficacy between 90% and 100% at a concentration of 500 ppm of active ingredient: 1-003; I-020; I-030; I-040; I-062; I-065; I-084; I-105; I-128; I-153; I-198; I-226; I-261; I-291; I-300; I-338; I-401; I-440; I-495; I-573; I-575; I-629; I-637; I-638; I-639; I-660; I-663; I-664; I-668; I-669; I-670; I-673; I-682; I-691; I-692; I-697; I-715; I-739; I-786; I-787; I-792; I-793; I-818; I-1022; I-1023; I-1026; I-1038; I-1057; I-1063; I-1068; I-1169; I-1282; I-1284; I-1302; I-1303; I-1351; I-1356; I-1358; I-1453; I-1548; II-031.

Example: In Vivo Preventive Test on Uromyces appendiculatus (Bean Rust)

| | |
|---|---|
| Solvent: | 5% by volume of Dimethyl sulfoxide |
| | 10% by volume of Acetone |
| Emulsifier: | 1 μl of Tween ® 80 per mg of active ingredient |

The active ingredients are made soluble and homogenized in a mixture of Dimethyl sulfoxide/Acetone/Tween® 80 and then diluted in water to the desired concentration.

The young plants of bean are treated by spraying the active ingredient prepared as described above. Control plants are treated only with an aqueous solution of Acetone/Dimethyl sulfoxide/Tween® 80.

After 24 hours, the plants are contaminated by spraying the leaves with an aqueous suspension of Uromyces appendiculatus spores. The contaminated bean plants are incubated for 24 hours at 20° C. and at 100% relative humidity and then for 10 days at 20° C. and at 70-80% relative humidity.

The test is evaluated 11 days after the inoculation. 0% means an efficacy which corresponds to that of the control plants while an efficacy of 100% means that no disease is observed.

In this test the following compounds according to the invention showed efficacy between 70% and 79% at a concentration of 500 ppm of active ingredient: I-020; I-029; I-095; I-125; I-164; I-407; I-559; I-566; I-567; I-609; I-612; I-614; I-652; I-672; I-745; I-779; I-781; I-850; I-857; I-1006; I-1007; I-1046; I-1114; I-1116; I-1283; I-1300; I-1423; II-076; II-077; II-082.

In this test the following compounds according to the invention showed efficacy between 80% and 89% at a concentration of 500 ppm of active ingredient: I-089; I-101; I-158; I-261; I-339; I-565; I-610; I-611; I-667; I-669; I-671; I-677; I-684; I-743; I-1057; I-1105; I-1107; I-1110; I-1150; I-1155; I-1156; I-1279; I-1282; I-1285; I-1303; I-1358; I-1409; I-1545; II-028.

In this test the following compounds according to the invention showed efficacy between 90% and 100% at a concentration of 500 ppm of active ingredient: 1-003; I-004; I-017; I-134; I-316; I-400; I-404; I-604; I-1043; I-1061; I-1063; I-1075; I-1106; I-1109; I-1169; I-1222; I-1421; I-1525; I-1544; I-1549; II-060; II-085.

Example: In Vivo Preventive Test on Alternaria solani (Tomatoes)

| | |
|---|---|
| Solvent: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for preventive activity, young plants are sprayed with the preparation of active compound at the stated rate of application. After the spray coating has dried on, the plants are inoculated with an aqueous spore suspension of Alternaria solani. The plants are then placed in an incubation cabinet at approximately 20° C. and a relative atmospheric humidity of 100%.

The test is evaluated 3 days after the inoculation. 0% means an efficacy which corresponds to that of the untreated control while an efficacy of 100% means that no disease is observed.

In this test the following compounds according to the invention showed efficacy between 70% and 79% at a concentration of 250 ppm of active ingredient: 1-070; I-082; I-110; I-356; I-381; I-382; I-383; I-838; II-003; II-058.

In this test the following compounds according to the invention showed efficacy between 80% and 89% at a concentration of 250 ppm of active ingredient: I-007; I-045; I-061; I-065; I-087; I-108; I-159; I-163; I-304; I-324; I-377; I-385; I-663; I-664; I-762; I-836; I-1056; I-1072; II-061; II-085.

In this test the following compounds according to the invention showed efficacy between 90% and 100% at a concentration of 250 ppm of active ingredient: I-003; I-005; I-006; I-008; I-009; I-015; I-016; I-020; I-028; I-030; I-031; I-041; I-055; I-062; I-066; I-074; I-077; I-085; I-086; I-089; I-091; I-093; I-094; I-095; I-105; I-109; I-120; I-123; I-125; I-128; I-129; I-130; I-132; I-133; I-134; I-141; I-145; I-164; I-169; I-179; I-181; I-186; I-193; I-202; I-228; I-229; I-232; I-239; I-243; I-245; I-250; I-251; I-260; I-261; I-272; I-275; I-278; I-282; I-289; I-305; I-306; I-315; I-339; I-353; I-400; I-401; I-405; I-448; I-450; I-467; I-484; I-489; I-495; I-528; I-529; I-565; I-583; I-589; I-602; I-604; I-610; I-620; I-621; I-633; I-634; I-637; I-638; I-643; I-645; I-651; I-653; I-662; I-667; I-669; I-673; I-688; I-708; I-715; I-717; I-732; I-752; I-753; I-755; I-767; I-769; I-783; I-786; I-808; I-818; I-841; I-850; I-984; I-1014; I-1015; I-1018; I-1021; I-1046; I-1051; I-1058; I-1140; I-1169; I-1302; I-1409; I-1413; I-1416; I-1453; I-1563; II-028; II-044.

Example: In Vivo Preventive Test on Venturia inaequalis (Apples)

| | |
|---|---|
| Solvent: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for preventive activity, young plants are sprayed with the preparation of active compound at the stated rate of application. After the spray coating has dried on, the plants are inoculated with an aqueous conidia suspension of the causal agent of apple scab (Venturia inaequalis) and then remain for 1 day in an incubation cabinet at approximately 20° C. and a relative atmospheric humidity of 100%.

The plants are then placed in a greenhouse at approximately 21° C. and a relative atmospheric humidity of approximately 90%.

The test is evaluated 10 days after the inoculation. 0% means an efficacy which corresponds to that of the untreated control, while an efficacy of 100% means that no disease is observed.

In this test the following compounds according to the invention showed efficacy between 70% and 79% at a concentration of 250 ppm of active ingredient: I-169; I-304; I-667.

In this test the following compounds according to the invention showed efficacy between 80% and 89% at a concentration of 250 ppm of active ingredient: I-044; I-163; I-282; I-315; I-316; I-377; I-450; I-489; I-839; I-849; I-1563; II-003.

In this test the following compounds according to the invention showed efficacy between 90% and 100% at a concentration of 250 ppm of active ingredient: I-003; I-005; I-006; I-007; I-008; I-009; I-015; I-016; I-020; I-028; I-030; I-031; I-041; I-045; I-061; I-062; I-065; I-066; I-074; I-077; I-082; I-085; I-086; I-089; I-091; I-093; I-094; I-108; I-109; I-120; I-123; I-125; I-128; I-129; I-130; I-133; I-134; I-141; I-145; I-159; I-164; I-179; I-181; I-186; I-193; I-202; I-228; I-229; I-232; I-239; I-243; I-245; I-250; I-251; I-260; I-261; I-272; I-275; I-278; I-289; I-305; I-306; I-324; I-339; I-353; I-356; I-380; I-381; I-382; I-383; I-385; I-405; I-448; I-467; I-484; I-556; I-565; I-583; I-589; I-602; I-604; I-610; I-620; I-621; I-633; I-634; I-637; I-638; I-643; I-645; I-651; I-653; I-662; I-663; I-664; I-669; I-673; I-688; I-708; I-715; I-717; I-732; I-752; I-753; I-755; I-762; I-767; I-769; I-783; I-786; I-808; I-818; I-836; I-838; I-841; I-850; I-984; I-1014; I-1015; I-1018; I-1021; I-1046; I-1051; I-1056; I-1058; I-1072; I-1140; I-1169; I-1302; I-1409; I-1413; I-1416; I-1453; II-016; II-044; II-058; II-085.

LENGTHY TABLES
The patent application contains a lengthy table section. A copy of the table is available in electronic form from the USPTO web site (http://seqdata.uspto.gov/?pageRequest=docDetail&DocID=US20190110476A1). An electronic copy of the table will also be available from the USPTO upon request and payment of the fee set forth in 37 CFR 1.19(b)(3).

1. A product comprising one or more compounds of the formula (G) and/or salt thereof, 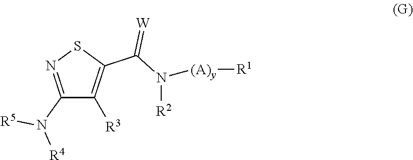
in which
A is CR⁶R⁷,
W is O or S,
R¹ is hydrogen, (C₁-C₁₂)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, NR¹³R¹⁴, R¹³R¹⁴N—(C₁-C₆)-alkyl, (C₁-C₆)-alkoxy, (C₁-C₆)-haloalkoxy, (C₁-C₆)-haloalkoxy-(C₁-C₃)-alkyl, (C₁-C₆)-alkoxy-(C₁-C₃)-alkyl, (C₁-C₆)-alkoxy-(C₂-C₆)-alkoxy-(C₁-C₃)-alkyl, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-haloalkylthio-(C₁-C₃)-alkyl, (C₁-C₄)-haloalkylsulphoxy-(C₁-C₃)-alkyl, (C₁-C₄)-haloalkylsulphonyl-(C₁-C₃)-alkyl, (C₃-C₁₂)-cycloalkyl, (C₃-C₈)-cycloalkenyl, (C₃-C₁₂)-cycloalkyl-(C₁-C₆)-alkyl, (C₃-C₈)-cycloalkenyl-(C₁-C₆)-alkyl, (C₃-C₈)-cycloalkoxy, (C₃-C₈)-cycloalkyl-(C₁-C₆)-alkoxy, aryl, aryl-(C₁-C₃)-alkyl, heteroaryl, heteroaryl-(C₁-C₃)-alkyl, heterocyclyl, heterocyclyl-(C₁-C₃)-alkyl, aryloxy, heteroaryloxy, heterocyclyloxy, a bicyclic or a heterobicyclic residue, wherein each of the last-mentioned 17 residues is unsubstituted or is substituted by one or more residues from the group consisting of halogen, nitro, hydroxyl, cyano, NR¹³R¹⁴, (C₁-C₄)-alkyl, /r″₁- (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-al koxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-hydroxycarbonyl, hydroxycarbonyl-(C₁-C₄)-alkyl, R¹³R¹⁴N-carbonyl, and wherein heterocyclyl has q oxo groups, and wherein each of the aforementioned heterocyclic residues, in addition to the carbon atoms, has in each case p ring members from the group consisting of N(R¹²)ₘ, O and S(O)ₙ,
R² is hydrogen, (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₄)-alkylsulphonyl, (C₁-C₄)-haloalkylsulphonyl, (C₁-C₆)-alkylcarbonyl, (C₂-C₆)-alkenylcarbonyl, (C₂-C₆)-alkynylcarbonyl, (C₁-C₆)-haloalkylcarbonyl, (C₂-C₆)-haloalkenylcarbonyl, (C₂-C₆)-haloalkynylcarbonyl, (C₁-C₆)-alkoxycarbonyl, di((C₁-C₆)-alkyl)aminocarbonyl, (C₃-C₈)-cycloalkyl, (C₃-C₈)-cycloalkyl-(C₁-C₆)-alkyl, (C₃-C₈)-cycloalkylcarbonyl, (C₃-C₈)-cycloalkyl-(C₁-C₆)-alkylcarbonyl, heteroarylcarbonyl, or arylcarbonyl, wherein each of the last-mentioned 6 residues is unsubstituted or is substituted by one or more residues from the group consisting of halogen, nitro, hydroxyl, cyano, NR¹³R¹⁴, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl,
or
R¹ and R², together with the nitrogen atom and (A)_{y} attached thereto, form a 5- or 6-membered heterocyclic or heteroaromatic ring, which comprises in each case, in addition to the carbon atoms and the nitrogen atom, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups, R³ is hydrogen, halogen, azido, isocyanate, isothiocyanate, nitro, cyano, hydroxyl, NR¹³R¹⁴, tri(C₁-C₆)-alkylsilyl, (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₁-C₆)-haloalkoxy, (C₁-C₆)-haloalkoxy-(C₁-C₃)-alkyl, (C₁-C₆)-alkoxy-(C₁-C₃)-alkyl, (C₁-C₆)-alkoxy-(C₂-C₆)-alkoxy-(C₁-C₃)-alkyl, (C₁-C₆)-alkylcarbonyloxy, (C₁-C₆)-haloalkylcarbonyloxy, (C₂-C₆)-alkenylcarbonyloxy, (C₂-C₆)-alkynylcarbonyloxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-haloalkylthio-(C₁-C₃)-alkyl, (C₁-C₄)-haloalkylsulphoxy-(C₁-C₃)-alkyl, (C₁-C₄)-haloalkylsulphonyl-(C₁-C₃)-alkyl, (C₁-C₆)-alkoxycarbonyl, (C₁-C₆)-haloalkoxycarbonyl, (C₂-C₆)-alkenyloxycarbonyl, (C₂-C₆)-haloalkenyloxycarbonyl, (C₂-C₆)-alkynyloxycarbonyl, (C₂-C₆)-haloalkynyloxycarbonyl, (C₁-C₆)-alkylcarbonyl, (C₁-C₆)-haloalkylcarbonyl, (C₂-C₆)-alkenylcarbonyl, (C₂-C₆)-haloalkenylcarbonyl, (C₂-C₆)-alkynylcarbonyl, (C₂-C₆)-haloalkynylcarbonyl, R¹³R¹⁴N-carbonyl, arylthio, arylsulphoxy, arylsulphonyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₃-C₈)-cycloalkoxy, aryl, aryloxy, arylcarbonyloxy, aryl-(C₁-C₃)-alkyl, heteroaryl, heteroaryloxy, heteroaryl-(C₁-C₃)-alkyl, heterocyclyl, heterocyclyloxy, or heterocyclyl-(C₁-C₃)-alkyl, wherein each of the last-mentioned 18 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl, and wherein heterocyclyl has q oxo groups,
R⁴, R⁵ are each independently hydrogen, (C₁-C₁₂)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, (C₁-C₆)-alkoxy-(C₁-C₃)-alkyl, (C₁-C₆)-alkoxy-(C₂-C₆)-alkoxy-(C₁-C₃)-alkyl, (C₁-C₆)-alkoxy-(C₁-C₃)-alkylcarbonyl, (C₁-C₆)-alkoxy-(C₂-C₆)-alkoxy-(C₁-C₃)-alkylcarbonyl, (C₁-C₆)-alkoxycarbonyl, (C₁-C₆)-haloalkoxycarbonyl, (C₂-C₆)-al kenyloxycarbonyl, (C₂-C₆)-haloalkenyloxycarbonyl, (C₂-C₆)-alkynyloxycarbonyl, (C₂-C₆)-haloalkynyloxycarbonyl, (C₁-C₆)-alkylcarbonyl, (C₁-C₆)-haloalkylcarbonyl, (C₂-C₆)-alkenylcarbonyl, (C₂-C₆)-haloalkenylcarbonyl, (C₂-C₆)-alkynylcarbonyl, (C₂-C₆)-haloalkynylcarbonyl, R¹³R¹⁴N-carbonyl, (C₁-C₄)-alkylthio, (C₁-C₄)-haloalkylthio, (C₁-C₈)-alkylthiocarbonyl, (C₁-C₈)-haloalkylthiocarbonyl, (C₁-C₄)-alkylsulphoxy, (C₁-C₄)-haloalkylsulphoxy, (C₁-C₄)-alkylsulphonyl, (C₁-C₄)-haloalkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-alkylthio-(C₁-C₃)-alkylcarbonyl, (C₁-C₄)-alkylsulphoxy-(C₁-C₃)-alkylcarbonyl, (C₁-C₄)-alkylsulphonyl-(C₁-C₃)-alkylcarbonyl, (C₁-C₁₂)-alkylcarbonyl, (C₁-C₁₂)-haloalkylcarbonyl, (C₂-C₁₂)-alkenylcarbonyl, (C₂-C₁₂)-haloalkenylcarbonyl, (C₂-C₁₂)-alkynylcarbonyl, (C₂-C₁₂)-haloalkynylcarbonyl, (C₁-C₁₂)-alkoxycarbonylcarbonyl, (C₁-C₁₂)-alkoxycarbonyl-(C₁-C₃)-alkylcarbonyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₃-C₈)-cycloalkylcarbonyl, (C₃-C₈)-cycloalkenylcarbonyl, (C₃-C₈)-cycloalkyl-(C₁-C₆)-alkylcarbonyl, (C₃-C₈)-cycloalkenyl-(C₁-C₆)-alkylcarbonyl, aryl, aryl-(C₁-C₃)-alkyl, heteroaryl, heteroaryl-(C₁-C₃)-alkyl, heterocyclyl, heterocyclyl-(C₁-C₃)-alkyl, arylcarbonyl, aryl-(C₁-C₆)-alkylcarbonyl, heteroarylcarbonyl, heteroaryl-(C₁-C₆)-alkylcarbonyl, heterocyclylcarbonyl, or heterocyclyl-(C₁-C₆)-alkylcarbonyl, wherein each of the last-mentioned 20 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl, and wherein heterocyclyl has q oxo groups,
or
NR⁴R⁵ is —N═CR⁸R⁹ or —N═S(O)ₙR¹⁰R¹¹,
R⁶, R⁷ are each independently hydrogen, cyano, halogen, (C₁-C₆)-alkyl, (C₂-C₆)-alkenyl, (C₂-C₆)-alkynyl, or (C₃-C₈)-cycloalkyl,
or
R⁶ and R⁷, together with the carbon atom to which they are attached, form a 3-6-membered carbocyclic or heterocyclic ring, which comprises in each case, in addition to the carbon atoms, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
R⁸, R⁹ are each independently hydrogen, (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₁-C₆)-haloalkoxy, (C₁-C₆)-haloalkoxy-(C₁-C₃)-alkyl, (C₂-C₆)-alkenyloxy, (C₂-C₆)-haloalkenyloxy, (C₂-C₆)-alkynyloxy, (C₂-C₆)-haloalkynyloxy, NR¹³R¹⁴, (C₁-C₆)-alkoxy-(C₁-C₃)-alkyl, halogen-(C₁-C₆)-alkoxy-(C₁-C₆)-alkyl, (C₁-C₆)-alkoxy-(C₂-C₆)-alkoxy-(C₁-C₃)-alkyl, (C₁-C₄)-akylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, aryl, aryl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 10 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
or
R⁸ and R⁹, together with the carbon atom to which they are attached, form a 3- to 8-membered unsaturated, partially saturated or saturated ring, which comprises in each case, in addition to the carbon atoms, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-(C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
R¹⁰, R¹¹ are each independently (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₆)-alkoxy-(C₁-C₃)-alkyl, halogen-(C₁-C₆)-alkoxy-(C₁-C₆)-alkyl, (C₁-C₆)-alkoxy-(C₂-C₆)-alkoxy-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, aryl, aryl-(C₁-C₃)-alkyl, heteroaryl, heteroaryl-(C₁-C₃)-alkyl, heterocyclyl or heterocyclyl-(C₁-C₃)-alkyl, wherein each of the last-mentioned 10 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and wherein heterocyclyl has q oxo groups,
or
R¹⁰ and R¹¹, together with the sulphur atom to which they are attached, form a 3- to 8-membered unsaturated, partially saturated or saturated ring, which comprises in each case, in addition to the carbon atoms and in addition to the sulphur atom, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
R¹² is hydrogen, (C₁-C₁₂)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, (C₃-C₈)-cycloalkyl, (C₃-C₈)-halocycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₁-C₁₂)-alkylcarbonyl or (C₁-C₁₂)-haloalkylcarbonyl,
R¹³, R¹⁴ are each independently hydrogen, (C₁-C₁₂)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, (C₁-C₁₂)-alkylcarbonyl, (C₂-C₁₂)-alkenylcarbonyl, (C₂-C₁₂)-alkynylcarbonyl, (C₁-C₁₂)-haloalkylcarbonyl, (C₁-C₄)-alkylsulphonyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₃-C₈)-cycloalkylcarbonyl, (C₃-C₈)-cycloalkenylcarbonyl, (C₃-C₈)-cycloalkyl-(C₁-C₆)-alkylcarbonyl, (C₃-C₈)-cycloalkenyl-(C₁-C₆)-alkylcarbonyl, aryl, arylcarbonyl, arylsulphonyl, hetaryl, hetarylcarbonyl, hetarylsulphonyl, heterocyclyl, heterocyclylcarbonyl, heterocyclylsulphonyl, wherein each of the last-mentioned 17 residues is unsubstituted or is substituted by one or more residues from the group consisting of halogen, nitro, hydroxyl, cyano, NH₂, (C₁-C₆)-alkylamine, (C₁-C₆)-dialkylamine, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl and wherein heterocyclyl has q oxo groups,
or
R¹³ and R¹⁴, together with the nitrogen atom to which they are attached, form a 3- to 8-membered unsaturated, partially saturated or saturated ring, which comprises in each case, in addition to the carbon atoms and in addition to the nitrogen atom, p ring members from the group consisting of N(R¹²)ₘ, O and S(O)ₙ and wherein said ring is unsubstituted or is substituted by one or more residues from the group consisting of halogen, nitro, hydroxyl, cyano, NH₂, (C₁-C₆)-alkylamine, (C₁-C₆)-dialkylamine, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl and has q oxo groups,
n is independently selected from 0, 1 or 2,
m is independently selected from 0 or 1,
p is independently selected from 0, 1, 2 or 3,
q is independently selected from 0, 1 or 2,
y is 0 or 1,
for controlling harmful microorganisms in crop protection and/or in the protection of materials, optionally for controlling fungi.

2. Compound of the formula (G) and/or a salt thereof, 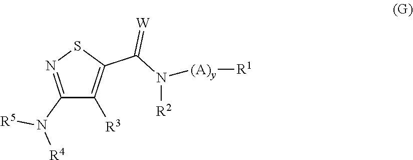
in which
A is CR⁶R⁷,
W is O or S,
R¹ is hydrogen, (C₁-C₁₂)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, NR¹³R¹⁴, R¹³R¹⁴N—(C₁-C₆)-alkyl, (C₁-C₆)-alkoxy, (C₁-C₆)-haloalkoxy, (C₁-C₆)-haloalkoxy-(C₁-C₃)-alkyl, (C₁-C₆)-alkoxy-(C₁-C₃)-alkyl, (C₁-C₆)-alkoxy-(C₂-C₆)-alkoxy-(C₁-C₃)-alkyl, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-haloalkylthio-(C₁-C₃)-alkyl, (C₁-C₄)-haloalkylsulphoxy-(C₁-C₃)-alkyl, (C₁-C₄)-haloalkylsulphonyl-(C₁-C₃)-alkyl, (C₃-C₁₂)-cycloalkyl, (C₃-C₈)-cycloalkenyl, (C₃-C₁₂)-cycloalkyl-(C₁-C₆)-alkyl, (C₃-C₈)-cycloalkenyl-(C₁-C₆)-alkyl, (C₃-C₈)-cycloalkoxy, (C₃-C₈)-cycloalkyl-(C₁-C₆)-alkoxy, aryl, aryl-(C₁-C₃)-alkyl, heteroaryl, heteroaryl-(C₁-C₃)-alkyl, heterocyclyl, heterocyclyl-(C₁-C₃)-alkyl, aryloxy, heteroaryloxy, heterocyclyloxy, a bicyclic or a heterobicyclic residue, wherein each of the last-mentioned 17 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl, and wherein heterocyclyl has q oxo groups, and wherein each of the aforementioned heterocyclic residues, in addition to the carbon atoms, has in each case p ring members from the group consisting of N(R¹²)ₘ, O and S(O)ₙ,
R² is hydrogen, (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₄)-alkylsulphonyl, (C₁-C₄)-haloalkylsulphonyl, (C₁-C₆)-alkylcarbonyl, (C₂-C₆)-alkenylcarbonyl, (C₂-C₆)-alkynylcarbonyl, (C₁-C₆)-haloalkylcarbonyl, (C₂-C₆)-haloalkenylcarbonyl, (C₂-C₆)-haloalkynylcarbonyl, (C₁-C₆)-alkoxycarbonyl, di((C₁-C₆)-alkyl)aminocarbonyl, (C₃-C₈)-cycloalkyl, (C₃-C₈)-cycloalkyl-(C₁-C₆)-alkyl, (C₃-C₈)-cycloalkylcarbonyl, (C₃-C₈)-cycloalkyl-(C₁-C₆)-alkylcarbonyl, heteroarylcarbonyl, or arylcarbonyl, wherein each of the last-mentioned 6 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl,
or
R¹ and R², together with the nitrogen atom and (A)_{y} attached thereto form a 5- or 6-membered heterocyclic or heteroaromatic ring, which comprises in each case, in addition to the carbon atoms and the nitrogen atom, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
R³ is hydrogen, halogen, azido, isocyanate, isothiocyanate, nitro, cyano, hydroxyl, NR¹³R¹⁴, tri(C₁-C₆)-alkylsilyl, (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₁-C₆)-haloalkoxy, (C₁-C₆)-haloalkoxy-(C₁-C₃)-alkyl, (C₁-C₆)-alkoxy-(C₁-C₃)-alkyl, (C₁-C₆)-alkoxy-(C₂-C₆)-alkoxy-(C₁-C₃)-alkyl, (C₁-C₆)-alkylcarbonyloxy, (C₁-C₆)-haloalkylcarbonyloxy, (C₂-C₆)-alkenylcarbonyloxy, (C₂-C₆)-alkynylcarbonyloxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-haloalkylthio-(C₁-C₃)-alkyl, (C₁-C₄)-haloalkylsulphoxy-(C₁-C₃)-alkyl, (C₁-C₄)-haloalkylsulphonyl-(C₁-C₃)-alkyl, (C₁-C₆)-alkoxycarbonyl, (C₁-C₆)-haloalkoxycarbonyl, (C₂-C₆)-alkenyloxycarbonyl, (C₂-C₆)-haloalkenyloxycarbonyl, (C₂-C₆)-alkynyloxycarbonyl, (C₂-C₆)-haloalkynyloxycarbonyl, (C₁-C₆)-alkylcarbonyl, (C₁-C₆)-haloalkylcarbonyl, (C₂-C₆)-alkenylcarbonyl, (C₂-C₆)-haloalkenylcarbonyl, (C₂-C₆)-alkynylcarbonyl, (C₂-C₆)-haloalkynylcarbonyl, R¹³R¹⁴N-carbonyl, arylthio, arylsulphoxy, arylsulphonyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₃-C₈)-cycloalkoxy, aryl, aryloxy, arylcarbonyloxy, aryl-(C₁-C₃)-alkyl, heteroaryl, heteroaryloxy, heteroaryl-(C₁-C₃)-alkyl, heterocyclyl, heterocyclyloxy, or heterocyclyl-(C₁-C₃)-alkyl, wherein each of the last-mentioned 18 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl, and wherein heterocyclyl has q oxo groups,
R⁴, R⁵ are each independently hydrogen, (C₁-C₁₂)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, (C₁-C₆)-alkoxy-(C₁-C₃)-alkyl, (C₁-C₆)-alkoxy-(C₂-C₆)-alkoxy-(C₁-C₃)-alkyl, (C₁-C₆)-alkoxy-(C₁-C₃)-alkylcarbonyl, (C₁-C₆)-alkoxy-(C₂-C₆)-alkoxy-(C₁-C₃)-alkylcarbonyl, (C₁-C₆)-alkoxycarbonyl, (C₁-C₆)-haloalkoxycarbonyl, (C₂-C₆)-al kenyloxycarbonyl, (C₂-C₆)-haloalkenyloxycarbonyl, (C₂-C₆)-alkynyloxycarbonyl, (C₂-C₆)-haloalkynyloxycarbonyl, (C₁-C₆)-alkylcarbonyl, (C₁-C₆)-haloalkylcarbonyl, (C₂-C₆)-alkenylcarbonyl, (C₂-C₆)-haloalkenylcarbonyl, (C₂-C₆)-alkynylcarbonyl, (C₂-C₆)-haloalkynylcarbonyl, R¹³R¹⁴N-carbonyl, (C₁-C₄)-alkylthio, (C₁-C₄)-haloalkylthio, (C₁-C₈)-alkylthiocarbonyl, (C₁-C₈)-haloalkylthiocarbonyl, (C₁-C₄)-alkylsulphoxy, (C₁-C₄)-haloalkylsulphoxy, (C₁-C₄)-alkylsulphonyl, (C₁-C₄)-haloalkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-alkylthio-(C₁-C₃)-alkylcarbonyl, (C₁-C₄)-alkylsulphoxy-(C₁-C₃)-alkylcarbonyl, (C₁-C₄)-alkylsulphonyl-(C₁-C₃)-alkylcarbonyl, (C₁-C₁₂)-alkylcarbonyl, (C₁-C₁₂)-haloalkylcarbonyl, (C₂-C₁₂)-alkenylcarbonyl, (C₂-C₁₂)-haloalkenylcarbonyl, (C₂-C₁₂)-alkynylcarbonyl, (C₂-C₁₂)-haloalkynylcarbonyl, (C₁-C₁₂)-alkoxycarbonylcarbonyl, (C₁-C₁₂)-alkoxycarbonyl-(C₁-C₃)-alkylcarbonyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₃-C₈)-cycloalkylcarbonyl, (C₃-C₈)-cycloalkenylcarbonyl, (C₃-C₈)-cycloalkyl-(C₁-C₆)-alkylcarbonyl, (C₃-C₈)-cycloalkenyl-(C₁-C₆)-alkylcarbonyl, aryl, aryl-(C₁-C₃)-alkyl, heteroaryl, heteroaryl-(C₁-C₃)-alkyl, heterocyclyl, heterocyclyl-(C₁-C₃)-alkyl, arylcarbonyl, aryl-(C₁-C₆)-alkylcarbonyl, heteroarylcarbonyl, heteroaryl-(C₁-C₆)-alkylcarbonyl, heterocyclylcarbonyl, or heterocyclyl-(C₁-C₆)-alkylcarbonyl, wherein each of the last-mentioned 20 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl, and wherein heterocyclyl has q oxo groups,
or
NR⁴R⁵ is —N═CR⁸R⁹ or —N═S(O)ₙR¹⁰R¹¹,
R⁶, R⁷ are each independently hydrogen, cyano, halogen, (C₁-C₆)-alkyl, (C₂-C₆)-alkenyl, (C₂-C₆)-alkynyl, or (C₃-C₈)-cycloalkyl,
or
R⁶ and R⁷, together with the carbon atom to which they are attached, form a 3-6-membered carbocyclic or heterocyclic ring, which comprises in each case, in addition to the carbon atoms, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
R⁸, R⁹ are each independently hydrogen, (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₁-C₆)-haloalkoxy, (C₁-C₆)-haloalkoxy-(C₁-C₃)-alkyl, (C₂-C₆)-alkenyloxy, (C₂-C₆)-haloalkenyloxy, (C₂-C₆)-alkynyloxy, (C₂-C₆)-haloalkynyloxy, NR¹³R¹⁴, (C₁-C₆)-alkoxy-(C₁-C₃)-alkyl, halogen-(C₁-C₆)-alkoxy-(C₁-C₆)-alkyl, (C₁-C₆)-alkoxy-(C₂-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, aryl, aryl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 10 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
or
R⁸ and R⁹, together with the carbon atom to which they are attached, form a 3- to 8-membered unsaturated, partially saturated or saturated ring, which comprises in each case, in addition to the carbon atoms, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-(C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
R¹⁰, R¹¹ are each independently (C₁-C₆)-alkyl, (C₁-C₆)-haloalkyl, (C₂-C₆)-alkenyl, (C₂-C₆)-haloalkenyl, (C₂-C₆)-alkynyl, (C₂-C₆)-haloalkynyl, (C₁-C₆)-alkoxy-(C₁-C₃)-alkyl, halogen-(C₁-C₆)-alkoxy-(C₁-C₆)-alkyl, (C₁-C₆)-alkoxy-(C₂-C₆)-alkoxy-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, aryl, aryl-(C₁-C₃)-alkyl, heteroaryl, heteroaryl-(C₁-C₃)-alkyl, heterocyclyl or heterocyclyl-(C₁-C₃)-alkyl, wherein each of the last-mentioned 10 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and wherein heterocyclyl has q oxo groups,
or
R¹⁰ and R¹¹, together with the sulphur atom to which they are attached, form a 3- to 8-membered unsaturated, partially saturated or saturated ring, which comprises in each case, in addition to the carbon atoms and in addition to the sulphur atom, p ring members from the group consisting of N(R¹²)ₘ, O and S(O)ₙ and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl, R¹³R¹⁴N-carbonyl and has q oxo groups,
R¹² is hydrogen, (C₁-C₁₂-alkyl, (C₁-C₁₂)-haloalkyl, (C₂-C₁₂)-alkenyl, (C₂-C₁₂)-haloalkenyl, (C₂-C₁₂)-al kynyl, (C₂-C₁₂)-haloalkynyl, (C₃-C₈)-cycloalkyl, (C₃-C₈)-halocycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₁-C₁₂)-alkylcarbonyl or (C₁-C₁₂)-haloalkylcarbonyl,
R¹³, R¹⁴ are each independently hydrogen, (C₁-C₁₂-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, alkylcarbonyl, (C₂-C₁₂)-alkenylcarbonyl, (C₂-C₁₂)-alkynylcarbonyl, haloalkylcarbonyl, (C₁-C₄)-alkylsulphonyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₃-C₈)-cycloalkylcarbonyl, (C₃-C₈)-cycloalkenylcarbonyl, (C₃-C₈)-cycloalkyl-(C₁-C₆)-alkylcarbonyl, (C₃-C₈)-cycloalkenyl-(C₁-C₆)-alkylcarbonyl, aryl, arylcarbonyl, arylsulphonyl, hetaryl, hetarylcarbonyl, hetarylsulphonyl, heterocyclyl, heterocyclylcarbonyl, heterocyclylsulphonyl, wherein each of the last-mentioned 17 residues is unsubstituted or is substituted by one or more residues from the group consisting of halogen, nitro, hydroxyl, cyano, NH₂, (C₁-C₆)-alkylamine, (C₁-C₆)-dialkylamine, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl and wherein heterocyclyl has q oxo groups,
or
R¹³ and R¹⁴, together with the nitrogen atom to which they are attached, form a 3- to 8-membered unsaturated, partially saturated or saturated ring, which comprises in each case, in addition to the carbon atoms and in addition to the nitrogen atom, p ring members from the group consisting of N(R¹²)ₘ, O and S(O)ₙ and wherein said ring is unsubstituted or is substituted by one or more residues from the group consisting of halogen, nitro, hydroxyl, cyano, NH₂, (C₁-C₆)-alkylamine, (C₁-C₆)-dialkylamine, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl and has q oxo groups,
n is independently selected from 0, 1 or 2,
m is independently selected from 0 or 1,
p is independently selected from 0, 1, 2 or 3,
q is independently selected from 0, 1 or 2,
y is 0 or 1,
with the proviso that:
the compound of formula (G) is not 3-amino-5-(morpholin-4-ylcarbonothioyI)-1,2-thiazole-4-carbonitrile,
and
y is 1, if R¹ is a substituted 4-heptafluoroisopropylphenyl residue, a substituted 4-(nonafluoro-2-butyl)phenyl residue, a substituted 4-(1,1,2,3,3,3-hexafluoropropoxy)phenyl residue, a 2-bromo-4-methyl-6-(heptafluoroisopropyl)pyridin-3-yl residue or a 2-bromo-4-methyl-6-(2,2,2-trifluoro-1-trifluoromethylethoxy)pyridin-3-yl residue.

3. Compound of the formula (G) according to claim 2 and/or a salt thereof, in which
A is CR⁶R⁷,
W is O or S,
R¹ is hydrogen, (C₁-C₆)-alkyl, (C₁-C₆)-haloalkyl, (C₂-C₆)-alkenyl, (C₂-C₆)-alkynyl, NR¹³R¹⁴, R¹³R¹⁴N-(C₁-C₆)-alkyl, (C₁-C₆)-alkoxy, (C₁-C₆)-haloalkoxy, (C₁-C₆)-haloalkoxy-(C₁-C₃)-alkyl, (C₁-C₆)-alkoxy-(C₁-C₃)-alkyl, (C₁-C₆)-alkoxy-(C₂-C₆)-alkoxy-(C₁-C₃)-alkyl, (C₁-C₄)-alkylthio, (C₁-C₄)-alkylsulphoxy, (C₁-C₄)-alkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₆)-cycloalkyl, (C₃-C₆)-cycloalkenyl, (C₃-C₆)-cycloalkyl-(C₁-C₃)-alkyl, (C₃-C₆)-cycloalkenyl-(C₁-C₃)-alkyl, (C₃-C₆)-cycloalkoxy, phenyl, heteroaryl, heterocyclyl, phenoxy, heteroaryloxy, heterocyclyloxy or a carbobicyclic residue, wherein each of the last-mentioned 12 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
R² is hydrogen, (C₁-C₆)-alkyl, (C₂-C₆)-alkenyl, (C₂-C₆)-alkynyl, (C₁-C₄)-alkylsulphonyl, (C₁-C₄)-haloalkylsulphonyl, (C₁-C₆)-alkylcarbonyl, (C₂-C₆)-alkenylcarbonyl, (C₂-C₆)-alkynylcarbonyl, (C₁-C₆)-alkoxycarbonyl, di((C₁-C₆)-alkyl)aminocarbonyl, (C₃-C₈)-cycloalkylcarbonyl, heteroarylcarbonyl or phenylcarbonyl, wherein each of the last-mentioned 3 residues is unsubstituted or is substituted by one or more residues from the group consisting of halogen, nitro, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl,
R³ is halogen, (C₁-C₄)-alkyl, (C₁-C₄)-haloalkyl, (C₂-C₄)-alkenyl, (C₂-C₄)-haloalkenyl, (C₂-C₄)-alkynyl, (C₁-C₆)-alkoxy, (C₁-C₆)-haloalkoxy, (C₁-C₆)-haloalkoxy-(C₁-C₃)-alkyl, (C₁-C₄)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₃-C₈)-cycloalkyl, phenyl, phenyloxy, phenylthio, phenylsulphoxy, phenylsulphonyl, wherein each of the last-mentioned 6 residues is unsubstituted or is substituted by one or more residues from the group consisting of halogen, (C₁-C₄)-alkyl, (C₁-C₄)-haloalkyl, (C₁-C₄)-alkoxy, (C₁-C₄)-haloalkoxy, R⁴, R⁵ are each independently hydrogen, (C₁-C₆)-alkyl, (C₁-C₆)-haloalkyl, (C₂-C₆)-alkenyl, (C₂-C₆)-alkynyl, (C₁-C₆)-alkoxy-(C₁-C₃)-alkyl, (C₁-C₆)-alkoxy-(C₁-C₃)-alkylcarbonyl, (C₁-C₄)-alkylthio, (C₁-C₄)-haloalkylthio, (C₁-C₄)-alkylthiocarbonyl, (C₁-C₄)-haloalkylthiocarbonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-alkylthio-(C₁-C₃)-alkylcarbonyl, (C₁-C₄)-alkylsulphoxy-(C₁-C₃)-alkylcarbonyl, (C₁-C₄)-alkylsulphonyl-(C₁-C₃)-alkylcarbonyl, (C₁-C₆)-alkylcarbonyl, (C₁-C₆)-haloalkylcarbonyl, (C₂-C₆)-alkenylcarbonyl, (C₂-C₆)-alkynylcarbonyl, (C₁-C₆)-alkoxycarbonylcarbonyl, (C₁-C₆)-alkoxycarbonyl-(C₁-C₃)-alkylcarbonyl, (C₁-C₆)-alkoxycarbonyl, (C₁-C₆)-haloalkoxycarbonyl, (C₂-C₆)-alkenyloxycarbonyl, (C₃-C₆)-cycloalkyl, (C₃-C₆)-cycloalkyl-(C₁-C₆)-alkyl, (C₃-C₆)-cycloalkylcarbonyl, (C₃-C₆)-cycloalkyl-(C₁-C₆)-alkylcarbonyl, phenyl, phenyl-(C₁-C₃)-alkyl, heteroaryl, heteroaryl-(C₁-C₃)-alkyl, heterocyclyl, heterocyclyl-(C₁-C₃)-alkyl, phenylcarbonyl, phenyl-(C₁-C₆)-alkylcarbonyl, hetarylcarbonyl, hetaryl-(C₁-C₆)-alkylcarbonyl, heterocyclylcarbonyl, heterocyclyl-(C₁-C₆)-alkylcarbonyl, wherein each of the last-mentioned 16 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
or
NR⁴R⁵ is —N═CR⁸R⁹ or —N═S(O)ₙR¹⁹R¹¹,
R⁶, R⁷ are each independently hydrogen or (C₁-C₆)-alkyl,
R⁸, R⁹ are each independently hydrogen, (C₁-C₆)-alkyl, (C₁-C₆)-haloalkyl, (C₂-C₆)-alkenyl, (C₂-C₆)-alkynyl, (C₁-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₆)-alkoxy, (C₁-C₆)-haloalkoxy, (C₂-C₆)-alkenyloxy, NR¹³R¹⁴, (C₃-C₈)-cycloalkyl, (C₃-C₈)-cycloalkyl-(C₁-C₆)-alkyl, phenyl, phenyl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 8 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
or
R⁸ and R⁹, together with the carbon atom to which they are attached, form a 3- to 6-membered unsaturated, partially saturated or saturated ring, which comprises in each case, in addition to the carbon atoms, p ring members from the group consisting of N(R¹²)ₘ, O and S(O)ₙ, and wherein said ring is unsubstituted or is substituted by one or more residues from the group consisting of halogen, nitro, hydroxyl, cyano, NR¹³R¹⁴, (C₁-(C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
R¹⁰, R¹¹ are each independently, (C₁-C₆)-alkyl, (C₂-C₆)-alkenyl, (C₂-C₆)-alkynyl, (C₁-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₈)-cycloalkyl, (C₃-C₈)-cycloalkyl-(C₁-C₆)-alkyl, phenyl, phenyl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 8 residues is unsubstituted or is substituted by one or more residues from the group consisting of halogen, nitro, hydroxyl, cyano, NR¹³R¹⁴, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
or
R¹⁰ and R¹¹, together with the sulphur atom to which they are attached, form a 3- to 6-membered unsaturated, partially saturated or saturated ring, which comprises in each case, in addition to the carbon atoms and in addition to the sulphur atom, p ring members from the group consisting of N(R¹²)ₘ, O and S(O)ₙ, and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy or (C₁-C₄)-alkylsulphonyl, and wherein heterocyclyl has q oxo groups,
R¹² is hydrogen, (C₁-C₆)-alkyl or (C₁-C₆)-alkylcarbonyl,
R¹³, R¹⁴ are each independently hydrogen, (C₁-C₆)-alkyl, (C₁-C₆)-alkylcarbonyl, (C₁-C₄)-alkylsulphonyl, phenyl, phenylcarbonyl, wherein each of the last-mentioned two residues is unsubstituted or is substituted by one or more residues from the group consisting of halogen, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl,
or
R¹³ and R¹⁴, together with the nitrogen atom to which they are attached, form a 3- to 8-membered unsaturated, partially saturated or saturated ring, which comprises in each case, in addition to the carbon atoms and in addition to the nitrogen atom, p ring members from the group consisting of N(R¹²)ₘ, O and S(O)ₙ, and wherein said ring is unsubstituted or is substituted by one or more residues from the group consisting of halogen, hydroxyl, (C₁-C₄)-alkyl, (C₁-C₄)-haloalkyl, (C₁-C₄)-alkoxy, (C₁-C₄)-haloalkoxy, and has q oxo groups,
n is independently selected from 0, 1 or 2,
m is independently selected from 0 or 1,
p is independently selected from 0, 1 or 2,
q is independently selected from 0, 1 or 2,
y is 0 or 1,
with the proviso that:
y is 1, if R¹ is a substituted 4-heptafluoroisopropylphenyl residue, a substituted 4-(nonafluoro-2-butyl)phenyl residue, a substituted 4-(1,1,2,3,3,3-hexafluoropropoxy)phenyl residue, a 2-bromo-4-methyl-6-(heptafluoroisopropyl)pyridin-3-yl residue or a 2-bromo-4-methyl-6-(2,2,2-trifluoro-1-trifluoromethylethoxy)pyridin-3-yl residue.

4. Compound of the formula (G) according to claim 2, and/or a salt thereof, in which
A is CR⁶R⁷,
W is O or S,
R¹ is (C₁-C₆)-alkyl, (C₁-C₆)-haloalkyl, (C₂-C₆)-alkenyl, (C₂-C₆)-alkynyl, NR¹³R¹⁴, R¹³R¹⁴N—(C₁-C₆)-alkyl, (C₁-C₆)-alkoxy, (C₁-C₆)-haloalkoxy, (C₁-C₆)-alkoxy-(C₁-C₃)-alkyl, (C₁-C₆)-alkoxy-(C₂-C₆)-alkoxy-(C₁-C₃)-alkyl, (C₁-C₄)-alkylthio, (C₁-C₄)-alkylsulphoxy, (C₁-C₄)-alkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₆)-cycloalkyl, (C₃-C₆)-cycloalkenyl, (C₃-C₆)-cycloalkyl-(C₁-C₃)-alkyl, (C₃-C₆)-cycloalkenyl-(C₁-C₃)-alkyl, (C₃-C₆)-cycloalkoxy, phenyl, heteroaryl, heterocyclyl, phenoxy, heteroaryloxy, heterocyclyloxy or a carbobicyclic residue, wherein each of the last-mentioned 12 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
R² is hydrogen, (C₁-C₆)-alkyl, (C₂-C₆)-alkenyl, (C₂-C₆)-alkynyl, (C₁-C₄)-alkylsulphonyl, (C₁-C₄)-haloalkylsulphonyl, (C₁-C₆)-alkylcarbonyl, (C₂-C₆)-alkenylcarbonyl, (C₂-C₆)-alkynylcarbonyl, (C₁-C₆)-alkoxycarbonyl, di((C₁-C₆)-alkyl)aminocarbonyl, (C₃-C₈)-cycloalkylcarbonyl, heteroarylcarbonyl or phenylcarbonyl, wherein each of the last-mentioned 3 residues is unsubstituted or is substituted by one or more residues from the group consisting of halogen, nitro, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl,
R³ is halogen, (C₁-C₄)-haloalkyl, (C₂-C₄)-alkynyl, (C₁-C₄)-alkoxy, (C₁-C₄)-haloalkoxy, methylthio, (C₃-C₈)-cycloalkyl, phenyl, phenyloxy, wherein each of the last-mentioned 3 residues is unsubstituted or is substituted by one or more residues from the group consisting of halogen, (C₁-C₄)-alkyl, (C₁-C₄)-haloalkyl, (C₁-C₄)-alkoxy, (C₁-C₄)-haloalkoxy, R⁴, R⁵ are each independently hydrogen, (C₁-C₆)-alkyl, (C₁-C₆)-haloalkyl, (C₂-C₆)-alkenyl, (C₂-C₆)-alkynyl, (C₁-C₆)-alkoxy-(C₁-C₃)-alkyl, (C₁-C₆)-alkoxy-(C₁-C₃)-alkylcarbonyl, (C₁-C₄)-alkylthio, (C₁-C₄)-haloalkylthio, (C₁-C₄)-alkylthiocarbonyl, (C₁-C₄)-haloalkylthiocarbonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₄)-alkylthio-(C₁-C₃)-alkylcarbonyl, (C₁-C₄)-alkylsulphoxy-(C₁-C₃)-alkylcarbonyl, (C₁-C₄)-alkylsulphonyl-(C₁-C₃)-alkylcarbonyl, (C₁-C₆)-alkylcarbonyl, (C₁-C₆)-haloalkylcarbonyl, (C₂-C₆)-alkenylcarbonyl, (C₂-C₆)-alkynylcarbonyl, (C₁-C₆)-alkoxycarbonylcarbonyl, (C₁-C₆)-alkoxycarbonyl-(C₁-C₃)-alkylcarbonyl, (C₁-C₆)-alkoxycarbonyl, (C₁-C₆)-haloalkoxycarbonyl, (C₂-C₆)-alkenyloxycarbonyl, (C₃-C₆)-cycloalkyl, (C₃-C₆)-cycloalkyl-(C₁-C₆)-alkyl, (C₃-C₆)-cycloalkylcarbonyl, (C₃-C₆)-cycloalkyl-(C₁-C₆)-alkylcarbonyl, phenyl, phenyl-(C₁-C₃)-alkyl, heteroaryl, heteroaryl-(C₁-C₃)-alkyl, heterocyclyl, heterocyclyl-(C₁-C₃)-alkyl, phenylcarbonyl, phenyl-(C₁-C₆)-alkylcarbonyl, hetarylcarbonyl, hetaryl-(C₁-C₆)-alkylcarbonyl, heterocyclylcarbonyl, heterocyclyl-(C₁-C₆)-alkylcarbonyl, wherein each of the last-mentioned 16 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
or
NR⁴R⁵ is —N═CR⁸R⁹ or —N═S(O)ₙR¹⁰R¹¹,
R⁶, R⁷ are each independently hydrogen or (C₁-C₄)-alkyl,
R⁸, R⁹ are each independently hydrogen, (C₁-C₆)-alkyl, (C₁-C₆)-haloalkyl, (C₂-C₆)-alkenyl, (C₂-C₆)-alkynyl, (C₁-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₆)-alkoxy, (C₁-C₆)-haloalkoxy, (C₂-C₆)-alkenyloxy, NR¹³R¹⁴, (C₃-C₈)-cycloalkyl, (C₃-C₈)-cycloalkyl-(C₁-C₆)-alkyl, phenyl, phenyl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 8 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
or
R⁸ and R⁹, together with the carbon atom to which they are attached, form a 3- to 6-membered unsaturated, partially saturated or saturated ring, which comprises in each case, in addition to the carbon atoms, p ring members from the group consisting of N(R¹²)ₘ, O and S(O)ₙ, and wherein said ring is unsubstituted or is substituted by one or more residues from the group consisting of halogen, nitro, hydroxyl, cyano, NR¹³R¹⁴, (C₁-(C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
R¹⁰, R¹¹ are each independently, (C₁-C₆)-alkyl, (C₂-C₆)-alkenyl, (C₂-C₆)-alkynyl, (C₁-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₈)-cycloalkyl, (C₃-C₈)-cycloalkyl-(C₁-C₆)-alkyl, phenyl, phenyl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 8 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
or
R¹⁰ and R¹¹, together with the sulphur atom to which they are attached, form a 3- to 6-membered unsaturated, partially saturated or saturated ring, which comprises in each case, in addition to the carbon atoms and in addition to the sulphur atom, p ring members from the group consisting of N(R¹²)ₘ, O and S(O)ₙ, and wherein said ring is unsubstituted or is substituted by one or more residues from the group consisting of halogen, nitro, hydroxyl, cyano, NR¹³R¹⁴, C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy or (C₁-C₄)-alkylsulphonyl, and wherein heterocyclyl has q oxo groups,
R¹² is hydrogen, (C₁-C₆)-alkyl or (C₁-C₆)-alkylcarbonyl,
R¹³, R¹⁴ are each independently hydrogen, (C₁-C₆)-alkyl, (C₁-C₆)-alkylcarbonyl, (C₁-C₄)-alkylsulphonyl, phenyl, phenylcarbonyl, wherein each of the last-mentioned two residues is unsubstituted or is substituted by one or more residues from the group consisting of halogen, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl,
or
R¹³ and R¹⁴, together with the nitrogen atom to which they are attached, form a 3- to 8-membered unsaturated, partially saturated or saturated ring, which comprises in each case, in addition to the carbon atoms and in addition to the nitrogen atom, p ring members from the group consisting of N(R¹²)ₘ, O and S(O)ₙ, and wherein said ring is unsubstituted or is substituted by one or more residues from the group consisting of halogen, hydroxyl, (C₁-C₄)-alkyl, (C₁-C₄)-haloalkyl, (C₁-C₄)-alkoxy, (C₁-C₄)-haloalkoxy, and has q oxo groups,
n is independently selected from 0, 1 or 2,
m is independently selected from 0 or 1,
p is independently selected from 0, 1 or 2,
q is independently selected from 0 or 1,
y is 0 or 1,
with the proviso that:
y is 1, if R¹ is a substituted phenyl residue or a substituted pyridin-3-yl residue.

5. Compound of the formula (G) according to claim 2, and/or a salt thereof, in which
A is CR⁶R⁷,
W is O or S,
R¹ is (C₁-C₆)-alkyl, (C₁-C₆)-haloalkyl, (C₂-C₆)-alkenyl, (C₂-C₆)-alkynyl, NR¹³R¹⁴, R¹³R¹⁴N—(C₁-C₆)-alkyl, (C₁-C₆)-alkoxy, (C₁-C₆)-haloalkoxy, (C₁-C₆)-alkoxy-(C₁-C₃)-alkyl, (C₁-C₆)-alkoxy-(C₂-C₆)-alkoxy-(C₁-C₃)-alkyl, (C₁-C₄)-alkylthio, (C₁-C₄)-alkylsulphoxy, (C₁-C₄)-alkylsulphonyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₆)-cycloalkyl, (C₃-C₆)-cycloalkenyl, (C₃-C₆)-cycloalkyl-(C₁-C₃)-alkyl, (C₃-C₆)-cycloalkenyl-(C₁-C₃)-alkyl, (C₃-C₆)-cycloalkoxy, phenyl, heteroaryl, heterocyclyl, phenoxy, heteroaryloxy, heterocyclyloxy or a carbobicyclic residue, wherein each of the last-mentioned 12 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
R² is hydrogen, (C₁-C₆)-alkylcarbonyl, (C₂-C₆)-alkenylcarbonyl, (C₂-C₆)-alkynylcarbonyl, (C₁-C₆)-alkoxycarbonyl, (C₃-C₆)-cycloalkylcarbonyl, heteroarylcarbonyl, or phenylcarbonyl, wherein each of the last-mentioned 3 residues is unsubstituted or is substituted by one or more residues from the group consisting of halogen, nitro, cyano, NR¹³R¹⁴, (C₁C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl,
R³ is halogen, methyl, difluoromethyl (CHF₂), trifluoromethyl (CF₃) or (C₂-C₃)-alkynyl, R⁴, R⁵ are each independently hydrogen, (C₂-C₆)-alkynyl, (C₁-C₄)-alkylthio, (C₁-C₄)-haloalkylthio, (C₁-C₄)-alkylthiocarbonyl, (C₁-C₄)-haloalkylthiocarbonyl, (C₁-C₆)-alkoxy-(C₁-C₃)-alkylcarbonyl, (C₁-C₄)-alkylthio-(C₁-C₃)-alkylcarbonyl, (C₁-C₄)-alkylsulphoxy-(C₁-C₃)-alkylcarbonyl, (C₁-C₄)-alkylsulphonyl-(C₁-C₃)-alkylcarbonyl, (C₁-C₆)-alkylcarbonyl, (C₁-C₆)-haloalkylcarbonyl, (C₂-C₆)-alkenylcarbonyl, (C₂-C₆)-alkynylcarbonyl, (C₁-C₆)-alkoxycarbonylcarbonyl, (C₁-C₆)-alkoxycarbonyl-(C₁-C₃)-alkylcarbonyl, (C₁-C₆)-alkoxycarbonyl, (C₁-C₆)-haloalkoxycarbonyl, (C₂-C₆)-alkenyloxycarbonyl, (C₃-C₆)-cycloalkylcarbonyl, (C₃-C₆)-cycloalkyl-(C₁-C₆)-alkylcarbonyl, phenylcarbonyl, phenyl-(C₁-C₆)-alkylcarbonyl, hetarylcarbonyl, hetaryl-(C₁-C₆)-alkylcarbonyl, heterocyclylcarbonyl, heterocyclyl-(C₁-C₆)-alkylcarbonyl, wherein each of the last-mentioned 8 residues is unsubstituted or is substituted by one or more residues from the group consisting of halogen, nitro, hydroxyl, cyano, NR¹³R¹⁴, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
or
NR⁴R⁵ is —N═CR⁸R⁹ or —N═S(O)ₙR¹⁹R¹¹,
R⁶ is hydrogen,
R⁷ is hydrogen or methyl,
R⁸, R⁹ are each independently hydrogen, (C₁-C₆)-alkyl, (C₁-C₆)-haloalkyl, (C₂-C₆)-alkenyl, (C₂-C₆)-alkynyl, (C₁-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₁-C₆)-alkoxy, (C₁-C₆)-haloalkoxy, (C₂-C₆)-alkenyloxy, NR¹³R¹⁴, (C₃-C₈)-cycloalkyl, (C₃-C₈)-cycloalkyl-(C₁-C₆)-alkyl, phenyl, phenyl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 8 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
or
R⁸ and R⁹, together with the carbon atom to which they are attached, form a 3- to 6-membered unsaturated, partially saturated or saturated ring, which comprises in each case, in addition to the carbon atoms, p ring members from the group consisting of N(R¹²)ₘ, O and S(O)ₙ, and wherein said ring is unsubstituted or is substituted by one or more residues from the group consisting of halogen, nitro, hydroxyl, cyano, NR¹³R¹⁴, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
R¹⁰, R¹¹ are each independently, (C₁-C₆)-alkyl, (C₂-C₆)-alkenyl, (C₂-C₆)-alkynyl, (C₁-C₆)-alkoxy-(C₁-C₃)-alkyl, (C₁-C₄)-alkylthio-(C₁-C₃)-alkyl, (C₁-C₄)-alkylsulphoxy-(C₁-C₃)-alkyl, (C₁-C₄)-alkylsulphonyl-(C₁-C₃)-alkyl, (C₃-C₈)-cycloalkyl, (C₃-C₈)-cycloalkyl-(C₁-C₆)-alkyl, phenyl, phenyl-(C₁-C₃)-alkyl, heteroaryl, heteroaryl-(C₁-C₃)-alkyl, heterocyclyl, heterocyclyl-(C₁-C₃)-alkyl, wherein each of the last-mentioned 8 residues is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl, and wherein heterocyclyl has q oxo groups,
or
R¹⁰ and R¹¹, together with the sulphur atom to which they are attached, form a 3- to 6-membered unsaturated, partially saturated or saturated ring, which comprises in each case, in addition to the carbon atoms and in addition to the sulphur atom, p ring members from the group consisting of N(R¹²)ₘ, O and S(O)ₙ, and wherein said ring is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy or (C₁-C₄)-alkylsulphonyl, and wherein heterocyclyl has q oxo groups,
R¹² is hydrogen, (C₁-C₆)-alkyl or (C₁-C₆)-alkylcarbonyl,
R¹³, R¹⁴ are each independently hydrogen, (C₁-C₆)-alkyl, (C₁-C₆)-alkylcarbonyl, (C₁-C₄)-alkylsulphonyl, phenyl, phenylcarbonyl, wherein each of the last-mentioned two residues is unsubstituted or is substituted by one or more residues from the group consisting of halogen, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, (C₁-C₄)-alkylsulphonyl,
or
R¹³ and R¹⁴, together with the nitrogen atom to which they are attached, form a 3- to 8-membered unsaturated, partially saturated or saturated ring, which comprises in each case, in addition to the carbon atoms and in addition to the nitrogen atom, p ring members from the group consisting of N(R¹²)ₘ, O and S(O)ₙ, and wherein said ring is unsubstituted or is substituted by one or more residues from the group consisting of halogen, hydroxyl, (C₁-C₄)-alkyl, (C₁-C₄)-haloalkyl, (C₁-C₄)-alkoxy, (C₁-C₄)-haloalkoxy, and has q oxo groups,
n is independently selected from 0, 1 or 2,
m is independently selected from 0 or 1,
p is independently selected from 0, 1 or 2,
q is independently selected from 0 or 1,
y is 0 or 1,
with the proviso that:
y is 1, if R¹ is a substituted phenyl residue or a substituted pyridin-3-yl residue.

6. Compound of the formula (G) according to claim 2, and/or a salt thereof, in which
R³ is halogen, trifluoromethyl or ethynyl.

7. Compound of the formula (G) according to claim 2, and/or a salt thereof, in which y is 1.

8. Compound of the formula (G) according to claim 2, and/or a salt thereof, in which y is 0.

9. Compound of the formula (G) according to claim 2, and/or a salt thereof, in which
n is independently selected from 0, 1 or 2, optionally independently selected from 0 or 1,
m is independently selected from 0 or 1, optionally m is 0,
p is independently selected from 0, 1 or 2, optionally p is independently selected from 0 or 1, and
q is independently selected from 0 or 1, optionally q is 0.

10. A product for controlling harmful microorganisms in crop protection and/or in the protection of materials, optionally for controlling fungi
comprising a according compound of the formula (G) and/or salt thereof as defined in claim 2.

11. Composition, comprising one or more compounds of the formula (G) and/or salts thereof as defined in claim 2,
and one or more further substances selected from groups (i) and/or (ii):
(i) one or more further agrochemically active substances, optionally selected from the group consisting of further fungicides, insecticides, acaricides, nematicides, herbicides, safeners, fertilizers and/or plant growth regulators,
(ii) one or more formulation auxiliaries customary in crop protection.

12. Method for controlling fungi and/or for controlling one or more plant diseases caused by fungal plant pathogens, comprisng applying an effective amount of
one or more compounds of the formula (G) and/or salts thereof, as defined in claim 2,
or
a composition thereof according to claim 11,
to the fungi, plant, to a portion of the plant and/or to plant seed.

13. Compound of the formula (Z-A), (Z-B) and/or a salt thereof, 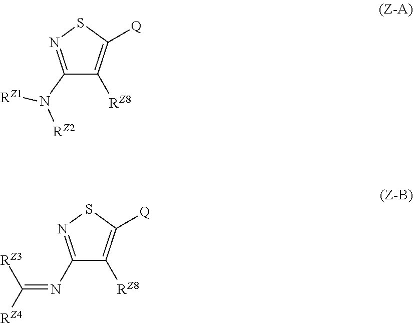
wherein
Q is hydrogen, CN, COCl, COF, CO₂H and salts thereof, CONR¹³R¹⁴, and CO₂R^{q}, wherein R^{q} is (C₁-C₉)-alkyl or (C₁-C₉)-haloalkyl,
R^{Z8} is selected from the group consisting of H, F, Cl, Br, I, CH₃, CH₂F, CHF₂ and CF₃,
R^{Z1} and R^{Z2} are each independently hydrogen, CN, CH₂aryl, X—C(═Y)—, wherein Y is NH, O or S and X is NH₂, OH, SH, (C₁-C₈)-alkyl, (C₁-C₈)-haloalkyl, (C₁-C₈)-alkoxy, (C₁-C₈)-haloalkoxy, (C₁-C₆)-alkylthio, HN(C₁-C₈)-alkyl, or aryl, wherein each aryl is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, and (C₁-C₄)-alkylsulphonyl,
R^{Z3} is hydrogen, (C₁-C₈)-alkyl, (C₁-C₈)-haloalkyl, (C₁-C₈)-alkoxy, (C₁-C₈)-haloalkoxy, (C₁-C₆)-alkylthio, or aryl, wherein aryl is unsubstituted or is substituted by one or more residues from the group consisting of halogen, nitro, hydroxyl, cyano, NR¹³R¹⁴, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, and (C₁-C₄)-alkylsulphonyl,
R^{Z4} is (C₁-C₈)-alkyl, (C₁-C₈)-haloalkyl, (C₁-C₈)-alkoxy, (C₁-C₈)-haloalkoxy, (C₁-C₆)-alkylthio, or aryl, wherein aryl is unsubstituted or is substituted by one or more residues from the group consisting of halogen, nitro, hydroxyl, cyano, NR¹³R¹⁴, C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylsulphoxy, and (C₁-C₄)-alkylsulphonyl,
wherein R¹³ and R¹⁴ are each independently hydrogen, (C₁-C₁₂)-alkyl, (C₁-C₁₂)-haloalkyl, (C₂-C₁₂)-alkenyl, (C₂-C₁₂)-haloalkenyl, (C₂-C₁₂)-alkynyl, (C₂-C₁₂)-haloalkynyl, (C₁-C₁₂)-alkylcarbonyl, (C₂-C₁₂)-alkenylcarbonyl, (C₂-C₁₂)-alkynylcarbonyl, (C₁-C₁₂)-haloalkylcarbonyl, (C₁-C₄)-alkylsulphonyl, (C₃-C₈)-cycloalkyl, (C₃-C₈)-cycloalkenyl, (C₃-C₈)-cycloalkyl-(C₁-C₆)-alkyl, (C₃-C₈)-cycloalkenyl-(C₁-C₆)-alkyl, (C₃-C₈)-cycloalkylcarbonyl, (C₃-C₈)-cycloalkenylcarbonyl, (C₃-C₈)-cycloalkyl-(C₁-C₆)-alkylcarbonyl, (C₃-C₈)-cycloalkenyl-(C₁-C₆)-alkylcarbonyl, aryl, arylcarbonyl, arylsulphonyl, hetaryl, hetarylcarbonyl, hetarylsulphonyl, heterocyclyl, heterocyclylcarbonyl, heterocyclylsulphonyl, wherein each of the last-mentioned 17 residues is unsubstituted or is substituted by one or more residues from the group consisting of halogen, nitro, hydroxyl, cyano, NH₂, (C₁-C₆)-alkylamine, (C₁-C₈)-dialkylamine, (C₁-C₄)-alkyl, (C₁-C₄)-haloalkyl, (C₁-C)-alkoxy, (C₁-C)-haloalkoxy, (C₁-C₄)-alkylthio, (C₁-C)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C₄)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl and wherein heterocyclyl has q oxo groups,
or
R¹³ and R¹⁴, together with the nitrogen atom to which they are attached, form a 3- to 8-membered unsaturated, partially saturated or saturated ring, which comprises in each case, in addition to the carbon atoms and in addition to the nitrogen atom, p ring members from the group consisting of N(R¹²)ₘ, O and S(O)ₙ and wherein said ring is unsubstituted or is substituted by one or more residues from the group consisting of halogen, nitro, hydroxyl, cyano, NH₂, (C₁-C₆)-alkylamine, (C₁-C₆)-dialkylamine, (C₁-C₄)-alkyl, (C₁-C)-haloalkyl, (C₁-C₄)-alkoxy, (C₁-C₄)-haloalkoxy, (C₁-C)-alkylthio, (C₁-C₄)-alkylsulphoxy, (C₁-C₄)-alkylsulphonyl, (C₁-C₄)-haloalkylthio, (C₁-C₄)-haloalkylsulphoxy, (C₁-C₄)-haloalkylsulphonyl, (C₁-C₄)-alkoxycarbonyl, (C₁-C)-haloalkoxycarbonyl, (C₁-C₄)-alkylcarboxy, (C₃-C₆)-cycloalkyl, (C₃-C₆)-cycloalkyl-(C₁-C₆)-alkyl, (C₁-C₄)-alkoxycarbonyl-(C₁-C₄)-alkyl, hydroxycarbonyl, hydroxycarbonyl-(C₁-C₄)-alkyl and has q oxo groups,
n is independently selected from 0, 1 or 2,
m is independently selected from 0 or 1,
p is independently selected from 0, 1, 2 or 3,
q is independently selected from 0, 1 or 2,
y is 0 or 1.

14. Compound of the formula (Z-1), (Z-2), (Z-3), (Z-4), (Z-5), (Z-6) and/or a salt thereof, 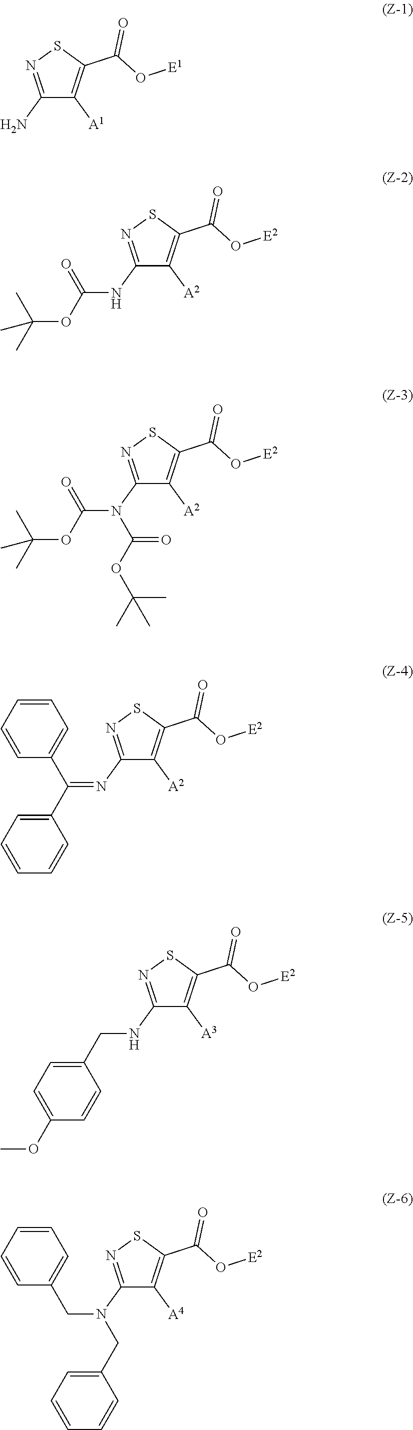
wherein
A¹ is selected from the group consisting of H, F, Cl, Br and I,
A² is selected from the group consisting of F, Cl, Br and I,
A³ is H or Cl,
A⁴ is H or Br,
E¹ is selected from the group consisting of H, methyl, ethyl and iso-propyl,
E² is selected from the group consisting of H, methyl, ethyl, iso-propyl, and tert.-butyl.

15. Process for preparing a compound of the formula (G) as defined in claim 2, and/or a salt thereof, comprising obtaining
(a) a compound of formula (G) wherein W is oxygen in a chemical synthesis comprising reacting a compound of formula (E-II) 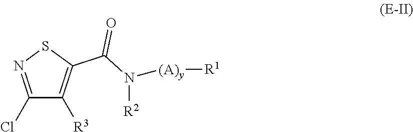
in which R¹, R², R³, A and y each have the meaning as defined in formula (G), and wherein R³ optionally represents a halogen atom,
with HNR⁴R⁵, wherein R⁴ and R⁵ each have the meaning as defined in formula (G),
or
(b) a compound of formula (G) wherein W is oxygen is obtained in a chemical synthesis comprising reacting a compound of formula (E-VIII) or of formula (E-XXVII) 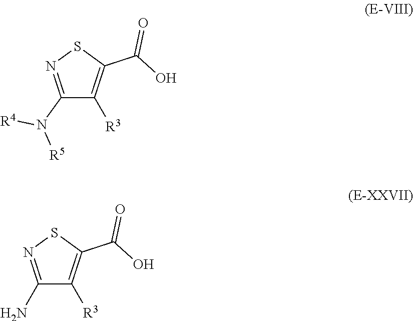
in which R³ has the meaning as defined in formula (G), and wherein R⁴ and R⁵ each have the meaning as defined in formula (G),
with a compound of formula (E-XXXII) 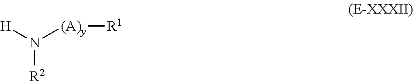
wherein y, A, R¹ and R² each have the meaning as defined in formula (G),
or
(c) a compound of formula (G), wherein W is sulphur is obtained in a chemical synthesis comprising reacting a compound of formula (E-V) 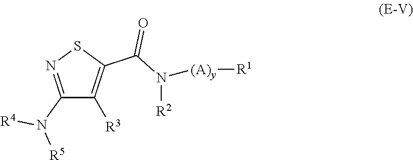
in which R¹, R², R³, R⁴, R⁵, A and y each have the meaning as defined in formula (G), and wherein R³ optionally represents a halogen atom,
with a thionation agent.
